(19) United States
(12) Patent Application Publication
(10) Pub. No.: US 2020/0323991 A1
(43) Pub. Date: Oct. 15, 2020

(54) PBD CONJUGATES FOR TREATING DISEASES

(71) Applicant: ENDOCYTE, INC., West Lafayette, IN (US)

(72) Inventors: Iontcho Radoslavov VLAHOV, West Lafayette, IN (US); Christopher Paul LEAMON, West Lafayette, IN (US); Longwu QI, San Mateo, CA (US); Ning ZOU, West Lafayette, IN (US); Kevin Yu WANG, Zionsville, IN (US); Albert E. FELTEN, Lafayette, IN (US); Garth L. PARHAM, Largo, FL (US); Fei YOU, West Lafayette, IN (US); Hari Krishna R. SANTHAPURAM, West Lafayette, IN (US); Spencer J. HAHN, West Lafayette, IN (US); Joseph Anand REDDY, West Lafayette, IN (US); Yingjuan J. LU, West Lafayette, IN (US); Leroy W. WHEELER, II, West Lafayette, IN (US)

(21) Appl. No.: 16/088,986

(22) PCT Filed: Mar. 29, 2017
(86) PCT No.: PCT/US2017/024770

§ 371 Date: Sep. 27, 2018

Related U.S. Application Data
(60) Provisional application No. 62/396,409, filed on Sep. 19, 2016.
(60) Provisional application No. 62/323,282, filed on Apr. 15, 2016.
(60) Provisional application No. 62/314,688, filed on Mar. 29, 2016.

(51) Int. Cl. A61K 47/55; A61K 31/5517

(52) U.S. Cl. CPC A61K 47/551; A61K 31/5517

(57) ABSTRACT

The present disclosure relates to pyrrolobenzodiazepine (PBD) prodrugs and conjugates thereof. The present disclosure also relates to pharmaceutical compositions of the conjugates described herein, methods of making and methods of using the same.

### CROSS REFERENCE TO RELATED APPLICATIONS

This application claims priority under 35 U.S.C. § 119(e) to U.S. Provisional Application Ser. No. 62/314,688, filed Mar. 29, 2016, U.S. Provisional Application Ser. No. 62/323,282, filed Apr. 15, 2016, and U.S. Provisional Application Ser. No. 62/396,409, filed Sep. 19, 2016, in which all of which are incorporated herein by reference in their entirety.

### TECHNICAL FIELD

The present disclosure relates to pyrrolobenzodiazepine (PBD) prodrugs and conjugates thereof. The present disclosure also relates to pharmaceutical compositions of the conjugates described herein, methods of making and methods of using the same.

### BACKGROUND

The mammalian immune system provides a means for the recognition and elimination of pathogenic cells, such as tumor cells, and other invading foreign pathogens. While the immune system normally provides a strong line of defense, there are many instances where pathogenic cells, such as cancer cells, and other infectious agents evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they lack sufficient selectivity to preferentially destroy pathogenic cells, and therefore, may also harm normal host cells, such as cells of the hematopoietic system, and other non-pathogenic cells. The adverse side effects of these anticancer drugs highlight the need for the development of new therapies selective for pathogenic cell populations and with reduced host toxicity.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diptheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991).

Another approach for targeting populations of pathogenic cells, such as cancer cells or foreign pathogens, in a host is to enhance the host immune response against the pathogenic cells to avoid the need for administration of compounds that may also exhibit independent host toxicity. One reported strategy for immunotherapy is to bind antibodies, for example, genetically engineered multimeric antibodies, to the surface of tumor cells to display the constant region of the antibodies on the cell surface and thereby induce tumor cell killing by various immune-system mediated processes (De Vita, V. T., Biologic Therapy of Cancer, 2d ed. Philadelphia, Lippincott, 1995; Soulillou, J. P., U.S. Pat. No. 5,672,486). However, these approaches have been complicated by the difficulties in defining tumor-specific antigens.

Folate plays important roles in nucleotide biosynthesis and cell division, intracellular activities which occur in both malignant and certain normal cells. The folate receptor has a high affinity for folate, which, upon binding the folate receptor, impacts the cell cycle in dividing cells. As a result, folate receptors have been implicated in a variety of cancers (e.g., ovarian, endometrial, lung and breast) which have been shown to demonstrate high folate receptor expression. In contrast, folate receptor expression in normal tissues is limited (e.g., kidney, liver, intestines and placenta). This differential expression of the folate receptor in neoplastic and normal tissues makes the folate receptor an ideal target for small molecule drug development. The development of folate conjugates represents one avenue for the discovery of new treatments that take advantage of differential expression of the folate receptor. There is a great need for the development of folate conjugates, methods to identify folate receptor positive cancers, and methods to treat patients with folate receptor positive cancers.

### SUMMARY

In one embodiment (referred to herein as embodiment 1), the present disclosure provides a conjugate, or a pharmaceutically acceptable salt thereof, comprising a binding ligand (B), one or more linkers (L), at least one releasable group, a first drug (D¹) and a second drug (D²), wherein B is covalently attached to at least one L, at least one L is covalently attached to at least one of the first drug or the second drug, at least one of the first drug or the second drug is a PBD, and the one or more linkers comprises at least one releasable linker (L^{r}) of the formula

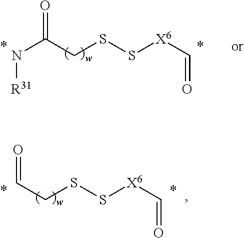

wherein
each R³¹ and R^{31′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³², —OC(O)R³², —OC(O)NR³²R³², —OS(O)R³², —OS(O)₂R³², —SR³², —S(O)R³², —S(O)₂R³², —S(O)NR³²R^{32′}, —S(O)₂NR³²R³², —OS(O)NR³²R³², —OS(O)₂NR³²R³², —NR³²R³², —NR³²C(O)R³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each X⁶ is independently selected from the group consisting of —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— and —C₆-C₁₀ aryl-(C₁-C₆ alkyl)-NR^{31′}—, wherein each hydrogen atom in —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— or —C₆-C₁₀ aryl-(C₁-C₆ alkyl)-NR^{31′} is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}; and
each R³², R^{32′}, R³³, R^{33′}, R³⁴, R^{34′}, R³⁵ and R^{35′} are independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl;
each w is independently an integer from 1 to 4; and
   each * represents a covalent bond to the rest of the conjugate.

In some aspects of embodiment 1, at least one of the first drug or the second drug is a PBD of the formula

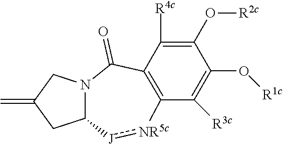

wherein
J is —C(O)—, —CR^{13c}═ or —(CR^{13c}R^{13c})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R^{6c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R^{7c′}, —OS(O)NR^{7c}R^{7c′}, —OS(O)₂NR^{7c}R^{7c′}, —NR^{7c}R^{7c′}, —NR^{7c}C(O)R^{8c}, —NR^{7c}C(O)OR^{8c}, —NR^{7c}C(O)NR^{8c}R^{8c′}, —NR^{7c}S(O)R^{8c′}, —NR^{7c}S(O)₂R^{8c}, —NR^{7c}S(O)NR^{8c}R^{8c′}, —NR^{7c}S(O)₂NR^{8c}R^{8c}, —C(O)R^{7c}, —C(O)OR⁷ or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c}, —OS(O)R^{9c}, —OS(O)₂R^{9c}, —SR^{9c}, —S(O)R^{9c}, —S(O)₂R^{9c}, —S(O)NR^{9c}R^{9c}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{10c}, —NR^{9c}C(O)OR^{10c}, —NR^{9c}C(O)NR^{10c}R^{10c′}, —NR^{9c}S(O)R^{10c′}, —NR^{9c}S(O)₂R^{9c′}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c}.

In some aspects of embodiment 1, each releasable group comprises at least one cleavable bond. In some aspects of embodiment 1, each cleavable bond is broken under physiological conditions. In some aspects of embodiment 1, the conjugate further comprises a releasable group that is not disulfide bond. In some aspects of embodiment 1, the releasable group that is not disulfide bond is a group within the structure of at least one of D¹ or D². In some aspects of embodiment 1, one of D¹ or D² is a PBD pro-drug, and the releasable group is a group within the structure of the PBD pro-drug. In some aspects of embodiment 1, the one or more linkers (L) are independently selected from the group consisting of AA, L¹, L², L³ and L^{r}, and combinations thereof.

In another embodiment (referred to herein as embodiment 2), the present disclosure provides a conjugate, or a pharmaceutically acceptable salt thereof, comprising a binding ligand (B), one or more linkers (L), at least one releasable group, a first drug (D¹) and a second drug (D²), wherein B is covalently attached to at least one L, at least one L is covalently attached to at least one of the first drug or the second drug, and at least one of the first drug or the second drug is a PBD.

In some aspects of embodiment 2, each releasable group comprises at least one cleavable bond. In some aspects of embodiment 2, each cleavable bond is broken under physiological conditions. In some aspects of embodiment 2, the conjugate comprises at least one releasable group that is not disulfide bond. In some aspects of embodiment 2, the releasable group is a group within the structure of at least one of D¹ or D². In some aspects of embodiment 2, one of D¹ or D² is a PBD pro-drug, and the releasable group is a group within the structure of the PBD pro-drug. In some aspects of embodiment 2, at least one releasable group is a disulfide bond. In some aspects of embodiment 2, the one or more linkers (L) are independently selected from the group consisting of AA, L¹, L², L³ and L^{r}, and combinations thereof.

In one aspect, the present disclosure provides conjugates comprising a binding ligand, a linker and a drug, having the formula B-(AA)_{z1}-L²-(L³)_{z2}-(AA)_{z3}-(L¹)_{z4}-(L⁴)_{z5}-D¹-L⁵-D², B-(AA)_{z10}-L²-D², B-(AA)_{z11}-L²-D¹-L⁵-D¹-L²-(AA)_{z12}-B or B-L¹-AA-L¹-AA-L¹-L²-(L³)_{z6}-(L⁴)_{z7}-(AA)_{z8}-(L⁴)_{z9}-D¹-L⁵-D²,

wherein each of B, AA, L¹, L², L³, L⁴, L⁵, D¹, D², z1, z2, z3, z4, z5, z6, z7, z8, z9, z10, z11 and z12 are defined as described herein; or a pharmaceutically acceptable salt thereof.

In another embodiment, the disclosure provides pharmaceutical compositions comprising a therapeutically effective amount of the conjugates described herein, or a pharmaceutically acceptable salt thereof, and at least on excipient.

In another embodiment, the disclosure provides a method of treating abnormal cell growth in a mammal, including a human, the method comprising administering to the mammal a therapeutically effective amount of any of the conjugates or compositions described herein. In some aspects of these embodiments, the abnormal cell growth is cancer. In some aspects of these embodiments, the cancer is folate receptor positive triple negative breast cancer. In some aspects of these embodiments, the cancer is folate receptor negative triple negative breast cancer. In some aspects of these embodiments, the cancer is ovarian cancer. In some aspects of these embodiments, the method further comprises concurrently treatment with anti-CTLA-4 treatment. In some aspects of these embodiments, the method further comprises concurrently treatment with anti-CTLA-4 treatment for the treatment of ovarian cancer.

In another embodiment, the disclosure provides a conjugate, or a pharmaceutically acceptable salt thereof, as described herein for use in a method of treating cancer in a patient.

In some aspects, the method comprises administering to the patient a therapeutically effective amount of any of the conjugates described herein. In some aspects of these embodiments, the cancer is folate receptor positive triple negative breast cancer. In some aspects of these embodiments, the cancer is folate receptor negative triple negative breast cancer. In some aspects of these embodiments, the cancer is ovarian cancer. In some aspects of these embodiments, the method further comprises concurrently treatment with anti-CTLA-4 treatment. In some aspects of these embodiments, the method further comprises concurrently treatment with anti-CTLA-4 treatment for the treatment of ovarian cancer.

The conjugates of the present disclosure can be described as embodiments in any of the following enumerated clauses. It will be understood that any of the embodiments described herein can be used in connection with any other embodiments described herein to the extent that the embodiments do not contradict one another.

1. A conjugate, or a pharmaceutically acceptable salt thereof, comprising a binding ligand (B), one or more linkers (L), at least one releasable group, a first drug (D¹) and a second drug (D²), wherein B is covalently attached to at least one L, at least one L is covalently attached to at least one of the first drug or the second drug, at least one of the first drug or the second drug is a PBD, and the one or more linkers comprises at least one releasable linker (L^{r}) of the formula

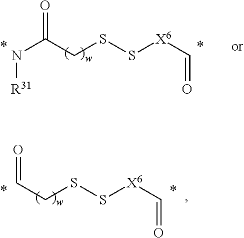

wherein
each R³¹ and R^{31′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′} —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each X⁶ is independently selected from the group consisting of —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— and —C₆-C₁₀ aryl-(C₁-C₆ alkyl)-NR^{31′}—, wherein each hydrogen atom in —C₁-C₆ alkyl-, —C₆-C₁₀ aryl-(C₁-C₆alkyl)-, —C₁-C₆ alkyl-O—, —C₆-C₁₀ aryl-(C₁-C₆ alkyl)-O—, —C₁-C₆ alkyl-NR^{31′}— or —C₆-C₁₀ aryl-(C₁-C₆ alkyl)-NR^{31′} is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}; and
each R³², R^{32′}, R³³, R^{33′}, R³⁴, R^{34′}, R³⁵ and R^{35′} are independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl;
each w is independently an integer from 1 to 4; and
   each * represents a covalent bond to the rest of the conjugate.

2. The conjugate of clause 1, wherein at least one of the first drug or the second drug is a PBD of the formula

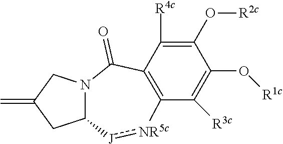

wherein
J is —C(O)—, —CR^{13c}═ or —(CR^{13c}R^{13c′})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R^{6c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R′, —OS(O)NR^{7c}R′, —OS(O)₂NR^{7c}R^{7c′}, —NR^{7c}R^{7c′}, —NR^{7c}C(O)R^{8c}, —NR^{7c}C(O)OR^{8c}, —NR^{7c}C(O)NR^{8e}R^{8c′}, —NR^{7c}S(O)R^{8c}, —NR^{7c}S(O)₂R^{8c}, —NR^{7c}S(O)NR^{8e}R^{8c′} —NR^{7c}S(O)₂NR^{8c}R^{8c′}, —C(O)R^{7c}, —C(O)OR^{7c} or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c}, —OS(O)R^{9c}, —OS(O)₂R^{9c}, —SR^{9c}, —S(O)R^{9c}, —S(O)₂R^{9c}, —S(O)NR^{9c}R^{9c′}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{10c}, —NR^{9c}C(O)OR^{10c}, —NR^{9c}C(O)NR^{10c}R^{10c′}, —NR^{9c}S(O)R^{9c′}, —NR^{9c}S(O)₂R^{10c}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c}.

3. The conjugate of clause 1 or 2, or a pharmaceutically acceptable salt thereof, wherein each releasable group comprises at least one cleavable bond.

4. The conjugate of clause 3, or a pharmaceutically acceptable salt thereof, wherein each cleavable bond is broken under physiological conditions.

5. The conjugate of any one of the preceding clauses, or a pharmaceutically acceptable salt thereof, further comprising a releasable group that is not disulfide bond.

6. The conjugate of clause 5, or a pharmaceutically acceptable salt thereof, wherein the releasable group that is not disulfide bond is a group within the structure of at least one of D¹ or D².

7. The conjugate of any one of the preceding clauses, or a pharmaceutically acceptable salt thereof, wherein one of D¹ or D² is a PBD pro-drug, and the releasable group is a group within the structure of the PBD pro-drug.

8. The conjugate of any one of the preceding clauses, or a pharmaceutically acceptable salt thereof, wherein the one or more linkers (L) are independently selected from the group consisting of AA, L¹, L², L³ and L^{r}, and combinations thereof.

9. The conjugate of any one of the preceding clauses, or a pharmaceutically acceptable salt thereof, wherein B is of the formula

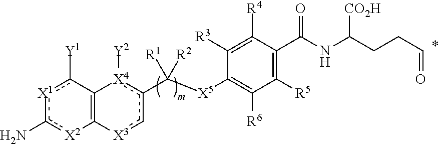

wherein
R¹ and R² in each instance are independently selected from the group consisting of H, halogen, C₁-C₆ alkyl, C₂-C₆ alkenyl, C₂_C₆ alkynyl, —OR⁷, —SR⁷ and —NR⁷R^{7′}, wherein each hydrogen atom in C₁-C₆ alkyl, C₂-C₆ alkenyl and C₂_C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂_C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂_C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂_C₆ alkynyl;
X¹ is —NR¹¹—, ═N—, —N═, —C(R¹¹)═ or ═C(R¹¹)—;
X² is —NR^{11′}— or ═N—;
X³ is —NR^{11″}—, —N═ or —C(R^{11′})═;
X⁴ is —N═ or —C═;
X⁵ is NR¹² or CR¹²R^{12′};
Y¹ is H, —OR¹³, —SR¹³ or —NR¹³R^{13′} when X¹ is —N═ or —C(R¹¹)═, or Y¹ is ═O when X¹ is —NR¹¹—, ═N— or ═C(R¹¹)—;
Y² is H, C₁-C₆ alkyl, C₂-C₆ alkenyl, —C(O)R¹⁴, —C(O)OR¹⁴, —C(O)NR¹⁴R^{14′} when X⁴ is —C═, or Y² is absent when X⁴ is —N═;
R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
   wherein * represents a covalent bond to the rest of the conjugate.

10. The conjugate of any one of the preceding clauses, or a pharmaceutically acceptable salt thereof, wherein the one or more linkers (L) comprises at least one AA selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

11. The conjugate of any one of the preceding clauses, or a pharmaceutically acceptable salt thereof, wherein wherein the one or more linkers (L) comprises at least one AA selected from the group consisting of L-arginine, L-aspartic acid, L-cysteine, D-arginine, D-aspartic acid, and D-cysteine.

12. The conjugate of any one of the preceding clauses, or a pharmaceutically acceptable salt thereof, wherein, when the one or more linkers (L) comprises a first spacer linker (L¹), the first spacer linker is of the formula

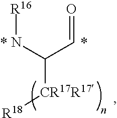

wherein
R¹⁶ is selected from the group consisting of H, C₁-C₆ alkyl, C₂-C₆ alkenyl, C₂_C₆ alkynyl, —C(O)R 9, —C(O)OR¹⁹ and —C(O)NR¹⁹R^{19′}, wherein each hydrogen atom in C₁-C₆ alkyl, C₂-C₆ alkenyl and C₂_C₆ alkynyl is independently optionally substituted by halogen, C₁-C₆ alkyl, C₂-C₆ alkenyl, and C₂_C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂_C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴,
   —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
   each * represents a covalent bond to the rest of the conjugate.

13. The conjugate of any one of the preceding clauses, or a pharmaceutically acceptable salt thereof, wherein when the one or more linkers (L) comprises at least one second spacer linker (L²), each second spacer linker is independently selected from the group consisting of C₁-C₆ alkyl, —OC₁-C₆ alkyl, —SC₁-C₆ alkyl, 3- to 7-membered heterocycloalkyl, C₆-C₁₀ aryl, 5- to 7 membered heteroaryl, —NR³⁶(CR^{36′}R³⁶″)ᵣ—S-(succinimid-1-yl)-, —(CR^{36′}R^{36″})ᵣC(O)NR³⁶—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ—, —C(O)O(CR³⁹R^{39′})ᵣ, —NR³⁹C(O)(CR^{39′}R^{39″})ᵣ, —NR³⁹C(O)(CR^{39′}R^{39″})ᵣS—, (CH₂)ᵣNR³⁹—, —NR³⁹(CH₂)ᵣ—, —NR³⁹(CH₂)ᵣS—, —NR³⁹(CH₂)ᵣNR^{39′}—, —(OCR³⁹R^{39′}CR³⁹R^{39′})ᵣC(O)—, —(OCR³⁹R^{39′}CR³⁹R^{39′}CR³⁹R^{39′})ᵣC(O)—, —OC(O)(CR⁴⁴R^{44′})ₜ—, —C(O)(CR⁴⁴R^{44′})ₜ—, —NR⁴²CR⁴³R^{43′}CR⁴³R^{43′}(OCR⁴⁴R^{44′}CR⁴⁴R^{44′})ₜ—, —CR⁴³R^{43′}CR⁴³R^{43′}(OCR⁴⁴R^{44′}CR⁴⁴R^{44′})ₜNR⁴²—, —NR⁴²C₆-C₁₀ aryl(C₁-C₆ alkyl)OC(O)—, —C(O)CR⁴³R^{43′}R⁴³R^{43′}(OCR⁴⁴R^{44′}CR⁴⁴R^{44′})ₜNR⁴²—, —NR⁴²CR⁴³R′CR⁴³R^{43′}(OCR⁴⁴R^{44′}CR⁴⁴R^{44′})ₜC(O)—, and —NR⁴²CR⁴³R^{43′}CR⁴³R^{43′}(CR⁴⁴═CR^{44′})ₜ—;
wherein
each R³⁶, R^{36′} and R^{36″} is independently selected from the group consisting of H, C₁-C₆ alkyl, C₂-C₆ alkenyl, C₂_C₆ alkynyl, C₃_C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰OR^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR⁴⁰ and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl; and
R⁴² is selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁵, —OC(O)R⁴⁵, —OC(O)NR⁴⁵R^{45′}, —OS(O)R⁴⁵, —OS(O)₂R⁴⁵, —SR⁴⁵, —S(O)R⁴⁵, —S(O)₂R⁴⁵, —S(O)NR⁴⁵R^{45′}, —S(O)₂NR⁴⁵R^{45′}, —OS(O)NR⁴⁵R^{45′}, —OS(O)₂NR⁴⁵R^{45′}, —NR⁴⁵R^{45′}, —NR⁴⁵C(O)R⁴⁶, —NR⁴⁵C(O)OR⁴⁶, —NR⁴⁵C(O)NR⁴⁶R^{46′}, —NR⁴⁵S(O)R⁴⁶, —NR⁴⁵S(O)₂R⁴⁶, —NR⁴⁵S(O)NR⁴⁶R^{46′}, —NR⁴⁵S(O)₂NR⁴⁶R^{46′}, —C(O)R⁴⁵, —C(O)OR⁴⁵ or —C(O)NR⁴⁵R^{45′},
each R⁴³, R^{43′}, R⁴⁴ and R^{44′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁷, —OC(O)R⁴⁷, —OC(O)NR⁴⁷R^{47′}, —OS(O)R⁴⁷, —OS(O)₂R⁴⁷, —SR⁴⁷, —S(O)R⁴⁷, —S(O)₂R⁴⁷, —S(O)NR⁴⁷R⁴⁷, —S(O)₂NR⁴⁷R^{47′}, —OS(O)NR⁴⁷R^{47′}, —OS(O)₂NR⁴⁷R^{47′}, —NR⁴⁷R^{47′}, —NR⁴⁷C(O)R⁴⁸, —NR⁴⁷C(O)OR⁴⁸, —NR⁴⁷C(O)NR⁴⁸R^{48′}, —NR⁴⁷S(O)R⁴⁸, —NR⁴⁷S(O)₂R⁴⁸, —NR⁴⁷S(O)NR⁴⁸R^{48′}, —NR⁴⁷S(O)₂NR⁴⁸R^{48′}, —C(O)R⁴⁷, —C(O)OR⁴⁷ or —C(O)NR⁴⁷R^{47′};
R⁴⁵, R^{45′}, R⁴⁶, R^{46′}, R⁴⁷, R^{47′}, R⁴⁸ and R^{48′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
r in each instance is an integer from 1 to 40; and
t is in each instance is an integer from 1 to 40.

14. The conjugate of any one of the preceding clauses, or a pharmaceutically acceptable salt thereof, wherein when the one or more linkers (L) comprises at least one third spacer linker (L³), each third spacer linker is independently selected from the group consisting of C₁-C₁₀ alkyl, C₂-C₁₀ alkenyl, C₂_C₁₀ alkynyl, —(CR⁴⁹R^{49′})ᵤC(O)—, —CH₂CH₂(OCR⁴⁹R^{49′}CR⁴⁹R^{49′})ᵤ—, —CH₂CH₂CH₂(OCR⁴⁹R^{49′}CR⁴⁹R^{49′}CR⁴⁹R^{49′})ᵤ—, —CH₂CH₂(OCR⁴⁹R^{49′}CR⁴⁹R^{49′})ᵤC(O)— and —CH₂CH₂(OCR⁴⁹R^{49′}CR⁴⁹R^{49′}CR⁴⁹R^{49′})ᵤC(O)—,

wherein
each R⁴⁹ and R^{49′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁵⁰, —OC(O)R⁵⁰, —OC(O)NR⁵⁰R^{50′}, —OS(O)R⁵⁰, —OS(O)₂R⁵⁰, —SR⁵⁰, —S(O)R⁵⁰, —S(O)₂R⁵⁰, —S(O)NR⁵⁰R^{50′}, —S(O)₂NR⁵⁰R^{50′}, —OS(O)NR⁵⁰R^{50′}, —OS(O)₂NR⁵⁰R^{50′}, —NR⁵⁰R^{50′}, —NR⁵⁰C(O)R⁵¹, —NR⁵⁰C(O)OR⁵¹, —NR⁵⁰C(O)NR⁵¹R^{51′}, —NR⁵⁰S(O)R⁵¹, —NR⁵⁰S(O)₂R⁵¹, —NR⁵⁰S(O)NR⁵¹R^{51′}, —NR⁵⁰S(O)₂NR⁵¹R^{51′}, —C(O)R⁵⁰, —C(O)OR⁵⁰ or —C(O)NR⁵⁰R^{50′};
R⁵⁰, R^{50′}, R⁵¹ and R^{51′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
u is in each instance 0, 1, 2, 3, 4 or 5.

15. The conjugate of any one of the preceding clauses, or a pharmaceutically acceptable salt thereof, wherein the first drug is of the formula

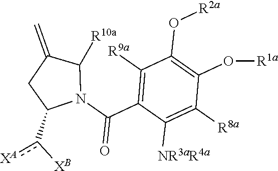

wherein
X^{A} is —OR^{6a}, ═N—OR^{5a} or —NR^{5a}R^{6a}—, provided that when the hash bond is a pi-bond, X^{A} is ═NR^{5a};
X^{B} is H or OR^{7a};
R^{1a}, R^{2a}, R^{3a} and R^{4a} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{11a}, —C(O)OR^{11a}, and —C(O)NR^{11a}R^{11a′} wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11a}, —OC(O)R^{11a}, —OC(O)NR^{11a}R^{11a′}, —OS(O)R^{11a}, —OS(O)₂R^{11a}, —SR^{11a}, —S(O)R^{11a}, —S(O)₂R^{11a}, —S(O)NR^{11a}R^{11a′}, —S(O)₂NR^{11a}R^{11a′}, —OS(O)NR^{11a}R^{11a′}, —OS(O)₂NR^{11a}R^{11a′}, —NR^{11a}R^{11a′}, —NR^{11a}C(O)R^{12a}, —NR^{11a}C(O)OR^{12a}, —NR^{11a}C(O)NR^{12a}R^{12a′}, —NR^{11a}S(O)R^{12a}, —NR^{11a}S(O)₂R^{12a}, —NR^{11a}S(O)NR^{12a}R^{12a′}, —NR^{11a}S(O)₂NR^{12a}R^{12a′}, —C(O)R^{11a}C(O)OR^{11a}, or —C(O)NR^{11a}R^{11a′}; or R^{1a} is a bond; or R^{4a} is a bond;
R^{5a}, R^{6a} and R^{7a} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{13a}, —C(O)OR^{13a} and —C(O)NR^{13a}R^{13a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{14a}, —OC(O)R^{14a}, —OC(O)NR^{14a}R^{14a′}, —OS(O)R^{14a}, —OS(O)₂R^{14a}, —SR^{14a}, —S(O)R^{14a}, —S(O)₂R^{14a}, —S(O)NR^{14a}R^{14a′}, —S(O)₂NR^{14a}R^{14a′}, —OS(O)NR^{14a}R^{14a′}, —OS(O)₂NR^{14a}R^{14a′}, —NR^{14a}R^{14a′}, —NR^{14a}C(O)R^{15a}, —NR^{14a}C(O)OR^{15a}, —NR^{14a}C(O)NR^{15a}R^{15a′}, —NR^{14a}S(O)R^{15a}, —NR^{14a}S(O)₂R^{15a}, —NR^{14a}S(O)NR^{15a}R^{15a′}, —NR^{14a}S(O)₂NR^{15a}R^{15a′}, —C(O)R^{14a}, —C(O)OR^{14a} or —C(O)NR^{14a}R^{14a′}, wherein R^{6a} and R^{7a} taken together with the atoms to which they are attached optionally combine to form a 3- to 7-membered heterocycloalkyl or a 3- to 7-membered heterocycloalkyl fused to a 6-membered aryl ring, or R^{5a} and R^{6a} taken together with the atoms to which they are attached optionally combine to form a 3- to 7-membered heterocycloalkyl or 5- to 7-membered heteroaryl, wherein each hydrogen atom in 3- to 7-membered heterocycloalkyl or 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{16a}, —OC(O)R^{16a}, —OC(O)NR^{16a}R^{16a′}, —OS(O)R^{16a}, —OS(O)₂R^{16a}, —SR^{16a}, —S(O)R^{16a}, —S(O)₂R^{16a}, —S(O)NR^{16a}R^{16a′}, —S(O)₂NR^{16a}R^{16a′}, —OS(O)NR^{16a}R^{16a′}, —OS(O)₂NR^{16a}R^{16a′}, —NR^{16a}R^{16a′}, —NR^{16a}C(O)R^{17a}, —NR^{16a}C(O)CH₂CH₂—, —NR^{16a}C(O)OR^{17a}, —NR^{16a}C(O)NR^{17a}R^{17a′}, —NR^{16a}S(O)R^{17a}, —NR^{16a}S(O)₂R^{17a}, —NR^{16a}S(O)NR^{17a}R^{17a′}, —NR S(O)₂NR^{17a}R^{17a′}, —C(O)R^{16a}, —C(O)OR^{16a} or —C(O)NR^{16a}R^{16a′}, and wherein when R^{5a} and R^{6a} taken together with the atoms to which they are attached form a 5- to 7-membered heteroaryl, one hydrogen atom in 5- to 7-membered heteroaryl is optionally a bond, or when R^{6a} and R^{7a} taken together with the atoms to which they are attached optionally combine to form a 3- to 7-membered heterocycloalkyl fused to a 6-membered aryl, one hydrogen atom in the 6-membered aryl ring is optionally a bond; or R^{sa} is a bond;
R^{8a} and R^{9a} are each independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{18a}, —OC(O)R^{18a}, —OC(O)NR^{18a}R^{18a′}, —OS(O)R^{18a}, —OS(O)₂R^{18a}, —SR^{18a}, —S(O)R^{18a}, —S(O)₂R^{18a}, —S(O)NR^{18a}R^{18a′}, —S(O)₂NR^{18a}R^{18a′}, —OS(O)NR^{18a}R^{18a′}, —OS(O)₂NR^{18a}R^{18a′}, —NR^{18a}R^{18a′}, —NR^{18a}C(O)R^{19a}, —NR^{18a}C(O)OR^{19a}, —NR^{18a}C(O)NR^{19a}R^{19a′}, —NR^{18a}S(O)R^{19a}, —NR^{18a}S(O)₂R^{19a}, —NR^{18a}S(O)NR^{19a}R^{19a′}, —NR^{18a}S(O)₂NR^{19a}R^{19a′}—C(O)R^{18a}, —C(O)OR^{18a} and —C(O)NR^{18a}R^{18a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{20a}, —OC(O)R^{20a}, —OC(O)NR^{20a}R^{20a′}, —OS(O)R^{20a}, —OS(O)₂R^{20a}, —SR^{20a}, —S(O)R^{20a}, —S(O)₂R^{20a}, —S(O)NR^{20a}R^{20a}, —S(O)₂NR^{20a}R^{20a′}, —OS(O)NR^{20a}R^{20a′}, —OS(O)₂NR^{20a}R^{20a′}, —NR^{20a}R^{20a′}, —NR^{20a}C(O)R^{21a}, —NR^{20a}C(O)OR^{21a}, —NR^{20a}C(O)NR^{21a}R^{21a′}, —NR^{20a}S(O)R^{21a}, —NR^{20a}S(O)₂R^{21a}, —NR^{20a}S(O)NR^{21a}R^{21a′}, —NR^{20a}S(O)₂NR^{21a}R^{21a′}—C(O)R^{20a}, —C(O)OR^{20a} or —C(O)NR^{20a}R^{20a′};
R^{10a} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{22a}, —OC(O)R^{22a}, —OC(O)NR^{22a}R^{22a′}, —OS(O)R^{22a}, —OS(O)₂R^{22a}, —SR^{22a}, —S(O)R^{22a}, —S(O)₂R^{22a}, —S(O)NR^{22a}R^{22a′}, —S(O)₂NR^{22a}R^{22a′}, —OS(O)NR^{22a}R^{22a′}, —OS(O)₂NR^{22a}R^{22a′}, —NR^{22a}R^{22a′}, —NR^{22a}C(O)R^{23a}, —NR^{22a}C(O)OR^{23a}, —NR^{22a}C(O)NR^{23a}R^{23a′}, —NR^{22a}S(O)R^{23a}, —NR^{22a}S(O)₂R^{23a}, —NR^{22a}S(O)NR^{23a}R^{23a′}, —NR^{22a}S(O)₂NR^{23a}R^{23a}, —C(O)R^{22a}, —C(O)OR^{23a} and —C(O)NR^{22a}R^{22a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{24a}, —OC(O)R^{24a}, —OC(O)NR^{24a}R^{24a′}, —OS(O)R^{24a}, —OS(O)₂R^{24a}, —SR^{24a}, —S(O)R^{24a}, —S(O)₂R^{24a}, —S(O)NR^{24a}R^{24a′}, —S(O)₂NR^{24a}R^{24a′}, —OS(O)NR^{24a}R^{24a′}—OS(O)₂NR^{24a}R^{24a′}, —NR^{24a}R^{24a′}, —NR^{24a}C(O)R^{25a}, —NR^{24a}C(O)OR^{25a}, —NR^{24a}C(O)NR^{25a}R^{25a′}, —NR^{24a}S(O)R^{25a}, —NR^{24a}S(O)₂R^{25a}, —NR^{24a}S(O)NR^{25a}R^{25a′}, —NR^{24a}S(O)₂NR^{25a}R^{25a′}, —C(O)R^{24a}, —C(O)OR^{24a} or —C(O)NR^{24a}R^{24a′}; and
each R^{11a}, R^{11a′}, R^{12a}, R^{12a′}, R^{13a}, R^{13a′}, R^{14a}, R^{14a′}, R^{15a}, R^{15a′}, R^{16a}, R^{16a′}, R^{17a}, R^{17a′}, R^{18a}, R^{18a′}, R^{19a}, R^{19a′}, R^{20a}, R^{20a′}, R^{21a}, R^{21a′}, R^{22a}, R^{22a′}, R^{23a}, R^{23a′}, R^{24a}, R^{24a′}, R^{25a} and R^{25a′} is independently selected from the group consisting of H, C₁-C₇ alkyl, C₂-C₇ alkenyl, C₂_C₇ alkynyl, C₃-C₁₃ cycloalkyl, 3- to 7-membered heterocycloalkyl, C₆-C₁₀ aryl, and 5- to 7-membered heteroaryl; and
provided that at least two of R, R^{4a}, R^{5a} are a bond; or when R^{5a} and R^{6a} taken together with the atoms to which they are attached optionally combine to form a 5- to 7-membered heteroaryl, one hydrogen atom in 5- to 7-membered heteroaryl is a bond and one of R^{1a} or R^{4a} is a bond.

16. The conjugate of any one of the preceding clauses, or a pharmaceutically acceptable salt thereof, wherein the first drug is covalently attached to the second drug by a third spacer linker (L³).

17. The conjugate of any one of the preceding clauses, or a pharmaceutically acceptable salt thereof, wherein the second drug is selected from the group consisting of

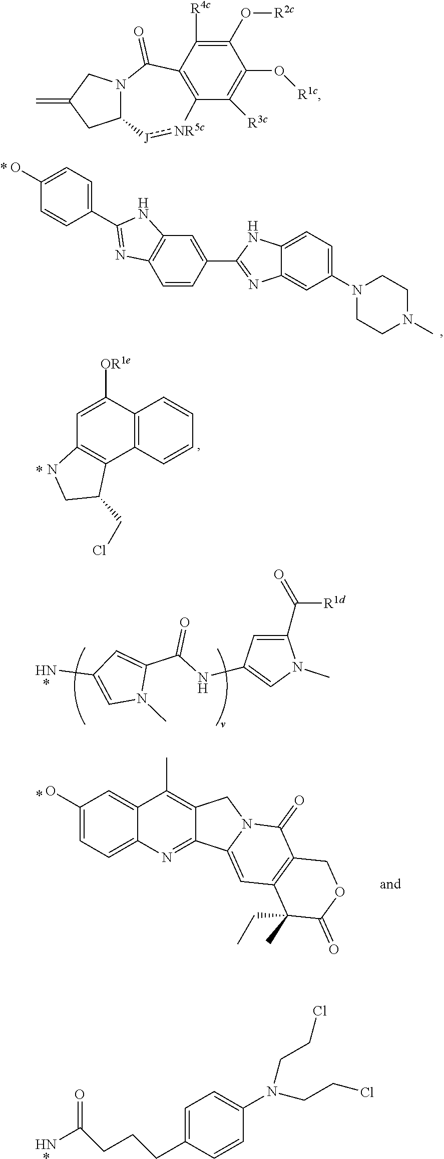

wherein
J is —C(O)—, —CR^{13c}═ or —(CR^{13c}R^{13c})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R⁶, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c′}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R^{7c′}, —OS(O)NR^{7c}R^{7c′}, —OS(O)₂NR^{7c}R^{7c′}, —NR^{7c}R^{7c′}, —NR^{7c}C(O)R^{8c}, —NR^{7c}C(O)OR^{8c}, —NR^{7c}(O)NR^{8c}R^{8c′}, —NR^{7c}S(O)R^{8c′}, —NR^{7c}S(O)₂R^{8c}, —NR^{7c}S(O)NR^{8c}R^{8c′}, —NR^{7c}S(O)₂NR^{8c}R^{8c′}, —C(O)R^{7c}, —C(O)OR^{7c} or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c′}, —OS(O)R^{9c}, —OS(O)₂R^{9c}, —SR^{9c}, —S(O)R^{9c}, —S(O)₂R^{9c}, —S(O)NR^{9c}R^{9c′}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{10c}, —NR^{9c}C(O)OR^{10c}, —NR^{9c}C(O)NR^{10c}R^{10c′}, —NR^{9c}S(O)R^{10c}, —NR^{9c}S(O)₂R^{10c}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c};
R^{1d} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{2d}, —SR^{2d} and —NR^{2d}R^{2d′},
R^{2d} and R^{2d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{3d}, —SR^{3d}, and —NR^{3d}R^{3d′}; —
R^{3d} and R^{3d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
R^{1e} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₆ alkynyl, C₃_C₆ cycloalkyl, 3- to 7-membered heterocycloalkyl, C₆-C₁₀ aryl, 5- to 7-membered heteroaryl, —OR^{2e}, —OC(O)R^{2e}, —OC(O)NR^{2e}R^{2e′}, —OS(O)R^{2e}, —OS(O)₂R^{2e}, —SR^{2e}, —S(O)R^{2e}, —S(O)₂R^{2e}, —S(O)NR^{2e}R^{2e′}, —S(O)₂NR^{2e}R^{2e′}, —OS(O)NR^{2e}R^{2e′}, —OS(O)₂NR^{2e}R^{2e′}, —NR^{2e}R^{2e′}, —NR^{2e}C(O)R^{3e}, —NR^{2e}C(O)OR^{3e}, —NR^{2e}C(O)NR^{3e}R^{3e′}, —NR^{2e}S(O)R^{3e}, —NR^{2e}S(O)₂R^{3e}, —NR^{2e}S(O)NR^{2e}R^{2e′}, —NR^{2e}S(O)₂NR^{3e}R^{3e′}, —C(O)R^{2e}, —C(O)OR^{2e} or —C(O)NR^{2e}R^{2e}.
each R^{2e}, R^{2e′}, R^{3e} and R^{3e′} is independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{4e}, —SR^{4e} or —NR^{4e}R^{4e′};
R^{4e}, and R^{4e′} are independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
v is 1, 2 or 3; and
each * represents a covalent bond to the rest of the conjugate.

18. The conjugate of any one of the preceding clauses, wherein the second drug is of the formula

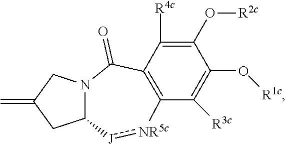

or a pharmaceutically acceptable salt thereof.

19. The conjugate of any one of the preceding clauses, wherein the second drug is of the formula

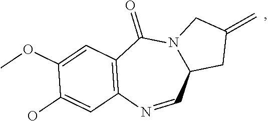

wherein * represents a covalent bond to the rest of the conjugate.

20. The conjugate of clause 8, having the formula B-(L¹)_{z1}-(AA)_{z2}-(L¹)_{z3}-(AA)_{z4}-(L¹)_{z5}-(AA)_{z6}-(L²)_{z7}-(L^{r})_{z8}-(L²)_{z9}-D-L³-D-(L²)_{y9}-(L^{r})_{y8}-(L²)_{y7}-(AA)_{y6}-(L¹)_{y5}-(AA)_{y4}-(L¹)_{y3}- (AA)_{y2}-(L¹)_{y1}-X,

wherein
z1 is an integer from 0 to 2, z2 is an integer from 0 to 3, z3 is an integer from 0 to 2, z4 is an integer from 0 to 3, z5 is an integer from 0 to 2, z6 is an integer from 0 to 3, z7 is an integer from 0 to 8, z8 is 1, z9 is an integer from 0 to 8, y1 is an integer from 0 to 2, y2 is an integer from 0 to 3, y3 is an integer from 0 to 2, y4 is an integer from 0 to 3, y5 is an integer from 0 to 2, y6 is 0 or 1, y7 is an integer from 0 to 8, y8 is 0 or 1; y9 is an integer from 0 to 8;
each D is independently D¹ or D²
X is H or B;
each B is independently a binding ligand;
each AA is independently an amino acid;
each L is independently a first spacer linker;
each L² is independently a second spacer linker;
each L³ is independently a third spacer linker; and
each L^{r} is independently a releasable linker;
   or a pharmaceutically acceptable salt thereof.

21. The conjugate of clause 20, or a pharmaceutically acceptable salt thereof, wherein y1 is 0, y2 is 0, y3 is 0, y4 is 0, y5 is 0, y6 is 0, y7 is 0, y8 is 0, y9 is 0 and X is H.

22. The conjugate of clause 20 or 21, or a pharmaceutically acceptable salt thereof, wherein z1 is 0, z2 is 2, z3 is 0, z4 is 1, z5 is 0 and z6 is 1.

23. The conjugate of clause 20 or 21, or a pharmaceutically acceptable salt thereof, wherein z1 is 0, z2 is 2, z3 is 0, z4 is 2, z5 is 0 and z6 is 1.

24. The conjugate of clause 20 or 21, or a pharmaceutically acceptable salt thereof, wherein z1 is 1, z2 is 1, z3 is 1, z4 is 1, z5 is 1 and z6 is 1.

25. The conjugate of clause 20 or 21, or a pharmaceutically acceptable salt thereof, wherein z1 is 1, z2 is 1, z3 is 1, z4 is 1, z5 is 1 and z6 is 0.

26. The conjugate of clause 20, or a pharmaceutically acceptable salt thereof, wherein z1 is 0, z2 is 2, z3 is 0, z4 is 1, z5 is 0, z6 is 1, y1 is 0, y2 is 2, y3 is 0, y4 is 1, y5 is 0 and y6 is 1.

27. The conjugate of clause 20, or a pharmaceutically acceptable salt thereof, wherein z1 is 0, z2 is 2, z3 is 0, z4 is 2, z5 is 0, z6 is 1, y1 is 0, y2 is 2, y3 is 0, y4 is 2, y5 is 0 and y6 is 1.

28. The conjugate of clause 26 or 27, or a pharmaceutically acceptable salt thereof, wherein y7 is 1.

29. The conjugate of clause 28, or a pharmaceutically acceptable salt thereof, wherein y8 is 0.

30. The conjugate of clause 29, or a pharmaceutically acceptable salt thereof, wherein y9 is 0.

31. The conjugate of clause 26 or 27, or a pharmaceutically acceptable salt thereof, wherein y7 is 0.

32. The conjugate of clause 31, or a pharmaceutically acceptable salt thereof, wherein y8 is 1.

33. The conjugate of clause 32, or a pharmaceutically acceptable salt thereof, wherein y9 is 0.

34. The conjugate of clause 26 or 27, or a pharmaceutically acceptable salt thereof, wherein y8 is 0.

35. The conjugate of any one of clauses 20 to 27, or a pharmaceutically acceptable salt thereof, wherein z7 is 6.

36. The conjugate of any one of clauses 20 to 27, or a pharmaceutically acceptable salt thereof, wherein z7 is 5.

37. The conjugate of any one of clauses 20 to 27, or a pharmaceutically acceptable salt thereof, wherein z7 is 4.

38. The conjugate of any one of clauses 20 to 27, or a pharmaceutically acceptable salt thereof, wherein z7 is 3.

39. The conjugate of any one of clauses 20 to 27, or a pharmaceutically acceptable salt thereof, wherein z7 is 2.

40. The conjugate of any one of clauses 20 to 27, or a pharmaceutically acceptable salt thereof, wherein z7 is 1.

41. The conjugate of any one of clauses 20 to 27, or a pharmaceutically acceptable salt thereof, wherein z7 is 0.

42. The conjugate of any one of clauses 20 to 41, or a pharmaceutically acceptable salt thereof, wherein z9 is 2.

43. The conjugate of any one of clauses 20 to 41, or a pharmaceutically acceptable salt thereof, wherein z9 is 1.

44. The conjugate of any one of clauses 20 to 41, or a pharmaceutically acceptable salt thereof, wherein z9 is 0.

45. The conjugate of any one of the preceding clauses, or a pharmaceutically acceptable salt thereof, wherein B is of the formula

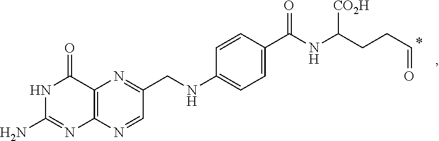 [SMILES: Nc1nc2ncc(CNc3ccc(C(=O)NC(CCC=O)C(=O)O)cc3)nc2c(=O)[nH]1]

or a pharmaceutically acceptable salt thereof.

46. The conjugate of any one of clauses 1 to 22, or a pharmaceutically acceptable salt thereof, comprising the formula

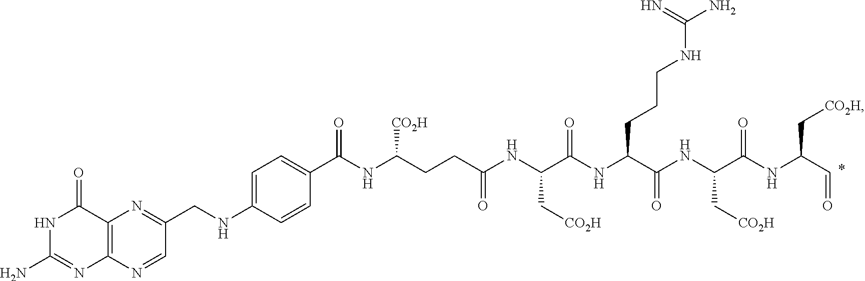 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@H](C=O)CC(=O)O]

wherein * represents a covalent bond to the rest of the conjugate.

47. The conjugate of any one of clauses 1 to 21 or 23, or a pharmaceutically acceptable salt thereof, comprising the formula

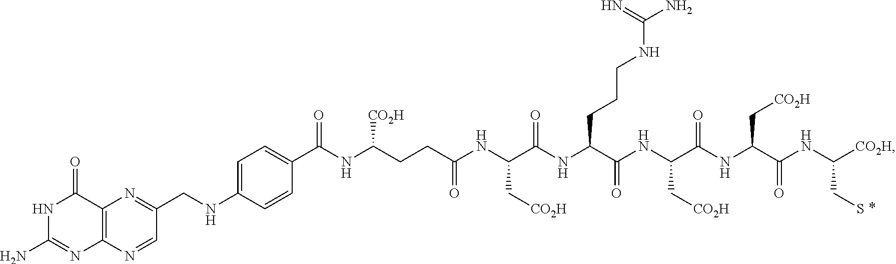 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

wherein * represents a covalent bond to the rest of the conjugate.

48. The conjugate of any one of clauses 1 to 21 or 25, or a pharmaceutically acceptable salt thereof, comprising the formula

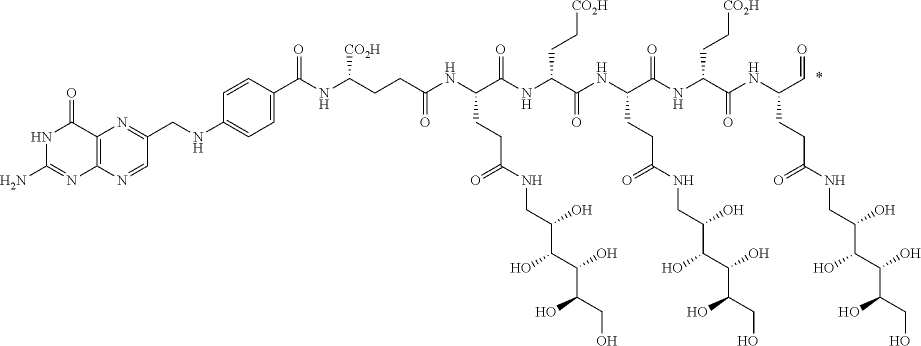 [SMILES: Nc1nc2ncc(CNc3ccc(C(=O)N[C@@H](CCC(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CCC(=O)O)C(=O)N[C@H](C=O)CCC(=O)NC[C@H](O)[C@@H](O)[C@H](O)[C@H](O)CO)C(=O)O)cc3)nc2c(=O)[nH]1]

wherein * represents a covalent bond to the rest of the conjugate.

48. The conjugate of any one of clauses 1 to 21 or 24, or a pharmaceutically acceptable salt thereof, comprising the formula

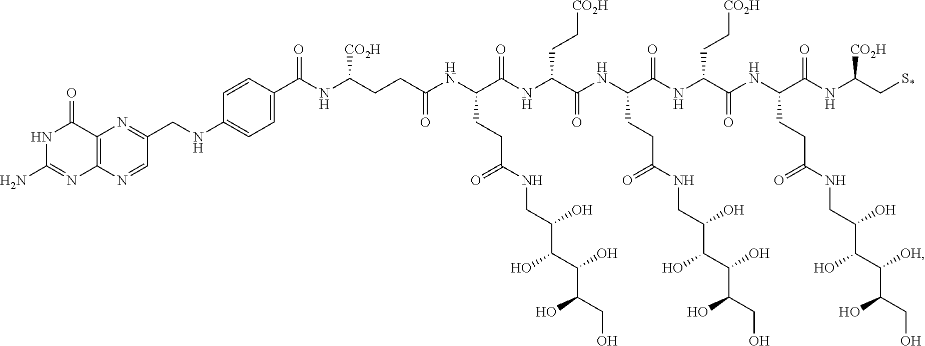

wherein * represents a covalent bond to the rest of the conjugate.

49. The conjugate of any one of the preceding clauses, or a pharmaceutically acceptable salt thereof, comprising the formula

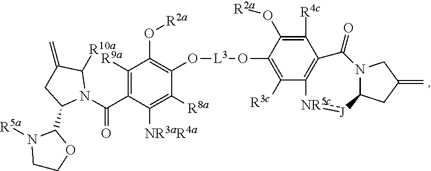

wherein R^{5a} is a covalent bond to the rest of the conjugate.

50. The conjugate of clause 49, any one of the preceding clauses, or a pharmaceutically acceptable salt thereof, comprising the formula

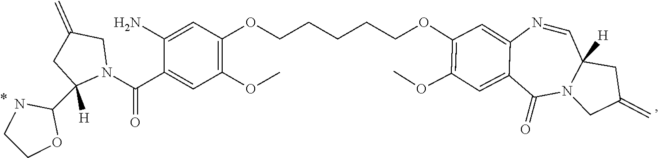

wherein * represents a covalent bond to the rest of the conjugate.

51. The conjugate of any one of clauses 1 to 48, or a pharmaceutically acceptable salt thereof, comprising the formula

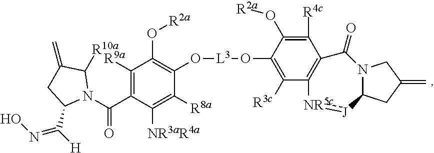

wherein R^{4a} is a covalent bond to the rest of the conjugate.

52. The conjugate of clause 51, or a pharmaceutically acceptable salt thereof, comprising the formula

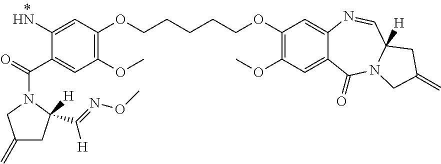

wherein * represents a covalent bond to the rest of the conjugate.

53. The conjugate of any one of clauses 1 to 48, or a pharmaceutically acceptable salt thereof, comprising the formula

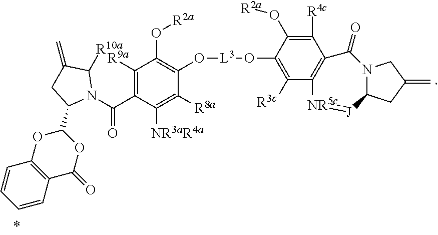

wherein * represents a covalent bond to the rest of the conjugate.

54. The conjugate of clause 53, or a pharmaceutically acceptable salt thereof, comprising the formula

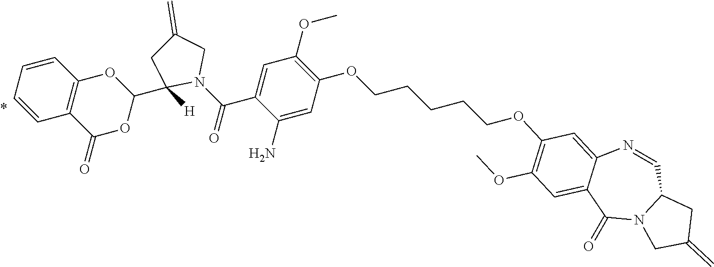 [SMILES: C=C1C[C@H]2C=Nc3cc(OCCCCCOc4cc(N)c(C(=O)N5CC(=C)C[C@H]5C5OC(=O)c6ccccc6O5)cc4OC)c(OC)cc3C(=O)N2C1]

wherein * represents a covalent bond to the rest of the conjugate.

55. The conjugate of any one of clauses 1 to 48, or a pharmaceutically acceptable salt thereof, comprising the formula

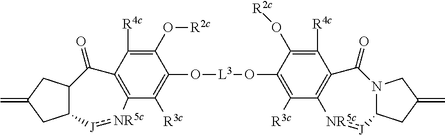

wherein at least one R^{5c} is a covalent bond to the rest of the conjugate.

56. The conjugate of clause 55, or a pharmaceutically acceptable salt thereof, comprising the formula

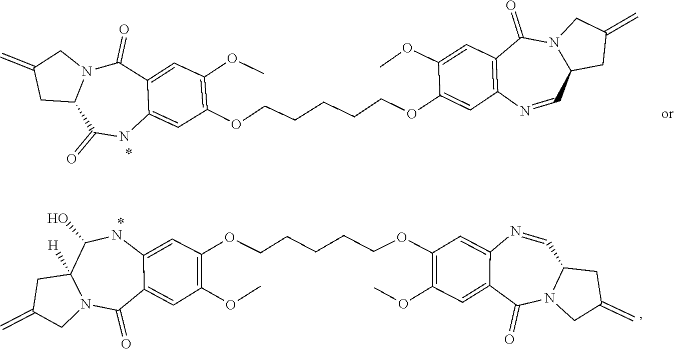

wherein * represents a covalent bond to the rest of the conjugate.

57. The conjugate of clause 55, or a pharmaceutically acceptable salt thereof, comprising the formula

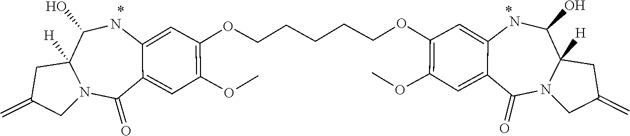

wherein * represents a covalent bond to the rest of the conjugate.

58. A conjugate selected from the group consisting of

or a pharmaceutically acceptable salt thereof.

59. A conjugate selected from the group consisting of

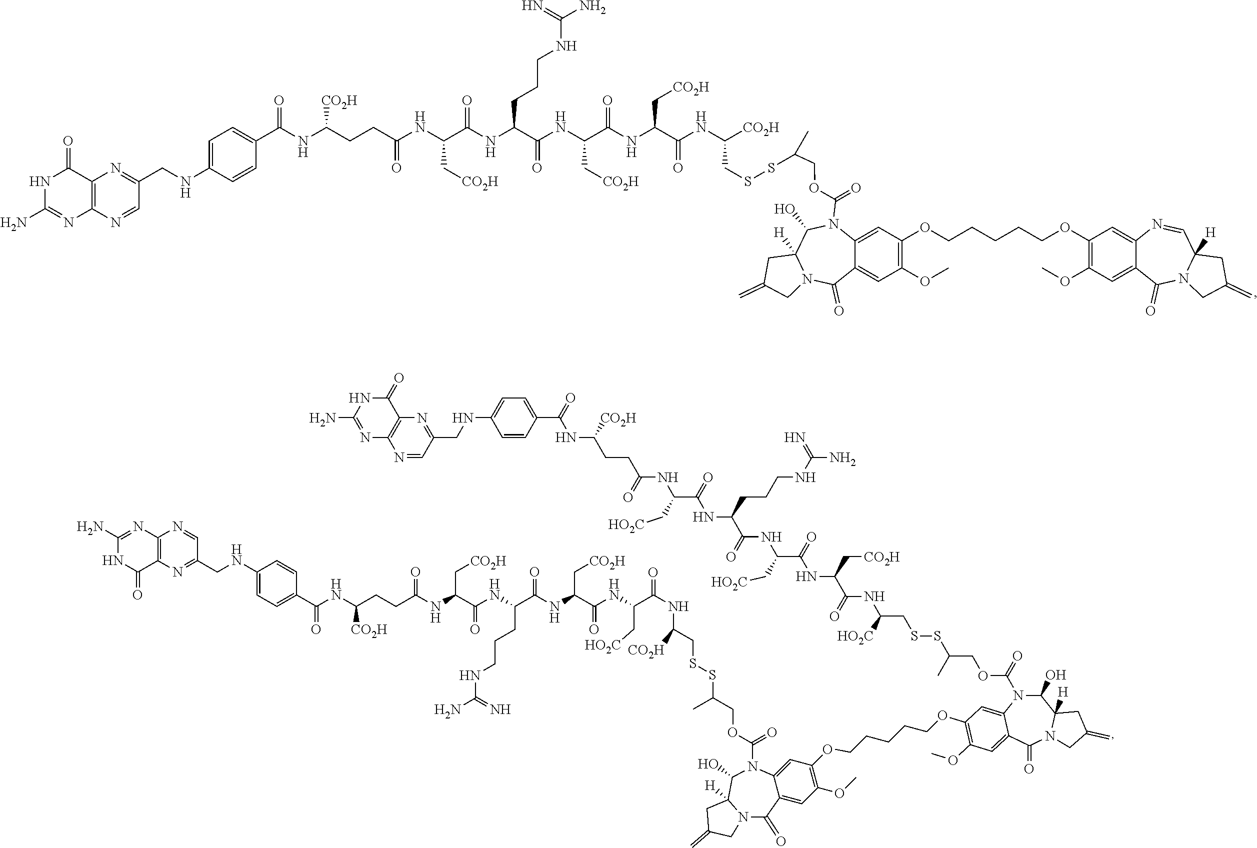

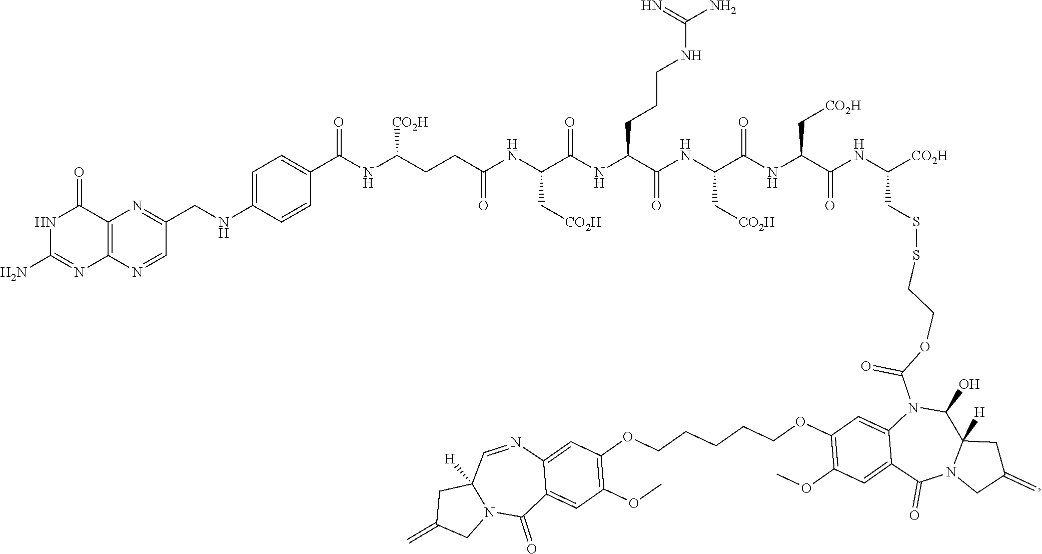 [SMILES: C=C1C[C@H]2C=Nc3cc(OCCCCCOc4cc5c(cc4OC)C(=O)N4CC(=C)C[C@H]4[C@H](O)N5C(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(=O)O)c(OC)cc3C(=O)N2C1]

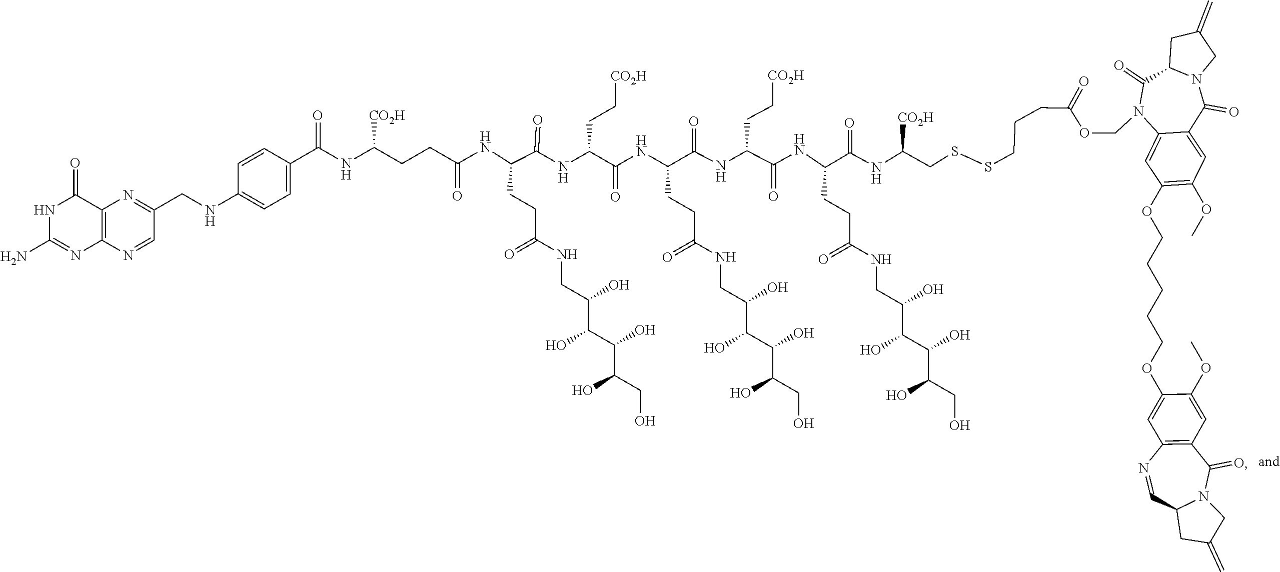 [SMILES: C=C1C[C@H]2C=Nc3cc(OCCCCCOc4cc5c(cc4OC)C(=O)N4CC(=C)C[C@H]4C(=O)N5COC(=O)CCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)c(OC)cc3C(=O)N2C1]

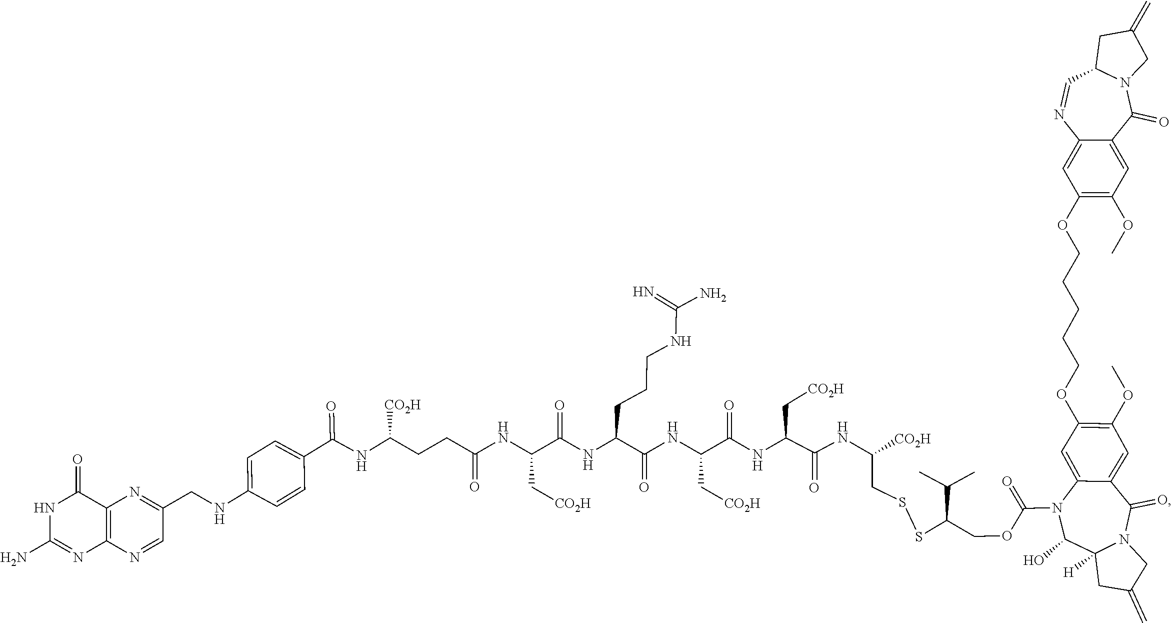 [SMILES: C=C1C[C@H]2C=Nc3cc(OCCCCCOc4cc5c(cc4OC)C(=O)N4CC(=C)C[C@H]4[C@H](O)N5C(=O)OC[C@@H](SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(=O)O)C(C)C)c(OC)cc3C(=O)N2C1]

or a pharmaceutically acceptable salt thereof.

60. A conjugate selected from the group consisting of

or a pharmaceutically acceptable salt thereof.

61. A pharmaceutical composition comprising a therapeutically effective amount of a conjugate according to any one of the preceding clauses, or a pharmaceutically acceptable salt thereof, and optionally at least one pharmaceutically acceptable excipient.

62. A method of treating abnormal cell growth in a patient, comprising

a. administering to the patient a therapeutically effective amount of a conjugate, or a pharmaceutically acceptable salt thereof, or pharmaceutical composition, of any one of the preceding clauses.

63. The method of clause 62, wherein the abnormal cell growth is cancer.

64. The method of clause 63. wherein the cancer is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

65. Use of a conjugate according to any one of clauses 1 to 60 in the preparation of a medicament for the treatment of cancer.

66. A conjugate according to any one of clauses 1 to 60 for use in a method of treating cancer in a patient.

67. The conjugate of clause 66, where the method comprises administering to the patient a therapeutically effective amount of a conjugate, or a pharmaceutically acceptable salt thereof.

68. The conjugate of clause 67, wherein the cancer is selected from the group consisting of lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

### BRIEF DESCRIPTION OF THE DRAWINGS

FIG. 1 is a chart that shows the percentage of ³H-thymidine incorporated into KB cells treated with Conjugate 9 (●) and with Conjugate 9 and excess folate (▪).

FIG. 2A is a chart that shows that Conjugate 9 (▪) dosed at 1 μmol/kg SIW for two weeks decreased KB tumor size in test mice compared to untreated control (●). The dotted line indicates the last dosing day.

FIG. 2B is a chart that shows % weight change for test mice dosed at 1 μmol/kg Conjugate 9 SIW for two weeks (▪) compared to untreated control (●).

FIG. 3 is a chart that shows the percentage of ³H-thymidine incorporated into KB cells treated with Conjugate 1 (●) and with Conjugate 1 and excess folate (▪).

FIG. 4A is a chart that shows that Conjugate 1 dosed at 0.5 μmol/kg SIW for two weeks (●) decreased KB tumor size in test mice compared to untreated control (▴). The dotted line indicates the last dosing day.

FIG. 4B is a chart that shows % weight change for test mice dosed at 0.5 μmol/kg Conjugate 1 SIW for two weeks (●) compared to untreated control (▴).

FIG. 5 is a chart that shows the percentage of ³H-thymidine incorporated into KB cells treated with Conjugate 2 (●) and with Conjugate 2 and excess folate (▪).

FIG. 6A is a chart that shows that Conjugate 2 dosed at 0.5 μmol/kg SIW for two weeks (▪) decreased KB tumor size in test mice compared to untreated control (●). The dotted line indicates the last dosing day.

FIG. 6B is a chart that shows % weight change for test mice dosed at 0.5 μmol/kg Conjugate 2 SIW for two weeks (▪) compared to untreated control (●).

FIG. 7 is a chart that shows the percentage of ³H-thymidine incorporated into KB cells treated with Conjugate 5 (●) and with Conjugate 5 and excess folate (▪).

FIG. 8A is a chart that shows that Conjugate 5 dosed at 0.5 μmol/kg SIW for two weeks (▴) decreased KB tumor size in test mice compared to untreated control (▪). The dotted line indicates the last dosing day.

FIG. 8B is a chart that shows % weight change for test mice dosed at 0.5 μmol/kg Conjugate 5 SIW for two weeks (▴) compared to untreated control (▪).

FIG. 9 is a chart that shows the percentage of ³H-thymidine incorporated into KB cells treated with Conjugate 3 (●) and with Conjugate 3 and excess folate (▪).

FIG. 10A is a chart that shows that Conjugate 3 dosed at 0.5 μmol/kg SIW for two weeks (▾) decreased KB tumor size in test mice compared to untreated control (●). The dotted line indicates the last dosing day.

FIG. 10B is a chart that shows % weight change for test mice dosed at 0.5 μmol/kg Conjugate 3 SIW for two weeks (▾) compared to untreated control (●).

FIG. 11 is a chart that shows the percentage of ³H-thymidine incorporated into KB cells treated with Conjugate 12 (▴) and with Conjugate 12 and excess folate (●).

FIG. 12 is a chart that shows the percentage of ³H-thymidine incorporated into KB cells treated with Conjugate 4 (●) and with Conjugate 4 and excess folate (▪).

FIG. 13A is a chart that shows that each Conjugate 12 dosed at 0.5 μmol/kg SIW for two weeks (▴) and Conjugate 4 dosed at 0.5 μmol/kg SIW for two weeks (♦) decreased KB tumor size in test mice compared to untreated control (●). The dotted line indicates the last dosing day.

FIG. 13B is a chart that shows % weight change for test mice dosed at 0.5 μmol/kg Conjugate 12 SIW for two weeks (▴) and test mice dosed at 0.5 μmol/kg Conjugate 4 SIW for two weeks (♦) compared to untreated control (●).

FIG. 14 is a chart that shows the percentage of ³H-thymidine incorporated into KB cells treated with Conjugate 16 (●) and with Conjugate 16 and excess folate (▪).

FIG. 15A is a chart that shows that Conjugate 16 dosed at 0.5 μmol/kg SIW for two weeks (●) decreased KB tumor size in test mice compared to untreated control (Δ). The dotted line indicates the last dosing day.

FIG. 15B is a chart that shows % weight change for test mice dosed at 0.5 μmol/kg Conjugate 16 SIW for two weeks (●) compared to untreated control (Δ).

FIG. 16 is a chart that shows the percentage of ³H-thymidine incorporated into KB cells treated with Conjugate 6 (●) and with Conjugate 6 and excess folate (▪).

FIG. 17A is a chart that shows that Conjugate 6 dosed at 0.5 μmol/kg SIW for two weeks (▾) decreased KB tumor size in test mice compared to untreated control (●). The dotted line indicates the last dosing day.

FIG. 17B is a chart that shows % weight change for test mice dosed at 0.5 μmol/kg Conjugate 6 SIW for two weeks (▾) compared to untreated control (●).

FIG. 18 is a chart that shows the percentage of ³H-thymidine incorporated into KB cells treated with Conjugate 15 (●) and with Conjugate 15 and excess folate (▪).

FIG. 19A is a chart that shows that Conjugate 15 dosed at 0.5 μmol/kg SIW for two weeks (♦) decreased KB tumor size in test mice compared to untreated control (●). The dotted line indicates the last dosing day.

FIG. 19B is a chart that shows % weight change for test mice dosed at 0.5 μmol/kg Conjugate 15 SIW for two weeks (♦) compared to untreated control (●).

FIG. 20 is a chart that shows the percentage of ³H-thymidine incorporated into KB cells treated with Conjugate 7 (●) and with Conjugate 7 and excess folate (▪).

FIG. 21A is a chart that shows that Conjugate 7 dosed at 0.5 μmol/kg SIW for two weeks (●) decreased KB tumor size in test mice compared to untreated control (●). The dotted line indicates the last dosing day.

FIG. 21B is a chart that shows % weight change for test mice dosed at 0.5 μmol/kg Conjugate 7 SIW for two weeks (●) compared to untreated control (●).

FIG. 22 is a chart that shows the percentage of ³H-thymidine incorporated into KB cells treated with Conjugate 8 (●) and with Conjugate 8 and excess folate (▪).

FIG. 23A is a chart that shows that Conjugate 8 dosed at 0.2 μmol/kg SIW for two weeks (▪) decreased KB tumor size in test mice compared to untreated control (●). The dotted line indicates the last dosing day.

FIG. 23B is a chart that shows % weight change for test mice dosed at 0.2 μmol/kg Conjugate 8 SIW for two weeks (▪) compared to untreated control (●).

FIG. 24 is a chart that shows the percentage of ³H-thymidine incorporated into KB cells treated with Conjugate 18 (●) and with Conjugate 18 and excess folate (▪).

FIG. 25 is a chart that shows the percentage of ³H-thymidine incorporated into KB cells treated with Conjugate 19 (●) and with Conjugate 19 and excess folate (▪).

FIG. 26 is a chart that shows the percentage of ³H-thymidine incorporated into KB cells treated with Conjugate 20 (●) and with Conjugate 20 and excess folate (▪).

FIG. 27A is a chart that shows that each Conjugate 18 dosed at 0.5 μmol/kg SIW for two weeks (▪), Conjugate 19 dosed at 0.5 μmol/kg SIW for two weeks (▴), and Conjugate 20 dosed at 0.5 μmol/kg SIW for two weeks (▾) decreased KB tumor size in test mice compared to untreated control (●). The dotted line indicates the last dosing day.

FIG. 27B is a chart that shows % weight change for test mice dosed at 0.5 μmol/kg Conjugate 18 SIW for two weeks (▪), test mice dosed at 0.5 μmol/kg Conjugate 19 SIW for two weeks (▴), and test mice dosed at 0.5 μmol/kg Conjugate 20 SIW for two weeks (▾) compared to untreated control (●).

FIG. 28 is a chart that shows the relative binding affinity of Conjugate 1 toward the folate receptor. The experiment shows that the relative binding affinity of Conjugate 1 was ˜4.2-fold lower than that of folic acid. (▪) folic acid (Control); (●) Conjugate 1.

FIG. 29 is a graph that shows that intact Conjugate 1 is not able to crosslink DNA while the reduced form (treated with DTT) releases the active PBD molecule, which can then crosslink with DNA. (▪) Conjugate 1 plus DTT; (♦) Conjugate 1 alone.

FIG. 30 is a chart that shows the percentage of ³H-thymidine incorporated into MDA-MB231 cells treated with Conjugate 1 (●) and with Conjugate 1 and excess folate (▪).

FIG. 31 is a chart showing that mice bearing paclitaxel resistant KB tumors dosed at 0.5 μmol/kg SIW for two weeks with Conjugate 5 (▴) had decreased tumor size compared to untreated control (▪). The dotted line indicates the last dosing day. n=5, Conjugate 5 {0,1,4} as {partial response, complete response, cure}.

FIG. 32 is a chart showing that mice bearing platinum resistant KB tumors dosed at 0.5 μmol/kg SIW for two weeks with Conjugate 5 (▪), and dosed at 2.0 μmol/kg BIW for two weeks with EC1456 (▾) had decreased tumor size compared to untreated control (●). The dotted line indicates the last dosing day. n=4, Conjugate 5 {0,0,4}; EC1446 {0,2,2} as {partial response, complete response, cure}.

FIG. 33 is a chart showing that mice bearing ST502 TNBC PDX tumors dosed at 0.3 μmol/kg BIW for two weeks with Conjugate 5 (▴) had decreased tumor size compared to untreated control (▪), while mice dosed at 2.0 μmol/kg BIW for two weeks with EC1456 (●) did not have decreased tumor size compared to untreated control (▪). The dotted line indicates the last dosing day. n=7, Conjugate 5 {0,0,7} as {partial response, complete response, cure}.

FIG. 34 is a chart showing that mice bearing ST070 ovarian PDX tumors dosed at 0.5 μmol/kg SIW for two weeks with Conjugate 5 (●) had decreased tumor size compared to untreated control (▪), while mice dosed at 4.0 μmol/kg SIW for two weeks with EC1456 (▴) or dosed at 15.0 mg/kg SIW for two weeks with paclitaxel (▾) did not have decreased tumor size. The dotted line indicates the last dosing day. n=7, Conjugate 5 {0,0,7} as {partial response, complete response, cure}.

FIG. 35 is a chart that shows the relative binding affinity of Conjugate 5 toward the folate receptor. The experiment shows that the relative binding affinity of Conjugate 5 was ˜1.9-fold lower than that of folic acid. (▪) folic acid (Control); (●) Conjugate 5.

FIG. 36 is a graph that shows that intact Conjugate 5 is not able to crosslink DNA while the reduced form (treated with DTT) releases the active PBD molecule, which can then crosslink with DNA. (●) Conjugate 5 plus DTT; (▪) Conjugate 1 without DTT.

FIG. 37A is a chart that shows that Conjugate 5 dosed at 0.1 μmol/kg SIW for two weeks (▪) and Conjugate 5 dosed at 0.15 μmol/kg SIW for two weeks (▴) decreased KB tumor size in test rats compared to untreated control (●). The dotted line indicates the last dosing day.

FIG. 37B is a chart that shows % weight change for test rats dosed at 0.1 μmol/kg Conjugate 5 SIW for two weeks (▪) and test mice dosed at 0.15 μmol/kg Conjugate 5 SIW for two weeks (▴) compared to untreated control (●).

FIG. 38 is a chart that shows that Conjugate 5 dosed at 0.27 μmol/kg BIW for two weeks (●) decreased TNBC PDX tumor size in test mice compared to untreated control (▪), whereas erubulin mesylate dosed at 1.0 μmol/kg SIW for two weeks (▴) did not decrease TNBC PDX tumor size.

FIG. 39 is a chart that shows that Conjugate 5 dosed at 0.27 μmol/kg BIW for two weeks (●) produced partial response in Endometrial PDX tumor size in test mice compared to untreated control (▪), whereas paclitaxel dosed at 15.0 mg/kg SIW for two weeks (▴) did not produce a partial response.

FIG. 40 is a chart that shows the percentage of ³H-thymidine incorporated into KB cells treated with Conjugate 22 (●) and with Conjugate 20 and excess folate (▪).

FIG. 41 is a chart that shows the percentage of ³H-thymidine incorporated into KB cells treated with Conjugate 24 (●) and with Conjugate 20 and excess folate (▪).

FIG. 42 is a chart that shows the percentage of ³H-thymidine incorporated into KB cells treated with Conjugate 25 (●) and with Conjugate 20 and excess folate (▪).

FIG. 43 is a chart that shows the percentage of ³H-thymidine incorporated into KB cells treated with Conjugate 26 (●) and with Conjugate 20 and excess folate (▪).

FIG. 44 is a chart that shows the percentage of ³H-thymidine incorporated into KB cells treated with Conjugate 27 (●) and with Conjugate 20 and excess folate (▪).

FIG. 45 is a chart that shows the percentage of ³H-thymidine incorporated into KB cells treated with Conjugate 28 (●) and with Conjugate 20 and excess folate (▪).

FIG. 46 is a chart that shows the percentage of ³H-thymidine incorporated into KB cells treated with Conjugate 31 (●) and with Conjugate 20 and excess folate (▪).

FIG. 47 is a chart that shows the percentage of ³H-thymidine incorporated into KB cells treated with Conjugate 32 (●) and with Conjugate 20 and excess folate (▪).

FIG. 48A is a chart that shows that Conjugate 17 dosed at 0.3 μmol/kg SIW (▾) {0,2,3}, decreased KB tumor size in test mice compared to untreated control (●) {0,0,0}.

FIG. 48B is a chart that shows % weight change for test mice dosed at 0.3 μmol/kg Conjugate 17 (▾) compared to untreated control (●).

FIG. 49A is a chart that shows that Conjugate 22 dosed at 0.3 μmol/kg SIW for two weeks (▴) {2,1,2} decreased KB tumor size in test mice compared to untreated control (●) {0,0,0}. The dotted line indicates the last dosing day.

FIG. 49B is a chart that shows % weight change for test mice dosed at 0.3 μmol/kg Conjugate 22 SIW for two weeks (▴) compared to untreated control (●).

FIG. 50A is a chart that shows that Conjugate 24 dosed at 0.3 μmol/kg SIW for two weeks (▪) {0,0,5} decreased KB tumor size in test mice compared to untreated control (●) {0,0,0}. The dotted line indicates the last dosing day.

FIG. 50B is a chart that shows % weight change for test mice dosed at 0.3 μmol/kg Conjugate 24 SIW for two weeks (▪) compared to untreated control (●).

FIG. 51A is a chart that shows that Conjugate 26 dosed at 0.3 μmol/kg SIW for two weeks (▪) {3,0,2} decreased KB tumor size in test mice compared to untreated control (●) {0,0,0}. The dotted line indicates the last dosing day.

FIG. 51B is a chart that shows % weight change for test mice dosed at 0.3 μmol/kg Conjugate 26 SIW for two weeks (▪) compared to untreated control (●).

FIG. 52A is a chart that shows that Conjugate 27 dosed at 0.3 μmol/kg SIW for two weeks (▪) {1,4,} decreased KB tumor size in test mice compared to untreated control (●) {0,0,0}. The dotted line indicates the last dosing day.

FIG. 52B is a chart that shows % weight change for test mice dosed at 0.3 μmol/kg Conjugate 27 SIW for two weeks (▪) compared to untreated control (●).

FIG. 53A is a chart that shows that Conjugate 28 dosed at 0.3 μmol/kg SIW for two weeks (▴) {0,0,5} decreased KB tumor size in test mice compared to untreated control (●) {0,0,0}. The dotted line indicates the last dosing day.

FIG. 53B is a chart that shows % weight change for test mice dosed at 0.3 μmol/kg Conjugate 28 SIW for two weeks (▴) compared to untreated control (●).

FIG. 54A is a chart that shows that Conjugate 30 dosed at 0.3 μmol/kg SIW for two weeks (▪) {0,0,3} decreased KB tumor size in test mice compared to untreated control (●) {0,0,0}. The dotted line indicates the last dosing day.

FIG. 54B is a chart that shows % weight change for test mice dosed at 0.3 μmol/kg Conjugate 30 SIW for two weeks (▪) compared to untreated control (●).

FIG. 55A is a chart that shows that Conjugate 32 dosed at 0.3 μmol/kg SIW for two weeks (◯) {0,5,0} decreased KB tumor size in test mice compared to untreated control (●) {0,0,0}. The dotted line indicates the last dosing day.

FIG. 55B is a chart that shows % weight change for test mice dosed at 0.3 μmol/kg Conjugate 32 SIW for two weeks (◯) compared to untreated control (●).

FIG. 56 is a chart showing a potent dose-dependent inhibition of cell proliferation with relative IC₅₀ values of ˜0.52 (72 h), 0.61 (96 h), and 0.17 (120 h) in ID8-CI15 ovarian cancer cells treated with Conjugate 5.

FIG. 57 is a graph showing that Conjugate 5 demonstrated a potent activity at all concentrations tested (1 nM, 10 nM and 100 nM) after a 2 h exposure and 9-day chase. The anti-tumor activity of Conjugate 5 was significantly reduced in the presence of excess amount of folic acid at both 1 nM and 10 nM concentrations.

FIG. 58 is a graph showing functional FR levels were measured on the IGROV1 human ovarian cancer cells: (a) hHLA+CD45− ascites cancer cells [FR+=6.04%; (b) ascites F480+CD11+ macs [FR+=52.6%]; (c) IGROV cell line control [FR+=98.5%].

FIG. 59A is chart showing the presence of CD4+ and CD8+ T cells quantitated in total peritoneal cells of the immunocompetent C57BL6 mice at 7 day intervals post IP injection of the mouse ovarian cell line, ID8-CL15 (FIG. 59A). The CD45+CD3e+CD8+CD4− T cells (▪) slowly increased in number from day 7 to day 42 post implantation. The CD45+CD3e+CD4+CD8− T cells (▴) also increased in number from day 7 to day 35.

FIG. 59B is a chart showing CD45− non bone-marrow derived ascites cells from ID8-CL15 implanted mice expressed very little functional FR (see FIG. 59B (▪)), whereas ascites macrophages expressed a significant amount of a functional FR (see FIG. 59B (●)).

FIG. 59C is a graph showing ascites macrophages expressed a significant amount of a functional FR.

FIG. 60A is a chart that shows that Conjugate 5 dosed at 100 nmol/kg BIW, 6 doses, first dose at day 7 (▴) increased survival time in test mice compared to untreated control (●) and anti-CTLA-5 alone dosed at 250 μg/dose BIW, 5 doses, and comparable to a significantly higher dose of comparator compound EC1456 (▾) 2000 nmol/kg BIW, 6 doses, first dose at day 7. FIG. 60A also shows that Conjugate 5 dosed with anti-CTLA-5, initiated at day 11, (◯) increased survival time in test mice compared to all other test animals. The dotted line indicates the last dosing day.

FIG. 60B is a chart that shows % weight change for test mice dosed with Conjugate 5 (▴), Conjugate 5+ anti-CTLA-5 (▪), EC1456 (▾) and anti-CTLA-5 (◯) compared to untreated control (●).

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, 0-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₅, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₅, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1,2,3, 4-tetrahydropyridinyl, and the like.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or ““hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″— group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″— group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″— group, where R″ is any R group as described in the various embodiments provided herein.

As used herein, “nitro” refers to a —NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts which counter ions which may be used in pharmaceuticals. Such salts include:
(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.
   Pharmaceutically acceptable salts are well known to those skilled in the art, and any such pharmaceutically acceptable salt may be contemplated in connection with the embodiments described herein

As used herein, “amino acid” (a.k.a. “AA”) means any molecule that includes an alpha-carbon atom covalently bonded to an amino group and an acid group. The acid group may include a carboxyl group. “Amino acid” may include molecules having one of the formulas:

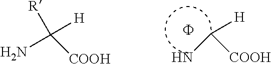

wherein R′ is a side group and Φ includes at least 3 carbon atoms. “Amino acid” includes stereoisomers such as the D-amino acid and L-amino acid forms. Illustrative amino acid groups include, but are not limited to, the twenty endogenous human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, phosphoethanolamine (PEA), sarcosine (SARC), taurine (TAU), carnosine (CARN), citrulline (CIT), anserine (ANS), 1,3-methyl-histidine (ME-HIS), alpha-amino-adipic acid (AAA), beta-alanine (BALA), ethanolamine (ETN), gamma-amino-butyric acid (GABA), beta-amino-isobutyric acid (BAIA), alpha-amino-butyric acid (BABA), L-allo-cystathionine (cystathionine-A; CYSTA-A), L-cystathionine (cystathionine-B; CYSTA-B), cystine, allo-isoleucine (ALLO-ILE), DL-hydroxylysine (hydroxylysine (I)), DL-allo-hydroxylysine (hydroxylysine (2)), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), D-carnosine (D-CARN), and the like. In connection with the embodiments described herein, amino acids can be covalently attached to other portions of the conjugates described herein through their alpha-amino and carboxy functional groups (i.e. in a peptide bond configuration), or through their side chain functional groups (such as the side chain carboxy group in glutamic acid) and either their alpha-amino or carboxy functional groups. It will be understood that amino acids, when used in connection with the conjugates described herein, may exist as zwitterions in a conjugate in which they are incorporated.

As used herein, “sugar” refers to carbohydrates, such as monosaccharides, disaccharides, or oligosaccharides. In connection with the present disclosure, monosaccharides are preferred. Non-limiting examples of sugars include erythrose, threose, ribose, arabinose, xylose, lyxose, allose, altrose, glucose, mannose, galactose, ribulose, fructose, sorbose, tagatose, and the like. It will be understood that as used in connection with the present disclosure, sugar includes cyclic isomers of amino sugars, deoxy sugars, acidic sugars, and combinations thereof. Non-limiting examples of such sugars include, galactosamine, glucosamine, deoxyribose, fucose, rhamnose, glucuronic acid, ascorbic acid, and the like. In some embodiments, sugars for use in connection with the present disclosure include

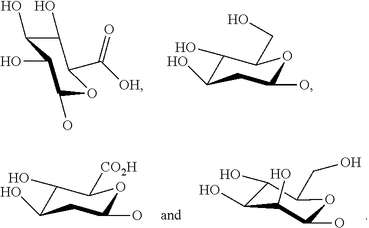

As used herein, “prodrug” refers to a compound that can be administered to a subject in a pharmacologically inactive form which then can be converted to a pharmacologically active form through a normal metabolic process, such as hydrolysis of an oxazolidine. It will be understood that the metabolic processes through which a prodrug can be converted to an active drug include, but are not limited to, one or more spontaneous chemical reaction(s), enzyme-catalyzed chemical reaction(s), and/or other metabolic chemical reaction(s), or a combination thereof. It will be appreciated that understood that a variety of metabolic processes are known in the art, and the metabolic processes through which the prodrugs described herein are converted to active drugs are non-limiting. A prodrug can be a precursor chemical compound of a drug that has a therapeutic effect on a subject.

As used herein, the term “releasable group” refers to a bond or bonds that can be broken (“a cleavable bond” or “cleavable bonds”) under physiological conditions, such as a pH-labile, acid-labile, base-labile, oxidatively labile, metabolically labile, biochemically labile, or enzyme-labile bond. It will be appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It will be appreciated that a releasable group can connect two adjacent atoms within a releasable linker and/or connect other linkers (e.g. AA, L¹, L², L³, etc), B and/or D, as described herein. Alternatively, a releasable group can form part of a drug or a prodrug, D, and/or connect a drug or pro-drug, D, to other linkers (e.g. AA, L¹, L², L³, etc), B and/or D, as described herein. In the case where a releasable group connects two adjacent atoms within a releasable linker, following breakage of the cleavable bond, such releasable linker is broken into two or more fragments. Alternatively, in the case where a releaseable group connects a linker (e.g.

AA, L¹, L², L³, etc) to another moiety, such as another linker, a drug or binding ligand, then such releasable linker becomes separated from such other moiety following breaking of the cleavable bond or cleavable bonds. Alternatively, in the case where a releaseable group is within a drug or prodrug, D, that is connected to a linker, another drug or a binding ligand, then following breaking of the cleavable bond or cleavable bonds, such linker, drug or binding ligand becomes separated from such drug or prodrug having the releaseable group within.

The lability of the releasable group can be adjusted by, for example, substituents at or near the cleavable bond, such as including alpha-branching adjacent to a cleavable disulfide bond, increasing the hydrophobicity of substituents on silicon in a moiety having silicon-oxygen bond that may be hydrolyzed, homologating alkoxy groups that form part of a ketal or acetal that may be hydrolyzed, and the like.

As used herein, the term “therapeutically effective amount” refers to an amount of a drug or pharmaceutical agent that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In one aspect, the therapeutically effective amount is that amount of an active which may treat or alleviate the disease or symptoms of the disease at a reasonable benefit/risk ratio applicable to any medical treatment. In another aspect, the therapeutically effective amount is that amount of an inactive prodrug which when converted through normal metabolic processes to produce an amount of active drug capable of eliciting the biological or medicinal response in a subject that is being sought.

It is also appreciated that the dose, whether referring to monotherapy or combination therapy, is advantageously selected with reference to any toxicity, or other undesirable side effect, that might occur during administration of one or more of the conjugates described herein. Further, it is appreciated that the co-therapies described herein may allow for the administration of lower doses of conjugates that show such toxicity, or other undesirable side effect, where those lower doses are below thresholds of toxicity or lower in the therapeutic window than would otherwise be administered in the absence of a cotherapy.

As used herein, “administering” includes all means of introducing the conjugates and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The conjugates and compositions described herein may be administered in unit dosage forms and/or formulations containing conventional nontoxic pharmaceutically-acceptable carriers, adjuvants, and/or vehicles.

As used herein “pharmaceutical composition” or “composition” refers to a mixture of one or more of the conjugates described herein, or pharmaceutically acceptable salts, solvates, hydrates thereof, with other chemical components, such as pharmaceutically acceptable excipients. The purpose of a pharmaceutical composition is to facilitate administration of a conjugate to a subject. Pharmaceutical compositions suitable for the delivery of conjugates described and methods for their preparation will be readily apparent to those skilled in the art. Such compositions and methods for their preparation may be found, for example, in ‘Remington's Pharmaceutical Sciences’, 19th Edition (Mack Publishing Company, 1995).

A “pharmaceutically acceptable excipient” refers to an inert substance added to a pharmaceutical composition to further facilitate administration of a conjugate such as a diluent or a carrier.

### DETAILED DESCRIPTION

In each of the foregoing and each of the following embodiments, it is to be understood that the formulae include and represent not only all pharmaceutically acceptable salts of the conjugates, but also include any and all hydrates and/or solvates of the conjugate formulae. It is appreciated that certain functional groups, such as the hydroxy, amino, and like groups form complexes and/or coordination conjugates with water and/or various solvents, in the various physical forms of the conjugates. Accordingly, the above formulae are to be understood to include and represent those various hydrates and/or solvates. It is also to be understood that the non-hydrates and/or non-solvates of the conjugate formulae are described by such formula, as well as the hydrates and/or solvates of the conjugate formulae.

The conjugates described herein can be expressed by the generalized descriptors B, L and D, where B is a cell surface receptor binding ligand (a.k.a. a “binding ligand”), L is a linker that may include a releasable group, L can be described by one or more of the linker groups AA, L¹, L², L³, or L^{r} as defined herein, and D represents one or more drugs (D¹ and D²). In the embodiments described herein, it will be appreciated that B is covalently attached to a linker (L) that comprises one or more (for example from 1 to 20) linker from one or more linker groups AA, L¹, L², L³, or L^{r}, which linker (L) is covalently attached to one or more drugs (D¹ or D²), and when the conjugate contains two drugs D¹ or D², the drugs D¹ and D² can be covalently attached to one another by one or more of AA, L¹, L² and L³, provided that one of D¹ or D² in the conjugate is a PBD drug.

The conjugates described herein in connection with embodiment 1 can be described by various general structures including but not limited to B-(L¹)_{z1}-(AA)_{z2}-(L¹)_{z3}-(AA)_{z4}-(L¹)_{z5}-(AA)_{z6}-(L²)_{z7}-(L^{r})_{z8}-(L²)_{z9}-D-L³-D-(L²)_{y9}-(L^{r})_{y8}-(L²)_{y7}-(AA)_{y6}-(L¹)_{y5}-(AA)_{y4}-(L¹)_{y3}- (AA)_{y2}-(L¹)_{y1}-X, wherein z1 is an integer from 0 to 2, z2 is an integer from 0 to 3, z3 is an integer from 0 to 2, z4 is an integer from 0 to 3, z5 is an integer from 0 to 2, z6 is an integer from 0 to 3, z7 is an integer from 0 to 8, z8 is 1, z9 is an integer from 0 to 8, y1 is an integer from 0 to 2, y2 is an integer from 0 to 3, y3 is an integer from 0 to 2, y4 is an integer from 0 to 3, y5 is an integer from 0 to 2, y6 is 0 or 1, y7 is an integer from 0 to 8, y8 is 0 or 1; y9 is an integer from 0 to 8; each D¹ is independently D¹ or D²; X is H or B; each B is independently a binding ligand; each AA is independently an amino acid; each L is independently a first spacer linker; each L² is independently a second spacer linker; each L³ is independently a third spacer linker; and each L^{r} is independently a releasable linker. The conjugates described herein can also be described by any of the formulae

B-(AA)_{z2}-(L²)_{z7}-L^{r}-D¹-L³-D²,

B-(AA)₄-(L²)₄-L^{r}-D¹-L³-D²,

B-(AA)₄-(L²)₅-L^{r}-D¹-L³-D²,

B-(AA)₅-(L²)₄-L^{r}-D¹-L³-D²,

B-(AA)₅-(L²)₅-L^{r}-D¹-L³-D²,

B-(AA)₄-L^{r}-D¹-L³-D²,

B-(L¹)_{z1}-(AA)_{z2}-(L¹)_{z3}-(AA)_{z4}-(L¹)_{z5}-(L²)_{z7}-L^{r}-D¹-L³-D²,

B-(L¹)_{z1}-(AA)_{z2}-(L¹)_{z3}-(AA)_{z4}-(L¹)_{z5}-(AA)_{z6}-(L²)_{z7}-L^{r}-D¹-L³-D²,

B-L¹-AA-L¹-AA-L¹-(L²)_{z7}-L^{r}-D¹-L³-D²,

B-L¹-AA-L¹-AA-L¹-AA-(L²)_{z7}-L^{r}-D¹-L³-D²,

B-L¹-AA-L¹-AA-L¹-(L²)₄-L^{r}-D¹-L³-D²,

B-L¹-AA-L¹-AA-L¹-(L²)₅-L^{r}-D¹-L³-D²,

B-L¹-AA-L¹-AA-L¹-AA-(L²)₃-L^{r}-D¹-L³-D²,

B-(AA)_{z2}-(L²)_{z7}-(L^{r})_{z8}-D-L³-D-L^{r}-(L²)_{y7}-(AA)_{y2}-B,

B-L¹-AA-L¹-AA-L¹-(L²)_{z7}-(L^{r})_{z8}-D-L³-D-L^{r}-(L²)_{y7}-L¹-AA-L¹-AA-L¹-B and

B-L¹-AA-L¹-AA-L¹-AA-(L²)_{z7}-(L^{r})_{z8}-D-L³-D-L^{r}-(L²)_{y7}-AA-L¹-AA-L¹-AA-L¹-B,

wherein B, AA, L¹, L², L³, L^{r}, D¹, D² z1, z2, z3, z4, z5, z6, z7 and y7 are as defined herein.

The conjugates described herein in connection with embodiment 2 can be described by various general structures including but not limited to B-(L¹)_{z1}-(AA)_{z2}-(L¹)_{z3}-(AA)_{z4}-(L¹)_{z5}-(AA)_{z6}-(L²)_{z7}-(L^{r})_{z8}-(L²)_{z9}-D-L³-D-(L²)_{y9}-(L^{r})_{y8}-(L²)_{y7}-(AA)_{y6}-(L¹)_{y5}-(AA)_{y4}-(L¹)_{y3}- (AA)_{y2}-(L¹)_{y1}-X, wherein z1 is an integer from 0 to 2, z2 is an integer from 0 to 3, z3 is an integer from 0 to 2, z4 is an integer from 0 to 3, z5 is an integer from 0 to 2, z6 is an integer from 0 to 3, z7 is an integer from 0 to 8, z8 is 0 or 1, z9 is an integer from 0 to 8, y1 is an integer from 0 to 2, y2 is an integer from 0 to 3, y3 is an integer from 0 to 2, y4 is an integer from 0 to 3, y5 is an integer from 0 to 2, y6 is 0 or 1, y7 is an integer from 0 to 8, y8 is 0 or 1; y9 is an integer from 0 to 8; each D is independently D¹ or D²; X is H or B; each B is independently a binding ligand; each AA is independently an amino acid; each L is independently a first spacer linker; each L² is independently a second spacer linker; each L³ is independently a third spacer linker; and each L^{r} is independently a releasable linker. The conjugates described herein can also be described by any of the formulae

B-(AA)_{z2}-(AA)_{z4}-(L²)_{z7}-L^{r}-D¹-L³-D²,

B-(AA)_{z2}-(AA)_{z4}-(L²)_{z7}-D¹-L³-D²,

B-(AA)₄-(L²)₄-L^{r}-D¹-L³-D²,

B-(AA)₄-(L²)₅-L^{r}-D¹-L³-D²,

B-(AA)₄-(L²)₇-D¹-L³-D²,

B-(AA)₄-(L²)₆-D¹-L³-D²,

B-(AA)₅-(L²)₄-L^{r}-D¹-L³-D²,

B-(AA)₅-(L²)₅-L^{r}-D¹-L³-D²,

B-(AA)₅-(L²)₇-D¹-L³-D²,

B-(AA)₅-(L²)₆-D¹-L³-D²,

B-(AA)₄-L^{r}-D¹-L³-D²,

B-(AA)₅-L²-D¹-L³-D²,

B-(L¹)_{z1}-(AA)_{z2}-(L¹)_{z3}-(AA)_{z4}-(L¹)_{z5}-(L²)_{z7}-(L^{r})_{z8}-D¹-L³-D²,

B-(L¹)_{z1}-(AA)_{z2}-(L¹)_{z3}-(AA)_{z4}-(L¹)_{z5}-(AA)_{z6}-(L²)_{z7}-(L^{r})_{z8}-D¹-L³-D²,

B-L¹-AA-L¹-AA-L¹-(L²)_{z7}-(L^{r})_{z8}-D¹-L³-D²,

B-L¹-AA-L¹-AA-L¹-AA-(L²)_{z7}-(L^{r})_{z8}-D¹-L³-D²,

B-L¹-AA-L¹-AA-L¹-(L²)₄-L^{r}-D¹-L³-D²,

B-L¹-AA-L¹-AA-L¹-(L²)₅-L^{r}-D¹-L³-D²,

B-L¹-AA-L¹-AA-L¹-(L²)₆-D¹-L³-D²,

B-L¹-AA-L¹-AA-L¹-(L²)₇-D¹-L³-D²,

B-L¹-AA-L¹-AA-L¹-AA-(L²)₃-L^{r}-D¹-L³-D²,

B-L¹-AA-L¹-AA-L¹-AA-(L²)₅-D¹-L³-D²,

B-(AA)_{z2}-(AA)_{z4}-(L²)_{z7}-(L^{r})_{z8}-D-L³-D-(L^{r})_{y8}-(L²)_{y7}-(AA)_{y2}-B,

B-L¹-AA-L¹-AA-L¹-(L²)_{z7}-(L^{r})_{z8}-D-L³-D-(L^{r})_{y8}-(L²)_{y7}-L¹-AA-L¹-AA-L¹-B,

B-L¹-AA-L¹-AA-L¹-AA-(L²)_{z7}-(L^{r})_{z8}-D-L³-D-(L^{r})_{y8}-(L²)_{y7}-AA-L¹-AA-L¹-AA-L¹-B,

B-L¹-AA-L¹-AA-L¹-(L²)_{z7}-D-L³-D-(L²)_{y7}-L¹-AA-L¹-AA-L¹-B,

B-L¹-AA-L¹-AA-L¹-AA-(L²)_{z7}-D-L³-D-(L²)_{y7}-AA-L¹-AA-L¹-AA-L¹-B,

wherein B, AA, L¹, L², L³, L^{r}, D¹, D² z1, z2, z3, z4, z5, z6, z7, z8, y7 and y8 are as defined herein.

The Binding Ligand

It will be appreciated that any of the descriptions of binding ligands provided herein can be used independently in connection with either embodiment 1 or embodiment 2. Specifically, neither embodiment 1 nor embodiment 2 requires any particular restriction on the identity of the binding ligand.

As used herein, the term cell surface receptor binding ligand (aka a “binding ligand”), generally refers to compounds that bind to and/or target receptors that are found on cell surfaces, and in particular those that are found on, over-expressed by, and/or preferentially expressed on the surface of pathogenic cells. Illustrative ligands include, but are not limited to, vitamins and vitamin receptor binding compounds.

Illustrative vitamin moieties include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity covalently attachment to the linker. Illustrative biotin analogs that bind to biotin receptors include, but are not limited to, biocytin, biotin sulfoxide, oxybiotin, and the like).

In some embodiments, the B is folate or derivative thereof. In some embodiments, the B is of the formula I

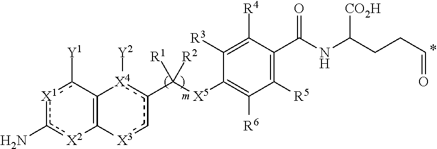

wherein
R¹ and R² in each instance are independently selected from the group consisting of H, halogen, C₁-C₆ alkyl, C₂-C₆ alkenyl, C₂_C₆ alkynyl, —OR⁷, —SR⁷ and —NR⁷R^{7′}, wherein each hydrogen atom in C₁-C₆ alkyl, C₂-C₆ alkenyl and C₂_C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂_C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂_C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂_C₆ alkynyl;
X¹ is —NR¹¹—, ═N—, —N═, —C(R¹¹)═ or ═C(R¹¹)—;
X² is —NR^{11′}— or ═N—;
X³ is —NR^{11″}—, —N═ or —C(R^{11′})═;
X⁴ is —N═ or —C═;
X⁵ is NR¹² or CR¹²R^{12′};
Y¹ is H, —OR¹³, —SR¹³ or —NR¹³R^{13′} when X¹ is —N═ or —C(R¹¹)═, or Y¹ is ═O when X¹ is —NR¹¹—, ═N— or ═C(R¹¹)—;
Y² is H, C₁-C₆ alkyl, C₂-C₆ alkenyl, —C(O)R¹⁴, —C(O)OR¹⁴, —C(O)NR¹⁴R^{14′} when X⁴ is —C═, or Y² is absent when X⁴ is —N═;
R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};
R¹⁵ and R^{15′} are each independently H or C₁-C₆ alkyl;
m is 1, 2, 3 or 4; and
* is a covalent bond to the rest of the conjugate.

It will be appreciate that when B is described according to the formula I, that both the D- and L- forms are contemplated. In some embodiments, B is of the formula Ia or Ib

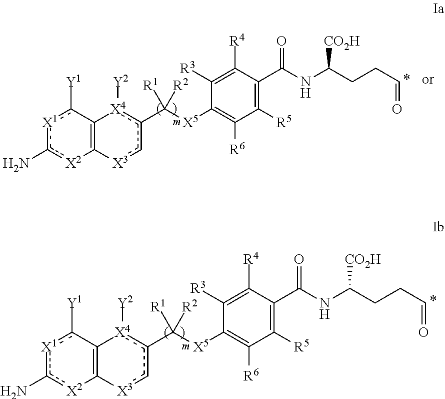

where each of R¹, R², R³, R⁴, R⁵, R⁶, Y¹, Y², X¹, X², X³, X⁴, X⁵, m and * are as defined for the formula I.

In some embodiments described herein, R¹ and R² are H. In some embodiments described herein, m is 1. In some embodiments described herein, R³ is H. In some embodiments described herein, R⁴ is H. In some embodiments described herein, R⁵ is H. In some embodiments described herein, R⁶ is H. In some embodiments described herein, R³, R⁴, R⁵ and R⁶ are H. In some embodiments described herein, X¹ is —NR¹¹, and R¹¹ is H. In some embodiments described herein, X² is ═N—. In some embodiments described herein, X³ is —N═. In some embodiments described herein, X⁴ is —N═. In some embodiments described herein, X is —NR¹¹, and R¹¹ is H; X² is ═N—; X³ is —N═; and X⁴ is —N═. In some embodiments described herein, X⁵ is NR¹², and R¹² is H. In some embodiments, Y¹ is ═O. In some embodiments, Y² is absent. In some embodiments, B is of the formula Ic

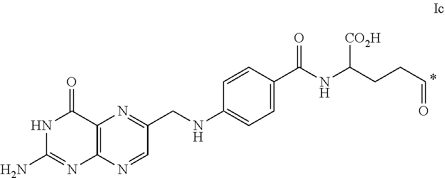 [SMILES: Nc1nc2ncc(CNc3ccc(C(=O)NC(CCC=O)C(=O)O)cc3)nc2c(=O)[nH]1]

wherein * is defined for formula I.

In some embodiments, B is of the formula Id

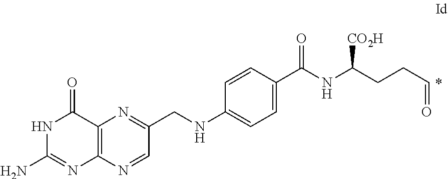 [SMILES: Nc1nc2ncc(CNc3ccc(C(=O)N[C@H](CCC=O)C(=O)O)cc3)nc2c(=O)[nH]1]

wherein * is defined for formula I.

It will be appreciated that in certain embodiments, the conjugates described herein can be represented by the exemplary formulae

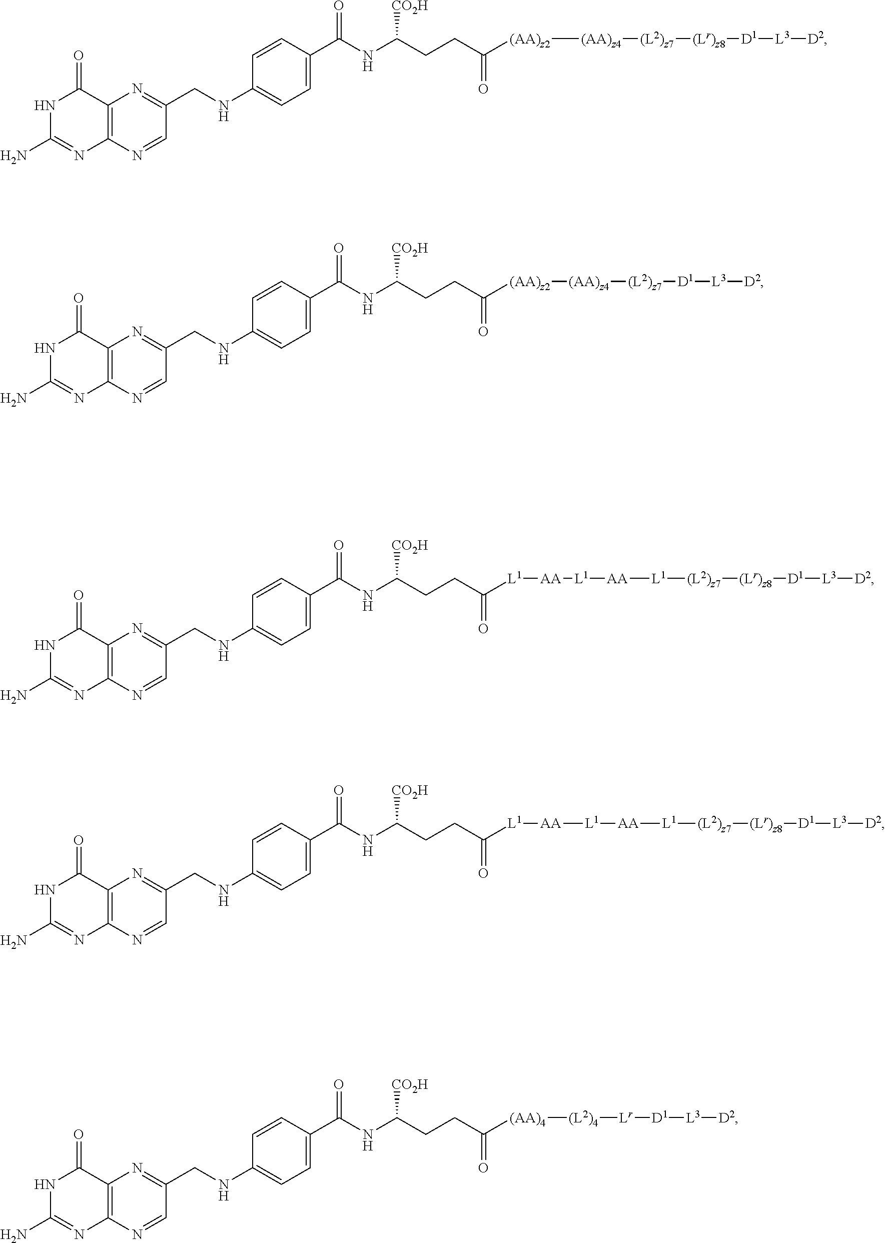

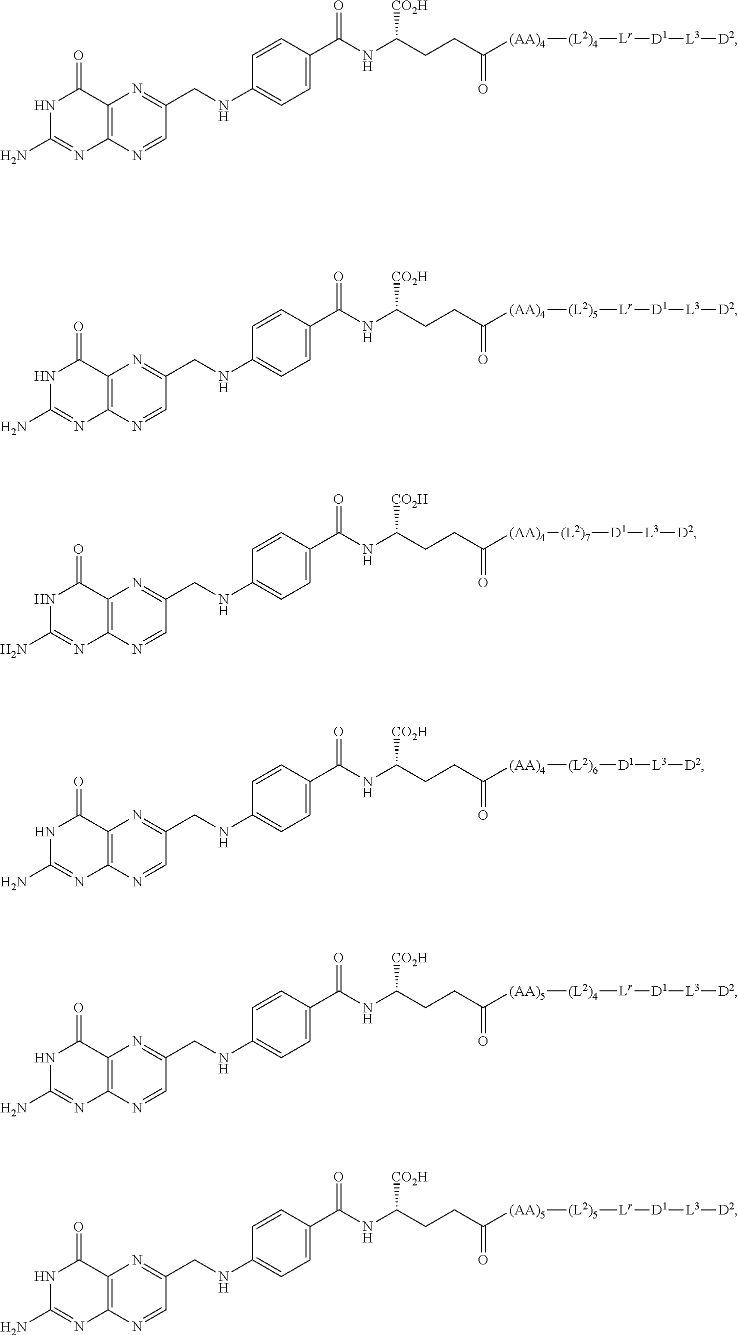

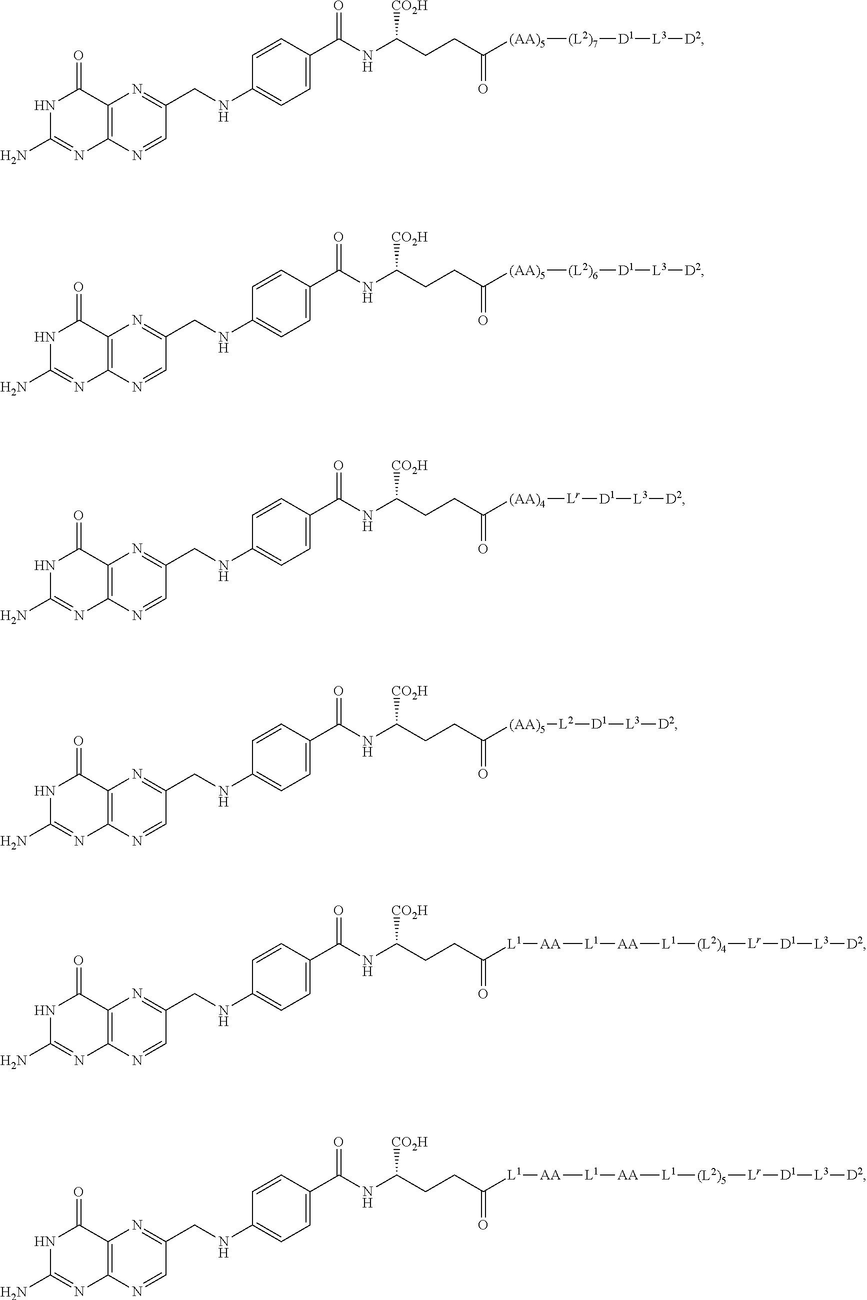

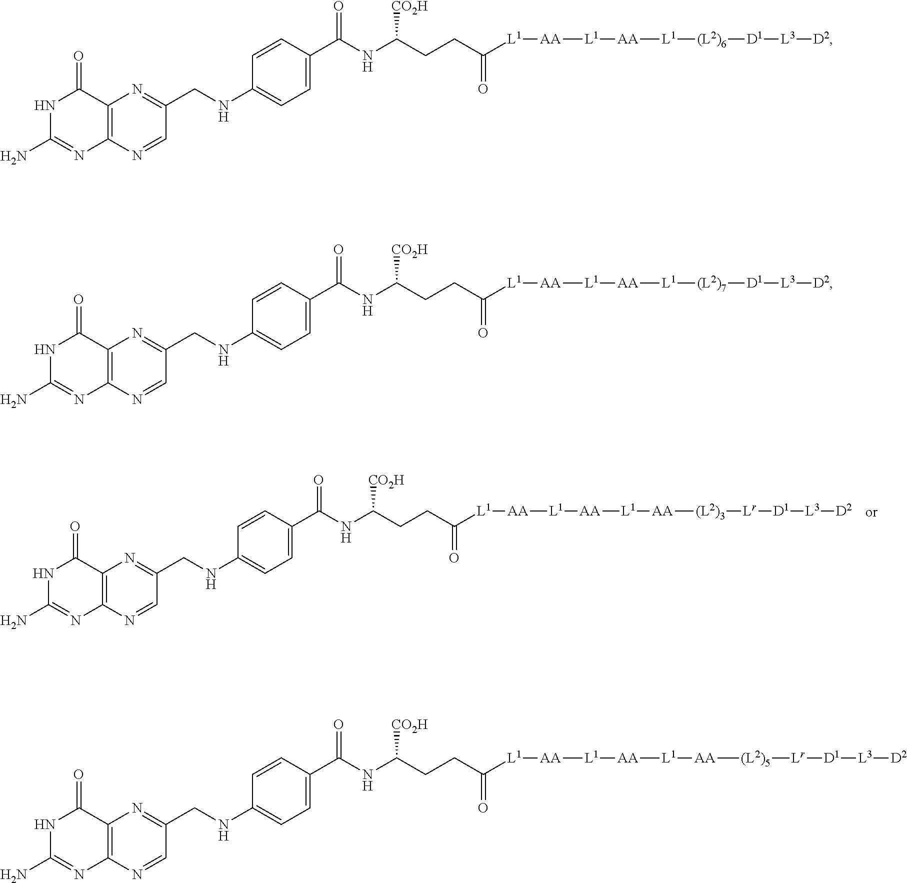

or a pharmaceutically acceptable salt thereof.

The Linker (L)

The linker (L) for connecting B, D¹ and or D², in the conjugates described herein can be any linker as defined herein comprising one or more of groups AA, L¹, L², L³, and/or L^{r}.

It will be appreciated that any of the descriptions of linkers AA, L¹, L² and L³ provided herein can be used independently in connection with either embodiment 1 or embodiment 2. Specifically, neither embodiment 1 nor embodiment 2 requires any particular restriction on the identity of the binding ligand. With respect to the linker L^{r}, it will be appreciated that at least one L^{r} of the formula

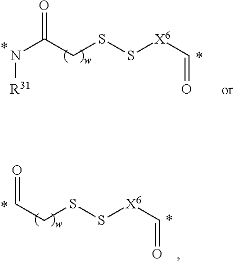

is included in the conjugates described by embodiment 1. However, it will be appreciated that independent of embodiment 1, any of the linkers described herein can be present or not present in conjugates described within embodiment 2. Specifically, embodiment 2 places no particular restriction on the identity of L^{r}.

AA is an amino acid as defined herein. In certain embodiments, AA is a naturally occurring amino acid. In certain embodiments, AA is in the L-form. In certain embodiments, AA is in the D-form. It will be appreciated that in certain embodiments, the conjugates described herein will comprise more than one amino acid as portions of the linker, and the amino acids can be the same or different, and can be selected from a group of amino acids. It will be appreciated that in certain embodiments, the conjugates described herein will comprise more than one amino acid as portions of the linker, and the amino acids can be the same or different, and can be selected from a group of amino acids in D- or L-form. In some embodiments, each AA is independently selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

In some embodiments, each AA is independently selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline. In some embodiments, each AA is independently selected from the group consisting of Asp, Arg, Glu and Cys. In some embodiments, z2 is 2, z4 is 2, and the sequence of AAs is -Asp-Arg-Asp-Asp-. In some embodiments, z2 is 2, z4 is 2, and z6 is 1, and the sequence of AAs is -Asp-Arg-Asp-Asp-Cys. In some embodiments, z2 is 2, z4 is 3, and the sequence of AAs is -Asp-Arg-Asp-Asp-Cys.

L¹ can be present or absent in the conjugates described herein. When L is present, L can be any group covalently attaching portions of the linker to the binding ligand, portions of the linker to one another, or to D¹, or to D². It will be understood that the structure of L is not particularly limited in any way. It will be further understood that L can comprise numerous functionalities well known in the art to covalently attach portions of the linker to the binding ligand, portions of the linker to one another, or to D¹, or to D², including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L is a linker of the formula II

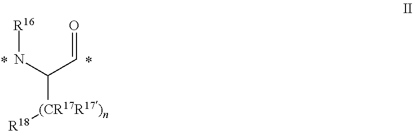

wherein
R¹⁶ is selected from the group consisting of H, D, C₁-C₆ alkyl, C₂-C₆ alkenyl, C₂_C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂_C₆ alkynyl is independently optionally substituted by halogen, C₁-C₆ alkyl, C₂-C₆ alkenyl, and C₂_C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, D, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂_C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, D, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26′})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰, and R^{30′} is independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
* is a covalent bond.

It will be appreciated that when L is described according to the formula II, that both the R- and S- configurations are contemplated. In some embodiments, L is of the formula IIa or IIb

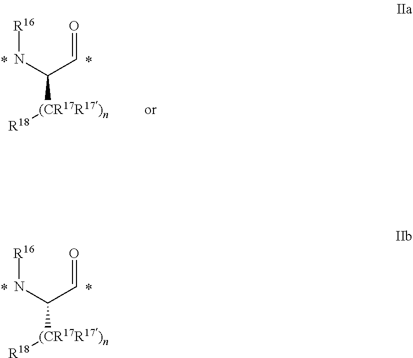

where each of R¹⁶, R¹⁷, R^{17′}, R¹⁸, n and * are as defined for the formula II.

In some embodiments, each L is selected from the group consisting of

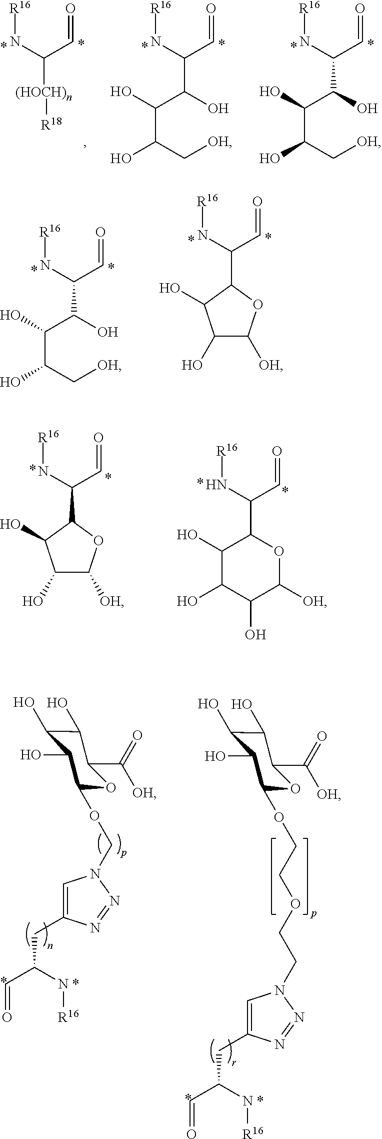

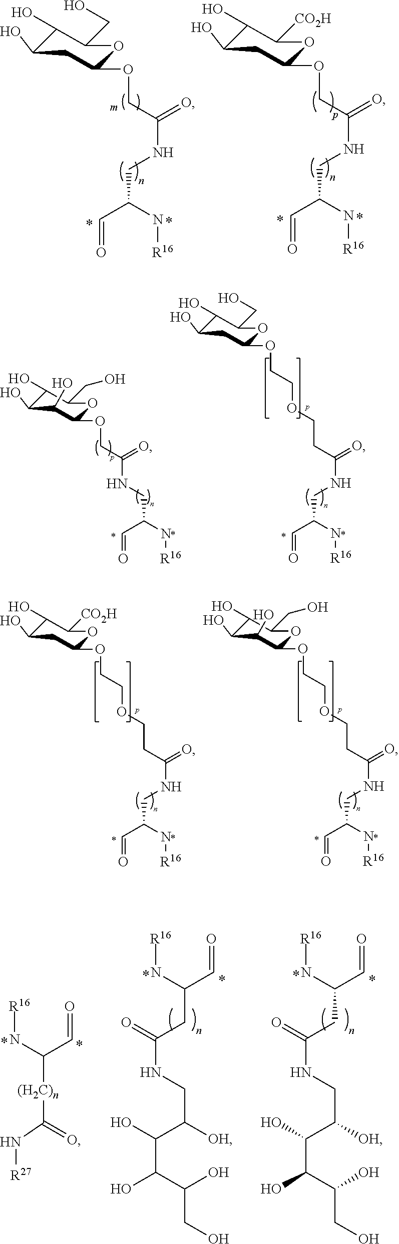

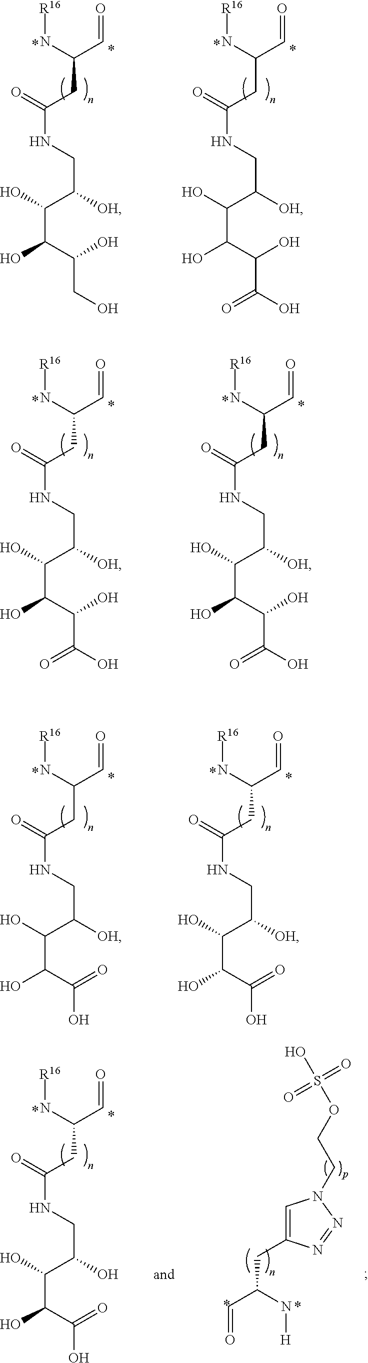

and combinations thereof,
wherein
R¹⁶ is selected from the group consisting of H, D, C₁-C₆ alkyl, C₂-C₆ alkenyl, C₂_C₆ alkynyl, —C(O)R 9, —C(O)OR¹⁹ and —C(O)NR¹⁹R^{19′}, wherein each hydrogen atom in C₁-C₆ alkyl, C₂-C₆ alkenyl and C₂_C₆ alkynyl is independently optionally substituted by halogen, C₁-C₆ alkyl, C₂-C₆ alkenyl, and C₂_C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R²¹, —NR²⁰S(O)₂NR²¹R^{21′}, —C(O)R²⁰, —C(O)OR²⁰ or —C(O)NR²⁰R^{20′};
R¹⁸ is selected from the group consisting of H, D, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26′})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each each, R¹⁹, R^{19′}, R²⁰, R^{20′}, R²¹, R^{21′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments, each L¹ is selected from the group consisting of

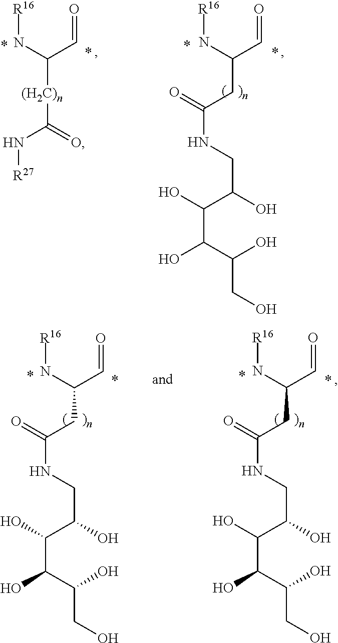

wherein R¹⁶ is defined as described herein, and each * represent a covalent bond to the rest of the conjugate.

In some embodiments, R¹⁶ is H. In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —R²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹;
each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;
R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);
R²⁸ is H or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments, each L is selected from the group consisting of

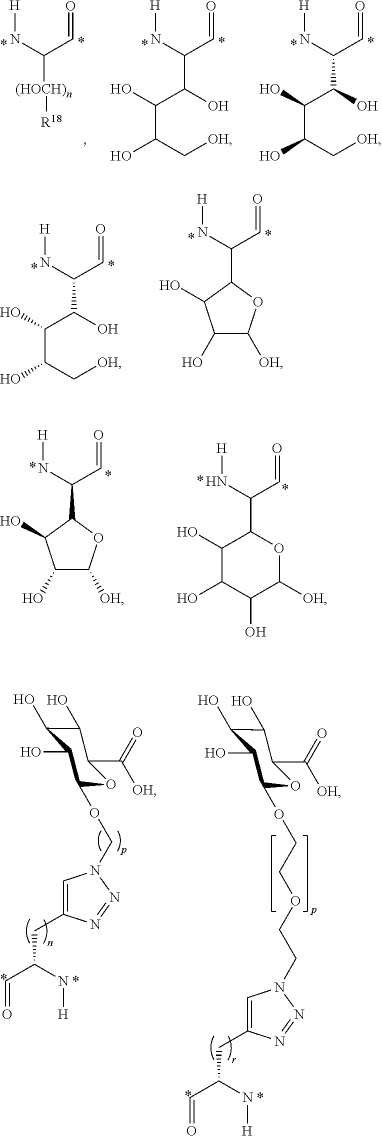

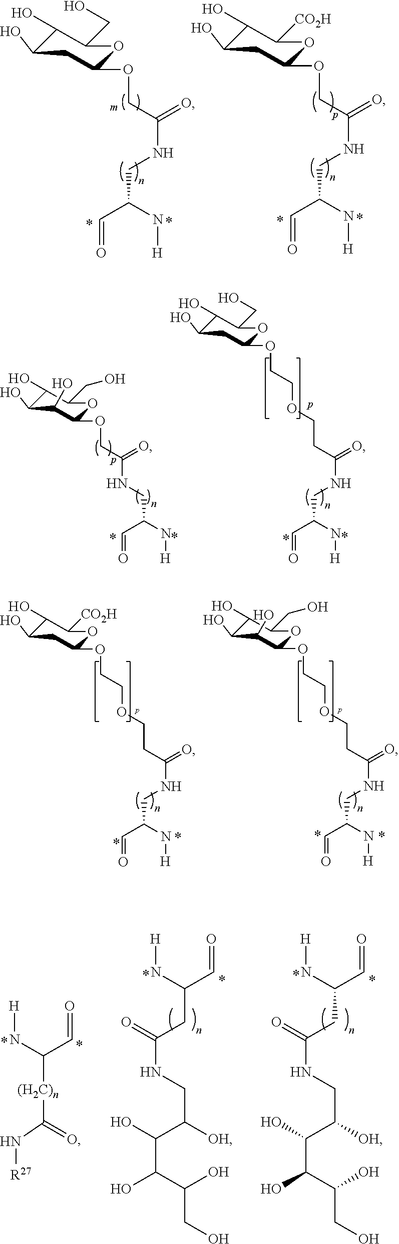

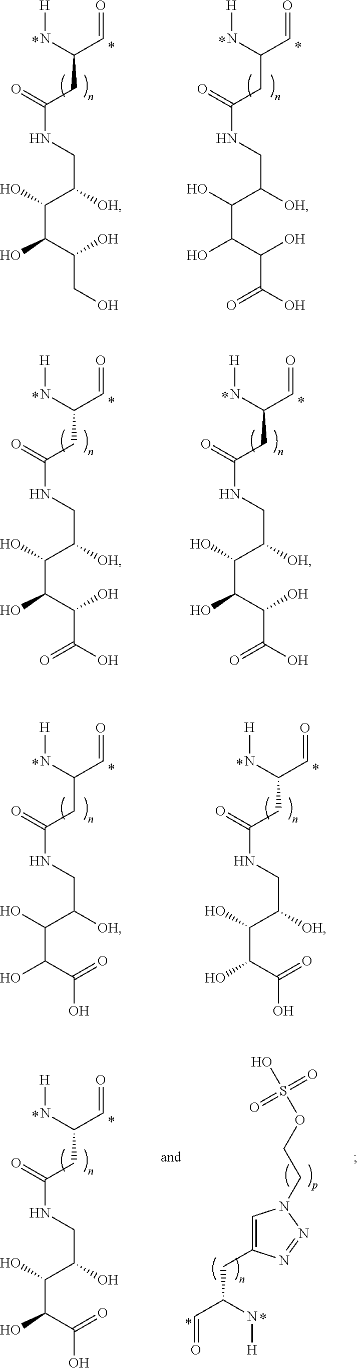

and combinations thereof,
wherein
R¹⁸ is selected from the group consisting of H, D, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′} —NR²⁹C(O)R³⁰, —NR²⁹C(O)OR³⁰, —NR²⁹C(O)NR³⁰R^{30′}, —NR²⁹S(O)R^{30′}, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰, and R^{30′} is independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R²⁹, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′} and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹;
each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;
R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);
R²⁸ is H or sugar;
n is 1, 2, 3, 4 or 5;
p is 1, 2, 3, 4 or 5;
q is 1, 2, 3, 4 or 5; and
each * represent a covalent bond to the rest of the conjugate.

In some embodiments of the conjugates described herein, L is present. In some embodiments of the conjugates described herein, L is absent. In some embodiments, z1 is 0. In some embodiments, z3 is 0. In some embodiments, z5 is 0. In some embodiments, z1 is 0, z3 is 0 and z5 is 0. In some embodiments, z1 is 1. In some embodiments, z3 is 1. In some embodiments, z5 is 1. In some embodiments, z1 is 1, z3 is 1 and z5 is 1.

L^{r} is a releasable linker. As used described herein, a “releasable linker” refers to a linker that includes at least one cleavable bond that can be broken under physiological conditions, such as a pH-labile, acid-labile, base-labile, oxidatively labile, metabolically labile, biochemically labile, or enzyme-labile bond.

It will be appreciated that a releasable linker includes a cleavable bond that can connect two adjacent atoms within the releasable linker. The lability of the cleavable bond can be adjusted by, for example, substituents at or near the cleavable bond, such as including alpha-branching adjacent to a cleavable disulfide bond, increasing the hydrophobicity of substituents on silicon in a moiety having silicon-oxygen bond that may be hydrolyzed, homologating alkoxy groups that form part of a ketal or acetal that may be hydrolyzed, and the like.

Illustrative releasable linkers described herein include linkers that include hemiacetals and sulfur variations thereof, acetals and sulfur variations thereof, hemiaminals, aminals, disulfides, hydrazines, and the like.

In connection with embodiment 1, at least one L^{r} of the formula

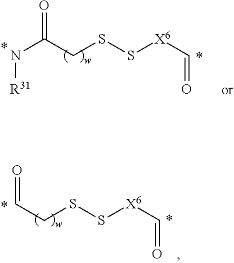

is included in the conjugates described by embodiment 1, wherein
each R³ and R^{3′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each X⁶ is independently selected from the group consisting of —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— and —C₆-C₁₀ aryl-(C₁-C₆ alkyl)-NR^{31′}—, wherein each hydrogen atom in —C₁-C₆ alkyl-, —C₆-C₁₀ aryl-(C₁-C₆alkyl)-, —C₁-C₆ alkyl-O—, —C₆-C₁₀ aryl-(C₁-C₆ alkyl)-O—, —C₁-C₆ alkyl-NR^{31′}— or —C₆-C₁₀ aryl-(C₁-C₆ alkyl)-NR^{31′} is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} are independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl;
each w is independently an integer from 1 to 4; and
each * represents a covalent bond to the rest of the conjugate.

In some embodiments, R³ is H. In some embodiments, R³⁶ is H. In some embodiments, X⁶ is C₁-C₆ alkyl. In some embodiments, X⁶ is C₁-C₆ alkyl. C₆-C₁₀ aryl-(C₁-C₆ alkyl).

In some aspects of embodiment 1, L^{r} is of the formula

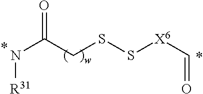

wherein R³¹, X⁶ and w are as described herein, and each * represents a covalent bond to the rest of the conjugate.

In some aspects of embodiment 1, L^{r} is of the formula

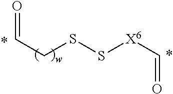

wherein X⁶ and w are as described herein, and each * represents a covalent bond to the rest of the conjugate.

In some aspects of embodiment 1, L^{r} is of the formula

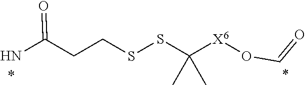

wherein each * represents a covalent bond to the rest of the conjugate.

In some aspects of embodiment 1, L^{r} is of the formula

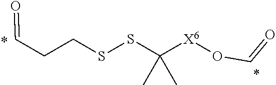

wherein each * represents a covalent bond to the rest of the conjugate.

In connection with embodiment 2, L^{r} can be present or absent, and when present, L^{r} can be selected from the group consisting of

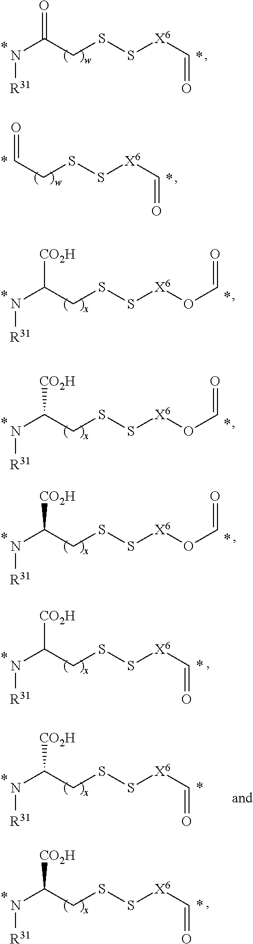

wherein
each R³ and R^{3′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R^{33′}, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each X⁶ is independently selected from the group consisting of —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— and —C₆-C₁₀ aryl-(C₁-C₆ alkyl)-NR^{31′}—, wherein each hydrogen atom in —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— or —C₆-C₁₀ aryl-(C₁-C₆ alkyl)-NR^{31′} is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR^{35′}, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′}—;
each R³², R^{32′}, R³³, R^{33′}, R³⁴, R^{34′}, R³⁵ and R^{35′} are independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl;
each w is independently an integer from 1 to 4;
each x is and integer from 1 to 3; and
each * represents a covalent bond to the rest of the conjugate.

In some embodiments, R³ is H. In some embodiments, R³⁶ is H. In some embodiments, X⁶ is C₁-C₆ alkyl. In some embodiments, X⁶ is C₁-C₆ alkyl. C₆-C₁₀ aryl-(C₁-C₆ alkyl).

In some aspects of embodiment 2, L^{r} is of the formula

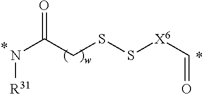

wherein R³¹, X⁶ and w are as described herein, and each * represents a covalent bond to the rest of the conjugate.

In some aspects of embodiment 2, L^{r} is of the formula

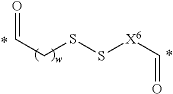

wherein X⁶ and w are as described herein, and each * represents a covalent bond to the rest of the conjugate.

In some aspects of embodiment 2, L^{r} is of the formula

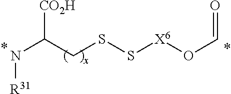

wherein R³¹, X⁶ and x are as described herein, and each * represents a covalent bond to the rest of the conjugate.

In some aspects of embodiment 2, L^{r} is of the formula

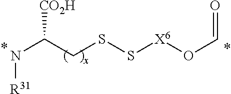

wherein R³¹, X⁶ and x are as described herein, and each * represents a covalent bond to the rest of the conjugate.

In some aspects of embodiment 2, L^{r} is of the formula

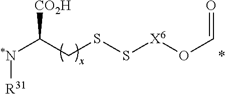

wherein R³¹, X⁶ and x are as described herein, and each * represents a covalent bond to the rest of the conjugate.

In some aspects of embodiment 2, L^{r} is of the formula

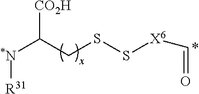

wherein R³¹, X⁶ and x are as described herein, and each * represents a covalent bond to the rest of the conjugate.

In some aspects of embodiment 2, L^{r} is of the formula

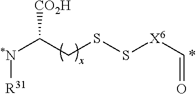

wherein R³¹, X⁶ and x are as described herein, and each * represents a covalent bond to the rest of the conjugate.

In some aspects of embodiment 2, L^{r} is of the formula

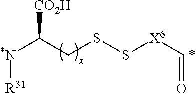

wherein R³¹, X⁶ and x are as described herein, and each * represents a covalent bond to the rest of the conjugate.

In some aspects of embodiment 2, L^{r} is of the formula

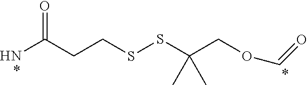

wherein each * represents a covalent bond to the rest of the conjugate.

In some aspects of embodiment 2, L^{r} is of the formula

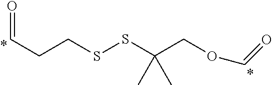

wherein each * represents a covalent bond to the rest of the conjugate.

In some aspects of embodiment 2, L² is of the formula

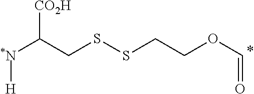

wherein each * represents a covalent bond to the rest of the conjugate.

In some aspects of embodiment 2, L² is of the formula

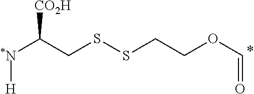

wherein each * represents a covalent bond to the rest of the conjugate.

In some aspects of embodiment 2, L² is of the formula

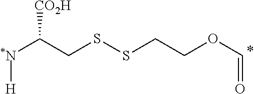

wherein each * represents a covalent bond to the rest of the conjugate.

In some aspects of embodiment 2, L² is of the formula

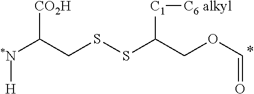

wherein each * represents a covalent bond to the rest of the conjugate. In some aspects, C₁-C₆ alkyl is methyl, ethyl, or isopropyl.

In some aspects of embodiment 2, L² is of the formula

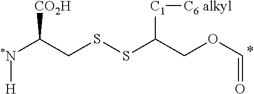

wherein each * represents a covalent bond to the rest of the conjugate. In some aspects, C₁-C₆ alkyl is methyl, ethyl, or isopropyl.

In some aspects of embodiment 2, L² is of the formula

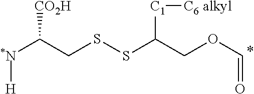

wherein each * represents a covalent bond to the rest of the conjugate. In some aspects, C₁-C₆ alkyl is methyl, ethyl, or isopropyl.

In some aspects of embodiment 2, L² is of the formula

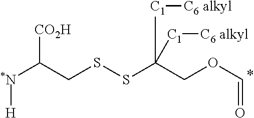

wherein each * represents a covalent bond to the rest of the conjugate. In some aspects, each C₁-C₆ alkyl is methyl.

In some aspects of embodiment 2, L² is of the formula

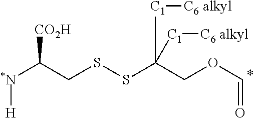

wherein each * represents a covalent bond to the rest of the conjugate. In some aspects, each C₁-C₆ alkyl is methyl.

In some aspects of embodiment 2, L² is of the formula

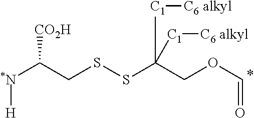

wherein each * represents a covalent bond to the rest of the conjugate. In some aspects, each C₁-C₆ alkyl is methyl.

In some aspects of embodiment 2, L² is of the formula

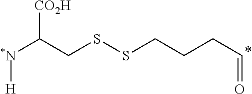

wherein each * represents a covalent bond to the rest of the conjugate.

In some aspects of embodiment 2, L² is of the formula

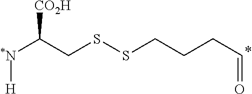

wherein each * represents a covalent bond to the rest of the conjugate.

In some aspects of embodiment 2, L² is of the formula

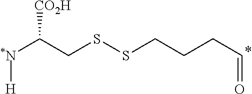

wherein each * represents a covalent bond to the rest of the conjugate.

In some aspects of embodiment 2, L² is of the formula

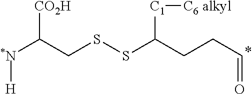

wherein each * represents a covalent bond to the rest of the conjugate. In some aspects, C₁-C₆ alkyl is methyl, ethyl, or isopropyl.

In some aspects of embodiment 2, L² is of the formula

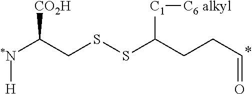

wherein each * represents a covalent bond to the rest of the conjugate. In some aspects, C₁-C₆ alkyl is methyl, ethyl, or isopropyl.

In some aspects of embodiment 2, L² is of the formula

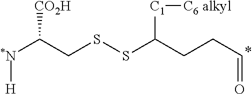

wherein each * represents a covalent bond to the rest of the conjugate. In some aspects, C₁-C₆ alkyl is methyl, ethyl, or isopropyl.

In some aspects of embodiment 2, L² is of the formula

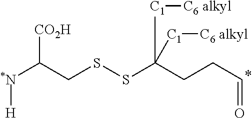

wherein each * represents a covalent bond to the rest of the conjugate. In some aspects, each C₁-C₆ alkyl is methyl.

In some aspects of embodiment 2, L² is of the formula

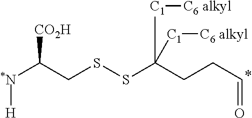

wherein each * represents a covalent bond to the rest of the conjugate. In some aspects, each C₁-C₆ alkyl is methyl.

In some aspects of embodiment 2, L² is of the formula

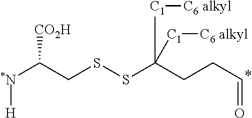

wherein each * represents a covalent bond to the rest of the conjugate. In some aspects, each C₁-C₆ alkyl is methyl.

L² can be present or absent in the conjugates described herein. When L² is present, L² can be any group covalently attaching portions of the linker to the binding ligand, portions of the linker to one another, or to D¹, or to D². It will be understood that the structure of L² is not particularly limited in any way. It will be further understood that L² can comprise numerous functionalities well known in the art to covalently attach portions of the linker to the binding ligand, portions of the linker to one another, or to D¹, or to D², including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L² is selected from the group consisting of C₁-C₆ alkyl, —OC₁-C₆ alkyl, —SC₁-C₆ alkyl, 3- to 7-membered heterocycloalkyl, C₆-C₁₀ aryl, 5- to 7-membered heteroaryl, —R³⁶(C^{36′}R^{36″})ₓ—S-(succinimid-1-yl)-, —(CR^{36′}R^{36″})ᵣC(O)NR³⁶—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ, —C(O)O(CR³⁹R^{39′})ᵣ—, —NR³⁹C(O)(CR^{39′}R^{39″})ᵣ—, —NR³⁹C(O)(CR^{39′}R^{39″})ᵣS—, —(CH₂)ᵣNR³⁹—, —NR³⁹(CH₂)ᵣ—, —NR³⁹(CH₂)ᵣS—. —NR³⁹(CH₂)ᵣNR^{39′}—, —(OCR³⁹R^{39′}CR³⁹R^{39′})ᵣC(O)—, —(OCR³⁹R^{39′}CR³⁹R^{39′}CR³⁹R^{39′})ᵣC(O)—, —OC(O)(CR⁴⁴R^{44′})ₜ—, —C(O)(CR⁴⁴R^{44′})ₜ—, —NR⁴²CR⁴³R^{43′}CR⁴³R^{43′}(OCR⁴⁴R^{44′}CR⁴⁴R^{44′})ₜ—, —CR⁴³R^{43′}CR⁴³R^{43′}(OCR⁴⁴R^{44′}CR⁴⁴R^{44′})ₜNR⁴²—, —NR⁴²C₆-C₁₀ aryl(C₁-C₆ alkyl)OC(O)—, —C(O)CR⁴³R^{43′}CR⁴³R^{43′}(OCR⁴⁴R^{44′}CR⁴⁴R^{44′})ₜNR⁴²—, —NR⁴²CR⁴³R^{43′}CR⁴³R^{43′}(OCR⁴⁴R^{44′}CR⁴⁴R^{44′})ₜC(O)—, and —NR⁴²CR⁴³R^{43′}CR⁴³R^{43′}(CR⁴⁴═CR^{44′})ₜ—;
wherein
each R³⁶, R^{36′} and R^{36″} is independently selected from the group consisting of H, C₁-C₆ alkyl, C₂-C₆ alkenyl, C₂_C₆ alkynyl, C₃_C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R 3, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰OR^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40′}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR and —C(O)NR⁴⁰R^{40′};
R⁴⁰, R^{40′}, R⁴¹ and R^{41′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl; and
R⁴² is selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁵, —OC(O)R⁴⁵, —OC(O)NR⁴⁵R^{45′}, —OS(O)R⁴⁵, —OS(O)₂R⁴⁵, —SR⁴⁵, —S(O)R⁴⁵, —S(O)₂R⁴⁵, —S(O)NR⁴⁵R^{45′}, —S(O)₂NR⁴⁵R^{45′}, —OS(O)NR⁴⁵R^{45′}, —OS(O)₂NR⁴⁵R^{45′}, —NR⁴⁵R^{45′}, —NR⁴⁵C(O)R⁴⁶, —NR⁴⁵C(O)OR⁴⁶, —NR⁴⁵C(O)NR⁴⁶R^{46′}, —NR⁴⁵S(O)R⁴⁶, —NR⁴⁵S(O)₂R⁴⁶, —NR⁴⁵S(O)NR⁴⁶R^{46′}, —NR⁴⁵S(O)₂NR⁴⁶R^{46′}, —C(O)R⁴⁵, —C(O)OR⁴⁵ or —C(O)NR⁴⁵R^{45′},
each R⁴³, R^{43′}, R⁴⁴ and R^{44′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁷, —OC(O)R⁴⁷, —OC(O)NR⁴⁷R^{47′}, —OS(O)R⁴⁷, —OS(O)₂R⁴⁷, —SR⁴⁷, —S(O)R⁴⁷, —S(O)₂R⁴⁷, —S(O)NR⁴⁷R^{47′}, —S(O)₂NR⁴⁷R^{47′}, —OS(O)NR⁴⁷R^{47′}, —OS(O)₂NR⁴⁷R^{47′}, —NR⁴⁷R^{47′}, —NR⁴⁷C(O)R^{47′}, —NR⁴⁷C(O)OR⁴⁸, —NR⁴⁷C(O)NR⁴⁸R^{48′}, —NR⁴⁷S(O)R⁴⁸, —NR⁴⁷S(O)₂R⁴⁸, —NR⁴⁷S(O)NR⁴⁸R^{48′}, —NR⁴⁷S(O)₂NR⁴⁸R^{48′}, —C(O)R⁴⁷, —C(O)OR⁴⁷ or —C(O)NR⁴⁷R^{47′};
R⁴⁵, R^{45′}, R⁴⁶, R^{46′}, R⁴⁷, R^{47′}, R⁴⁸ and R^{48′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
r in each instance is an integer from 1 to 40; and
t is in each instance is an integer from 1 to 40.

In some aspects of the conjugates described herein in connection with either embodiment 1 or embodiment 2, L² is present. In some aspects of the conjugates described herein in connection with either embodiment 1 or embodiment 2, L² is absent.

With respect to embodiment 1: In some aspects, z7 is 0. In some aspects, z7 is 1. In some aspects, z7 is 2. In some aspects, z7 is 3. In some aspects, z7 is 4. In some aspects, z7 is 5. In some aspects, z7 is 6. In some aspects, z7 is 7.

With respect to embodiment 2: In some aspects, z7 is 0. In some aspects, z7 is 1. In some aspects, z7 is 2. In some aspects, z7 is 3. In some aspects, z7 is 4. In some aspects, z7 is 5. In some aspects, z7 is 6. In some aspects, z7 is 7.

In some aspects of embodiment 1, at least one L² is a PEG linker. In some aspects, at least one L² is —(OCR³⁹R^{39′}CR³⁹R^{39′})ᵣC(O)—, r is 4, each R³⁹ is H, and each R^{39′} is H. In some aspects, at least one L² is —(OCR³⁹R^{39′}CR³⁹R^{39′})ᵣC(O), r is 12, each R is H, and each R^{39′} is H. In some aspects, at least one L² is —(OCR³⁹R^{39′}CR³⁹R^{39′})ᵣC(O)—, r is 36, each R³⁹ is H, and each R^{39′} is H. In some aspects, at least one L² is —NR⁴²CR⁴³R^{43′}CR⁴³R^{43′}(OCR⁴⁴R^{44′}CR⁴⁴R^{44′})ₜ—, t is 4, and each R⁴², R⁴³, R^{43′}, R⁴, and R^{44′} is H. In some aspects, at least one L² is —NR⁴²CR⁴³R^{43′}CR⁴³R^{43′}(OCR⁴⁴R^{44′}CR⁴⁴R^{44′})ₜ—, t is 12, and each R⁴², R⁴³, R^{43′}, R⁴, and R^{44′} is H. In some aspects, at least one L² is —NR⁴²CR⁴³R^{43′}CR⁴³R^{43′}(OCR⁴⁴R^{44′}CR⁴⁴R^{44′})ₜ—, t is 36, and each R⁴², R⁴³, R^{43′}, R⁴, and R^{44′} is H. In some aspects, at least one L² is —NR⁴²CR⁴³R^{43′}CR⁴³R^{43′}(OCR⁴⁴R^{44′}CR⁴⁴R^{44′})ₜC(O)—, t is 4, and each R⁴², R⁴³, R^{43′}, R⁴, and R^{44′} is H. In some aspects, at least one L² is —NR⁴²CR⁴³R^{43′}CR⁴³R^{43′}(OCR⁴⁴R^{44′}CR⁴⁴R^{44′})ₜC(O)—, t is 12, and each R⁴², R⁴³, R^{43′}, R⁴, and R^{44′} is H. In some aspects, at least one L² is —NR⁴²CR⁴³R^{43′}CR⁴³R^{43′}(OCR⁴⁴R^{44′}CR⁴⁴R^{44′})ₜC(O)—, t is 36, and each R⁴², R⁴³, R^{43′}, R⁴, and R^{44′} is H.

In some aspects of embodiment 2, at least one L² is a PEG linker. In some aspects, at least one L² is —(OCR³⁹R^{39′}CR³⁹R^{39′})ᵣC(O)—, r is 4, each R³⁹ is H, and each R^{39′} is H. In some aspects, at least one L² is —(OCR³⁹R^{39′}CR³⁹R^{39′})ᵣC(O)—, r is 12, each R is H, and each R^{39′} is H. In some aspects, at least one L² is —(OCR³⁹R^{39′}CR³⁹R^{39′})ᵣC(O)—, r is 36, each R³⁹ is H, and each R^{39′} is H. In some aspects, at least one L² is —NR⁴²CR⁴³R^{43′}CR⁴³R^{43′}(OCR⁴⁴R^{44′}CR⁴⁴R^{44′})ₜ—, t is 4, and each R⁴², R⁴³, R^{43′}, R⁴, and R^{44′} is H. In some aspects, at least one L² is —NR⁴²CR⁴³R^{43′}CR⁴³R^{43′} (OCR⁴⁴R^{44′}CR⁴⁴R^{44′})ₜ—, t is 12, and each R⁴², R⁴³, R^{43′}, R⁴, and R^{44′} is H. In some aspects, at least one L² is —NR⁴²CR⁴³R^{43′}CR⁴³R^{43′} (OCR⁴⁴R^{44′}CR⁴⁴R^{44′})ₜ—, t is 36, and each R⁴², R⁴³, R^{43′}, R⁴, and R^{44′} is H. In some aspects, at least one L² is —NR⁴²CR⁴³R^{43′}CR⁴³R^{43′} (OCR⁴⁴R^{44′}CR⁴⁴R^{44′})ₜC(O)—, t is 4, and each R⁴², R⁴³, R^{43′}, R⁴, and R^{44′} is H. In some aspects, at least one L² is —NR⁴²CR⁴³R^{43′}CR⁴³R^{43′} (OCR⁴⁴R^{44′}CR⁴⁴R^{44′})ₜC(O)—, t is 12, and each R⁴², R⁴³, R^{43′}, R⁴, and R^{44′} is H. In some aspects, at least one L² is —NR⁴²CR⁴³R^{43′}CR⁴³R^{43′} (OCR⁴⁴R^{44′}CR⁴⁴R^{44′})ₜC(O)—, t is 36, and each R⁴², R⁴³, R^{43′}, R⁴, and R^{44′} is H.

With respect to embodiment 1:

In some aspects, at least one L² is —(CR³⁹R^{39′})ᵣC(O)—. In some aspects, L² is —(CR³⁹R^{39′})ᵣC(O)—, r is 5, each R³⁹ is H, and each R^{39′} is H. In some aspects, L² is —(CR³⁹R^{39′})ᵣC(O)—, r is 4, each R³⁹ is H, and each R^{39′} is H. In some aspects, L² is —(CR³⁹R^{39′})ᵣC(O)—, r is 3, each R³⁹ is H, and each R^{39′} is H. In some aspects, L² is —(CR³⁹R^{39′})ᵣC(O)—, r is 2, each R³⁹ is H, and each R^{39′} is H.

In some aspects, at least one L² is —(CR^{36′}R^{36″})ᵣC(O)NR³⁶—. In some aspects, L² is —(CR^{36′}R^{36″})ᵣC(O)NR³⁶—, r is 5, each R³⁶, R³⁶, R^{36″} is H. In some aspects, L² is —(CR^{36′}R^{36″})ᵣC(O)NR³⁶—, r is 4, each R³⁶, R³⁶, R^{36″} is H. In some aspects, L² is —(CR^{36′}R^{36″})ᵣC(O)NR³⁶—, r is 3, each R³⁶, R³⁶, R^{36″} is H. In some aspects, L² is —(CR^{36′}R^{36″})ᵣC(O)NR³⁶—, r is 2, each R³⁶, R³⁶, R^{36″} is H.

In some aspects, at least one L² is —S(CR³⁹R^{39′})ᵣOC(O)—. In some aspects, r is 4. In some aspects, r is 3. In some aspects, r is 2. In some aspects, at least one L² is —NR³⁹C(O)(CR^{39′}R^{39″})ᵣS—. In some aspects, at least one L² is —NR³⁹C(O)(CR^{39′}R^{39″})ᵣS, r is 4, and each of R³⁹, R^{39′} and R^{39″} is H. In some aspects, at least one L² is —NR³⁹C(O)(CR^{39′}R^{39″})ᵣS—, r is 3, and each of R³⁹, R^{39′} and R^{39″} is H. In some aspects, at least one L² is —NR³⁹C(O)(CR^{39′}R^{39″})ᵣS—, r is 2, and each of R³⁹, R^{39′} and R^{39″} is H.

In some aspects, at least one L² is —(CH₂)ᵣNR³⁹—, r is 5 and R³⁹ is H. In some aspects, at least one L² is —(CH₂)ᵣNR³⁹—, r is 4 and R³⁹ is H. In some aspects, at least one L² is —(CH₂)ᵣNR³⁹—, r is 3 and R³⁹ is H. In some aspects, at least one L² is —(CH₂)ᵣNR³⁹—, r is 2 and R³⁹ is H.

In some aspects, at least one L² is —NR³⁹(CH₂)ᵣ, r is 5 and R³⁹ is H. In some aspects, at least one L² is —NR³⁹(CH₂)ᵣ, r is 4 and R³⁹ is H. In some aspects, at least one L² is —NR³⁹(CH₂)ᵣ—, r is 3 and R³⁹ is H. In some aspects, at least one L² is —NR³⁹(CH₂)ᵣ, r is 2 and R³⁹ is H.

In some aspects, at least one L² is

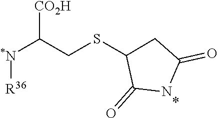

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R^{38′}, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.

In some aspects, at least one L² is

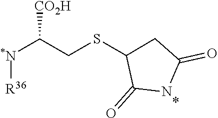

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.

In some aspects, at least one L² is

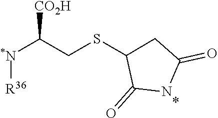

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R^{38′}, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.

With respect to embodiment 2:

In some aspects, at least one L² is —(CR³⁹R^{39′})ᵣC(O)—. In some aspects, L² is —(CR³⁹R^{39′})ᵣC(O)—, r is 5, each R³⁹ is H, and each R^{39′} is H. In some aspects, L² is —(CR³⁹R^{39′})ᵣC(O)—, r is 4, each R³⁹ is H, and each R^{39′} is H. In some aspects, L² is —(CR³⁹R^{39′})ᵣC(O)—, r is 3, each R³⁹ is H, and each R^{39′} is H. In some aspects, L² is —(CR³⁹R^{39′})ᵣC(O)—, r is 2, each R³⁹ is H, and each R^{39′} is H.

In some aspects, at least one L² is —(CR^{36′}R³)ᵣC(O)NR³⁶—. In some aspects, L² is —(CR^{36′}R³)ᵣC(O)NR³⁶—, r is 5, each R³⁶, R³⁶, R^{36″} is H. In some aspects, L² is —(CR^{36′}R³)ᵣ—C(O)NR³⁶—, r is 4, each R³⁶, R³⁶, R^{36″} is H. In some aspects, L² is —(CR^{36′}R³)ᵣC(O)NR³⁶—, r is 3, each R³⁶, R³⁶, R^{36″} is H. In some aspects, L² is —(CR^{36′}R³)ᵣC(O)NR³⁶—, r is 2, each R 36, R³⁶, R^{36″} is H.

In some aspects, at least one L² is —S(CR³⁹R^{39′})ᵣOC(O)—. In some aspects, r is 4. In some aspects, r is 3. In some aspects, r is 2. In some aspects, at least one L² is —NR³⁹C(O)(CR^{39′}R³⁹)ᵣS—. In some aspects, at least one L² is —NR³⁹C(O)(CR^{39′}R^{39″})ᵣS—, r is 4, and each of R³⁹, R^{39′} and R^{39″} is H. In some aspects, at least one L² is —NR³⁹C(O)(CR^{39′}R^{39″})ᵣS—, r is 3, and each of R³⁹, R^{39′} and R^{39″} is H. In some aspects, at least one L² is —NR³⁹C(O)(CR^{39′}R^{39″})ᵣS—, r is 2, and each of R³⁹, R^{39′} and R^{39″} is H.

In some aspects, at least one L² is —(CH₂)ᵣNR³⁹—, r is 5 and R³⁹ is H. In some aspects, at least one L² is —(CH₂)ᵣNR³⁹—, r is 4 and R³⁹ is H. In some aspects, at least one L² is —(CH₂)ᵣNR³⁹—, r is 3 and R³⁹ is H. In some aspects, at least one L² is —(CH₂)ᵣNR³⁹—, r is 2 and R³⁹ is H.

In some aspects, at least one L² is —NR³⁹(CH₂)ᵣ, r is 5 and R³⁹ is H. In some aspects, at least one L² is —NR 39(CH₂)ᵣ, r is 4 and R³⁹ is H. In some aspects, at least one L² is —NR³⁹(CH₂)ᵣ—, r is 3 and R³⁹ is H. In some aspects, at least one L² is —NR³⁹(CH₂)ᵣ—, r is 2 and R³⁹ is H.

In some aspects, at least one L² is

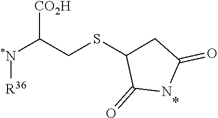

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.

In some aspects, at least one L² is

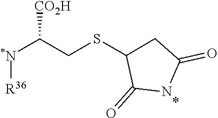

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.

In some aspects, at least one L² is

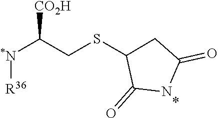

R³⁶ is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
* is a covalent bond. In some embodiments, R³⁶ is H.
   It will be appreciated that L³ can any linker covalently attaching D¹ to D². Specifically, the structure of L³ is not particularly limited in any way in connection with either embodiment 1 or embodiment 2. It will be further understood that L³ can comprise numerous functionalities well known in the art to covalently attach D¹ to D², including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L³ is selected from the group consisting of C₁-C₁₀ alkyl, C₂-C₁₀ alkenyl, C₂_C₁₀ alkynyl, —(CR⁴⁹R^{49′})ᵤC(O)—, —CH₂CH₂(OCR⁴⁹R^{49′}CR⁴⁹R^{49′})ᵤ—, —CH₂CH₂CH₂(OCR⁴⁹R^{49′}CR⁴⁹R^{49′}CR⁴⁹R^{49′})ᵤ—, —CH₂CH₂(OCR⁴⁹R^{49′}CR⁴⁹R^{49′})ᵤC(O)— and —CH₂CH₂(OCR⁴⁹R^{49′}CR⁴⁹R^{49′}CR⁴⁹R^{49′})ᵤC(O)—,
   wherein
each R⁴⁹ and R^{49′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁵⁰, —OC(O)R⁵⁰, —OC(O)NR⁵⁰R^{50′}, —OS(O)R⁵⁰, —OS(O)₂R⁵⁰, —SR⁵⁰, —S(O)R⁵⁰, —S(O)₂R⁵⁰, —S(O)NR⁵⁰R^{50′}, —S(O)₂NR⁵⁰R^{50′}, —OS(O)NR⁵⁰R^{50′}, —OS(O)₂NR⁵⁰R^{50′}, —NR⁵⁰R^{50′}, —NR⁵⁰C(O)R⁵¹, —NR⁵⁰C(O)OR⁵¹, —NR⁵⁰C(O)NR⁵¹R^{51′}, —NR⁵⁰S(O)R⁵¹, —NR⁵⁰S(O)₂R⁵¹, —NR⁵⁰S(O)NR⁵¹R^{51′}, —NR⁵⁰S(O)₂NR⁵¹R^{51′}, —C(O)R⁵⁰, —C(O)OR⁵⁰ or —C(O)NR⁵⁰R^{50′};
R⁵⁰, R^{50′}, R⁵¹ and R^{51′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
u is in each instance 0, 1, 2, 3, 4 or 5.

In some embodiments, L³ is C₁-C₆ alkyl. In some embodiments, L³ is —(CR⁴⁹R^{49′})ᵤC(O)—, wherein each R⁴⁹ and R^{49′} is H, and u is 3. In some embodiments, L is —(CR⁴⁹R^{49′})ᵤC(O)—, wherein each R⁴⁹ and R^{49′} is H, and u is 4. In some embodiments, L³ is —(CR⁴⁹R^{49′})ᵤC(O)—, wherein each R⁴⁹ and R^{49′} is H, and u is 5.

In some embodiments, the linker comprises the formula

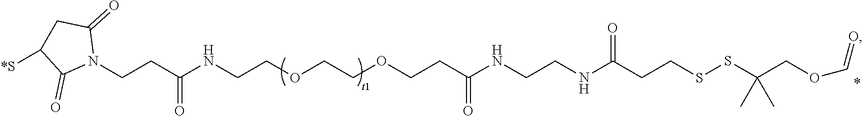

wherein t1 if an integer from 0 to 39, and each * represents a covalent bond to the rest of the conjugate.

In some embodiments, the linker comprises the formula

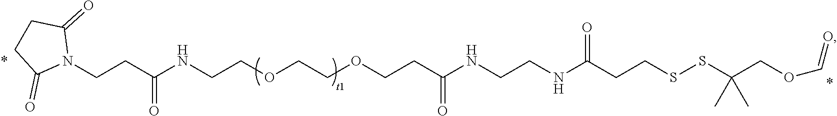

wherein t1 if an integer from 0 to 39, and each * represents a covalent bond to the rest of the conjugate.

In some embodiments, the linker is of the formula

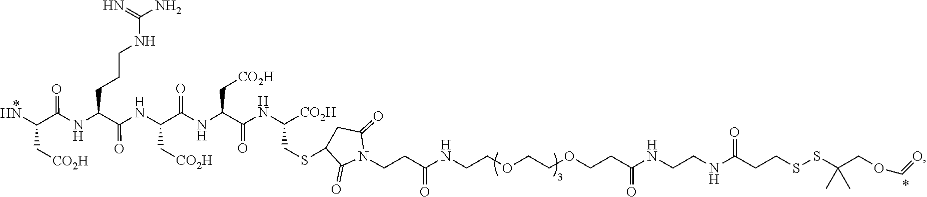

wherein each * represents a covalent bond to the rest of the conjugate.

In some embodiments, the linker is of the formula

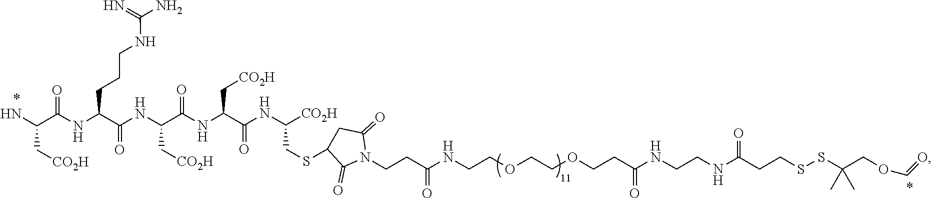

wherein each * represents a covalent bond to the rest of the conjugate.

In some embodiments, the linker is of the formula

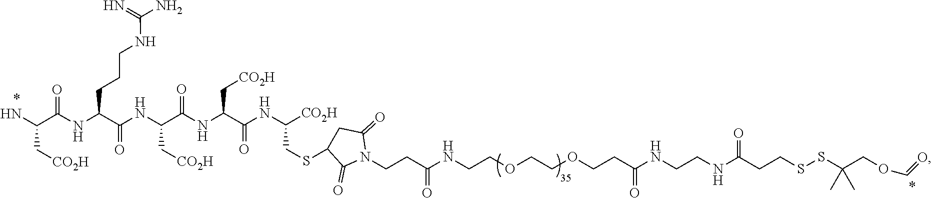

wherein each * represents a covalent bond to the rest of the conjugate.

In some embodiments, the linker is of the formula

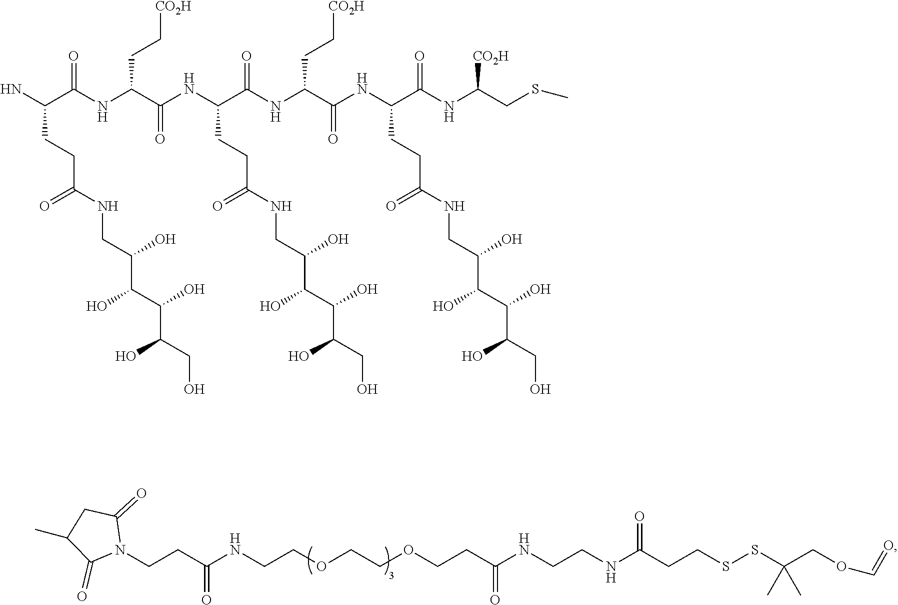

wherein each * represents a covalent bond to the rest of the conjugate.

In some embodiments, the linker is of the formula

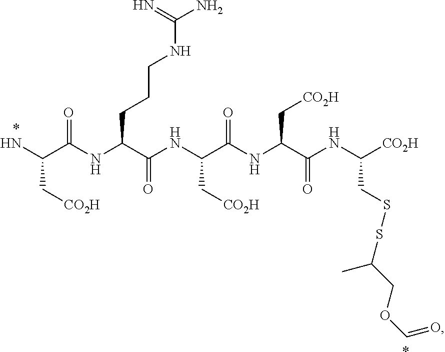

wherein each * represents a covalent bond to the rest of the conjugate.

In some embodiments, the linker is of the formula

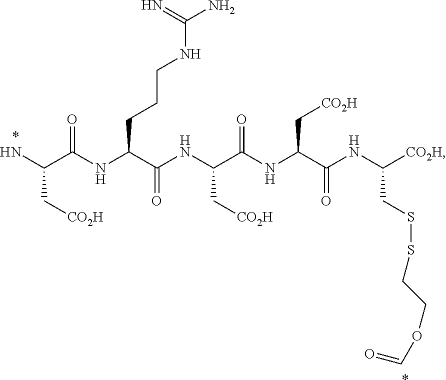

wherein each * represents a covalent bond to the rest of the conjugate.

In some embodiments, the linker is of the formula

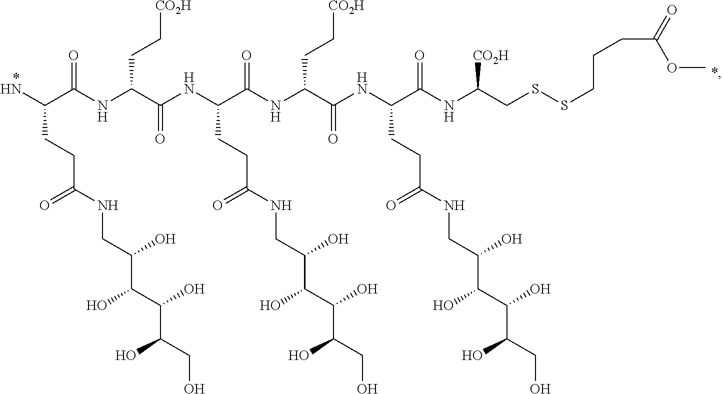

wherein each * represents a covalent bond to the rest of the conjugate.

In some embodiments, the linker comprises the formula

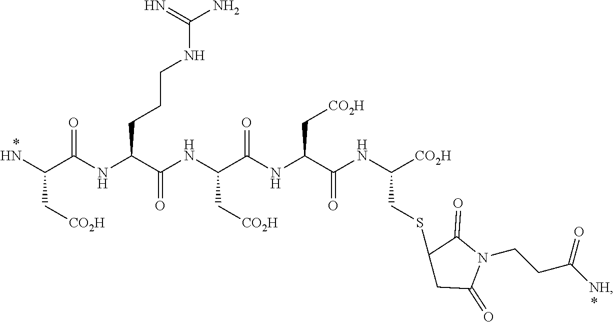

wherein each * represents a covalent bond to the rest of the conjugate.

In some embodiments, the linker is the formula

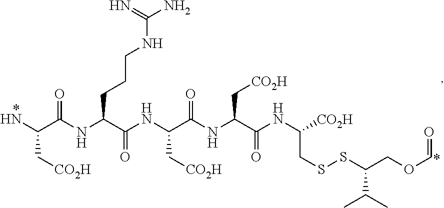

wherein each * represents a covalent bond to the rest of the conjugate.

In some embodiments, the linker is the formula

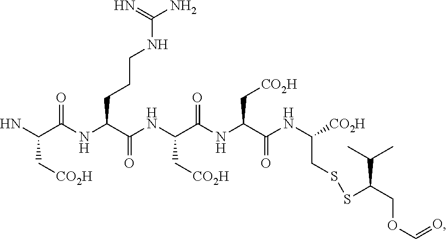 [SMILES: CC(C)[C@@H](COC=O)SSC[C@H](NC(=O)[C@H](CC(=O)O)NC(=O)[C@H](CC(=O)O)NC(=O)[C@H](CCCNC(=N)N)NC(=O)[C@@H](N)CC(=O)O)C(=O)O]

wherein each * represents a covalent bond to the rest of the conjugate.

In some embodiments, the linker is of the formula

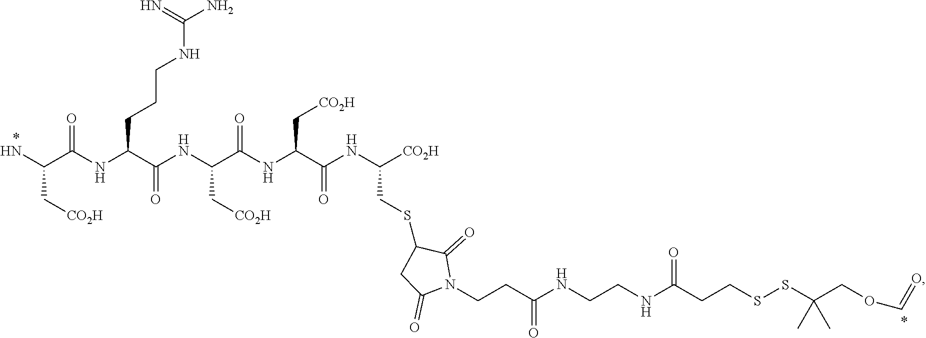

wherein each * represents a covalent bond to the rest of the conjugate.

In some embodiments, the linker is of the formula

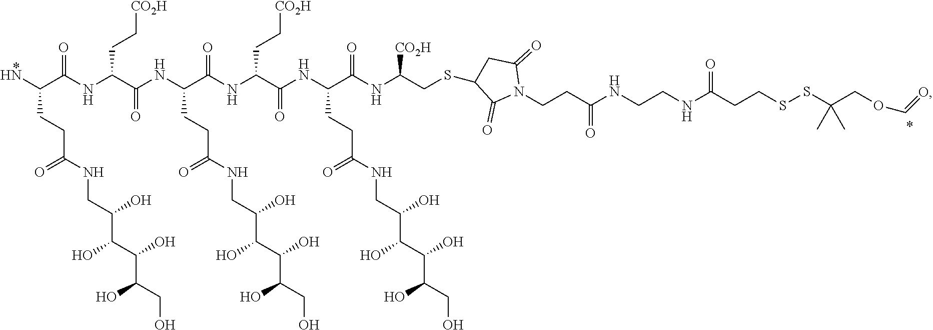

wherein each * represents a covalent bond to the rest of the conjugate.

Drugs

The conjugates described herein comprise the drugs D¹ and/or D², covalently attached to one or more linker portions of the linkers described herein, with the proviso that at least one drug D¹ or D² is a pyrrolobenzodiazepine (also referred to herein as a PBD). In some embodiments, both D¹ and D² are PBD drugs. In some embodiments, the drug comprises the formula -D¹-L³-D². In some embodiments, Drug comprises the structure -D¹-L³-D²-. In some embodiments, one of D¹ or D² is a PBD drug, and the other of D¹ or D² is a pyrrolobenzodiazepine pro-drug (also referred to herein as a PBD pro-drug or pro-PBD). It will be understood that such PBD prodrugs undergo conversion to a therapeutically active PBD compound through processes in the body after delivery of a conjugate as described herein. In some embodiments, at least one of the drugs incorporated into conjugates described herein is a PBD prodrug as described herein. It will be appreciated that the drugs are not particularly limited in any way with respect each either embodiment 1 or embodiment 2, with the proviso that at least one of D¹ or D² is a PBD. Accordingly, the description of drugs for use in connection with the present teachings apply equally to both embodiment 1 and embodiment 2.

In some embodiments, the first drug or the second drug is a PBD of the formula

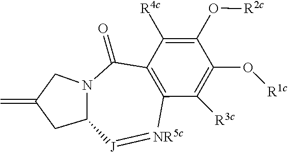

wherein
J, R^{1c}, R^{2c}, R^{3c}, R^{4c} and R^{5c} are each defined as described herein.
In some embodiments, the first drug is of the formula

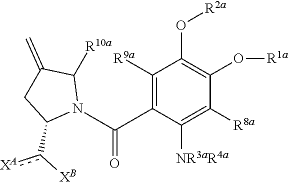

wherein
X^{A}, X^{B}, R^{1a}, R^{2a}, R^{3a}, R^{4a}, R^{8a}, R^{9a} and R^{10a} are as defined herein.

In some embodiments, the second drug is selected from the group consisting of

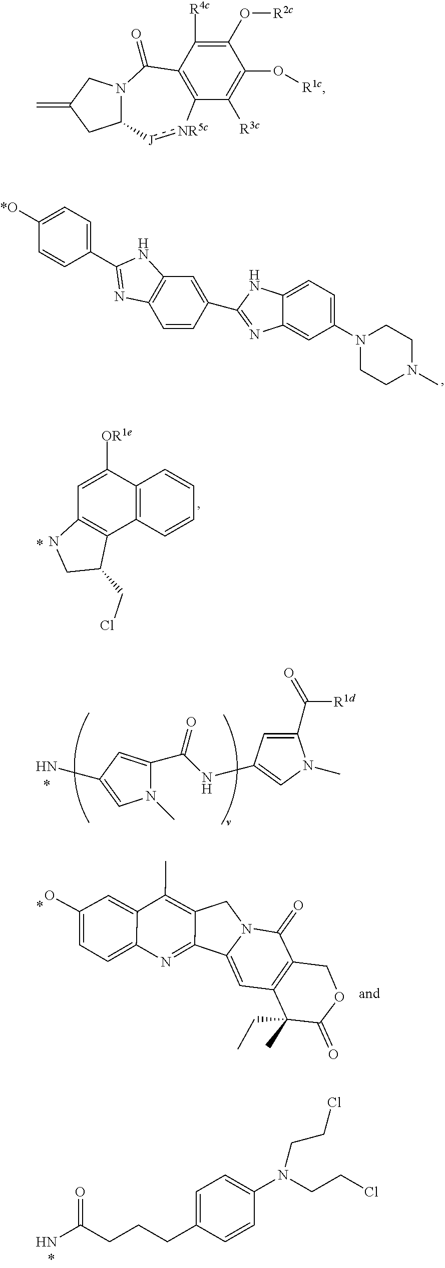

wherein
J is —C(O)—, —CR^{13c}═ or —(CR^{13c}R^{13c})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R⁶, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R^{7c′}, —OS(O)NR^{7c}R^{7c′}, —OS(O)₂NR^{7c}R^{7c′}, —NR^{7c}R^{7c′}, —NR^{7c}C(O)R^{8c}, —NR^{7c}C(O)OR^{8c}, —NR^{7c}C(O)NR^{8c}R^{8c′}, —NR^{7c}S(O)R^{8c}, —NR^{7c}S(O)₂R^{8c}, —NR^{7c}S(O)NR^{8c}R^{8c′}, —NR^{7c}S(O)₂NR^{8c}R^{8c′}, —C(O)R^{7c}, —C(O)OR^{7c} or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c′}, —OS(O)R^{9c}, —OS(O)₂R^{9c}, —SR^{9c}, —S(O)R^{9c}, —S(O)₂R^{9c}, —S(O)NR^{9c}R^{9c′}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{10c}, —NR^{9c}C(O)OR^{10c}, —NR^{9c}C(O)NR^{10c}R^{10c′}, —NR^{9c}S(O)R^{10c}, —NR^{9c}S(O)₂R^{10c}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, OS(O)R^{11c}, —OS(O)₂R^{11c}, —SR^{11c}, —S(O)R^{11c}, —S(O)₂R^{11c}, —S(O)NR^{11c}R^{11c}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c};
R^{1d} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{2d}, —SR^{2d} and —NR^{2d}R^{2d′},
R^{2d} and R^{2d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{3d}, —SR^{3d}, and —NR^{3d}R^{3d′};
R^{3d} and R^{3d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
R^{1e} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₆ alkynyl, C₃_C₆ cycloalkyl, 3- to 7-membered heterocycloalkyl, C₆-C₁₀ aryl, 5- to 7-membered heteroaryl, —OR^{2e}, —OC(O)R^{2e}, —OC(O)NR^{2e}R^{2e′}, —OS(O)R^{2e}, —OS(O)₂R^{2e}, —SR^{2e}, —S(O)R^{2e}, —S(O)₂R^{2e}, —S(O)NR^{2e}R^{2e′}, —S(O)₂NR^{2e}R^{2e′}, —OS(O)NR^{2e}R^{2e′}, —OS(O)₂NR^{2e}R^{2e′}, —NR^{2e}R^{2e′}, —NR^{2e}C(O)R^{3e}, —NR^{2e}C(O)OR^{3e}, —NR^{2e}C(O)NR^{3e}R^{3e′}, —NR^{2e}S(O)R^{3e}, —NR^{2e}S(O)₂R^{3e}, —NR^{2e}S(O)NR^{2e}R^{2e′}, —NR^{2e}S(O)₂NR^{3e}R^{3e′}, —C(O)R^{2e}, —C(O)OR^{2e} or —C(O)NR^{2e}R^{2e}.
each R^{2e}, R^{2e′}, R^{3e} and R^{3e′} is independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{4e}, —SR^{4e} or —NR^{4e}R^{4e′};
R^{4e}, and R^{4e′}, are independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
v is 1, 2 or 3; and
each * represents a covalent bond to the rest of the conjugate.

In some embodiments, the drug comprises the formula

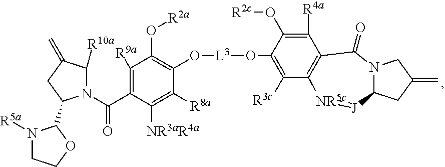

wherein R^{5a} is a covalent bond to the rest of the conjugate.

In some embodiments, the drug comprises the formula

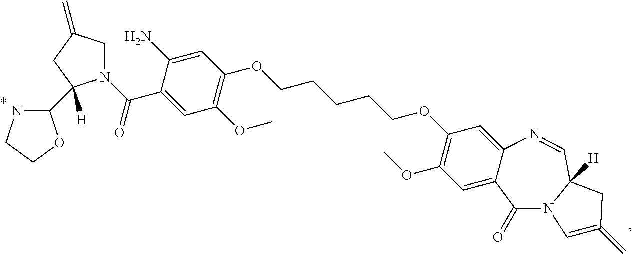

wherein * represents a covalent bond to the rest of the conjugate.

In some embodiments, the drug comprises the formula

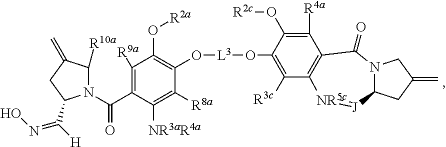

wherein R^{4a} is a covalent bond to the rest of the conjugate.

In some embodiments, the drug comprises the formula

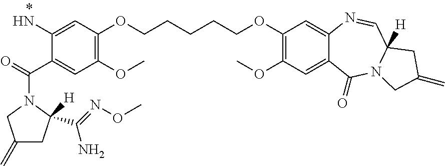

wherein * represents a covalent bond to the rest of the conjugate.

In some embodiments, the drug comprises the formula

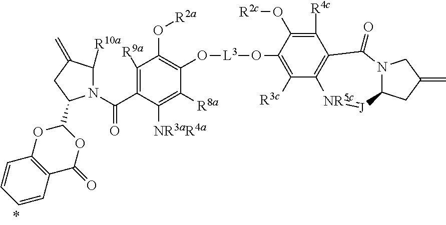

wherein * represents a covalent bond to the rest of the conjugate.

In some embodiments, the drug comprises the formula

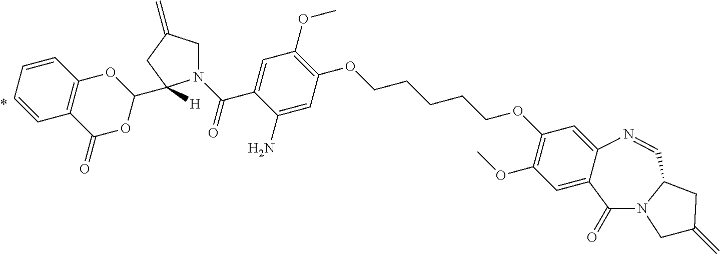 [SMILES: C=C1C[C@H]2C=Nc3cc(OCCCCCOc4cc(N)c(C(=O)N5CC(=C)C[C@H]5C5OC(=O)c6ccccc6O5)cc4OC)c(OC)cc3C(=O)N2C1]

wherein * represents a covalent bond to the rest of the conjugate.

In some embodiments, the drug comprises the formula

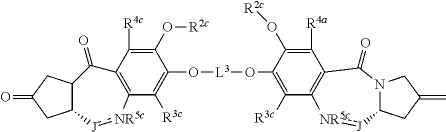

wherein at least one R^{5c} is a covalent bond to the rest of the conjugate.

In some embodiments, the drug comprises the formula

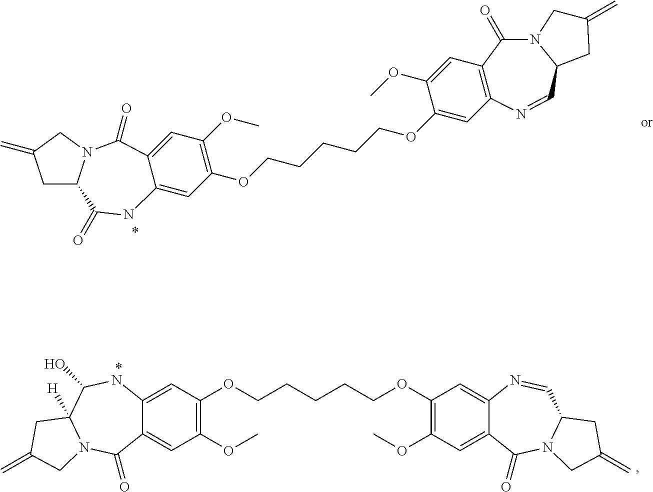

wherein * represents a covalent bond to the rest of the conjugate.

In some embodiments, the drug comprises the formula

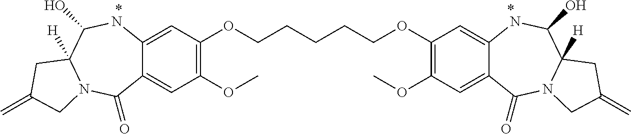

wherein * represents a covalent bond to the rest of the conjugate.

The conjugates described herein can be used for both human clinical medicine and veterinary applications. Thus, the host animal harboring the population of pathogenic cells and treated with the conjugates described herein can be human or, in the case of veterinary applications, can be a laboratory, agricultural, domestic, or wild animal. The conjugates described herein can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The conjugate, compositions, methods, and uses described herein are useful for treating diseases caused at least in part by populations of pathogenic cells, which may cause a variety of pathologies in host animals. As used herein, the term “pathogenic cells” or “population of pathogenic cells” generally refers to cancer cells, infectious agents such as bacteria and viruses, bacteria- or virus-infected cells, inflammatory cells, activated macrophages capable of causing a disease state, and any other type of pathogenic cells that uniquely express, preferentially express, or overexpress cell surface receptors or cell surface antigens that may be bound by or targeted by the conjugates described herein. Pathogenic cells can also include any cells causing a disease state for which treatment with the conjugates described herein results in reduction of the symptoms of the disease. For example, the pathogenic cells can be host cells that are pathogenic under some circumstances such as cells of the immune system that are responsible for graft versus host disease, but not pathogenic under other circumstances.

Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The conjugates described herein can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas; including associated cancers resistant to treatment modalities, such as therapeutic agents. Resistant cancers include but are not limited to paclitaxel resistant cancers, and platinum resistant cancers, such as those cancers resistant to platinum drugs, such as cisplatin, carboplatin, oxaplatin, nedaplatin, and the like. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, stomach and lung cancers. In some embodiments, the cancer cell population produces a cancer, such as lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

In some embodiments, the cancer is folate receptor positive triple negative breast cancer. In some embodiments, the cancer is folate receptor negative triple negative breast cancer. In some embodiments, the cancer is ovarian cancer. In some embodiments, the method further comprises concurrently treatment with anti-CTLA-4 treatment. In some embodiments, the method further comprises concurrently treatment with anti-CTLA-4 treatment for the treatment of ovarian cancer.

The disclosure includes all pharmaceutically acceptable isotopically-labelled conjugates, and their Drug(s) incorporated therein, wherein one or more atoms are replaced by atoms having the same atomic number, but an atomic mass or mass number different from the atomic mass or mass number which predominates in nature.

Examples of isotopes suitable for inclusion in the conjugates, and their Drug(s) incorporated therein, include isotopes of hydrogen, such as ²H and 3H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled conjugates, and their Drug(s) incorporated therein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, and ¹³N, can be useful in Positron Emission Topography (PET) studies for examining substrate receptor occupancy. Isotopically-labeled conjugates, and their Drug(s) incorporated therein, can generally be prepared by conventional techniques known to those skilled in the art or by processes analogous to those described in the accompanying Examples using an appropriate isotopically-labeled reagents in place of the non-labeled reagent previously employed.

The conjugates and compositions described herein may be administered orally. Oral administration may involve swallowing, so that the conjugate or composition enters the gastrointestinal tract, or buccal or sublingual administration may be employed by which the conjugate or composition enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The conjugates and compositions described herein may also be used in fast-dissolving, fast-disintegrating dosage forms such as those described in Expert Opinion in Therapeutic Patents, 11 (6), 981-986, by Liang and Chen (2001). For tablet dosage forms, depending on dose, the conjugate may make up from 1 weight % to 80 weight % of the dosage form, more typically from 5 weight % to 60 weight % of the dosage form. In addition to the conjugates and compositions described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a conjugate as described herein, a film-forming polymer, a binder, a solvent, a humectant, a plasticizer, a stabilizer or emulsifier, a viscosity-modifying agent and a solvent. Some components of the formulation may perform more than one function.

Solid formulations for oral administration may be formulated to be immediate and/or modified release. Modified release formulations include delayed-, sustained-, pulsed-, controlled-, targeted and programmed release. Suitable modified release formulations for the purposes of the disclosure are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The conjugates described herein can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous.

Suitable devices for parenteral administration include needle (including micro-needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of conjugates described herein used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus conjugates described herein can be formulated as a solid, semi-solid, or thixotropic liquid for administration as an implanted depot providing modified release of the active compound. Examples of such formulations include drug-coated stents and poly(lactic-coglycolic) acid (PGLA) microspheres. The conjugates described herein can also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release. The conjugates described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurized container, pump, spray, atomizer, or nebulizer contains a solution or suspension of the conjugates(s) of the present disclosure comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the conjugate is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or hydroxypropylmethylcellulose), blisters and cartridges for use in an inhaler or insufflator may be formulated to contain a powder mix of the conjugate described herein, a suitable powder base such as lactose or starch and a performance modifier such as Iso-leucine, mannitol, or magnesium stearate.

The lactose may be anhydrous or in the form of the monohydrate, preferably the latter. Other suitable excipients include dextran, glucose, maltose, sorbitol, xylitol, fructose, sucrose and trehalose. A typical formulation may comprise a conjugate of the present disclosure, propylene glycol, sterile water, ethanol and sodium chloride. Alternative solvents which may be used instead of propylene glycol include glycerol and polyethylene glycol.

The conjugates described here can be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present disclosure that two or more pharmaceutical compositions, at least one of which contains a conjugate as described herein, may conveniently be combined in the form of a kit suitable for co-administration of the compositions. Thus the kit of the present disclosure comprises two or more separate pharmaceutical compositions, at least one of which contains a conjugate as described herein, and means for separately retaining said compositions, such as a container, divided bottle, or divided foil packet. An example of such a kit is the familiar blister pack used for the packaging of tablets, capsules and the like. The kit of the present disclosure is particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

### EXAMPLES

### Chemical Examples

It is to be understood that the conjugates described herein were prepared according to the processes described herein and/or conventional processes. Illustratively, the stereocenters of the conjugates described herein may be substantially pure (S), the substantially pure (R), or any mixture of (S) and (R) at any asymmetric carbon atom, and each may be used in the processes described herein. Similarly, the processes described in these illustrative examples may be adapted to prepare other conjugates described herein by carrying out variations of the processes described herein with routine selection of alternative starting materials and reagents. It is also to be understood that radicals of these examples are included in the PBD prodrugs, poly-PBD prodrugs, mixed PBDs, and conjugates described herein.

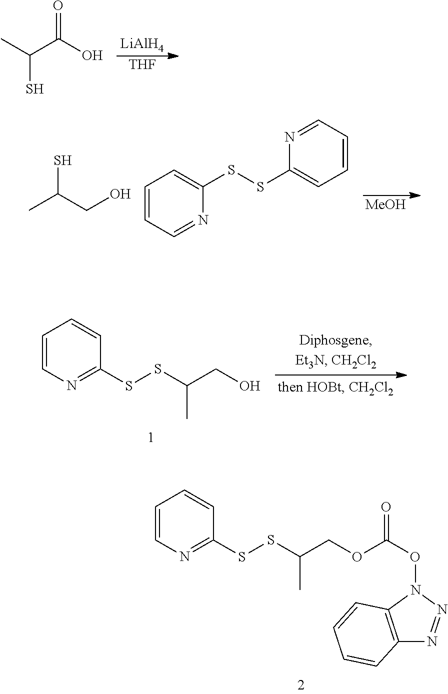

### Example 1: Prepararion of Compound 2

### Step 1: Preparation of 2-Thiopropanol

2-Mercaptopropionic acid (1 mL, 11.27 mmol) in anhydrous THF (35 mL) was treated with 2 M LiAlH₄ in THF (11.3 mL, 22.5 mmol) and heated at reflux for 2 h. The reaction mixture was cooled to 0° C. 2 N HCl was added dropwise while maintaining an internal temperature below 30° C. until the evolution of bubbles ceased. The reaction mixture was stirred for 1 h and filtered through a pad of Celite. The filtrate was concentrated in vacuo and used without further purification.

### Step 2: Prepararion of Compound 1

2-Mercaptopropanol was dissolved in MeOH (10 mL) and added dropwise to a solution of 2,2′-dipyridyl disulfide (3.00 g, 14.0 mmol) in MeOH (10 mL). The reaction mixture was stirred for 30 min at room temperature and then concentrated under vacuum. The residue was dissolved in 3 mL of CH₂Cl₂ and purified via silica chromatography (0-40% EtOAc/pet. ether) to yield the desired product as a colorless oil, (332.7 mg, 17% over two steps); LC/MS (ESI-QMS): m/z=202 (M+H).

### Step 3: Prepararion of Compound 2

Compound 1 (111 mg, 0.549 mmol) and Et₃N (76.5 μL, 0.549 mmol) were dissolved in CH₂Cl₂ (15 mL) and added dropwise to a solution of diphosgene (36.5 μL, 0.302 mmol) in CH₂Cl₂ (0.5 mL) at 0° C. The reaction mixture was stirred for 30 min at 0° C. and monitored by TLC (40% EtOAc/pet. ether). A solution of 1-Hydroxybenzotriazole hydrate (74.2 mg, 0.549 mmol) in CH₂Cl₂ (2 mL) followed by Et₃N (41.2 μL, 0.544 mmol) was added to the reaction mixture at 0° C. The reaction mixture was allowed to warm to room temperature and stirred for 3 h. After the reaction was carried out to completion, reaction mixture was concentrated and purified via silica chromatography (0-40% EtOAc/Pet. ether). The desired product was obtained as a white solid (116.7 mg, 59% over two steps); LC/MS (ESI-QMS): m/z=363 (M+H), H NMR (500 MHz, CDCl₃) δ 8.43 (m, 1H), 8.22 (d, J=8.31 Hz, 1H), 8.01 (d, J=8.80 Hz, 1H), 7.76 (m, 1H), 7.65 (td, J=7.83, 1.60 Hz, 1H), 7.56 (t, J=7.82 Hz, 1H), 7.08 (m, 1H), 4.69 (dd, J=11.25, 5.87 Hz, 1H), 4.58 (dd, J=11.25, 6.84 Hz, 1H), 3.45 (m, 1H), 1.49 (d, J=7.33 Hz 3H).

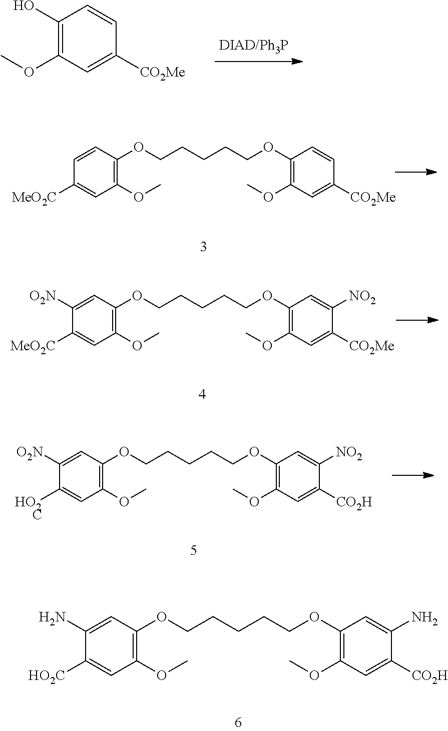

### Example 4: Preparation of Compound 6

### Step 1: Preparation of Compound 3

Methyl vanillate (2.18 g, 11.98 mmol) and Ph₃P (4.71 g, 17.97 mmol) in THF (20 mL) was cooled to 0° C. and to which was added DIAD (2.59 mL, 13.18 mmol) dropwise. The reaction was stirred at 0° C. for 1 hr. 1,5-petanediol (0.6 mL, 5.75 mmol) in THF (20 mL) was added over 30 min. The reaction was stirred overnight and prESIpitate formed and was collected with filtration. The filtrate was concentrated to form more solid. The solid was combined and triturated with MeOH (5 mL) to give qite clean product Compound 3 1.74 g in yield of 70%. ¹H NMR (CDCl₃, 6 in ppm): 7.66 (m 2H), 7.62 (m, 2H), 6.87 (m, 2H), 4.10 (m, 4H), 3.89 (m, 12H), 1.95 (m, 4H), 1.69 (m, 2H). ¹³C NMR: 166.88, 152.50, 148.86, 132.12, 132.04, 131.88, 128.52, 128.42, 123.50, 122.55, 112.35, 111.46, 68.67, 56.03, 51.93, 28.73, 22.52, 21.92.

### Step 2: Preparation of Compound 4

Compound 3 (201.2 mg, 0.465 mmol) in Ac₂O (1.2 mL) was cooled to 0° C. and then Cu(NO₃)₂.3H₂O (280.3 mg, 1.16 mmol) was added slowly and after 1 hr, the ice-bath was removed. The reaction was stirred at r.t. for 4 hrs. The reaction was poured into ice water and stirred for 1 h till yellow precipitate formed and was collected with filtration. The solid was washed with more cold water (2 mL, 3×) and air-dried. 198.4 mg of Compound 4 was obtained in yield of 82%. LCMS: [M+NH₄]+m/z=540.

### Step 3: Preparation of Compound 5

Compound 4 (198.4 mg) was dissolved in THF (2 mL) and treated with aq. NaOH (2 mL, 1 M) and heated to 40° C. for 3 hrs. The solvent was removed in vacuo. The aqueous phase was acidified to pH 1 with concentrated HCl to form precipitate, which was collected by filtration and was washed with H₂O (1 mL, 3×). The solid was air-dried to give the acid 187.7 mg of Compound 5 in quantitative yield. LCMS: [M+NH₄]+m/z=512.

### Step 4: Preparation of Compound 6

Acid Compound 5 was dissolved in 0.5 M aq. NaOH (6 mL) and hydrogenation was carried out with Pd/C (10%, 4.82 mg) under H₂ (45 PSI) in the hydrogenation parr. The reaction was shook for 5 hrs and the filtered through a pad of celite and the filtrate was adjusted to pH 2-3 with concentrated HCl while stirring. The formed precipitate was isolated by filtration and washed with H₂O (1 mL, 3×). The solid was dried in a desiccator with the presence of P₂O₅ under high vacuum overnight. Compound 6 was obtained 34.2 mg as a brown solid in the yield of 81%. LCMS: [M−H]⁻ m/z=433.

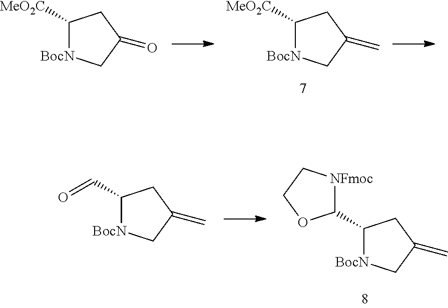

### Example 3: Preparation of Compound 8

### Step 1: Preparation of Compound 7

(S)-1-tert-butyl 2-methyl 4-oxopyrrolidine-1,2-dicarboxylate was converted to Compound 7 by Wittig reaction: Ph₃PCH₃Br (917.8 mg, 2.57 mmol) in THF (30 mL) was treated with KO^{t}Bu (1 M in THF, 2.57 μL, 2.57 mmol) at 0° C. by dropwise addition. The reaction was kept at room temperature for 2 hrs. Into the stirred solution was added the ketone (250 mg, 1.028 mmol) in THF 20 mL) at 0-10° C. The reaction was then stirred at room temperature for overnight. The reaction was quenched with H₂O/EtOAc (1:1, 40 mL) after most of the THF was removed in vacuo. The aq. phase was extracted with EtOAc (20 mL, 3×) and the organic phase was washed with H₂O, followed by brine, and dried over anhydrous Na₂SO₄ and concentrated. The residue was purified with CombiFlash in 0-50% EtOAc/p-ether to afford the Compound 7 77.2 mg, in yield of 31%. LCMS: [M-Boc+H]+m/z=142.

### Step 2: Preparation of Aldehyde Intermediate

(S)-1-tert-butyl 2-methyl 4-methylenepyrrolidine-1,2-dicarboxylate (353.2 mg, 1.46 mmol) in DCM/toluene (1:3, 9.8 mL) was treated with Dibal (1 M in toluene, 2 eq, 2.92 mmol) dropwise at −78° C. under argon. The reaction was stirred at −78° C. for ca. 4 hrs. Then the reaction was quenched with addition of 60 μL of MeOH at −78° C. followed by 5% HCl (0.5 mL) and EtOAc (18 mL). The cold bath was removed and the reaction was stirred for 30 min. The EtOAc layer was separated and washed with brine, dried over anhydrous Na₂SO₄ and concentrated to give the crude aldehyde intermediate.

### Step 3: Preparation of Compound 8

The crude aldehyde was redissolved in dry DCM (10 mL) and treated with ethanolamine (106 μL, 1.75 mmol) in the presence of anhydrous MgSO₄ (5 mmol, mg) at r.t. (room temperature) under Ar. The reaction was stirred for 1 hr. Then into this reaction mixture was added FmocCl (755.4 mg, 2.92 mmol) and TEA (611 μL, 4.38 mmol) and the reaction was stirred for overnight at r.t. under Ar. The reaction was purified with CombiFlash in 0-50% EtOAc/petroleum ether to provide Compound 8 334.2 mg, 46% for 3 steps. LCMS: [M+H]+m/z=477. ¹H NMR (CD₃OD, 6 in ppm): 7.81 (d, J=7.5 Hz, 2H), 7.60 (d, J=7 Hz, 2H), 7.40 (m, 2H), 7.32 (m, 2H), 4.96 (br, 2H), 4.60 (br, 1H), 4.23 (t, J=5.5 Hz, 1H), 3.97 (br, 2H), 3.73 (br, m, 3H), 2.50 (br, 2H), 1.47 (s, 1H), 1.39 (s, 9H).

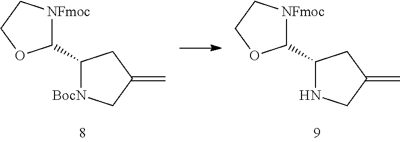

### Example 4: Preparation of Compound 9

Compound 8 was deprotected in TFA/DCM (1:1) at r.t. for 30 min, the solvent was removed in vacuo. The product (Compound 9) was used for the coupling reaction with Compound 6 without further purification. LCMS: [M+H]+m/z=377.

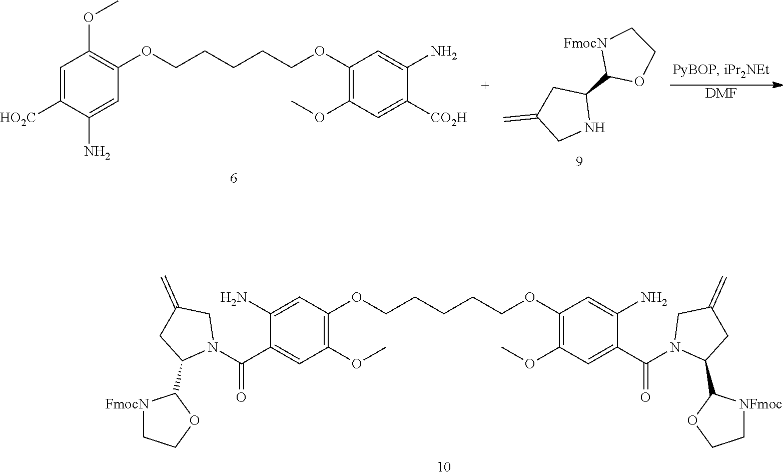

### Example 5: Preparation of Compound 10

Under argon, Compound 6 (482 mg, 1.11 mmol), Compound 9 (878 mg, 2.33 mmol), and PyBOP (1.21 g, 2.33 mmol) were dissolved in DMF (12 mL) and treated with Pr₂NEt (773 μL, 4.34 mmol) at room temperature. The reaction was completed within 1 h and purified by preparative HPLC (10-100% ACN/50 mM NH₄HCO₃ buffer, pH7). The product was extracted from the buffer solution with CH₂Cl₂ and concentrated under reduced pressure to afford Compound 10 (556 mg, 44%); LC/MS (ESI-QMS) m/z=1151.96 (M+H)+, H NMR (500 MHz, DMSO-d6 w/2 drops D₂O) δ 7.84 (d, J=6.5 Hz, 2H), 7.80 (d, J=8.0 Hz, 2H), 7.39 (t, J=7.5 Hz, 2H), 7.32 (t, J=7.0 Hz, 2H), 6.24 (s, 2H), 4.80-5.14 (m, 2H), 3.80-4.20 (m, 6H), 3.52-3.68 (m, 4H), 3.51 (s, 6H), 3.35 (m, 2H), 2.96 (m, 1H), 2.55 (t, J=6.0 Hz, 1H), 2.48 (m, 2H), 1.74 (br, 2H), 1.50 (br, 2H).

### Example 6: Preparation of Compound 11 and 12

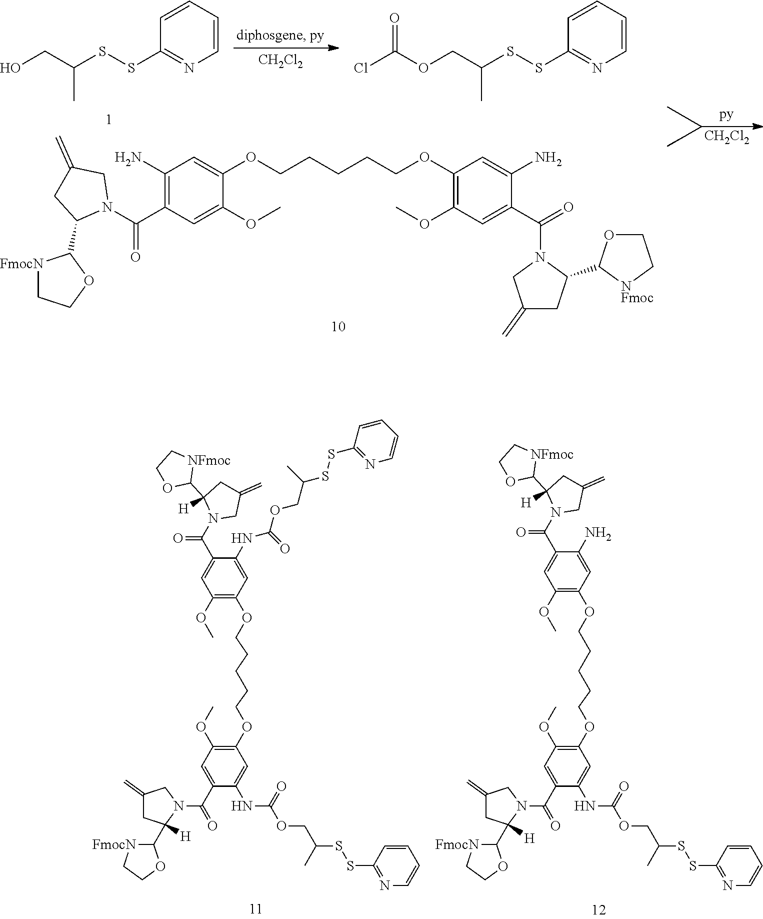

A solution of Compound 1 (18.9 mg, 0.094 mmol) and pyridine (15.2 μL, 0.190 mmol) in CH₂Cl₂ (0.5 mL) was added dropwise to a solution of diphosgene (6.23 μL, 0.052 mmol) in CH₂Cl₂ (0.2 mL) at 0° C. The reaction mixture was allowed to stir for 15-30 min. The resulting chloroformate solution was slowly transferred to a solution of Compound 10 (108.1 mg, 0.94 mmol) in CH₂Cl₂ (0.5 mL) at 0° C. The reaction was stirred for an additional 15 min and then quenched with water (0.5 mL). The organic phase was removed, and the product was extracted further with EtOAc (3 mL×3). The organic layers were combined, washed with brine, dried over anhydrous Na₂SO₄, and concentrated in vacuo. The residue was further purified via silica chromatography (0-100% EtOAc/pet. ether) to provide Compound 11 (23.2 mg, 15%) and Compound 12 (43.2 mg, 34%). Compound 11: LC/MS: (ESI-QMS): m/z==1607 (M+H), ¹H NMR (500 MHz, CDCl₃+ one drop of CD₃OD) δ 8.39 (d, J=3.91 Hz, 2H), 7.78 (d, J=7.82 Hz, 2H), 7.66 (m, 9H), 7.45 (m, 4H), 7.32 (t, J=7.34 Hz, 3H), 7.24 (m, 5H), 7.14 (br, 1H), 7.05 (t, J=5.86 Hz, 2H), 4.94 (br, 6H), 4.30 (br, 2H), 4.13 (m, 6H), 3.95 (br, 6H), 3.89 (br, 2H), 3.62 (m, 8H), 3.50 (br, 2H), 3.31 (m, 2H), 3.17 (br, 6H), 2.60 (br, 2H), 1.85 (s, br, 4H), 1.58 (s, br, 2H), 1.30 (m, 6H); Compound 12: LC/MS: (ESI-QMS): m/z=1380 (M+H), ¹H NMR (500 MHz, CDCl₃+ one drop of CD₃OD) δ 8.33 (br, 1H), 7.70 (m, 6H), 7.60 (d, J=1.46 Hz, 2H), 7.49 (m, 3H), 7.32 (m, 4H), 7.25 (m, 5H), 7.00 (m, 1H), 6.6-6.9 (br, 2H), 4.95 (br, 6H), 4.31 (br, 4H), 3.9-4.2 (m, 12H), 3.54 (m, 10H), 3.50 (br, 2H), 3.32 (m, 1H), 3.20 (m, 1H), 2.90 (br, 3H), 2.60 (m, br, 4H), 1.82 (br, 4H), 1.58 (br, 2H), 1.30 (m, br, 3H).

### Example 7: Preparation of Compound 13 and 14

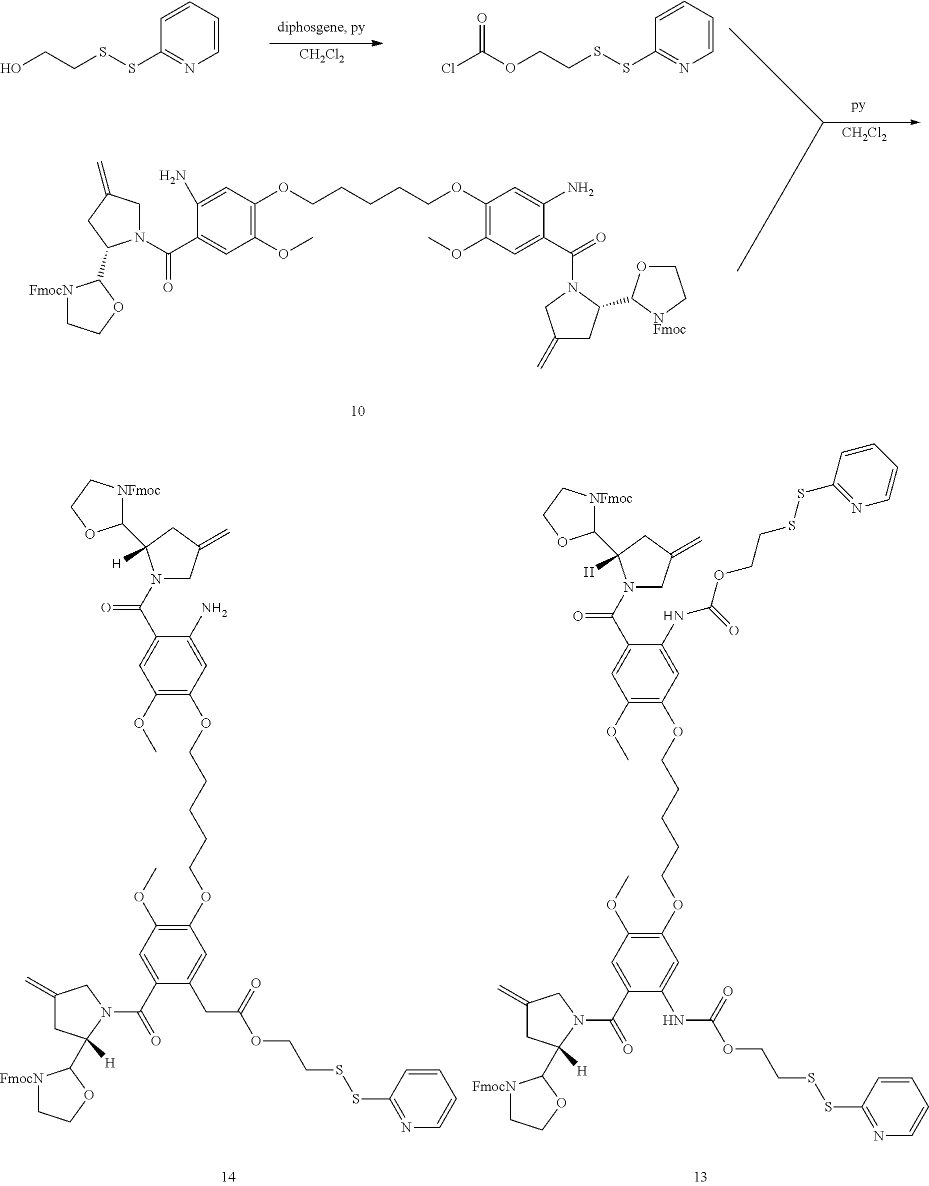

Compound 13 and Compound 14 were synthesized by following the procedure for Compound 12 and Compound 11 from 2-(2-Pyridyldithio)ethanol in lieu of Compound 1. Compound 14: LC/MS (ESI-QMS): m/z=1364 (M+H); Compound 13: LC/MS (ESI-QMS): m/z=1578 (M+H).

### Example 8: Preparation of Compound 15

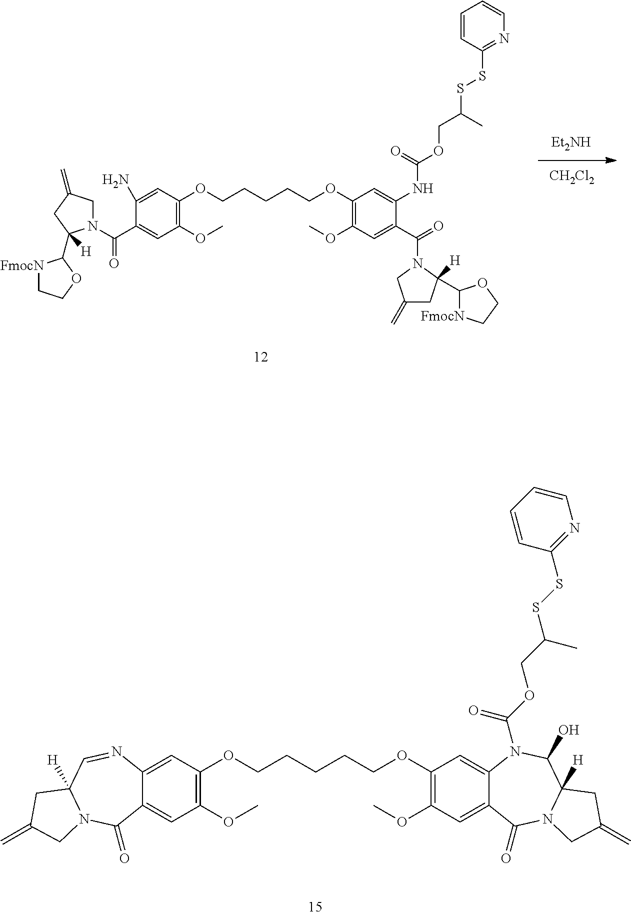

Compound 12 (12.0 mg, 0.0087 mmol) was dissolved in CH₂Cl₂ (1 mL) and treated with diethylamine (0.25 mL, 2.42 mol) at room temperature under argon. The reaction was stirred for 30 min and concentrated in vacuo. The crude product Compound 15 was used without any further purification; LC/MS (ESI-QMS): m/z=830 (M+H).

### Example 9: Preparation of Conjugate 1

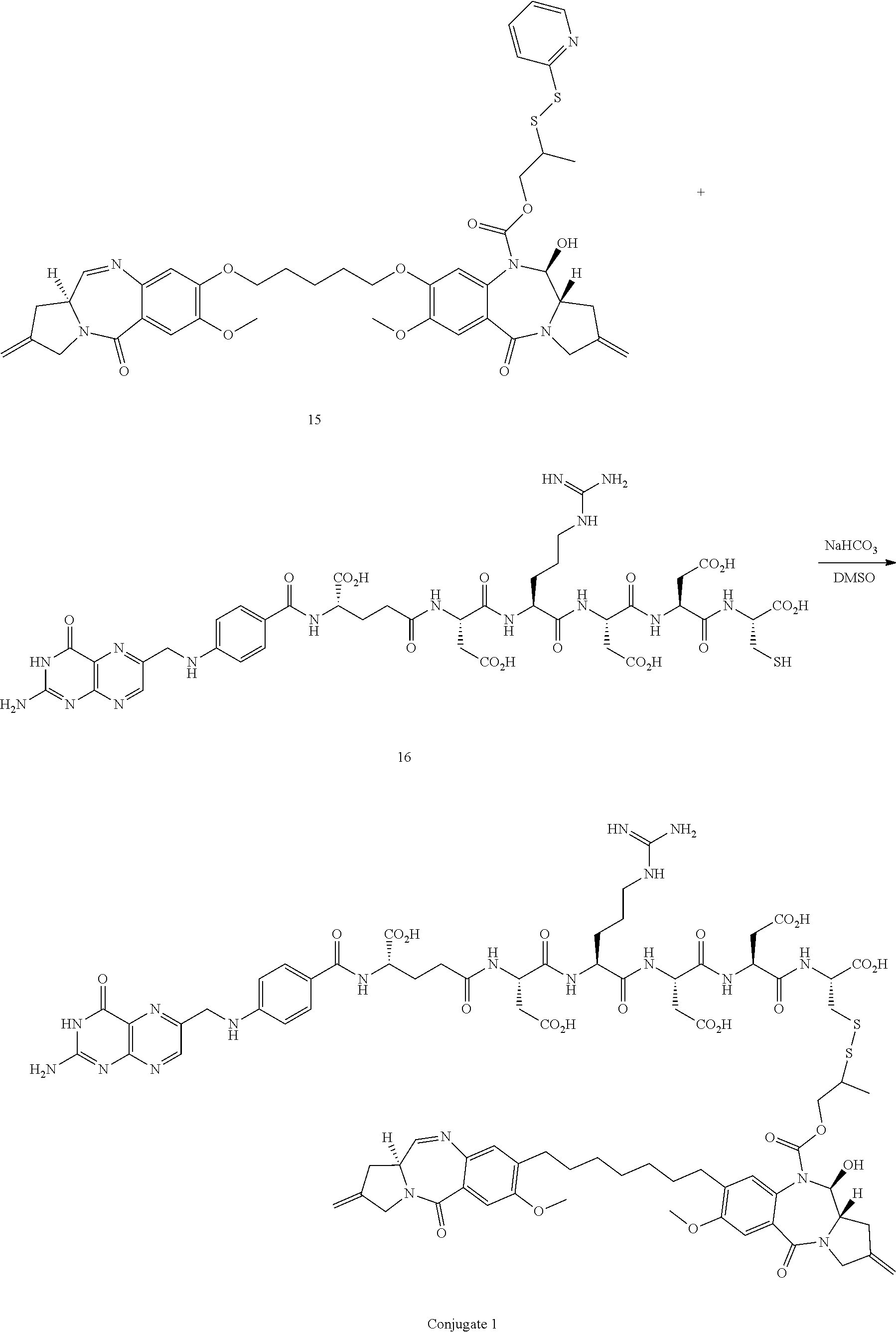

### Step 1: Preparation of Compound 16

Compound 16 is obtainable by the methods disclosed in PCT/US2011/037134 (WO2011146707), incorporated herein by reference.

### Step 2: Preparation of Conjugate 1

Compound 16 (11.4 mg, 0.011 mmol) was dissolved in water (0.5 mL) and the pH was adjusted to 7 with saturated NaHCO₃. Compound 15 (0.0087 mmol) in DMSO (0.2 mL) was added to the reaction mixture and stirred for 1 h at room temperature under argon. The reaction was purified via preparative HPLC (10-100% MeCN/0.1% TFA) to yield Conjugate 1 (1.5 mg, 10% over two steps): LC/MS (ESI-QMS): m/z=1765 (M+H), 883 (M+2H).

### Example 10: Preparation of Compound 18

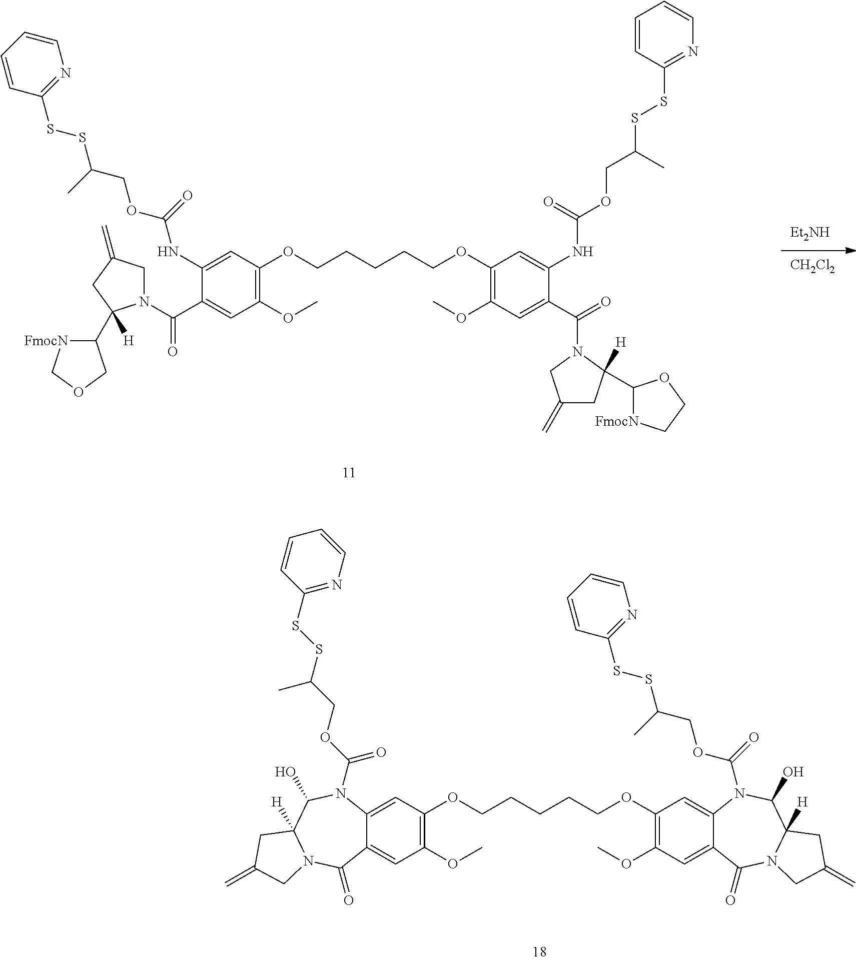

Compound 18 was synthesized by following the procedure for Compound 15 from Compound 11 in lieu of Compound 12: LC/MS (ESI-QMS): m/z=1075 (M+H).

### Example 11: Preparation of Conjugate 2

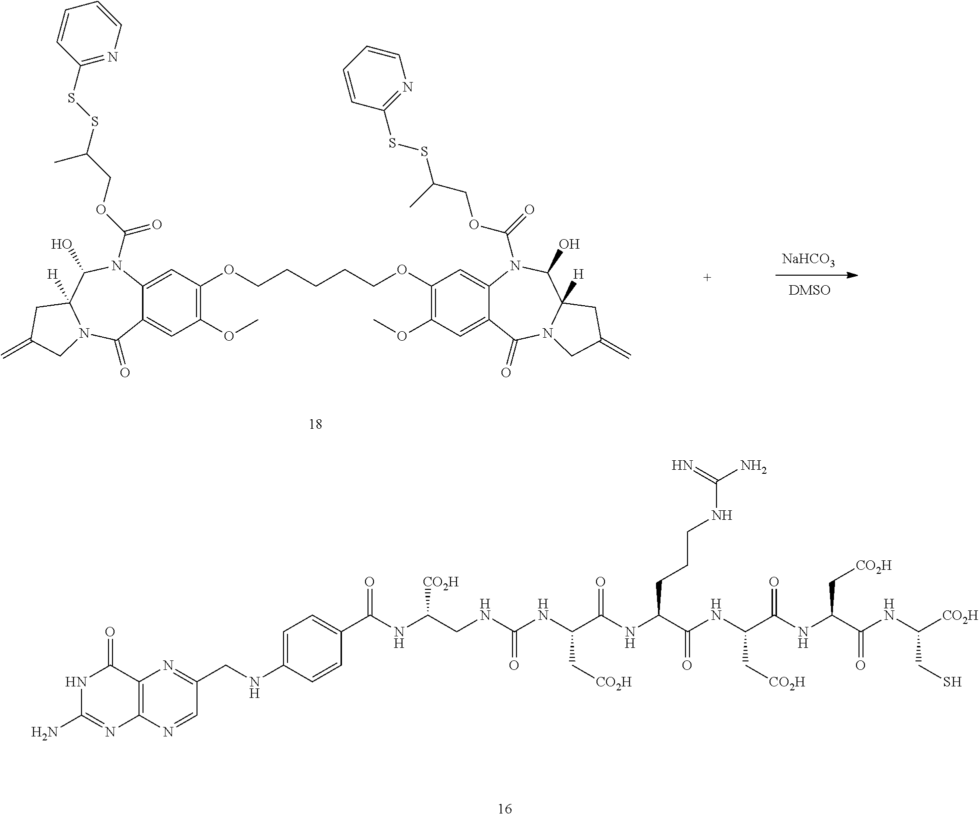

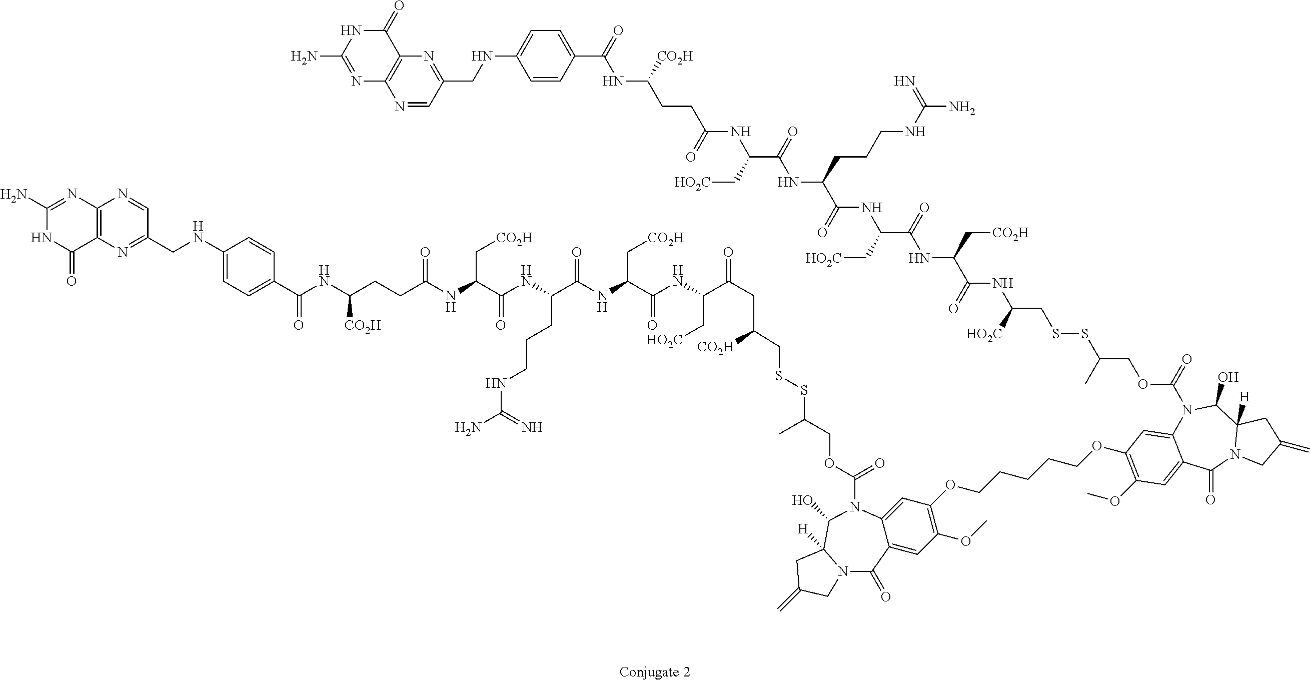 [SMILES: C=C1C[C@H]2[C@H](O)N(C(=O)OCC(C)SSC[C@H](CC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c3ccc(NCc4cnc5nc(N)[nH]c(=O)c5n4)cc3)C(=O)O)C(=O)O)c3cc(OCCCCCOc4cc5c(cc4OC)C(=O)N4CC(=C)C[C@H]4[C@H](O)N5C(=O)OCC(C)SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(=O)O)c(OC)cc3C(=O)N2C1]

Conjugate 2 (9.6 mg, yield 34% over two steps) was synthesized by following the procedure for Compound 17 from Compound 18 in lieu of Compound 15: LC/MS (ESI-QMS): m/z==983 (M+3H), ¹H NMR (500 MHz, DMSO-d₆+ drops of D₂O) δ 8.60 (s, 2H), 7.56 (d, J=6.60 Hz, 4H), 7.01 (s, 2H), 6.82 (br, 2H), 6.60 (d, J=7.70 Hz, 4H), 5.37 (s, 2H), 5.09 (br, 4H), 4.85 (d, J=8.17 Hz, 2H), 4.48 (m, 12H), 4.0-4.3 (m, br, 12H), 3.70 (s, 10H), 3.40 (m, br, 8H), 3.00 (br, 10H), 2.80 (m, 8H), 2.63 (m, 4H), 2.10 (br, 8H), 1.92 (br, 4H), 1.85 (s, 6H), 1.74 (br, 6H), 1.45 (m, br, 14H), 1.12 (m, br, 8H), 0.90 (br, 6H).

### Example 12: Preparation of Conjugate 3

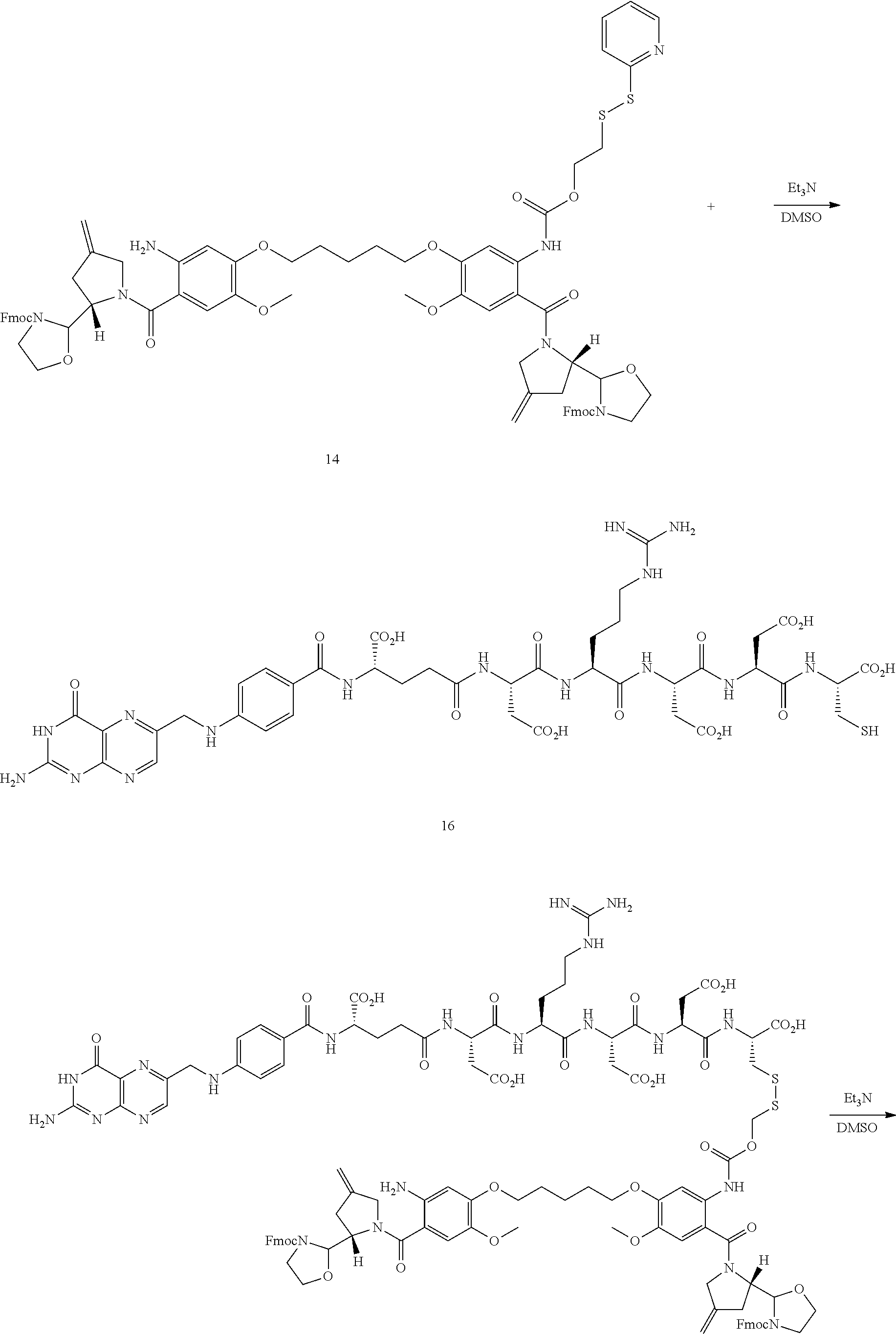

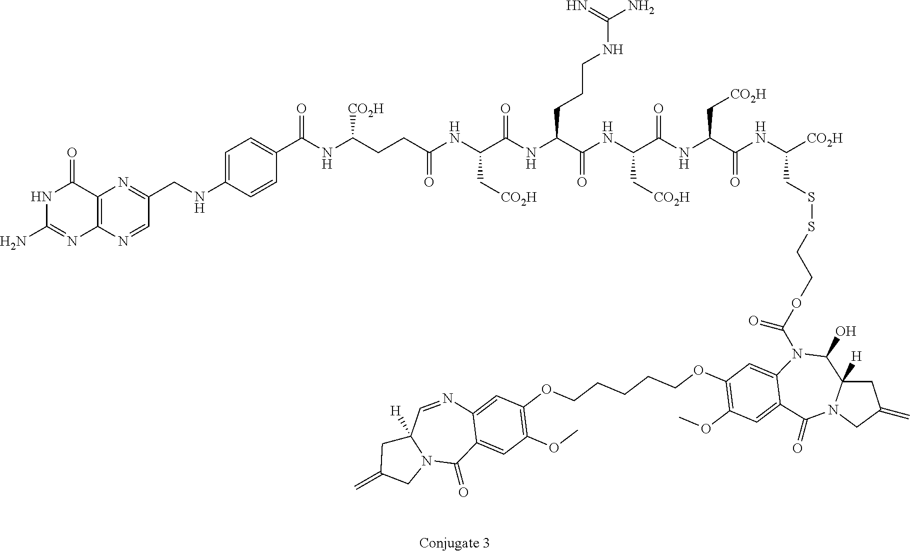 [SMILES: C=C1C[C@H]2C=Nc3cc(OCCCCCOc4cc5c(cc4OC)C(=O)N4CC(=C)C[C@H]4[C@H](O)N5C(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(=O)O)c(OC)cc3C(=O)N2C1]

A solution of Compound 14 (11.2 mg, 0.00820 mmol) in DMSO (0.2 mL) was added to a solution of Compound 16 (8.58 mg, 0.0082 mmol) in DMSO (0.3 mL) at room temperature under argon. The reaction was treated with Et₃N (6.8 μL, 0.049 mmol), and stirred for 1 h. Diethylamine (0.2 mL) was then added, and the reaction mixture was allowed to stir for an additional 30 min before the crude material was purified via preparative HPLC (10-100% MeCN/NH₄HCO₃ buffer, pH 7.4) to yield the desired product (4.8 mg, yield 33% over two steps): LC/MS (ESI-QMS): m/z==876 (M+2H), H NMR (500 MHz, D₂O) δ 8.44 (m, 1H), 7.49 (d, J=8.07 Hz, 2H), 6.98 (m, 2H), 6.75 (br, 1H), 6.55 (d, J=8.44 Hz, 2H), 6.38 (br, 1H), 6.02 (br, 1H), 5.5 (m, 1H), 5.08 (s, 4H), 4.95 (m, 2H), 4.58 (m, 3H), 4.49 (m, 3H), 4.35 (br, 4H), 3.95 (m, 4H), 3.80 (m, 3H), 3.70 (m, 5H), 3.66 (s, 2H), 3.62 (s, 2H), 3.5 (m, 2H), 3.10 (br, 1H), 2.82 (m, br, 6H), 2.50 (m, 4H), 2.29 (m, 3H), 2.03 (m, br, 2H), 1.91 (m, br, 2H), 1.75 (br, 1H), 1.62 (br, 6H), 1.39 (br, 6H).

### Example 13: Preparation of Compound 23

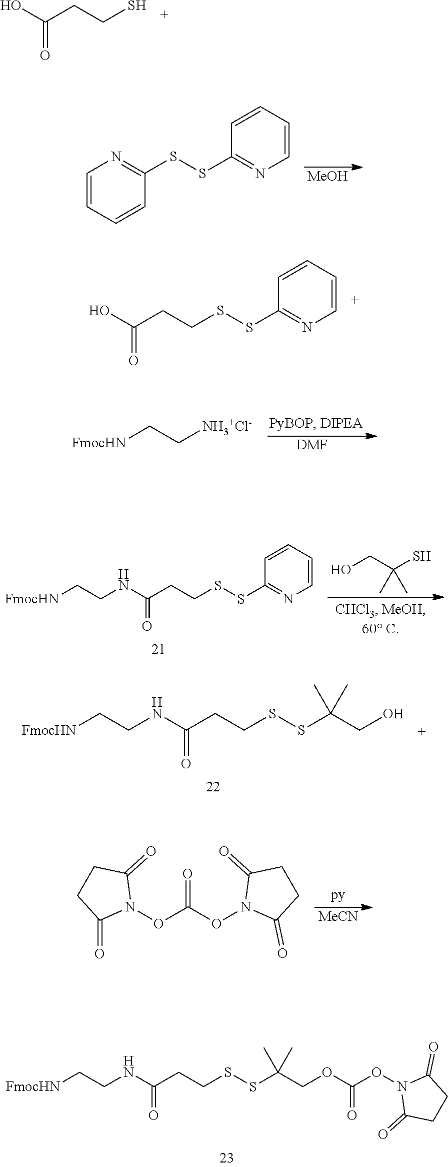

### Step 1: Preparation of 3-(2-Pyridyldithio)propionic acid

2,2′-dipyridyl disulfide (8.70 g, 39.5 mmol) was dissolved in MeOH (150 mL) and purged with argon for 20 minutes. 3-Mercaptopropionic acid (2.10 g, 19.8 mmol) was dissolved in MeOH (35 mL) and purged under argon for 15 min. The 3-mercaptopropionic acid solution was added slowly to the 2,2′-dipyridyl disulfide solution using an addition funnel. The reaction was monitored by LC/MS, and after complete consumption of 3-mercaptopropionic acid, the reaction mixture was concentrated and loaded onto a 120 g C18 column. The purification was carried out with MeCN/H₂O (0-100%). The fractions were analyzed on LC/MS, and fractions containing the desired product were combined and evaporated under reduced pressure. An oil phase was observed on the bottom of the flask during concentration. This oily residue was separated from the aqueous phase and dried under high vacuum to yield the desired product as colorless solid (2.4 g). The aqueous phase was extracted with EtOAc in order to separate additional product. The organic extract was washed with brine, dried over Na₂SO₄, and concentrated in vacuo to yield the desired product (0.5 g). 3-(2-Pyridyldithio)propionic acid was isolated as a white solid (2.9 g, 68%); LC/MS (ESI-QMS): m/z=216.25 (M+H), H NMR (CD₃OD): 8.39 (m, 1H), 7.84 (m, 1H), 7.79 (m, 1H), 7.21 (m, 1H), 4.87 (br, 1H), 3.03 (t, J=6.8 Hz, 2H), 2.70 (t, J=6.8 Hz, 2H). ¹³C NMR (CD₃OD): 173.53, 159.82, 148.97, 137.74, 120.99, 119.81, 33.50, 32.96.

### Step 2: Preparation of Compound 21

To a solution of N-Fmoc-ethylenediamine hydrochloride (500 mg, 1.57 mmol), 3-(2-Pyridyldithio)propionic acid (338 mg, 1.57 mmol), and ⁱPr₂NEt (839 uL, 4.71 mmol) in DMF (7.85 mL) was added PyBOP (950 mg, 1.57 mmol) in one portion. The reaction mixture was stirred for 5 min at room temperature and then concentrated under high vacuum. Water was added to the crude mixture (50 mL) and extracted with ethyl acetate (3×30 mL). The combined organic layers were dried over sodium sulfate, filtered, and evaporated to dryness to yield a pale yellow oil. The product was further purified via silica chromatography (0-80% EtOAc/pet. ether). The product was isolated as a white solid with 86% purity according to HPLC (633 mg, 84.1%): LC/MS (ESI-QMS): m/z=480.56 (M+H), ¹H NMR (500 MHz, CDCl₃) δ 8.44 (d, J=4.9, 1H), 7.75 (d, J=7.3, 2H), 7.59 (m, 3H), 7.40 (t, J=7.3, 2H), 7.30 (t, J=7.3, 2H), 7.09 (t, J=5.9, 1H), 6.98 (s, 1H), 4.56 (d, J=6.8, 2H), 4.17 (t, J=6.8, 1H), 3.43 (m, 2H), 3.40 (m, 2H), 3.08 (t, J=6.4, 2H), 2.60 (t, J=6.4, 2H).

### Step 3: Preparation of Compound 22

In a dry flask, Compound 21 (318 mg, 0.664 mmol, 1.0 equiv.) and 2-mercapto-2-methyl-propan-1-ol (92 mg, 0.863 mmol, 1.3 equiv.) were dissolved in CHC₃:MeOH (1:3, 20 mL). The reaction mixture was stirred for 4 h at 60° C. and monitored until completion by LC/MS. The solvent was removed under reduced pressure to yield an oily residue, followed addition of water and subsequent extractions with EtOAc (3×). The organic extracts were combined, dried over Na₂SO₄, filtered, and concentrated under reduced pressure. The product was further purified using silica gel chromatography (CH₂Cl₂/MeOH, 0-4%) to yield Compound 22 (285 mg, 90%): LC/MS (ESI-QMS): m/z=475.18 (M+H), ¹H NMR (500 MHz CDCl₃) δ 7.78 (d, J=7.3 Hz, 2H), 7.67 (d, J=7.3 Hz, 2H), 7.40 (dd, J=14.7, 7.9 Hz, 2H), 7.32 (dd, J=14.7, 7.9 Hz, 2H), 6.38 (s, 1H), 5.35 (s, 1H), 4.40 (d, J=6.9 Hz, 2H), 4.21 (dd, J=13.7, 6.8 Hz, 1H), 3.47 (s, 2H), 3.42-3.31 (m, 4H), 2.82 (t, J=6.9 Hz, 2H), 2.58 (t, J=6.9 Hz, 2H), 1.25 (s, 6H).

### Step 4: Preparation of Compound 23

To a suspension of Compound 22 (0.552 mg, 1.16 mmol) in dry MeCN (12 mL) under argon was added N,N′-disuccinimidyl carbonate (0.358 g, 1.40 mmol) and pyridine (0.118 mL, 1.45 mmol) respectively. The reaction was allowed to stir at for 15 h room temperature in which the reaction turned into clear solution. LC/MS analysis confirmed that the reaction went to completion. The reaction mixture was concentrated and purified via silica chromatography (0-5% CH₂Cl₂/MeOH) to yield Compound 23 (0.68 g, 95%): LC/MS (ESI-QMS): m/z=616.24 (M+H), H NMR (500 MHz, CD3OD) δ 7.79 (d, J1=7.5 Hz, 2H), 7.64 (d, J1=7.0 Hz, 2H), 7.38 (dd, J1=8.0 Hz, J2=7.5 Hz, 2H), 7.30 (dd, J1=7.0 Hz, J2=7.5 Hz, 2H), 4.33 (d, J1=7.0 Hz, 2H), 4.28 (s, 2H), 4.19 (t, J1=7.0 Hz, J2=6.5 Hz, 1H), 3.20-3.30 (m, 4H), 2.91 (t, J1=7.0 Hz, J2=7.0 Hz, 2H), 2.80 (s, 4H), 2.56 (t, J1=7.5 Hz, J2=7.5 Hz, 2H), 1.31 (s, 6H); ¹³C NMR (125 MHz, CD3OD) δ 172.41, 169.81 (2C), 157.60, 151.59, 143.92 (2C), 141.19 (2C), 127.37 (2C), 126.74 (2C), 124.79 (2C), 119.53 (2C), 75.90, 66.40, 48.39 (2C), 39.83, 39.05, 35.58, 35.12, 24.98 (2C), 23.05 (2C).

### Example 14: Preparation of Compound 26

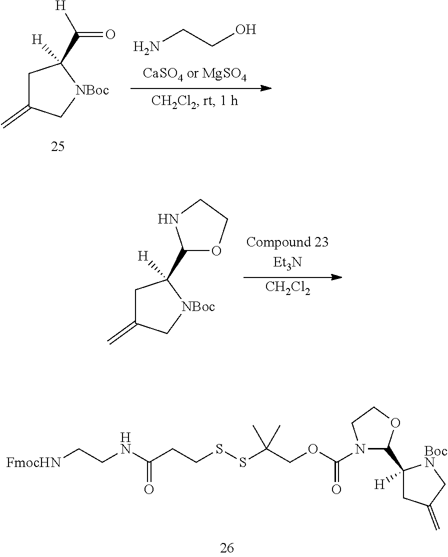

To a solution of the N-Boc-4-methylene-L-prolinal (44.36 mg, 0.2099 mmol) in dry CH₂Cl₂ (1 mL) was added anhydrous CaSO₄ (22 mg, 0.16 mmol) and ethanolamine (10.56 μL, 0.1750 mmol) respectively. The reaction was allowed to stir for 1 h at room temperature. In another flask, Compound 23 (108 mg, 0.180 mmol) was dissolved in dry CH₂Cl₂ (1 mL). The previous pyrrolidine solution was filtered and slowly added to the Compound 23 solution.

Et₃N (0.037 mL, 0.26 mmol) was added to the reaction mixture, and the resulting mixture was monitored via LC/MS. After stirring for 2 h, the reaction mixture was diluted with CH₂C₂, washed with sat. NH₄Cl_{(aq)}, dried over Na₂SO₄, and concentrated in vacuo. The residue was further purified silica chromatography (0-10% CH₂C₂/MeOH) to yield pure Compound 26 (83 mg, 63%): LC/MS (ESI-QMS): m/z=755.38 (M+H), ¹H NMR (500 MHz, CD₃OD) δ 7.79 (d, J1=8.0 Hz, 2H), 7.64 (d, J1=7.5 Hz, 2H), 7.38 (dd, J1=7.5 Hz, J2=7.5 Hz, 2H), 7.30 (dd, J1=7.5 Hz, J2=7.5 Hz, 2H), 5.13-5.20 (m*, 1H), 4.88-5.05 (m*, 2H), 4.36-4.60 (m*, 1H), 4.33 (d, J1=7.0 Hz, 2H), 4.20 (t, J1=7.0 Hz, J2=7.0 Hz, 1H), 3.98-4.10 (m*, 3H), 3.72-3.94 (m*, 4H), 3.36-3.50 (m*, 1H), 3.18-3.30 (m*, 4H), 2.91 (t, J1=7.5 Hz, J2=7.0 Hz, 2H), 2.70-2.40 (m*, 2H), 2.54 (t, J1=7.0 Hz, J2=7.0 Hz, 2H), 1.40-1.50 (m*, 9H), 1.26-1.38 (m*, 6H).

* Due to diasteromeric and/or rotameric nature of the compound

### Example 15: Preparation of Compound 28

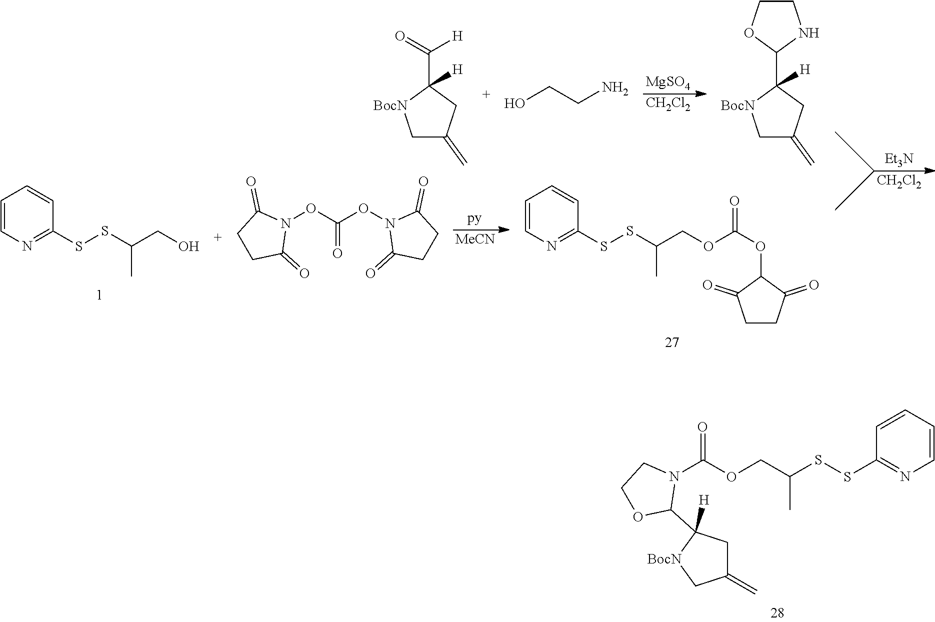

Compound 28 was synthesized by following the procedure for Compound 26 from Compound 27 in lieu of Compound 22: LC/MS (ESI-QMS): m/z=482 (M+H).

### Example 16: Preparation of Compound 29

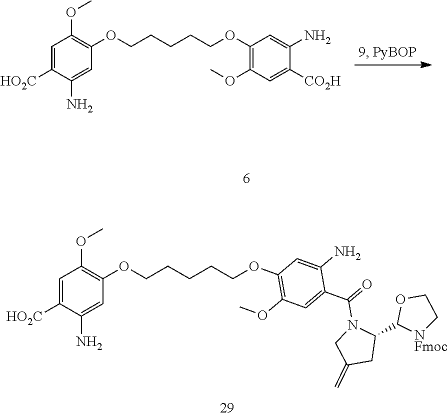

Compound 6 (42.0 mg, 0.097 mmol), Compound 9 (0.053 mmol), and PyBOP (29.0 mg, 0.056 mmol) were dissolved in DMF/DCM (0.5 mL/0.5 mL) and treated with DIPEA (74 μL, 0.43 mmol) at r.t. under Ar. The reaction was completed within 1 hr, then loaded onto CombiFlash column in 0-20% MeOH/DCM to afford the pure product Compound 29 (25.5 mg, 60%). LCMS: [M+H]+m/z=793.

### Example 17: Preparation of Compound 30

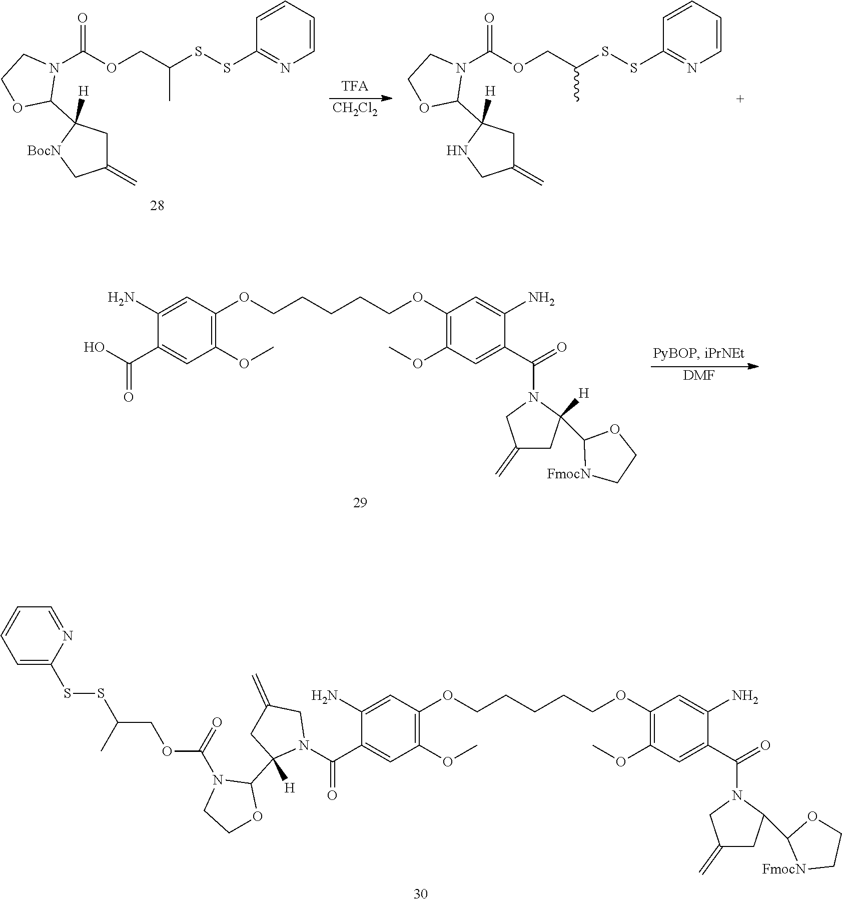

Compound 28 (26.1 mg, 0.0551 mmol) was added to TFA/CH₂Cl₂ (0.5 mL/0.5 mL) at and stirred for 30 min at room temperature. Then the solvent was removed in vacuo, and the residue was dissolved in CH₂Cl₂ (0.5 mL) and added to a solution of Compound 29 (43.6 mg, 0.0551 mmol) in DMF (0.3 mL). The reaction mixture was treated with PyBOP (47.77 mg, 0.0918 mmol) and ⁱPr₂NEt (31.98 μL, 0.184 mmol). The reaction was stirred at room temperature under argon for 2 h. The reaction mixture was then concentrated and purified via silica chromatography (0-10% MeOH/CH₂C₂) to yield Compound 30 (55.2 mg, 86%): LC/MS (ESI-QMS): m/z=1157 (M+H).

### Example 18: Preparation of Conjugate 4

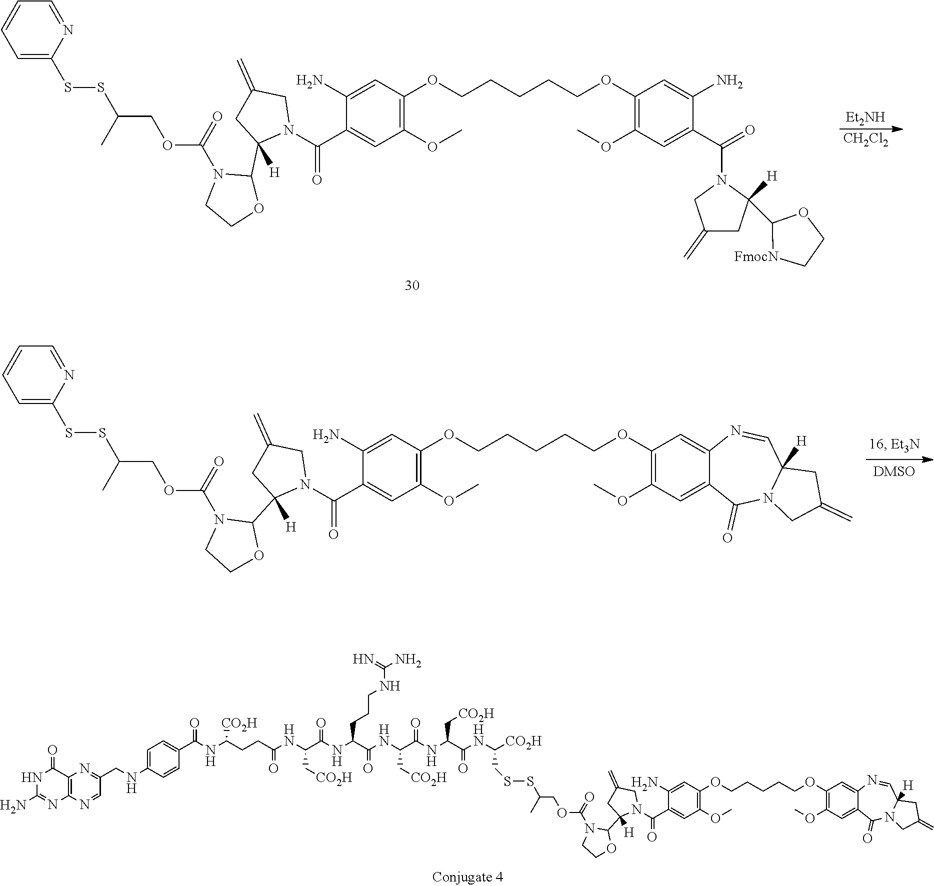

Compound 30 (27.6 mg, 0.0239 mmol) was dissolved in CH₂Cl₂ (0.5 mL) and treated with diethylamine (0.15 mL, 1.4 mmol) at room temperature under argon for 3 h. The reaction mixture was evaporated to dryness and dissolved in DMSO (0.5 mL). The resulted solution was added to the solution of Compound 16 (25.0 mg, 239 mmol) and Et₃N (20 μL, 140 mmol) in DMSO (2 mL) at room temperature under argon for 1. The product was purified with preparative HPLC (10-100% MeCN/NH₄HCO₃ buffer pH 7.4) to yield Conjugate 4 (8.1 mg, yield 19% over two steps). LC/MS (ESI-QMS): m/z=905 (M+2H), H NMR (500 MHz, D₂O+one drop of DMSO-d₆, the major fraction, the selected data) δ 8.59 (br, s, 1H), 7.55 (br, 2H), 7.03 (br, 1H), 6.65 (br, m, 3H), 6.50 (m, br, 1H), 6.35 (br, 1H), 5.00 (m, 6H).

### Example 19: Preparation of Compound 32

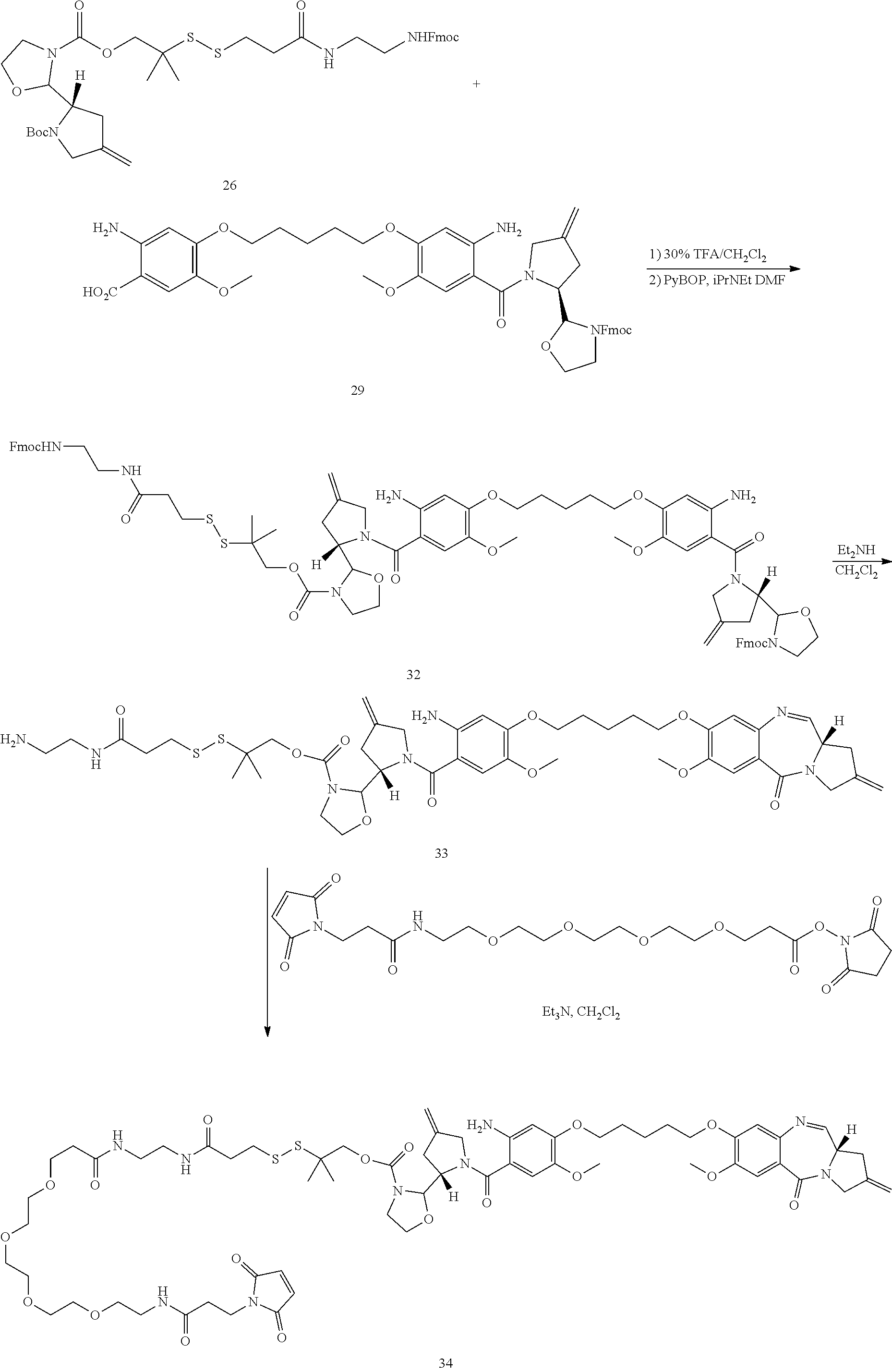

### Step 1: Preparation of Compound 32

In a flask, Compound 26 (95.0 mg, 0.126 mmol) was dissolved in 30% TFA/CH₂Cl₂ (10 mL) at 0° C. The reaction mixture was allowed to warm to room temperature and stirred for 1 h. Upon complete removal of the Boc protecting group, the solvent was removed under reduced pressure, and the crude residue was left under high vacuum for 3 h. In a dry flask, the crude TFA salt and Compound 29 (100 mg, 0.126 mmol) were dissolved in dry DMF (2.5 mL) under argon. To the reaction mixture was added PyBOP (131 mg, 0.252 mmol) and ⁱPr₂NEt (67 μl, 0.378 mmol) subsequently. After 3 h, the reaction was quenched by the addition of sat. NH₄Cl_{(aq)} and extracted with EtOAc (3×). The combined organic layers were dried over Na₂SO₄, filtered, and concentrated under reduced pressure. The product was purified using silica gel chromatography (0-8% MeOH/CH₂C₂) to yield Compound 32 (153 mg, 84.9%): LC/MS (ESI-QMS): m/z=1429.78 (M+H), ¹H NMR (500 MHz CDCl₃) δ Pivotal signals: δ 7.75-7.66 (m, 4H), 7.58-7.47 (m, 4H), 7.75-7.66 (m, 4H), 7.39-7.31 (m, 4H), 7.29-7.22 (m, 4H), 7.02-6.51 (m, 4H), 5.31-5.14 (m, 1H), 5.04-4.74 (m, 5H), 1.28-1.12 (m, 6H).

### Step 2: Preparation of Compound 33

Compound 32 (80.0 mg, 0.0559 mmol) in CH₂Cl₂ (2 mL) was treated with diethylamine (0.5 mL) at room temperature under argon. The reaction mixture was stirred for 1 h and concentrated in vacuo. The product Compound 33 was used in the next step without further purification: LC/MS (ESI-QMS): m/z=924, 925 (M+H).

### Step 3: Preparation of Compound 34

Compound 33 (0.0559 mmol) and Mal-PEG4-NHS ester (38.7 mg, 0.0754 mmol) in CH₂Cl₂ (3.5 mL) was treated with Et₃N (7.8 μL, 0.0559 mmol) at room temperature under argon. The reaction was monitored via LC/MS and went to completion within 3 h. The solvent was removed in vacuo, and the crude product Compound 34 was dissolved in DMSO (2 mL) for the conjugation. LC/MS (ESI-QMS): m/z=1323 (M+H).

### Example 20: Preparation of Compound 35 and 36

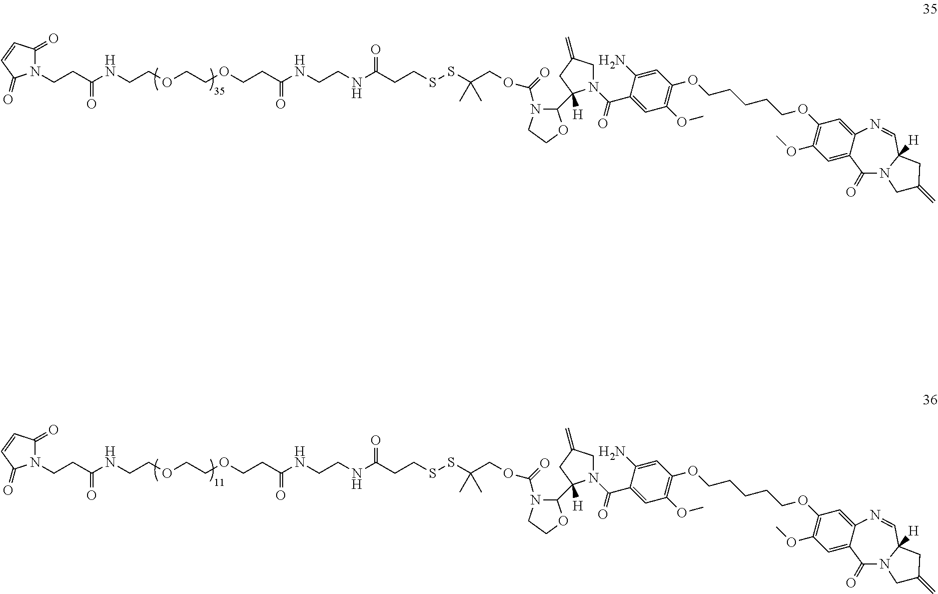

Compound 35 and Compound 36 were synthesized in the same method as for Compound 34. Compound 35: LC/MS (ESI-QMS): m/z=1367 (M+2H); Compound 36: LC/MS (ESI-QMS): m/z=838.7 (M+2H), 1676 (M+H). Mal-PEG4-NHS ester, Mal-PEG12-NHS ester, and Mal-PEG36-NHS ester were obtained from Quanta BioDesign Ltd.

### Example 21: Preparation of Conjugate 5

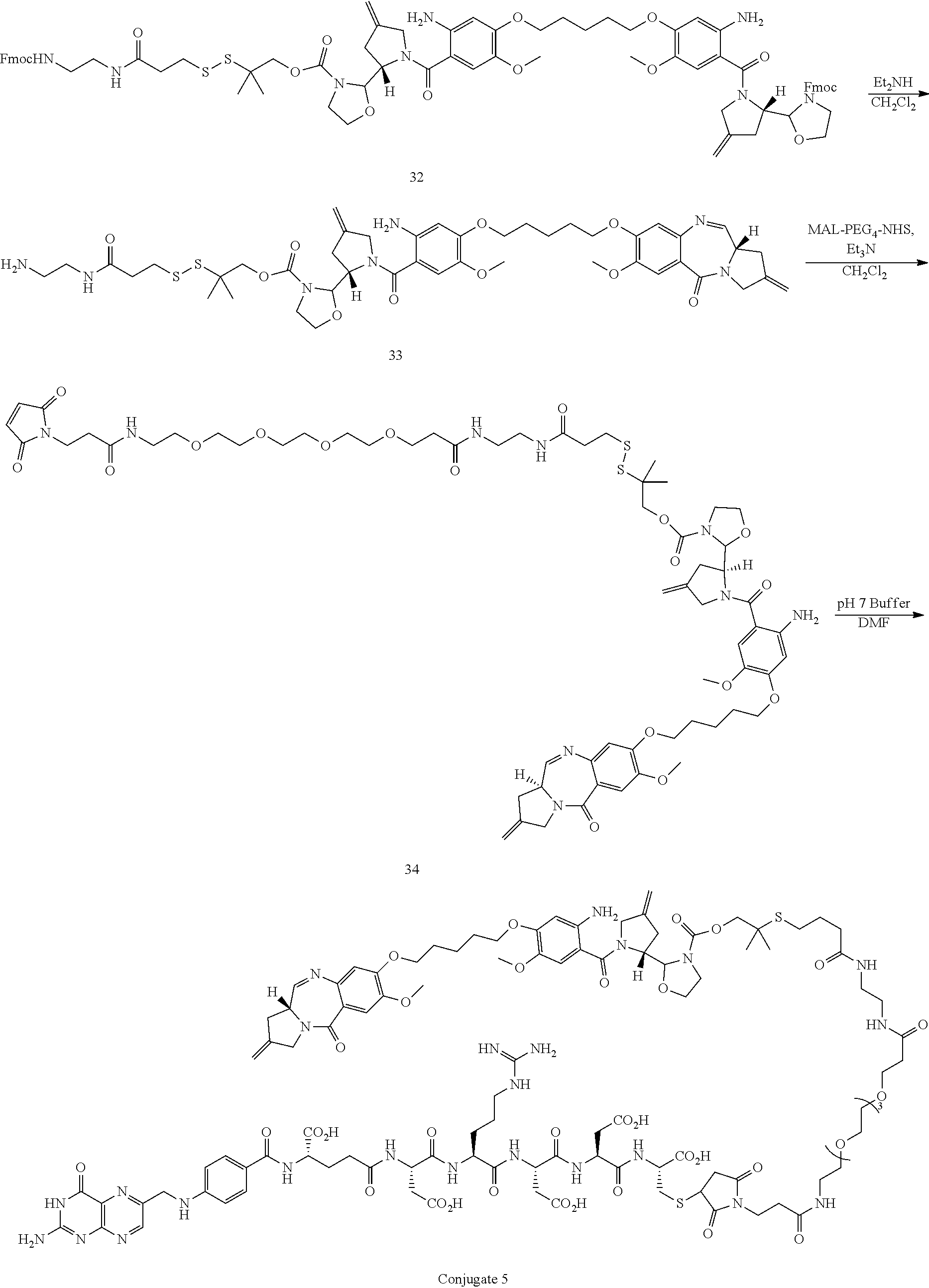

Compound 32 (23 mg, 0.016 mmol) and diethylamine (0.25 mL, 2.4 mmol) were dissolved in CH₂Cl₂ (0.6 mL), and the reaction mixture was stirred at room temperature under argon for 3 h. The reaction was monitored via LC/MS and after complete consumption of Compound 32, the solvent was removed under reduced pressure. The resulting residue was co-evaporated with CH₂Cl₂ twice and dried under high vacuum for 15 minutes. The resulting residue was dissolved in CH₂Cl₂ (0.5 mL), and Mal-PEG4-NHS ester (10.9 mg, 0.021 mmol) and Et₃N (3.0 μL, 0.021 mmol) were added. The reaction was stirred at room temperature under argon and monitored via LC/MS for production of Compound 34 (m/z=1323 and 662). After 1 h, the reaction mixture was evaporated, and the resulting residue was dissolved in DMF (2 mL). The solution was purged with argon. Compound 16 (22 mg, 0.021 mmol) was dissolved in pH 7 buffer (2 mL, 50 mM NH₄HCO₃), purged with argon, and added to the above Compound 34 solution. The reaction was stirred at room temperature while purging with argon. The reaction was monitored via LC/MS for the production of Conjugate 5 (m/z=791). After 2 hours, purification via preperative HPLC (10-100% MeCN/50 mM NH₄HCO₃ pH 7 buffer) yielded two sets of isomers: 1.9 mg of 1^{st} set of isomers with a shorter retention time and 7.4 mg of 2^{nd} set of isomers with a longer retention time. The desired product was obtained in a yield of 24% over three steps: LC/MS (ESI-QMS): m/z=791.25 (M+3H), Major Product: H NMR (DMSO-D6, selected data): 8.61 (s, 1H), 7.72 (d, NH), 7.55 (d, J=8.8 Hz, 2H), 7.30 (s, NH), 7.15 (s, ArH), 7.01 (s, ArH), 6.81 (s, NH), 6.60 (d, J=8.8 Hz, 2H+1H overlapped), 6.54 (s, ArH), 6.34 (s, N═CH), 6.32 (s, ArH), 5.11+5.06 (m, 2H), 4.96+4.92+4.85 (m, 3H), 3.66+3.62 (s+s, 3H), 3.61 (s, 3H), 3.55 (t, 3H), 3.35 (t, 3H), 1.21 (s, br, 6H). Minor Product: H NMR (DMSO-D6, selected data): 8.61 (s, 1H), 7.72 (d, NH), 7.55 (d, J=8.8 Hz, 2H), 7.29 (s, NH), 7.15 (s, ArH), 7.01 (s, ArH), 6.80 (s, NH), 6.60 (d, J=8.8 Hz, 2H+1H overlapped), 6.53 (s, ArH), 6.32 (s, N═CH), 6.31 (s, ArH), 5.11+5.06 (m, 2H), 4.94-4.85 (m, 3H), 3.66+3.62 (s+s, 3H), 3.61 (s, 3H), 3.55 (t, 3H), 3.35 (t, 3H), 1.20 (s, br, 6H).

### Example 22: Preparation of Conjugate 6

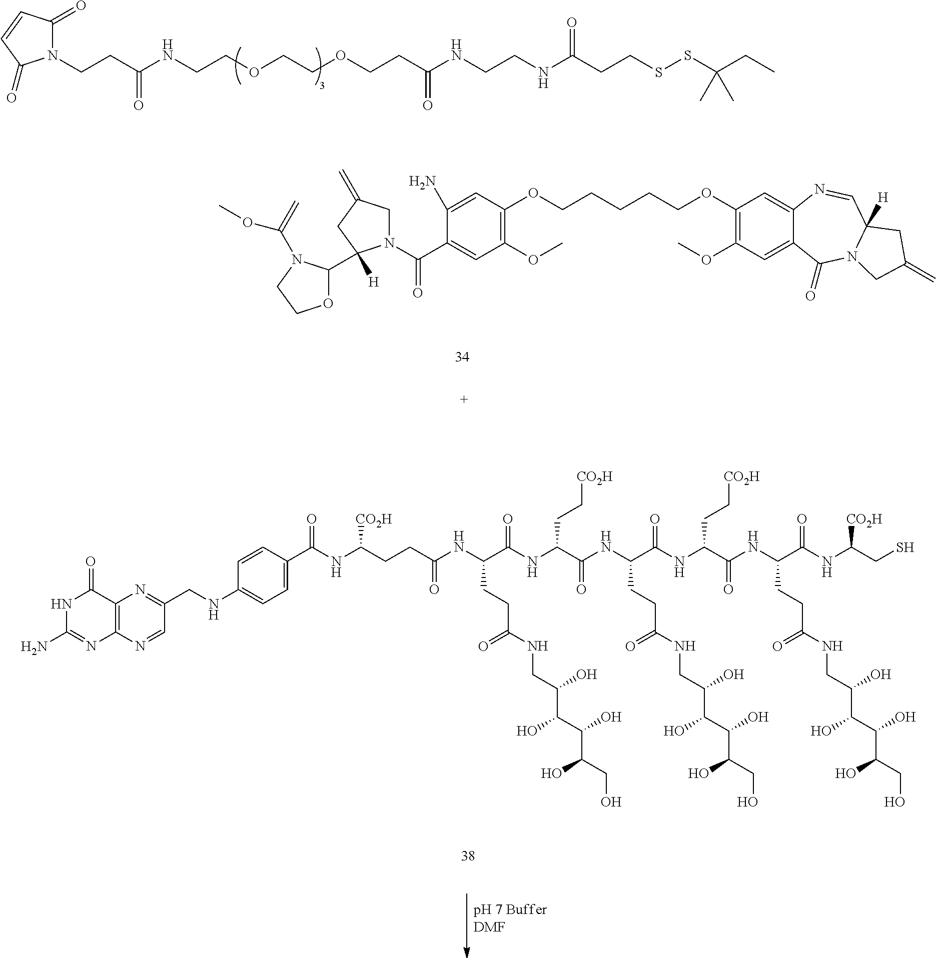

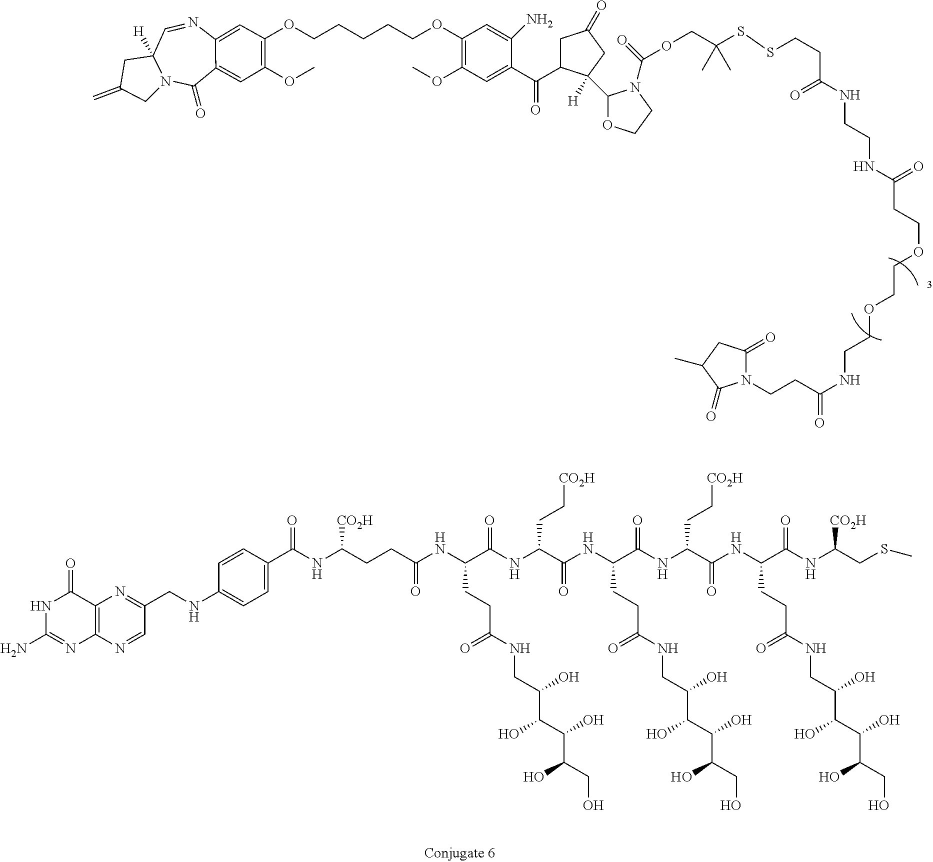

Conjugate 6 was synthesized by following the procedure for Conjugate 5 from Compound 34 in lieu of Compound 32: LC/MS (ESI-QMS): m/z=1502 (M+2H), 1001 (M+3H): ¹H NMR (500 MHz, DMSO-d₆+ drops of D₂₀₎ 6 The major fraction: 8.61 (s, 1H), 7.58 (d, J=8.32 Hz, 2H), 7.12 (s, 1H), 7.00 (s, 1H), 6.61 (d, J=8.31 Hz, 2H), 6.50 (s, 1H), 6.30 (m, 2H), 5.00 (m, 6H), 4.50 (m, 3H), 4.13 (m, br, 13H), 3.63 (s, 3H), 3.59 (m, 8H), 3.51 (m, 11H), 3.43 (m br, 15H), 3.35 (m, 9H), 3.20 (m, br, 5H), 3.15 (m, br, 3H), 3.03 (m, br, 9H), 2.80 (br, 4H), 2.61 (br, 2H), 2.40 (br, m, 6H), 2.26 (m, 4H), 2.10 (m, br, 11H), 1.90 (m, br, 8H), 1.74 (br m, 9H), 1.50 (br, 3H), 1.20 (m, br, 10H), The minor fraction: 8.60 (s, 1H), 7.59 (d, J=8.31 Hz, 2H), 7.11 (s, 1H), 7.00 (s, 1H), 6.62 (d, J=8.31 Hz, 2H), 6.50 (s, 1H), 6.29 (m, 2H), 5.08 (m, 2H), 4.90 (m, 4H), 4.50 (m, 3H), 4.00 (m, 12H), 3.65 (s, 3H), 3.59 (m, 8H), 3.53 (m, 12H), 3.49 (m, br, 17H), 3.35 (m, 10H), 3.20 (br, m, 6H), 3.10 (m, br, 3H), 3.08 (m, br, 10H), 2.78 (br, m, 4H), 2.39 (m, br, 5H), 2.25 (br, m, 5H), 2.15 (br, 6H), 2.10 (br, 7H), 1.93 (br, m, 5H), 1.85 (s, 5H), 1.73 (br, m, 7H), 1.50 (br, 3H), 1.25 (br, m, 8H).

### Example 23: Preparation of Conjugate 7 and Conjugate 8

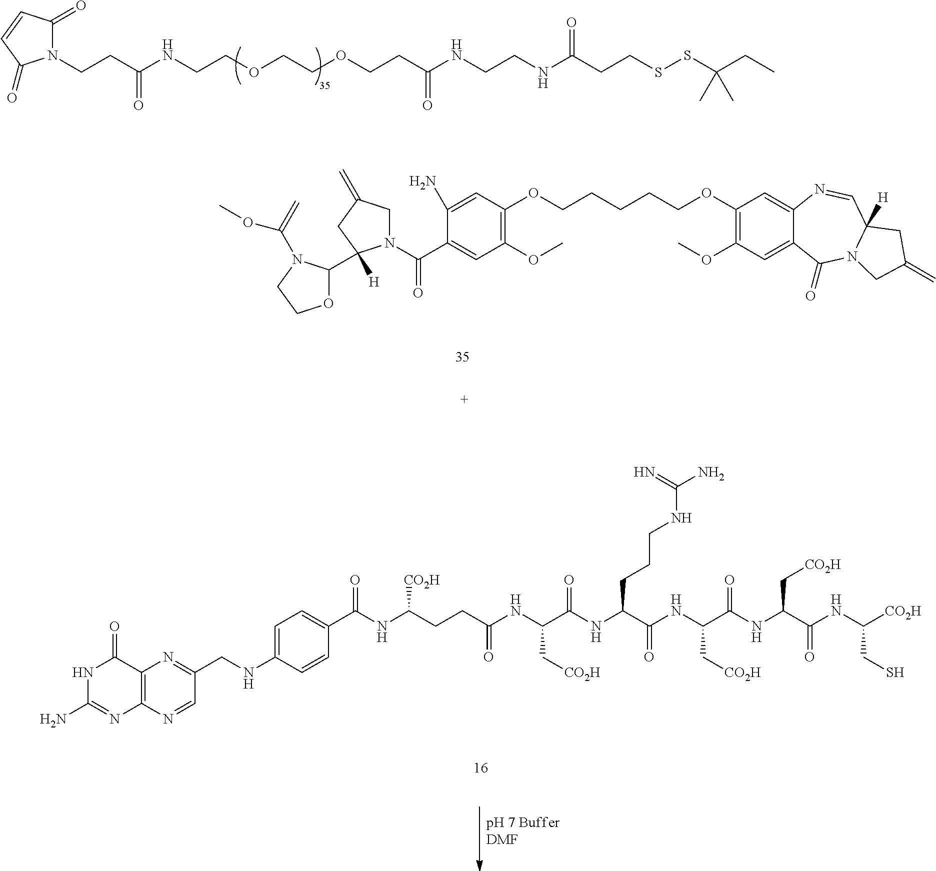

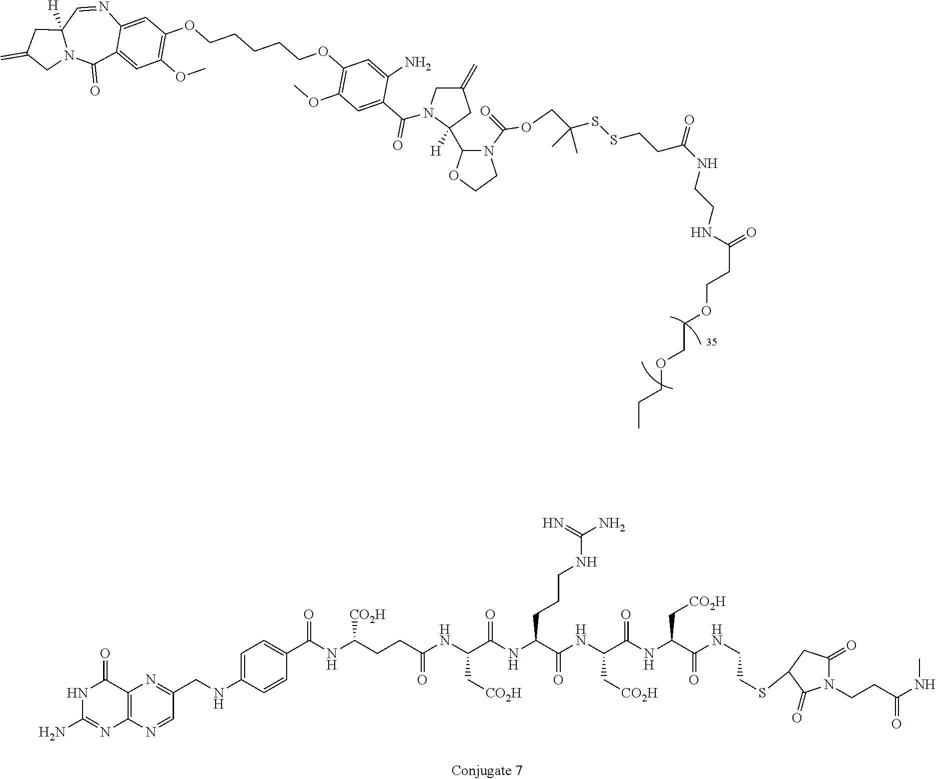

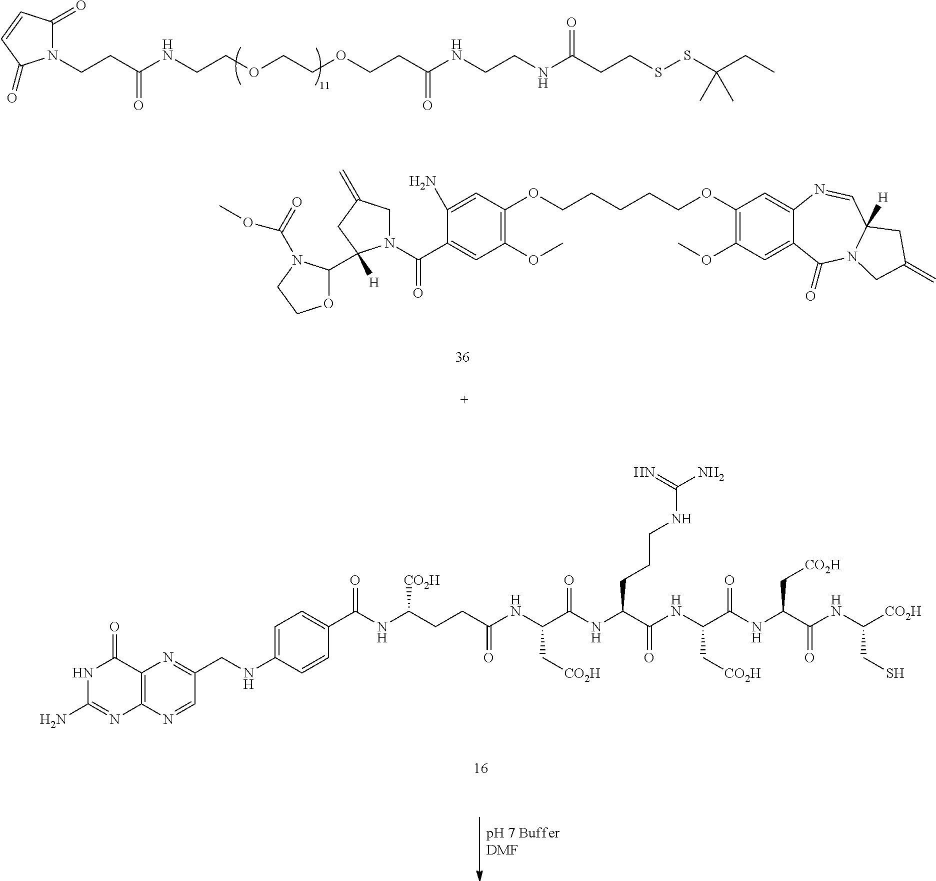

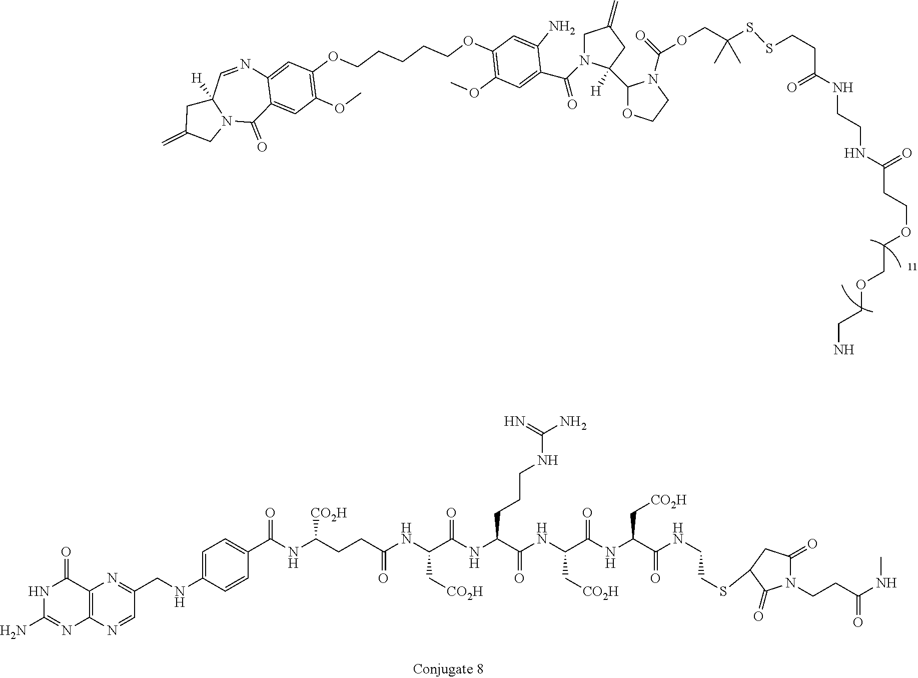

Conjugate 7 and Conjugate 8 were synthesized by following the procedure for Conjugate 5 from Compound 35 and Compound 36 respectively in lieu of Compound 32. Conjugate 7: LC/MS (ESI-QMS): m/z=1260 (M+3H), ¹H NMR (500 MHz, DMSO-d₆+ drops of D₂O, the major fraction) δ 8.61 (s, 1H), 7.51 (d, J=8.31 Hz, 2H), 7.12 (s, 1H), 7.00 (s, 1H), 6.60 (d, J=8.32 Hz, 2H), 6.50 (s, 1H), 6.32 (m, 2H), 5.00 (m, br, 6H), 4.50 (m, br, 7H), 4.00 (m, br, 20H), 3.60 (m, 4H), 3.50 (br, 134H), 3.30 (m, 2H), 3.13 (m, 2H), 3.05 (s, br, 5H), 2.95 (m, 1H), 2.80 (m, 3H), 2.62 (s, 2H), 2.39 (m, 5H), 2.24 (m, 5H), 2.04 (m, 2H), 1.89 (m, 2H), 1.79 (m, 4H), 1.67 (m, 1H), 1.50 (br, m, 4H), 1.20 (m, 8H) Conjugate 8: LC/MS (ESI-QMS): m/z=908 (M+3H), H NMR (500 MHz, DMSO-d₆+ drops of D₂O, the major fraction) δ 8.61 (s, 1H), 7.58 (d, J=8.31 Hz, 2H), 7.12 (s, 1H), 7.00 (s, 1H), 6.62 (d, J=8.80 Hz, 2H), 6.50 (s, 1H), 6.30 (m, 2H), 5.00 (m, 6H), 4.50 (m, 5H), 4.35 (m, 1H), 4.15 (m, 8H), 3.65 (s, 3H), 3.60 (m, 5H), 3.55 (m, 5H), 3.47 (s, br, 52H), 3.35 (m, 4H), 3.03 (m, 9H), 2.80 (br, m, 5H), 2.60 (br, m, 5H), 2.40 (m, 6H), 2.27 (m, 5H), 2.13 (br, 2H), 1.90 (m, br, 3H), 1.75 (m, br, 6H), 1.60 (br, m, 7H), 1.20 (br, m, 8H).

### Example 24: Preparation of Compound 42

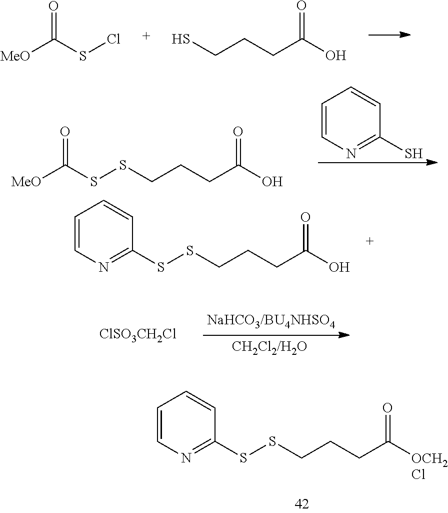

### Step 1: Preparation of 4-(Pyridin-2-yldisulfanyl)butanoic acid

A solution of 2.16 g (18.0 mmol) 4-mercaptobutyric acid in 4 mL THF was added to a solution of 2.33 g (18.4 mmol) methoxycarbonylsulfenyl chloride in 4 mL THF at 0° C. The reaction mixture was stirred at 0° C. for 30 min. Then 2.10 g (18.9 mmol) of 2-mercaptopyridine was added to the reaction mixture at 0° C. The resulting reaction mixture was allowed to warm to room temperature. The reaction was monitored by LC/MS. After the reaction was complete, the solvent was evaporated and the residue was dissolved in dichloromethane. Purification with CH₂C₂/methanol on Combiflash provided product with impurity. The fractions containing the desired product were combined and concentrated under vacuum. The resulting yellow oil was dissolved in CH₂Cl₂ and purified with silica chromatography (petroleum ether/EtOAc) to afford 1.00 g of 4-(pyridin-2-yldisulfanyl)butanoic acid (24%). LC/MS (ESI-QMS): m/z=230.27 (M+H).

### Step 2: Preparation of Compound 42

458 mg (2 mmol) of 4-(pyridin-2-yldisulfanyl)butanoic acid was mixed with NaHCO₃ (672 mg, 8 mmol) and Bu₄NHSO₄ (68 mg, 0.2 mmol) in 8 mL H₂O/8 mL CH₂C₂. The mixture was stirred vigorously at 0° C. for 10 min. Then the solution of 396 mg (2.4 mmol) of chloromethyl chlorosulfate in 2 mL CH₂Cl₂ was added to the above mixture. The reaction mixture was stirred vigorously and warmed up to room temperature. The reaction was monitored with LC/MS. After 2 hours, the organic layer was separated. The aqueous layer was washed with additional CH₂Cl₂. The organic solution was combined and washed with brine and dried over Na₂SO₄. The salt was filtered and the solvent was removed. Purification with petroleum ether/EtOAc on silica chromatography gave 300 mg of chloromethyl ester Compound 42 (54%). LC/MS (ESI-QMS): m/z=278.23 (M+H): ¹H NMR (500 MHz, CDCl3) δ 8.45 (m, 1H), 7.64 (m, 1H), 7.08 (m, 1H), 5.68 (s, 2H), 2.84 (m, 2H), 2.55 (m, 2H), 2.07 (m, 2H). ¹³C NMR (500 MHz, CDCl3) δ 170.85, 159.85, 149.73, 136.97, 120.75, 119.87, 68.60, 37.54, 32.26, 23.52.

### Example 25: Preparation of Compound 43

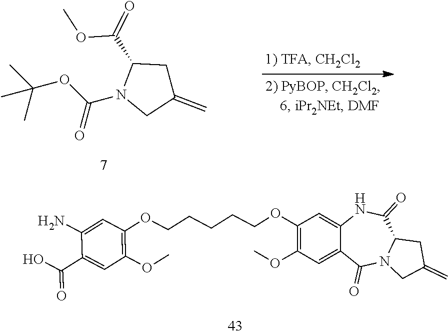 [SMILES: C=C1C[C@@H](C(=O)OC)N(C(=O)OC(C)(C)C)C1 ; C=C1C[C@H]2C(=O)Nc3cc(OCCCCCOc4cc(N)c(C(=O)O)cc4OC)c(OC)cc3C(=O)N2C1]

A solution of Compound 7 (35.3 mg, 0.146 mmol) in TFA (0.50 mL) and CH₂Cl₂ (0.75 mL) was stirred at ambient temperature for 30 min. The reaction mixture was concentrated under reduced pressure, co-evaporated with DCM (1 mL×3), and dried under vacuum for 1 h. The residue was dissolved with PyBOP (76.0 mg, 1.00 equiv.) in anhydrous CH₂Cl₂ (3.0 mL) and the resulting solution was transferred into a solution of Compound 6 (63.4 mg, 1.0 equiv.) in anhydrous DMF (3.0 mL). After addition of iPr₂NEt (0.20 mL, 7.9 equiv.), the reaction mixture was stirred at ambient temperature under argon for 90 min and loaded directly onto a CombiFlash system (silica gel column) eluting with 0-10% MeOH in CH₂Cl₂ to produce 37.5 mg Compound 43 as a white solid. LC/MS (ESI-QMS): m/z=524.29 (M+H).

### Example 26: Preparation of Compound 46

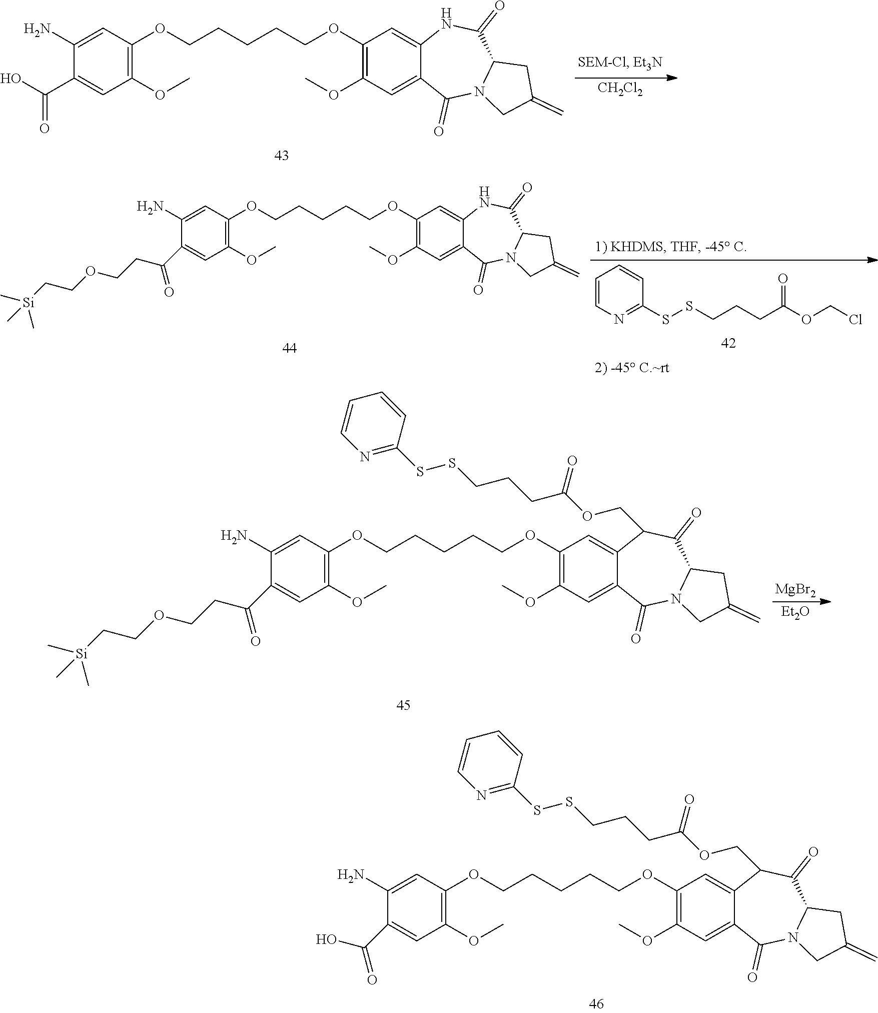

### Step 1: Preparation of Compound 44

2-(Trimethylsilyl)ethoxymethyl chloride (90.0 μL, 0.508 mmol) and Et₃N (50.0 μL, 0.359 mmol) were added in tandem to a solution of Compound 43 (86.7 mg, 0.165 mmol) in anhydrous CH₂Cl₂ (7.0 mL). After stirring at room temperature under argon for 2.5 h, the reaction mixture was concentrated under reduced pressure and purified via silica chromatography (0-70% EtOAc/pet. ether) to yield Compound 44 as a white solid (50.1 mg, 46.3%): LC/MS: (ESI-QMS): m/z=656.53 (M+H), H NMR (500 MHz, 298 K, DMSO-d6) δ 10.258 (s, 1H), 7.236 (s, 1H), 7.153 (s, 1H), 6.706 (s, 1H), 6.452 (s, 2H), 6.380 (s, 1H), 5.078 (s, 2H), 4.260 (m, 2H), 4.022 (m, 2H), 3.977 (m, 3H), 3.763 (m, 5H), 3.682 (s, 3H), 3.214 (d, J=15.0 Hz, 1H), 2.785 (m, 1H), 1.797 (m, 4H), 1.578 (m, 2H), 0.924 (t, J=3.0 Hz, 2H).

### Step 2: Preparation of Compound 45

0.5 M KHMDS in toluene (135 μL, 68.4 μmol) was added dropwise to a solution of Compound 44 (37.4 mg, 57.0 μmol) in anhydrous THF (2.5 mL) at −45° C. The reaction mixture was stirred at −45° C. under argon for 15 min, after which a solution of Compound 42 (23.0 mg, 79.8 μmol) in anhydrous THF (0.50 mL) was added. The reaction mixture was allowed to warm to room temperature and stirred under argon for 30 min. The reaction was then quenched with MeOH (0.5 mL), concentrated under reduced pressure, and purified via silica chromatography (0-80% EtOAc/pet. ether) to yield Compound 45 as a white solid (31.5 mg, 61.6%): LC/MS: (ESI-QMS): m/z=898.28 (M+H), ¹H NMR (500 MHz, 298 K, DMSO-d6) δ 8.411 (d, J=1.5 Hz, 1H), 7.788 (t, J=2.5 Hz, 1H), 7.719 (d, J=2.5 Hz, 1H), 7.197 (m, 3H), 7.015 (s, 1H), 6.447 (s, 2H), 6.372 (s, 1H), 5.950 (d, J=10.0 Hz, 1H), 5.576 (d, J=10.0 Hz, 1H), 5.096 (d, J=13.0 Hz, 2H), 4.386 (d, J=9.0 Hz, 1H), 4.176 (m, 2H), 3.951 (m, 4H), 3.815 (s, 3H), 3.775 (m, 2H), 3.636 (s, 3H), 3.161 (m, 1H), 2.818 (m, 2H), 2.412 (m, 2H), 1.811 (m, 4H), 1.560 (m, 2H), 0.912 (t, J=3.0 Hz, 2H).

### Step 3: Preparation of Compound 46

A suspension of Compound 45 (30.1 mg, 33.6 μmol) and MgBr₂ (12.4 mg, 67.2 μmol) in anhydrous Et₂O (2.0 mL) was stirred at ambient temperature under argon for 3 min. The reaction mixture was then diluted with anhydrous CH₂Cl₂ (5.0 mL), stirred at room temperature under argon for an additional 60 min, and concentrated under reduced pressure. The resulting residue was dissolved in a pre-mixed solution of formic acid (12.7 μL) in MeOH (9.5 mL), stirred at room temperature for 5 min, and loaded directly onto a preparative HPLC column for purification (10-100% MeCN/50 mM NH₄HCO₃ buffer, pH 7.0) to afford Compound 46 as a white solid (13.5 mg, 52.4%): LC/MS: (ESI-QMS): m/z=767.20 (M+H), ¹H NMR (500 MHz, 298 K, DMSO-d6) δ 8.412 (d, J=1.5 Hz, 1H), 7.795 (t, J=2.5 Hz, 1H), 7.722 (d, J=2.5 Hz, 1H), 7.196 (m, 3H), 7.016 (s, 1H), 6.303 (s, 1H), 5.949 (d, J=10.5 Hz, 1H), 5.579 (d, J=11.0 Hz, 1H), 5.095 (d, J=12.5 Hz, 2H), 4.387 (d, J=9.5 Hz, 1H), 4.208 (d, J=16.0 Hz, 1H), 4.095 (m, 1H), 4.022 (m, 2H), 3.922 (m, 2H), 3.815 (s, 3H), 3.619 (s, 3H), 3.161 (d, J=16.5 Hz, 1H), 2.785 (m, 2H), 2.450 (m, 2H), 1.827 (m, 4H), 1.556 (m, 2H).

### Example 27: Preparation of Compound 38

Compound 38 is obtainable by the methods disclosed in PCT/US2013/065079 (WO2014062697), incorporated herein by reference.

### Example 28: Preparation of Conjugate 9

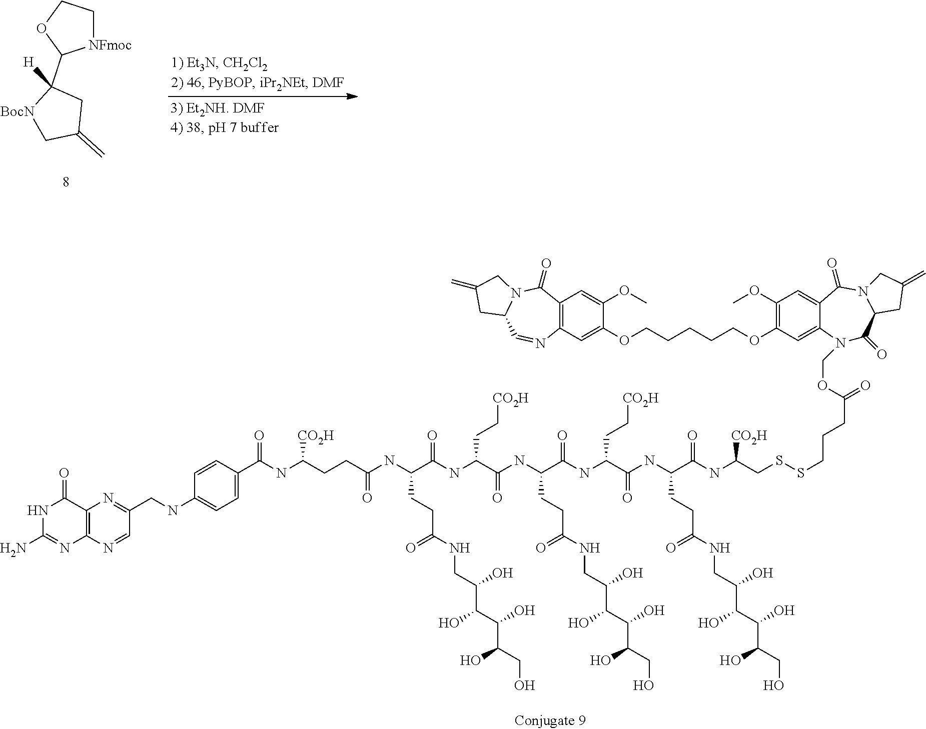

TFA (0.10 mL) was added to a solution of Compound 8 (3.7 mg, 7.67 μmol) in anhydrous CH₂Cl₂ (0.40 mL). The reaction mixture was stirred at room temperature under argon for 30 min and concentrated under reduced pressure. The residue was co-evaporated with CH₂Cl₂ (1 mL×3) and dried under high vacuum for 1 h. The crude residue was dissolved in anhydrous CH₂Cl₂ (1.0 mL) and transferred into a solution of Compound 46 (4.5 mg, 5.9 μmol) and PyBOP (3.7 mg, 7.1 μmol) in anhydrous DMF (1.0 mL). To the solution was then added ⁱPr₂NEt (10.3 μL, 59 μmol), and the reaction mixture was stirred at room temperature under argon for an additional 100 min. The CH₂Cl₂ was removed from the reaction mixture in vacuo after which diethylamine (0.10 mL) was added. The reaction mixture was stirred at room temperature under argon for 15 min and further diluted with DMF (3.5 mL). A solution of Compound 38 (11.6 mg, 6.5 μmol) in 50 mM NH₄HCO₃ buffer, pH 7.0 (4.5 mL) was then added. The reaction mixture was stirred at room temperature under argon for 20 min and purified via preparative HPLC 10-100% MeCN/50 mM NH₄HCO₃ buffer, pH7) to yield Conjugate 9 as a fluffy yellow solid (4.6 mg, 32% over three steps): LC/MS: (ESI-QMS): m/z=1206.43 (M+H), Selective H NMR (500 MHz, 298 K, DMSO-d6 with D₂O exchange) δ 8.602 (s, 1H), 7.588 (d, J=8.5 Hz, 2H), 7.148 (s, 1H), 6.945 (s, 1H), 6.623 (d, J=8.5 Hz, 2H), 5.914 (d, J=10.5 Hz, 1H), 5.501 (d, J=10.5 Hz, 1H), 5.076 (b, 3H), 4.938 (d, J=9.0 Hz, 1H).

### Example 29: Preparation of Compound 48

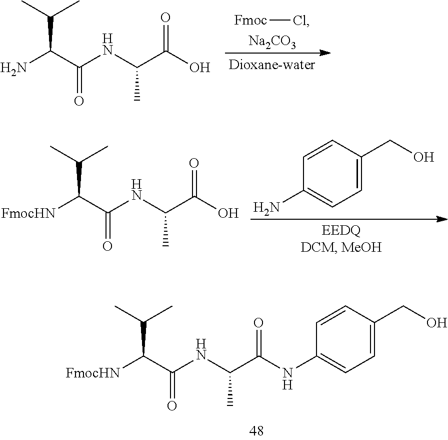

To a solution of Val-Ala-OH (1 g, 5.31 mM) in water (40 ml) was added Na₂CO₃ (1.42 g, 13.28 mM) and cooled to 0° C. before dioxane (40 mL) was added. A solution of Fmoc-C₁ (1.44 g, 5.58 mM) in dioxane (40 mL) was added dropwise over 10 min at 0° C. The reaction mixture was stirred at 0° C. for 2 h. Then the reaction mixture was allowed to stir at RT for 16 h. Dioxane was removed under vacuum, the reaction mixture diluted with water (450 mL), pH was adjusted to 2 using 1N HCl and extracted with EtOAc (3×250 mL). The combined organic layers were washed with brine, dried over MgSO₄, filtered, concentrated under reduced pressure and dried to yield Fmoc-Val-Ala-OH. This product was suspended in dry DCM (25 ml), PABA (0.785 g, 6.38 mM) and EEDQ (1.971 g, 7.97 mM) were added. The resulting mixture was treated under Argon with methanol until a clear solution was obtained. The reaction was stirred overnight and filtered. The filtrate was washed with diethyl ether (4×) and dried under high vacuum to yield Compound 48 (1.85 g, 68%). ¹H NMR (500 MHz, CD₃OD): δ 7.79 (d, J₁=8.0 Hz, 2H), 7.65 (t, J₁=7.0 Hz, J₂=7.5 Hz, 2H), 7.54 (d, J₁=8.0 Hz, 2H), 7.38 (t, J₁=7.5 Hz, J₂=7.5 Hz, 2H), 7.33-7.24 (m, 4H), 4.54 (s, 2H), 4.48 (q, J₁=14.0 Hz, J₂=7.0 Hz, 1H), 4.42-4.32 (m, 2H), 4.22 (t, J₁=7.0 Hz, J₂=6.5 Hz, 1H), 3.94 (d, J₁=7.0 Hz, 1H), 2.07 (m, 1H), 1.43 (d, J₁=7.5 Hz, 3H), 0.97 (d, J₁=7.0 Hz, 3H), 0.95 (d, J₁=7.0 Hz, 3H); LCMS (ESI): (M+H)′=Calculated for C₃₀H₃₃N₃O₅, 516.24; found 516.24.

### Example 30: Preparation of Compound 25

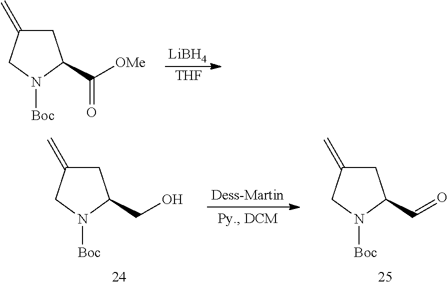

### Step 1: Preparation of Compound 24

To a mixture of 1-(tert-butyl) 2-methyl (S)-4-methylenepyrrolidine-1,2-dicarboxylate (Compound 7) (0.5 g, 2.07 mmol) in THF (10 mL) was added LiBH₄ (67.7 mg, 3.11 mmol) in portions at 0° C. under argon. The mixture was allowed to warm to room temperature over 2.5 hours. It was cooled to 0° C. and quenched with H₂O. The mixture was extracted with EtOAc (3×30 mL) and the organic phase was washed with H₂O, brine sequentially and dried over anhydrous MgSO₄. It was filtered and concentrated in vacuo. The crude product Compound 24 was used in next step without further purification.

### Step 1: Preparation of Compound 25

To a mixture of Compound 24 and pyridine (0.84 ml, 10.35 mmol) in dichloromethane (8 ml) was added Dess-Martin periodinane (1.2 g, 2.90 mmol) at 0° C. It was stirred at room temperature for 2 hours. The crude product was purified with CombiFlash in 0-40% EtOAc/p-ether to afford 0.26 g of Compound 25 in 59.3% yield. ¹H NMR (500 MHz, CDCl₃) (rotamers): δ 9.56 and 9.49 (s, 1H), 5.03 (m, 2H), 4.35-4.20 (m, 1H), 4.13-4.02 (m, 2H), 2.86-2.71 (m, 1H), 2.67-2.64 (m, 1H), 1.49 and 1.44 (s, 9H).

### Example 31: Preparation of Compound 50

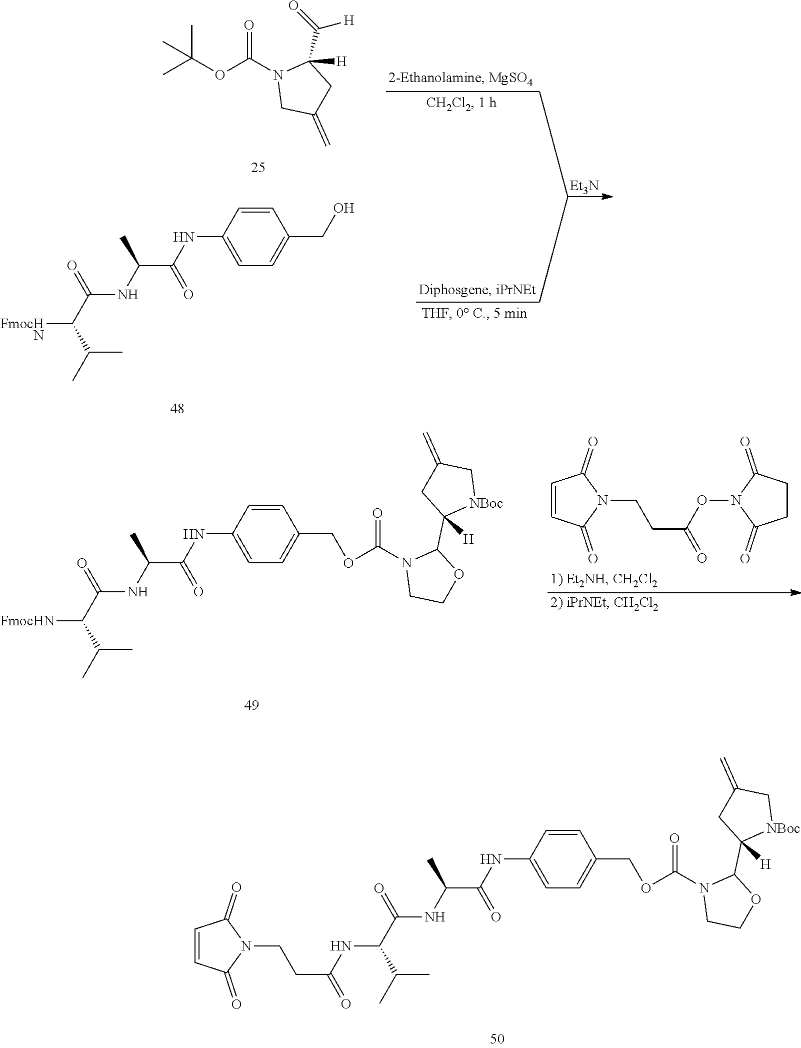

### Step 1: Preparation of Compound 49

A suspension of Compound 25 (288 mg, 1.35 mmol), 2-ethanolamine (45 μL, 0.749 mmol), and MgSO₄ (200 mg) in anhydrous CH₂Cl₂ (5.0 mL) was stirred at room temperature under argon for 1 h. The reaction mixture was passed through a sintered glass frit, and the filtrate was added to a pre-mixed solution of Compound 48 (386 mg, 0.749 mmol), diphosgene (55.0 μL, 0.457 mmol), and ⁱPr₂NEt (270 μL, 1.57 mmol) in anhydrous THF (20 mL) at 0° C. To the solution was added Et₃N (105 μL, 0.749 mmol), and the reaction mixture was stirred at 0° C. under argon for 5 min. The reaction mixture was allowed to warm to room temperature and stirred under argon for an additional 25 min. The solution was then concentrated under reduced pressure and purified via silica chromatography (0-70% EtOAc/pet. ether) to yield Compound 49 as a white solid (195 mg, 32.7%): LC/MS: (ESI-QMS): m/z=796.47 (M+H).

### Step 2: Preparation of Compound 50

Diethylamine (0.50 mL) was added to a solution of Compound 49 (62.3 mg, 78.3 μmol) in CH₂Cl₂ (2.0 mL). The reaction mixture was stirred at room temperature under argon for 2.5 h and concentrated under reduced pressure. The residue was co-evaporated with CH₂Cl₂ (2 mL×3), dried under high vacuum for 30 min, and dissolved in anhydrous CH₂Cl₂ (3.0 mL). To the solution was added in tandem maleimidopropionic acid NHS ester (25.0 mg, 94.2 μmol) and ⁱPr₂NEt (50.0 μL, 0.290 mmol). The reaction mixture was stirred at room temperature under argon for 1.5 h, concentrated under reduced pressure, and purified via silica chromatography (0-100% EtOAc/pet. ether) to yield Compound 50 as a white solid (53.5 mg, 94.2%): LC/MS: (ESI-QMS): m/z=743.85 (M+H), ¹H NMR (500 MHz, 298 K, DMSO-d6) δ 9.894 (s, 1H), 8.166 (d, J=8.5 Hz, 1H), 8.025 (d, J=8.5 Hz, 1H), 7.599 (d, J=8.5 Hz, 2H), 7.322 (b, 2H), 6.995 (s, 2H), 4.998 (m, 5H), 4.378 (m, 1H), 4.249 (m, 1H), 4.126 (t, J=8.0 Hz, 1H), 3.977-3.594 (m, 6H), 2.466 (m, 2H), 1.932 (m, 1H), 1.367 (m, 12H), 0.858 (m, 6H).

### Example 32: Preparation of Compound 51

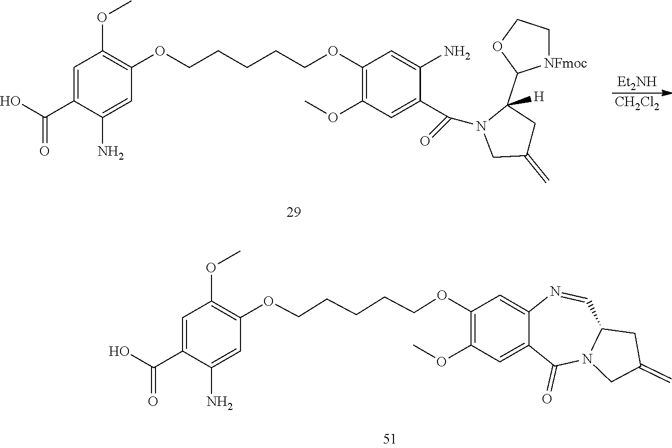

A solution of Compound 29 (105 mg, 0.132 mmol) and diethylamine (1.0 mL) in anhydrous CH₂Cl₂ (3.0 mL) was stirred at room temperature under argon for 90 min. The reaction mixture was concentrated under reduced pressure and dried under high vacuum to yield crude Compound 51 as a light brown solid (39.5 mg). The crude material was used without further purification. LC/MS: (ESI-QMS): m/z=510.41 (M+H).

### Example 33: Preparation of Conjugate 10

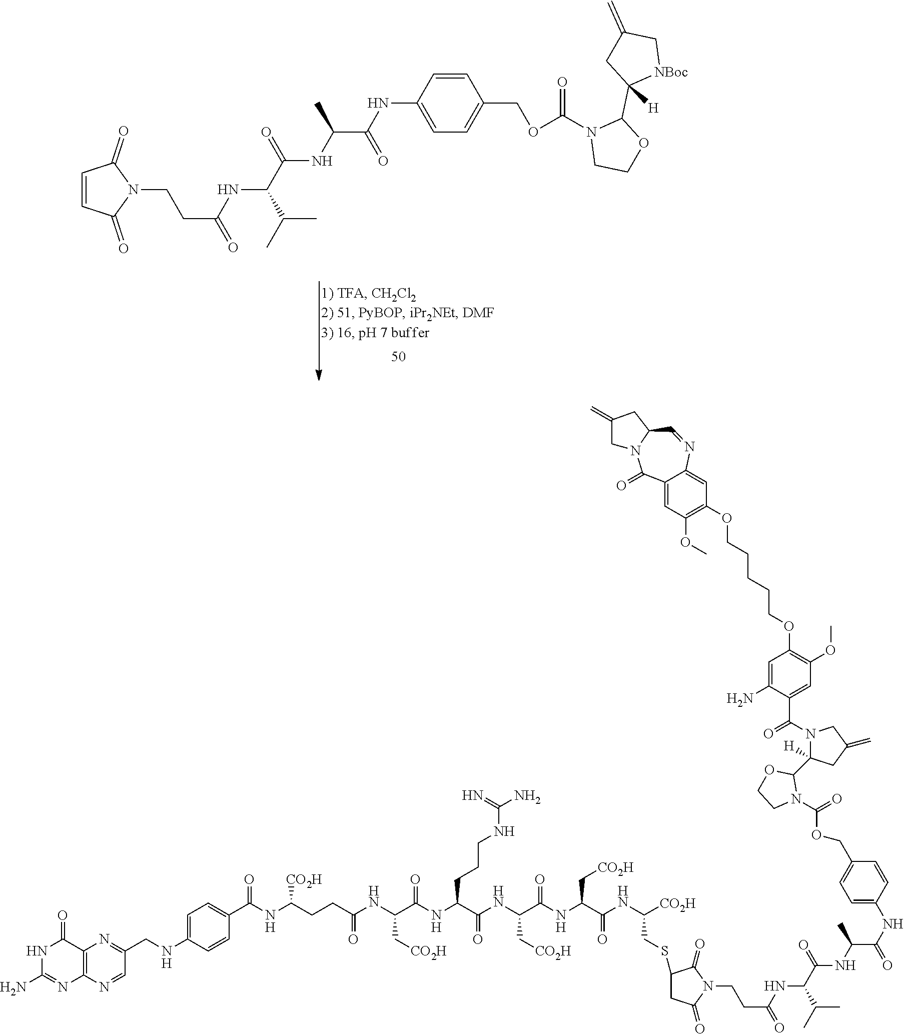 [SMILES: C=C1C[C@@H](C2OCCN2C(=O)OCc2ccc(NC(=O)[C@H](C)NC(=O)[C@@H](NC(=O)CCN3C(=O)C=CC3=O)C(C)C)cc2)N(C(=O)OC(C)(C)C)C1 ; C=C1C[C@H]2C=Nc3cc(OCCCCCOc4cc(N)c(C(=O)N5CC(=C)C[C@H]5C5OCCN5C(=O)OCc5ccc(NC(=O)[C@H](C)NC(=O)[C@@H](NC(=O)CCN6C(=O)CC(SC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c7ccc(NCc8cnc9nc(N)[nH]c(=O)c9n8)cc7)C(=O)O)C(=O)O)C6=O)C(C)C)cc5)cc4OC)c(OC)cc3C(=O)N2C1]

A solution of Compound 50 (7.5 mg, 10 μmol) in anhydrous TFA/CH₂Cl₂ (0.35 mL/1.0 mL) was stirred at room temperature under argon for 35 min, after which the reaction mixture was concentrated under reduced pressure. The resulting residue was co-evaporated with CH₂Cl₂ (2 mL×3), and dried under high vacuum for 1 h. A pre-mixed solution of Compound 51 (5.3 mg, 10 μmol) and PyBOP (5.7 mg, 11 mol) in anhydrous DMF (3.0 mL) was then added to the crude residue. To the solution ⁱPr₂NEt (8.7 μL, 50 μmol) was added, and the reaction mixture was stirred at room temperature under argon for 30 min. The solution was diluted with DMF (1.5 mL) and added a pre-mixed solution of Compound 16 (12.5 mg, 12 μmol) in 50 mM NH₄HCO₃ buffer, pH 7.0 (4.5 mL). The reaction mixture was stirred at room temperature under argon for 15 min and purified via preparative HPLC (10-100% MeCN/50 mM NH₄HCO₃ buffer, pH 7.0 to yield Conjugate 10 as a fluffy yellow solid (7.9 mg, 37%): LC/MS: (ESI-QMS): m/z=1080.16 (M+H). Selective H NMR (500 MHz, 298 K, DMSOd6) δ 8.671 (b, 1H), 7.637 (b, 2H), 7.478 (b, 2H), 7.102 (b, 4H), 6.782 (b, 4H), 6.603 (b, 1H), 6.411 (b, 1H).

### Example 34: Preparation of Compound 56

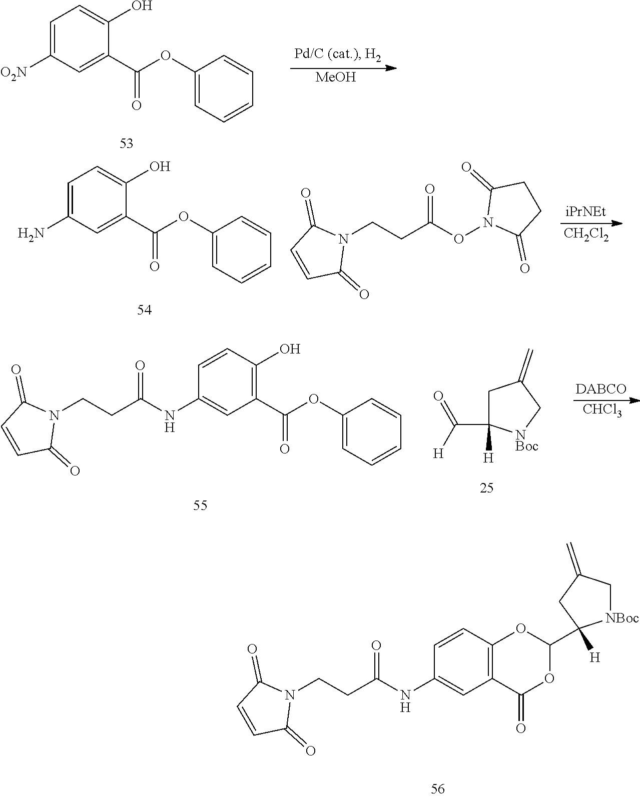

### Step 1: Preparation of Compound 54

Pd/C (10% w/w, 7.1 mg) was added to a solution of Compound 53 (Sigma-Aldrich; 57.8 mg, 0.223 mmol) in MeOH (3.0 mL) under argon. The headspace was evacuated and purged with hydrogen gas. The reaction mixture was stirred under hydrogen for 85 min. The reaction mixture was filtered through a pad of Celite, and the filtrate was concentrated under reduced pressure and dried under vacuum to yield Compound 54 as a light brown solid (50.1 mg, 98.0%). The crude product was used without further purification: LC/MS: (ESI-QMS): m/z=230.38 (M+H), H NMR (500 MHz, 298 K, DMSO-d6 with D₂O exchange) δ 7.454 (t, J=4.0 Hz, 2H), 7.316 (t, J=4.0 Hz, 1H), 7.233 (m, 3H), 6.918 (dd, J=9.0 Hz, 3.0 Hz, 1H), 6.786 (d, J=9.0 Hz, 1H).

### Step 2: Preparation of Compound 55

A solution of Compound 54 (49.5 mg, 0.216 mmol), maleimidopropionic acid NHS ester (115 mg, 0.432 mmol), and ⁱPr₂NEt (200 μL, 1.17 mol) in anhydrous CH₂Cl₂ (5.0 mL) was stirred at room temperature under argon for 2 h and purified via silica chromatography (0-70% EtOAc/pet. ether) to yield Compound 55 as an impure mixture (40.1 mg). The mixture was further purified via silica chromatography (0-2% MeOH/CH₂C₂) to afford Compound 55 as a white solid (21.5 mg, 26.2%): LC/MS: (ESI-QMS): m/z=381.54 (M+H), ¹H NMR (500 MHz, 298 K, DMSO-d6) δ 8.236 (d, J=2.5 Hz, 1H), 7.698 (dd, J=9.0, 2.5 Hz, 1H), 7.500 (m, 2H), 7.334 (m, 3H), 7.021 (s, 2H), 7.006 (d, J=9.0 Hz, 1H), 3.710 (t, J=7.0 Hz, 2H), 2.559 (t, J=6.5 Hz, 2H).

### Step 3: Preparation of Compound 56

A solution of Compound 55 (85.0 mg, 0.223 mmol), Compound 25 (95.0 mg, 0.446), and DABCO (80.1 mg, 0.714 mmol) in anhydrous CHCl₃ (0.75 mL) was stirred at room temperature under argon for 6 h and purified via silica chromatography (0-2% MeOH/CH₂Cl₂) to yield impure Compound 56 as a white solid (57.1 mg). The crude product was used without further purification. LC/MS: (ESI-QMS): m/z=496.44 (M+H), ¹H NMR (500 MHz, 298 K, CD₃OD) δ 8.094 (b, 1H), 7.769 (m, 1H), 7.067 (d, J=9.5 Hz, 1H), 6.818 (s, 2H), 5.900 (d, J=58.0 Hz, 1H), 4.569 (s, 2H), 4.203 (b, 1H), 4.173 (d, J=15.0 Hz, 1H), 4.027 (m, 1H), 3.867 (t, J=6.5 Hz, 2H), 2.908 (b, 2H), 2.653 (t, J=6.5 Hz, 2H).

### Example 35: Preparation of Conjugate 11

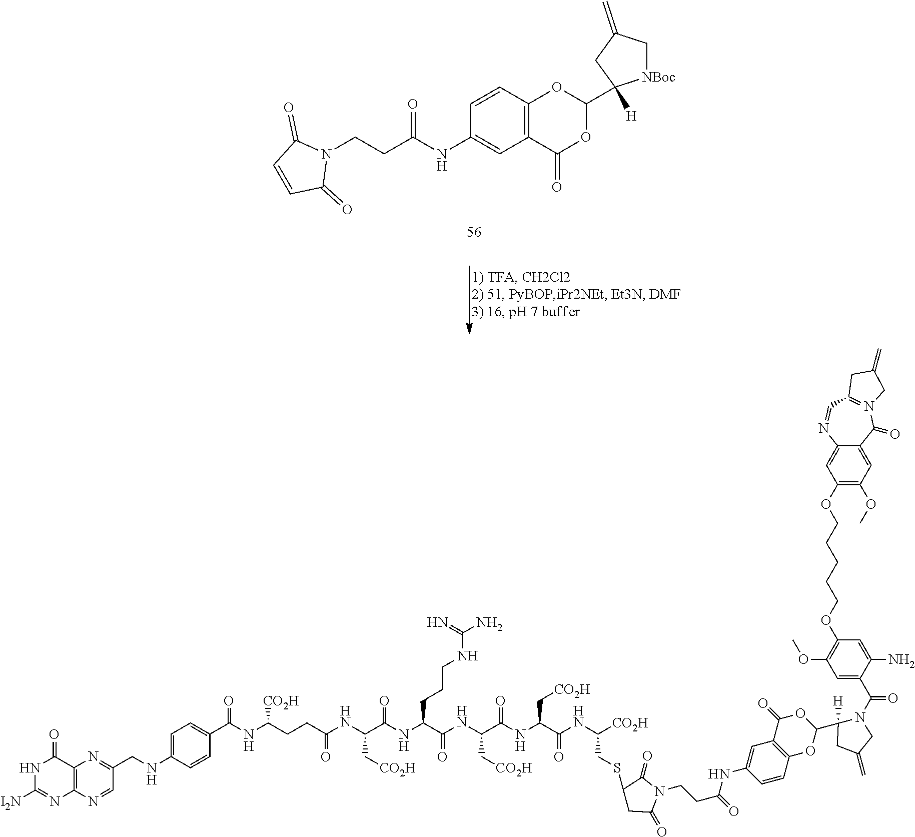

A solution of Compound 56 (16.9 mg, 34.0 μmol) in anhydrous TFA/CH₂Cl₂ (0.20 mL/1.0 mL) was stirred at room temperature under argon for 90 min and concentrated under reduced pressure. The residue was co-evaporated with CH₂Cl₂ (1.5 mL×3) and dried under vacuum for 1 h. A pre-mixed solution of Compound 51 (19.1 mg, 37.4 μmol) and PyBOP (21.2 mg, 40.8 μmol) in anhydrous DMF (3.0 mL) was added to the crude residue. To the solution was added ⁱPr₂NEt (30.0 μL, 170 μmol), and the reaction mixture was stirred at room temperature under argon for 30 min. Et₃N (15.0 μL, 102 μmol) was added to the reaction mixture and stirred at room temperature under argon for an additional 60 min. The solution was the diluted with DMF (1.5 mL) and to which was added and a pre-mixed solution of Compound 16 (43.1 mg, 40.8 μmol) in 50 mM NH₄HCO₃ buffer, pH 7.0, (4.5 mL). After stirring at room temperature under argon for 35 min, the reaction mixture was filtered, and the filtrate was purified via preparative HPLC (10-100%, MeCN/50 mM NH₄HCO₃ buffer, pH 7.0 to yield Conjugate 11 as a fluffy yellow solid (3.1 mg, 4.7% over three steps): LC/MS: (ESI-QMS): m/z=1934.06 (M+H), Selective ¹H NMR (500 MHz, 298 K, DMSOd6) δ 8.611 (s, 1H), 8.125 (b, 1H), 7.598 (b, 4H), 7.102 (b, 4H), 6.617 (b, 4H), 6.513 (s, 1H), 6.361 (s, 1H), 6.289 (b, 1H).

### Example 36: Preparation of Compound 58

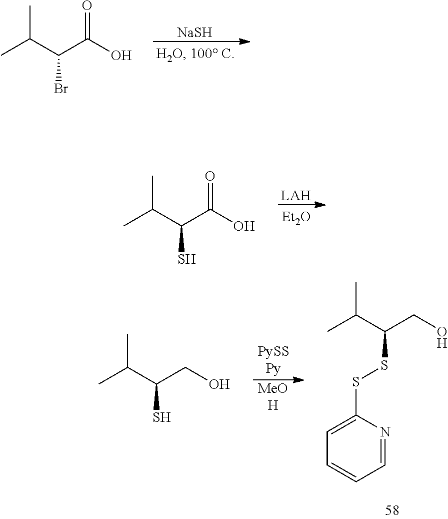

Water (4.5 mL) was added to a glass vial containing (R)-(+)-2-bromo-3-methylbutyric acid (958 mg, 5.29 mmol) and NaHS.XH₂O (1.01 g), and the vial was capped immediately. The resulting solution was stirred for 2 min at ambient temperature and for 3.5 h at 100° C. After allowing the reaction mixture to cool to ambient temperature, the cap of the vial was opened, and the solution was flushed with argon for 5 min. The solution was acidified (pH ˜2) with 2.0 N HCl and extracted with diethyl ether (35 mL×2). The organic layers were separated, combined, dried over Na₂SO₄, and filtered. The filtrate was added to a suspension of LAH (600 mg, 3.00 equiv.) in anhydrous diethyl ether (10 mL). After stirring at ambient temperature under argon for 30 min, the reaction mixture was cooled in an ice-bath and quenched with 1.0 N HCl (28 mL) at 0° C. The ice-bath was removed and the reaction mixture stirred at ambient temperature under argon for 15 min. The top clear solution of the reaction mixture was poured into a solution of aldrithiol (1.16 g, 1.00 equiv.) in MeOH (50 mL). The remaining gel-like material from the LAH reduction was washed with diethyl ether (50 mL) and added to the aldrithiol solution. Saturated aqueous NaHCO₃ solution (50 mL) was added to the aldrithiol solution until the pH reached ˜7.5 and the reaction mixture was stirred at ambient temperature under argon for 1.5 h. The solution was then filtered through a pad of Celite, and the filtrate was concentrated under reduced pressure to yield an oily residue, which was further purified by a CombiFlash system (silica gel column) eluting with 0-10% EtOAc in petroleum ether to yield 615 mg Compound 58 as a white solid: LC/MS: (ESI-QMS): m/z=230.07 (M+H), H NMR (500 MHz, 298 K, CDCl₃) δ 8.502 (d, J=5.0 Hz, 1H), 7.662 (m, 1H), 7.569 (t, J=7.5 Hz, 1H), 7.155 (m, 1H), 3.839 (m, 1H), 3.635 (m, 1H), 2.739 (m, 1H), 1.809 (m, 1H), 1.106 (d, J=7.0 Hz, 3H), 1.070 (d, J=7.0 Hz, 3H).

### Example 37: Preparation of Compound 59

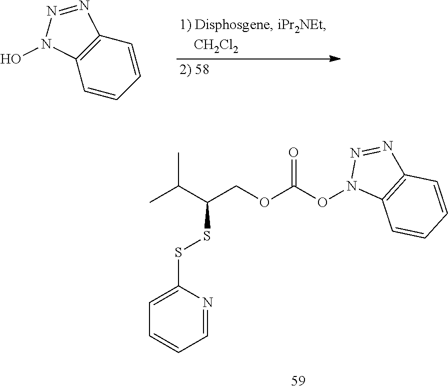 [SMILES: On1nnc2ccccc21 ; CC(C)[C@@H](COC(=O)On1nnc2ccccc21)SSc1ccccn1]

A solution of hydroxybenzotriazole (229 mg, 2.0 equiv.) in anhydrous CH₂Cl₂ (15 mL) was added slowly to a stirred solution of diphosgene (0.12 mL, 1.2 equiv.) in anhydrous CH₂Cl₂ (3.0 mL) at ambient temperature. To the resulting solution was added iPr₂NEt (0.75 mL, 5.0 equiv.). After stirring at ambient temperature under argon for 3 min, a solution of Compound 58 (196 mg, 0.855 mmol) in anhydrous CH₂Cl₂ (5.0 mL) was added to the reaction mixture. The reaction mixture was then stirred at ambient temperature under argon for 1 h, quenched with water (50 μL), stirred at ambient temperature for 5 min, and loaded directly onto a CombiFlash system for purification (Silica gel column) (Gradient 0-60% EtOAc in petroleum ether.) to afford 202 mg Compound 59 as a glass-like solid: LC/MS: (ESI-QMS): m/z=391.06 (M+H), ¹H NMR (500 MHz, 298 K, CDCl₃) δ 8.390 (d, J=1.0 Hz, 1H), 8.232 (d, J=8.5 Hz, 1H), 8.026 (d, J=7.5 Hz, 1H), 7.777 (m, 2H), 7.662 (m, 1H), 7.556 (t, J=7.5 Hz, 1H), 7.042 (m, 1H), 4.767 (m, 2H), 3.193 (m, 1H), 2.267 (m, 1H), 1.189 (d, J=7.0 Hz, 3H), 1.148 (d, J=7.0 Hz, 3H).

### Example 38: Preparation of Compound 60

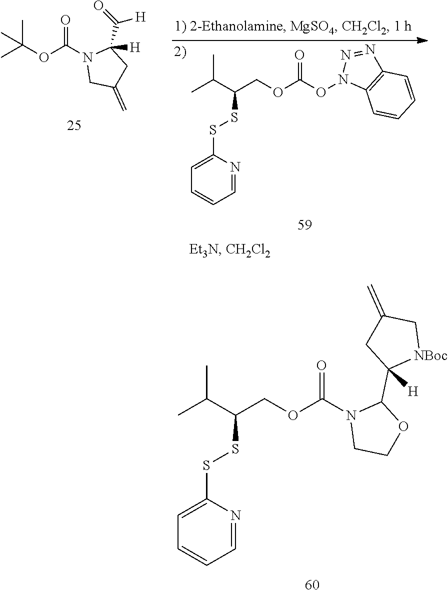 [SMILES: C=C1C[C@@H](C=O)N(C(=O)OC(C)(C)C)C1 ; CC(C)[C@@H](COC(=O)On1nnc2ccccc21)SSc1ccccn1 ; C=C1C[C@@H](C2OCCN2C(=O)OC[C@@H](SSc2ccccn2)C(C)C)N(C(=O)OC(C)(C)C)C1]

A suspension of Compound 25 (20.0 mg, 95.0 μmol), 2-ethanolamine (4.2 μL, 71.3 μmol), and MgSO₄ (90 mg) in anhydrous CH₂Cl₂ (0.35 mL) was stirred at room temperature under argon for 2 h. The reaction mixture was diluted with anhydrous CH₂Cl₂ (0.75 mL) and filtered through a sintered glass frit, and the filtrate was transferred to a small vial containing Compound 59 (37.0 mg, 95.0 μmol). To the resulting solution was added Et₃N (15.0 μL, 105 μmol). The reaction mixture was then stirred at room temperature under argon for 25 min and purified via silica chromatography (0-35%, EtOAc/pet. ether) to yield Compound 60 as a light beige solid (22.0 mg, 45.4%): LC/MS: (ESI-QMS): m/z=510.61 (M+H), ¹H NMR (500 MHz, 298 K, CD₂Cl₂) δ 8.415 (d, J=4.0 Hz, 1H), 7.749 (b, 1H), 7.665 (t, J=8.0 Hz, 1H), 7.098 (m, 1H), 5.109 (m, 1H), 4.936 (s, 1H), 4.905 (s, 1H), 4.315-3.776 (m, 10H), 3.378-3.254 (m, 1H), 3.019 (m, 1H), 2.704-2.387 (m, 2H), 2.115 (b, 1H), 1.408 (b, 9H), 1.113-1.044 (m, 6H). MS⁺ (ESI m/z) calculated for C₂₄H₃₆N₃O₅S₂: 510.20; found 510.61.

### Example 39: Preparation of Conjugate 12

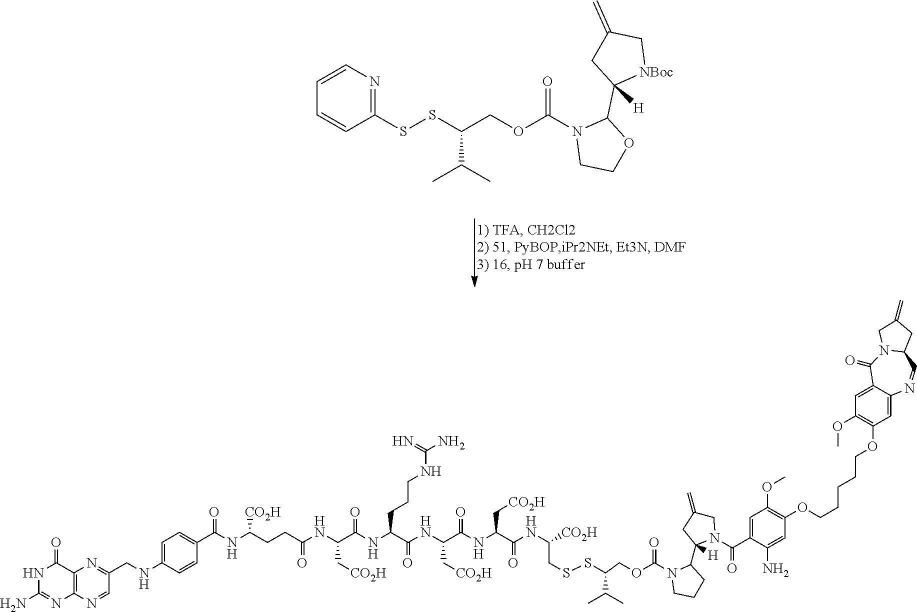 [SMILES: C=C1C[C@@H](C2OCCN2C(=O)OC[C@@H](SSc2ccccn2)C(C)C)N(C(=O)OC(C)(C)C)C1 ; C=C1C[C@H]2C=Nc3cc(OCCCCCOc4cc(N)c(C(=O)N5CC(=C)C[C@H]5C5CCCN5C(=O)OC[C@@H](SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)C(=O)O)C(C)C)cc4OC)c(OC)cc3C(=O)N2C1]

A solution of Compound 60 (7.6 mg, 15.0 μmol) in anhydrous TFA/CH₂Cl₂ (0.15 mL/0.75 mL) was stirred at room temperature under argon for 1.5 h and concentrated under reduced pressure. The residue was co-evaporated with CH₂Cl₂ (1 mL×3), and dried under high vacuum for 1 h. A pre-mixed solution of Compound 51 (8.4 mg, 16.5 μmol) and PyBOP (8.5 mg, 16.5 μmol) in anhydrous DMF (2.0 mL) was added to the crude residue. To the solution was then added ⁱPr₂NEt (15.6 μL, 90 μmol) and the reaction mixture was stirred at room temperature under argon for 1 h. The solution was diluted with DMF (1.5 mL) and to which was added a pre-mixed solution of Compound 16 (17.2 mg, 16.5 μmol) in 50 mM NH₄HCO₃ buffer, pH 7.0 (4.5 mL). The resulting cloudy solution was stirred at room temperature for 20 min, and then at 65° C. for 30 min. The reaction mixture was allowed to cool to room temperature, filtered, and purified via preparative HPLC (10-100%, MeCN/50 mM NH₄HCO₃ buffer, pH 7.0) to yield Conjugate 12 as a fluffy yellow solid (6.1 mg, 22% over three steps): LC/MS: (ESI-QMS): m/z=1834.18 (M+H), Selective H NMR (500 MHz, 298 K, D₂O) δ 8.699 (b, 1H), 7.690 (b, 2H), 7.418 (b, 1H), 7.236 (b, 1H), 7.146 (b, 1H), 6.798 (b, 2H), 6.553 (b, 1H), 6.403 (b, 1H).

### Example 40: Preparation of Conjugate 13

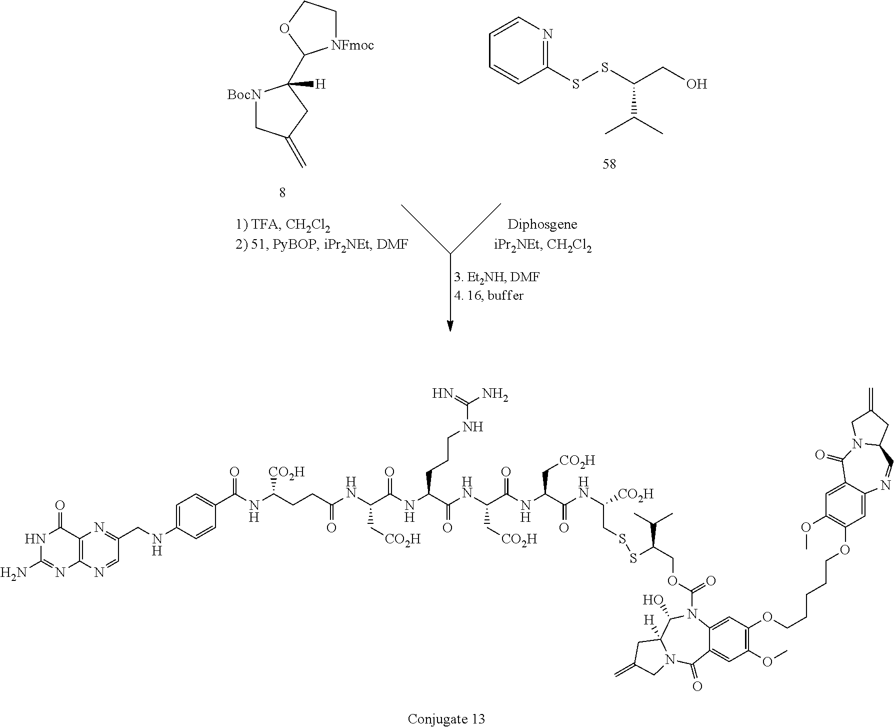

A solution of Compound 8 (43.9 mg, 92.1 μmol) in anhydrous TFA/CH₂Cl₂ (0.15 mL/0.85 mL) was stirred at room temperature under argon for 30 min and concentrated under reduced pressure. The residue was co-evaporated with CH₂Cl₂ (1.5 mL×3), dried under high vacuum for 1 h, and dissolved in anhydrous CH₂Cl₂ (1.5 mL). To the solution was added a pre-mixed solution of Compound 51 (44.6 mg, 87.5 μmol) and PyBOP (47.8 mg, 92.1 μmol) in anhydrous DMF (1.5 mL). To the solution was added ⁱPr₂NEt (80.0 μL, 460 μmol). The reaction mixture was stirred at room temperature under argon for 70 min and purified via silica chromatography (0-5% MeOH/CH₂Cl₂) to yield 39.9 mg of a beige solid containing mostly the desired product based on LC/MS analysis. In a separate flask, diphosgene (5.3 μL, 44.0 μmol) and ⁱPr₂NEt (45.0 μL, 259 μmol) were added in tandem to a solution of Compound 58 (10.1 mg, 44.0 μmol) in anhydrous CH₂Cl₂ (0.75 mL). The reaction mixture was stirred at room temperature under argon for 15 min, concentrated under reduced pressure, and concentrated under vacuum for 1 h. To the resulting residue was added a solution of the crude product (22.1 mg, 25 μmol) from the previous step in anhydrous CH₂Cl₂ (1.0 mL). The reaction mixture was stirred at room temperature under argon for 25 min, concentrated under reduced pressure, and concentrated under vacuum for 1 h. The residue was dissolved in anhydrous DMF (3.0 mL). Two-thirds of the volume was transferred to a glass vial and to which was added diethylamine (0.50 mL). The reaction mixture was stirred at room temperature under argon for 25 min, diluted with DMF (2.5 mL), and added to a pre-mixed solution of Compound 16 (25.0 mg, 23.9 μmol) in 50 mM NH₄HCO₃ buffer, pH 7.0 (4.5 mL). After stirring at 65° C. for 1 h, the reaction mixture was cooled to room temperature and filtered. The filtrate was purified via preparative HPLC (10-100%, MeCN/50 mM NH₄HCO₃ buffer, pH 7.0) to afford Conjugate 13 as a fluffy yellow solid (0.8 mg, 1% over three steps): LC/MS: (ESI-QMS): m/z=1808.43 (M+H).

### Example 41: Preparation of Conjugate 14

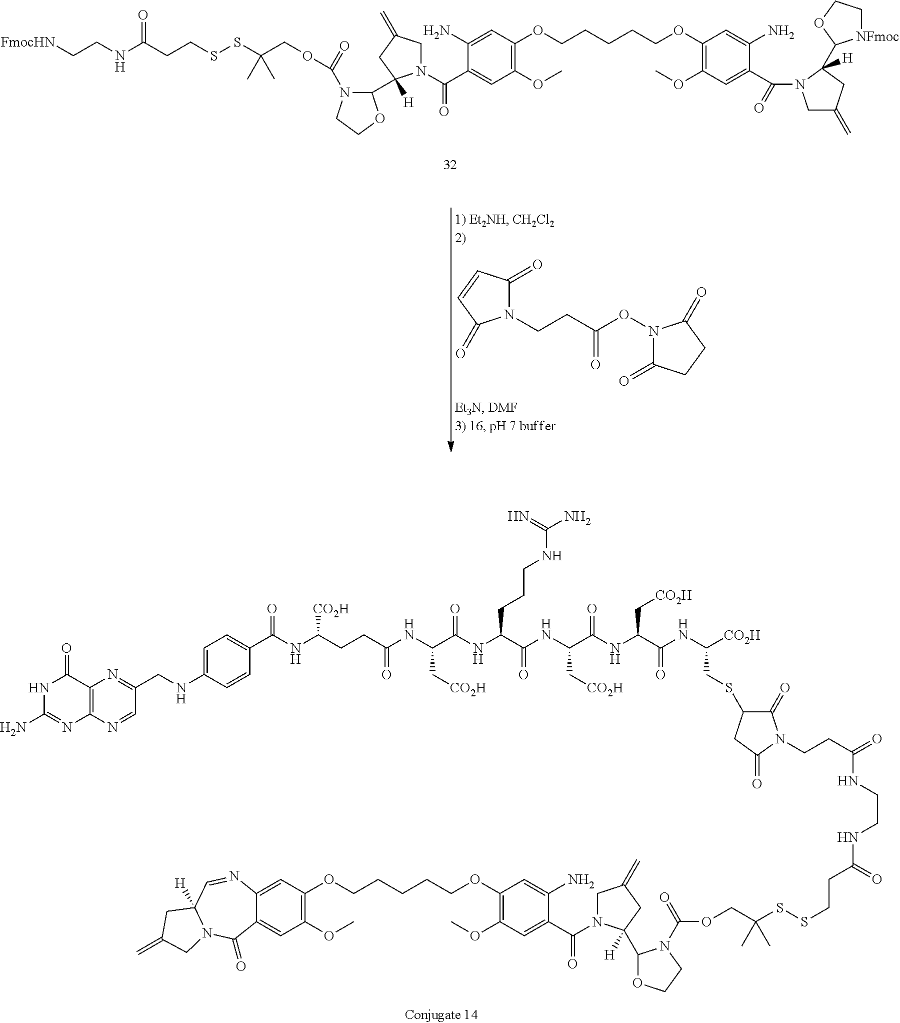

Diethylamine (0.50 mL) was added to a solution of Compound 32 (52.0 mg, 36.4 μmol) in anhydrous CH₂Cl₂ (1.0 mL). The reaction mixture was stirred at room temperature under argon for 100 min and concentrated under reduced pressure. The residue was co-evaporated with CH₂Cl₂ (2 mL×3), dried under high vacuum for 1 h, and dissolved in anhydrous DMF (2.0 mL). To the solution was added in tandem maleimidopropionic acid NHS ester (10.7 mg, 40.0 μmol) and Et₃N (10.1 μL, 72.8 μmol). The reaction mixture was stirred at room temperature under argon for 1 h, diluted with DMF (2.5 mL), and to which was added a solution of Compound 16 (49.5 mg, 47.3 μmol) in 50 mM NH₄HCO₃ buffer, pH 7.0 (5.0 mL). The reaction mixture was stirred at room temperature under argon for 15 min and purified via preparative HPLC (10-100%, MeCN/50 mM NH₄HCO₃ buffer, pH 7.0 to yield Conjugate 14 as a fluffy yellow solid (33.5 mg, 43.4%): LC/MS: (ESI-QMS): m/z=1061.58 (M+2H), Selective ¹H NMR (500 MHz, 298 K, DMSO-d6 with D₂O exchange) δ 8.554 (b, 1H), 7.484 (d, J=8.5 Hz, 2H), 7.023 (s, 1H), 6.979 (s, 1H), 6.586 (d, J=8.5 Hz, 2H), 6.457 (s, 1H), 6.325 (s, 1H), 6.165 (s, 1H).

### Example 42: Preparation of Conjugate 15

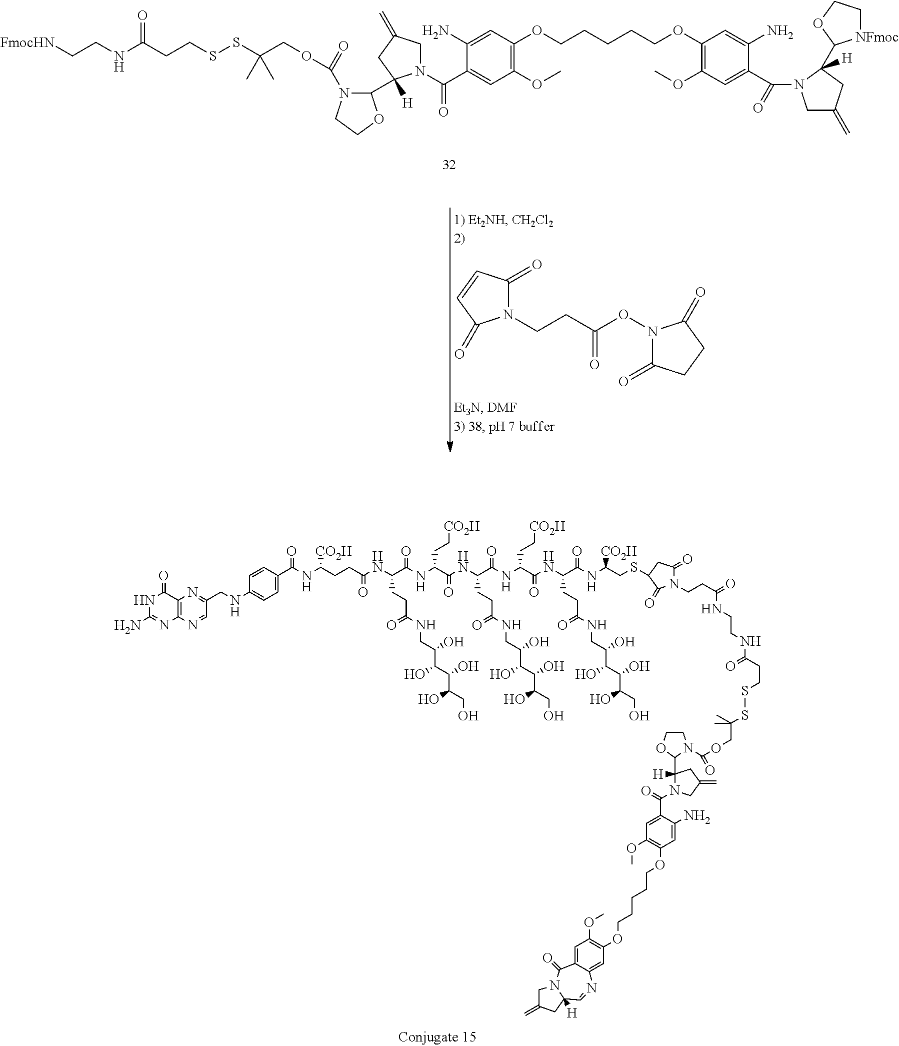

Diethylamine (0.50 mL) was added to a solution of Compound 32 (26.3 mg, 18.4 μmol) in anhydrous CH₂Cl₂ (1.0 mL). The reaction mixture was stirred at room temperature under argon for 165 min and then concentrated under reduced pressure. The residue was co-evaporated with CH₂Cl₂ (2 mL×3), dried under high vacuum for 1 h, and dissolved in anhydrous DMF (2.0 mL). To the solution was added maleimidopropionic acid NHS ester (5.4 mg, 20 μmol) and Et₃N (5.1 μL, 53 μmol) in tandem. The reaction mixture was stirred at room temperature under argon for 55 min, diluted with DMF (2.5 mL), and to which was added a solution of Compound 38 (40.1 mg, 23.9 μmol) in 50 mM NH₄HCO₃ buffer, pH 7.0, (4.5 mL).

The reaction mixture was stirred at room temperature under argon for 15 min and purified via preparative HPLC (10-100%, MeCN/50 mM NH₄HCO₃ buffer, pH 7.0 to yield Conjugate 15 as a fluffy yellow solid (20.8 mg, 41.0%): LC/MS: (ESI-QMS): m/z=1376.11 (M+2H), Selective ¹H NMR (500 MHz, 298 K, D₂O) δ 8.684 (b, 1H), 7.675 (b, 2H), 7.133 (s, 1H), 6.764 (b, 3H), 6.574 (b, 1H), 6.499 (b, 1H).

### Example 43: Preparation of Compound 66

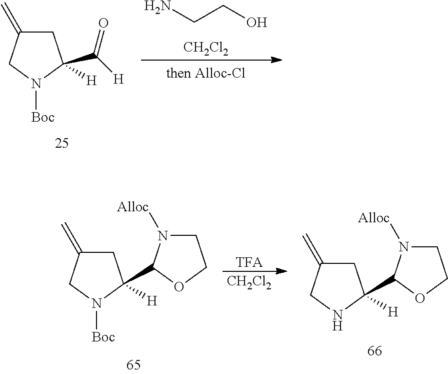

### Step 1: Preparation of Compound 65

A mixture of Compound 25 (0.108 g, 0.51 mmol), ethanolamine (32.8 mg, 0.54 mmol) and 4 Å molecular sieves in CH₂Cl₂ (5 mL) was stirred at room temperature for 3 hours. To the reaction mixture was added allyl chloroformate (57 μl, 0.54 mmol) at room temperature for 3 h. The reaction mixture was concentrated in vacuo, and the crude residue was purified via silica chromatography (0-50% EtOAc/pet. ether) to afford Compound 65 (0.14 g, 82%): ¹H NMR (500 MHz, CDCl₃) δ δ 5.94 (m, 1H), 5.31 (m, 1H), 5.24 (d, J=10.5 Hz, 2H), 4.96 (m, 2H), 4.60 (d, J=10.5 Hz, 2H), 4.15-4.06 (m, 2H), 3.88-3.82 (m, 4H), 3.52-3.28 (m, 1H), 2.64 (m, 1H), 2.54-2.42 (m, 1H), 1.44 (s, 9H).

### Step 2: Preparation of Compound 66

A mixture of Compound 65 (0.14 g, 0.41 mmol) in 20% TFA/CH₂Cl₂ solution (2 mL) was stirred at room temperature for 4 h. It was concentrated in vacuo. The crude product Compound 66 was used without further purification.

### Example 44: Preparation of Compound 68

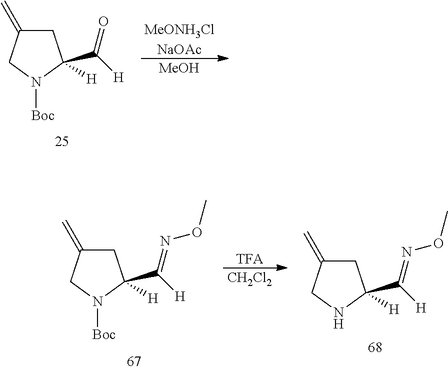

### Step 1: Preparation of Compound 67

A mixture of Compound 25 (0.193 g, 0.91 mmol), methoxyamine hydrochloride (76.3 mg, 0.91 mmol) and sodium acetate (0.3 g, 3.64 mmol) in MeOH (6 mL) was stirred at room temperature overnight. The reaction was quenched with water and extracted with EtOAc (3×30 mL). The combined organic phases were washed with H₂O and brine sequentially, dried over anhydrous MgSO₄, and concentrated in vacuo. The residue was further purified via silica chromatography (0-50% EtOAc/pet. ether) to afford the Compound 67 (0.15 g, 69%): ¹H NMR (500 MHz, CDCl₃), (E/Z isomers) δ 7.25 (s, 1H), 6.65 (s, 1H), 5.01-4.92 (m, 2H), 4.49 (m, 2H), 4.06-3.91 (m, 2H), 3.87 (s, 3H), 3.82 (s, 3H), 2.92 (m, 1H), 2.83 (m, 1H), 2.61 (d, J=15.5 Hz, 1H), 2.46 (dd, J₁=4.5 Hz, J₂=2 Hz, 1H), 1.46 (s, 9H).

### Step 2: Preparation of Compound 68

A mixture of Compound 67 (0.15 g, 0.62 mmol) in 20% TFA/CH₂Cl₂ solution was stirred at room temperature and monitored by TLC. After 4 h the solvent was removed under reduced pressure. The product Compound 68 was used without further purification.

### Example 45: Preparation of Compound 73

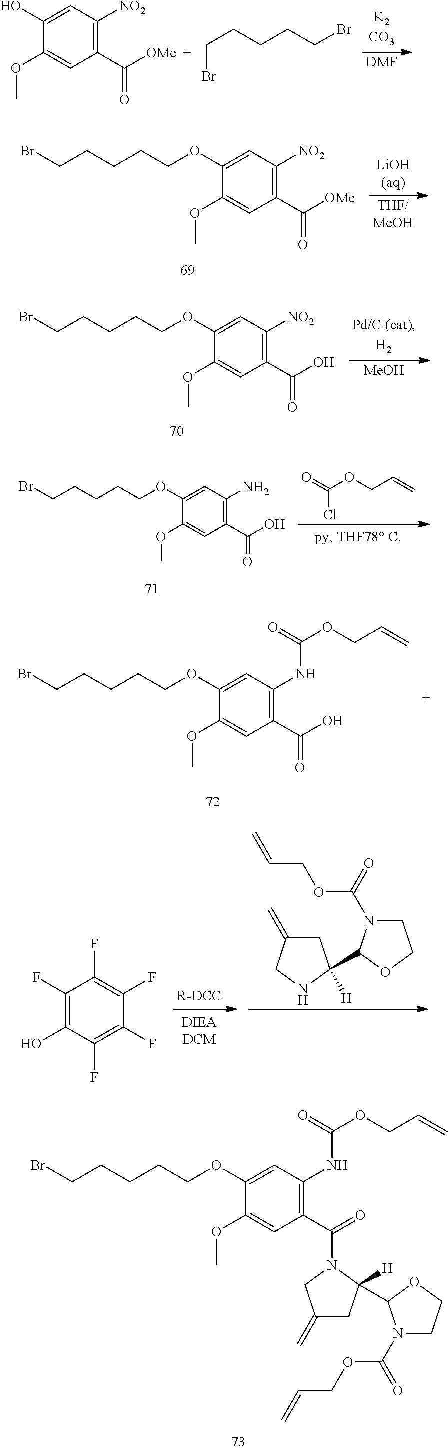

### Step 1: Preparation of Compound 69

To a mixture of methyl 4-hydroxy-5-methoxy-2-nitrobenzoate (0.34 g, 1.5 mmol) and potassium carbonate (0.21 g 1.5 mmol) in DMF (8 mL) was added 1,5-dibromopentane (1.72 g, 7.5 mmol) at room temperature under argon. The mixture was stirred room temperature overnight and then concentrated in vacuo. The crude product was purified via silica chromatography (0-50% EtOAc/pet. ether) to afford Compound 69 (0.52 g, 92%): ¹H NMR (500 MHz, CDCl₃) δ 7.43 (s, 1H), 7.07 (s, 1H), 4.10 (t, J=6.5 Hz, 2H), 3.95 (s, 3H), 3.91 (s, 3H), 3.44 (t, J=6 Hz, 2H), 1.93 (m, 4H), 1.67 (M, 2H).

### Step 2: Preparation of Compound 70

To a mixture of Compound 69 (0.52 g, 1.38 mmol) in THF/MeOH/H₂O (3 mL/1 mL/1 mL) was added 1 M LiOH_{(aq)} (6.9 mL, 6.9 mmol) at room temperature. The reaction was monitored via LC/MS and after complete consumption of Compound 69, the reaction mixture was adjusted to pH 7 with 1M HCl_{aq}) solution. The product was extracted with EtOAc (3×50 mL), dried over anhydrous MgSO₄, and filtered. The filtrate was concentrated in vacuo and the crude product was purified via silica chromatography (0-50% EtOAc/pet. ether) to afford the product as yellow solid: LC/MS: (ESI-QMS): m/z=364.25 (M+2H)¹H NMR (500 MHz, CDCl₃) δ 7.38 (s, 1H), 7.20 (s, 1H), 4.10 (t, J=6.5 Hz, 2H), 3.98 (s, 3H), 3.45 (t, J=6.5 Hz, 2H), 1.99-1.89 (m, 4H), 1.69-1.64 (m, 2H).

### Step 3: Preparation of Compound 71

A mixture of Compound 70 (0.43 g, 1.3 mmol) and 10% Pd/C in MeOH/EtOAc (5 mL/5 mL) was stirred under hydrogen atmosphere at room temperature for 3 h. The reaction mixture was then filtered through a plug of Celite, and the filtrated was concentrated in vacuo to give the product as dark brown solid. The crude product was used without further purification. LC/MS: (ESI-QMS): m/z=334.42 (M+2H).

### Step 4: Preparation of Compound 72

To a mixture of Compound 71 (0.154 g, 0.46 mmol) and pyridine (56.2 μl, 0.70 mmol) in THF (6 mL) was added ally chloroformate (61 mg, 0.51 mmol) at −78° C. under argon. The mixture was allowed to warm to room temperature overnight. The mixture was concentrated in vacuo and the crude product was purified with silica chromatography (0-80% EtOAc/pet. ether) to afford Compound 72 (0.13 g, 68%) as white solid: LC/MS: (ESI-QMS): m/z=418.37 (M+2H)¹H NMR (500 MHz, CDCl₃) δ 10.34 (s, 1H), 8.15 (s, 1H), 7.52 (d, J=2 Hz, 1H), 6.0 (m, 1H), 5.4 (d, J=17 Hz, 1H), 5.28 (d, J=10.5 Hz, 2H), 4.68 (d, J=5 Hz, 2H), 4.13 (dt, J₁=7.5 Hz, J₂=8 Hz, 2H), 3.78 (s, 3H), 3.44 (td, J₁=6.5 Hz, J₂=1.5 Hz, 2H), 1.91 (m, 3H), 1.64 (m, 1H), 1.46-1.37 (m, 2H), 0.92 (td, J₁=7.5 Hz, J₂=1.5 Hz, 2H).

### Step 5: Preparation of Compound 73

A mixture of Compound 72 (10 mg, 0.024 mmol), DCC loaded resin (2.3 mmol/g) (52 mg, 0.12 mmol) and pentafluorophenol (4.86 mg, 0.0264 mmol) in CH₂Cl₂ (1 mL) was stirred under argon at room atmosphere for 1 h. The reaction mixture was filtered through a sintered glass frit and concentrated in vacuo. The crude residue was dissolved in CH₂Cl₂ (1 mL) and Compound 66 (5.7 mg, 0.024 mmol) and ⁱPr₂NEt (12.6 μl, 0.072 mmol) in CH₂Cl₂ (1 mL) at room temperature under argon. The mixture was stirred at room temperature for 3 h. The crude product was purified via silica chromatography (0-60% EtOAc/pet. ether): LC/MS: (ESI-QMS): m/z=638.68 (M+2H), H NMR (500 MHz, CDCl₃) (mixture of diastereomers) δ 8.94 (s, 1H), 7.83 (s, 2H), 7.04 (s, 1H), 5.94 (m, 2H), 5.83 (m, 2H), 5.33 (dd, J₁=17 Hz, J₂=1 Hz, 3H), 5.28 (m, 2H), 5.23 (d, J=10 Hz, 4H), 5.11-4.97 (m, 9H), 4.67-4.56 (m, 8H), 4.51-4.39 (m, 4H), 4.20 (m, 3H), 4.14-4.05 (m, 7H), 3.97 (s, 3H), 3.95 (s, 3H), 3.43 (t, J=7 Hz, 4H), 2.69 (m, 2H), 2.60 (m, 2H), 1.97-1.83 (m, 9H), 1.62 (m, 6H), 1.25 (td, J₁=7.5 Hz, J₂=1.5 Hz, 2H).

### Example 46: Preparation of Compound 78

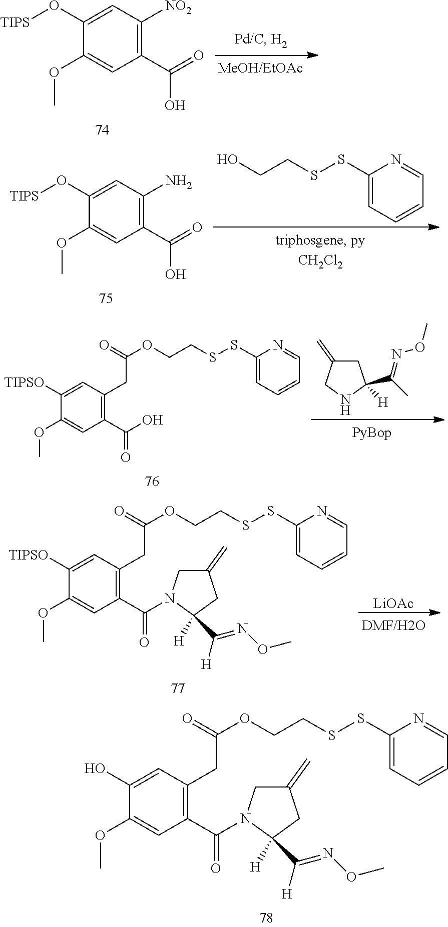

### Step 1: Preparation of Compound 75

A mixture of Compound 74 (0.410 g, 1.11 mmol) and 10% Pd/C (5%) in MeOH/EtOAc (5 mL/5 mL) was stirred under hydrogen atmosphere at room temperature for 3 h. The reaction mixture filtered through a pad of Celite, and the filtrate was concentrated in vacuo to give the product as yellow solid. The crude product was used without further purification: LC/MS: (ESI-QMS): m/z=340.26 (M+H), ¹H NMR (500 MHz, CDCl3): δ 7.34 (s, 1H), 6.28 (s, 1H), 3.75 (s, 3H), 1.27 (m, 3H), 1.10 (s, 9H), 1.08 (s, 9H).

### Step 2: Preparation of Compound 76

To a mixture of 2-(pyridin-2-yldisulfanyl)ethanol (65.2 mg, 0.35 mmol) and pyridine (61.9 μl, 0.77 mmol) in CH₂Cl₂ (1 mL) was added a solution of triphosgene (37.2 mg, 0.13 mmol) in CH₂Cl₂ (1 mL) at under argon. The mixture was stirred at 0° C. for 2 h, and then transferred to a mixture of Compound 75 (0.10 g, 0.29 mmol) and pyridine (51.6 μl, 0.64 mmol) in CH₂Cl₂ (1 mL) at 0° C. The reaction mixture was allowed to slowly warm to room temperature. After stirring for 3 h, the mixture was concentrated in vacuo and the crude product was purified via silica chromatography (0-60% EtOAc/pet. ether): LC/MS: (ESI-QMS): m/z=553.62 (M+H), ¹H NMR (500 MHz, CDCl₃): δ 10.38 (s, 1H), 8.76 (d, J=4.5 Hz, 2H), 8.47 (m, 2H), 8.0 (s, 1H), 7.75-7.71 (m, 1H), 7.69-7.61 (m, 1H), 7.53 (s, 1H), 7.08-7.05 (m, 1H), 4.41 (m, 2H), 3.81 (s, 3H), 3.12-3.05 (m, 2H), 1.33-1.28 (m, 3H), 1.16 (s, 9H), 1.10 (s, 9H).

### Step 3: Preparation of Compound 77

A mixture of Compound 76 (50.0 mg, 0.0900 mmol), Compound 68 (12.7 mg, 0.0900 mmol), PyBOP (70.2 mg, 0.140 mmol) and ⁱPr₂NEt (78.6 μL, 0.450 mmol) in DMSO (1 mL) was stirred at room temperature for 3 h under argon. The crude product was purified via silica chromatography (0-60% EtOAc/pet. ether): LC/MS: [(ESI-QMS): m/z=675.77 (M+H)

### Step 4: Preparation of Compound 78

To a mixture of Compound 77 (9.3 mg, 0.014 mmol) in DMF/H₂O (1 mL, 50:1 DMF/H₂O) was added lithium acetate (0.92 mg, 0.014 mmol) at room temperature. The reaction mixture was stirred at room temperature for 5 h. The mixture was concentrated in vacuo and the crude product was purified with preparative HPLC (10 to 100% MeCN/20 mM NH₄HCO₃ buffer, pH 7.4) to yield pure Compound 78: LC/MS: (ESI-QMS): m/z=519.57 (M+H), ¹H NMR (500 MHz, CDCl₃) δ 8.51 (d, J=4.5 Hz, 1H), 7.95 (m, 2H), 7.69 (m, 1H), 7.34 (s, 1H), 7.18 (dd, J₁=6.5 Hz, J₂=5 Hz, 1H), 6.84 (s, 1H), 6.77 (d, J=6 Hz, 1H), 5.06-4.99 (m, 2H), 4.38-4.35 (m, 2H), 4.14 (m, 2H), 3.88-3.85 (m, 2H), 3.10 (t, J=6.5 Hz, 2H), 2.89-2.84 (m, 2H).

### Example 47: Preparation of Conjugate 16

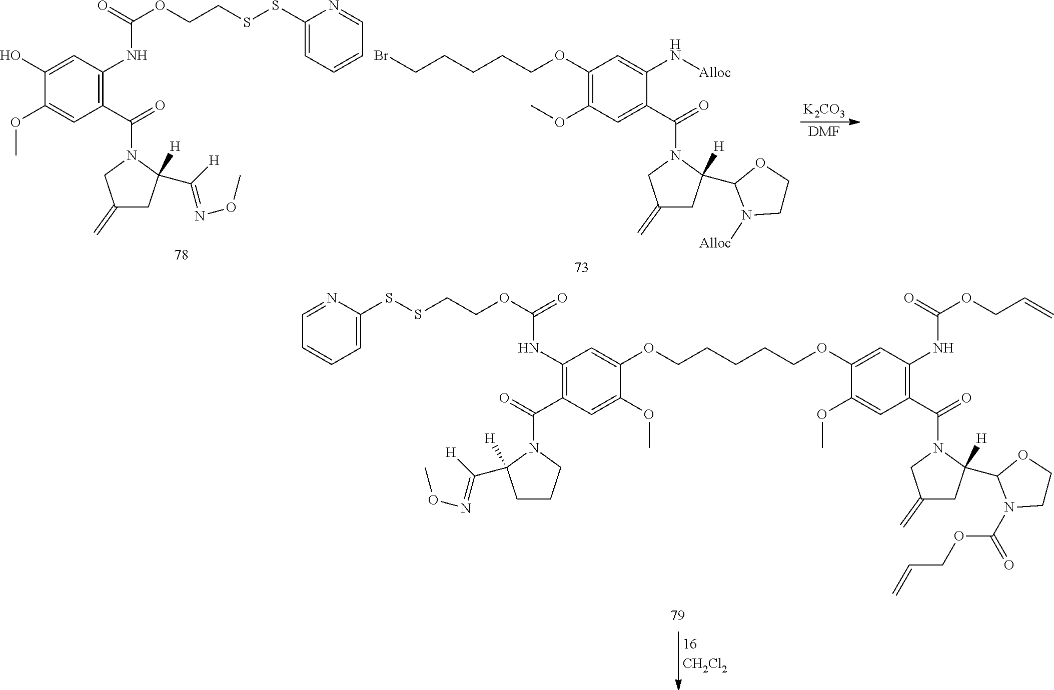

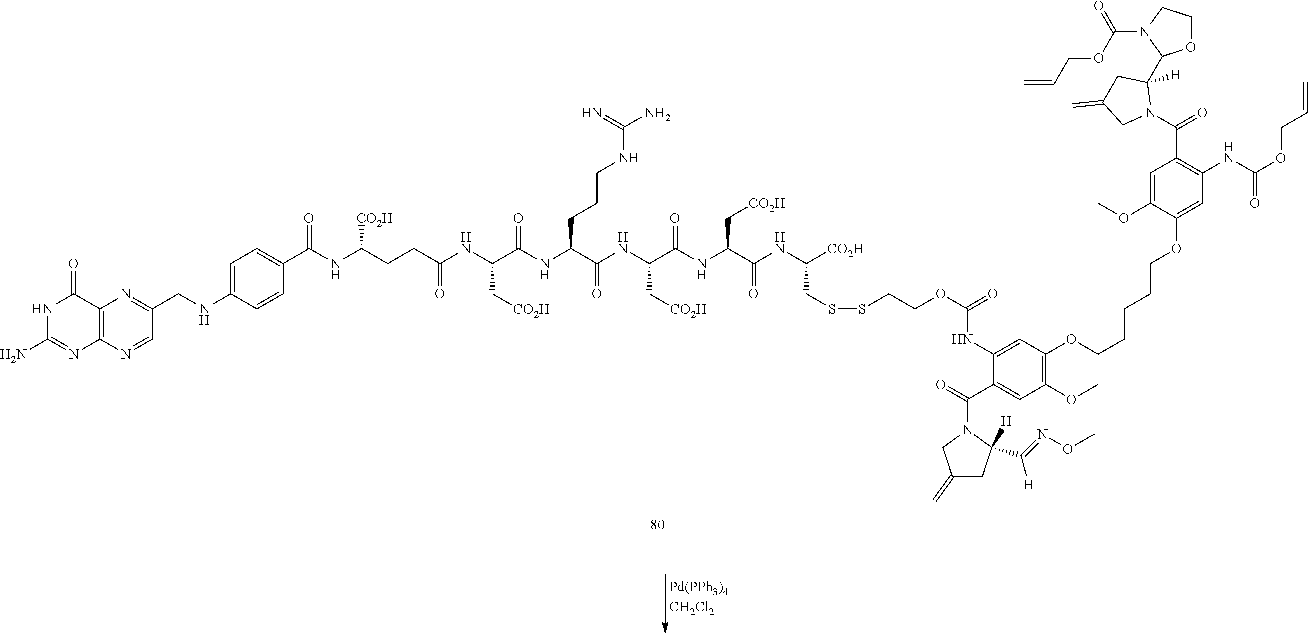 [SMILES: C=CCOC(=O)Nc1cc(OCCCCCOc2cc(NC(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c3ccc(NCc4cnc5nc(N)[nH]c(=O)c5n4)cc3)C(=O)O)C(=O)O)c(C(=O)N3CC(=C)C[C@H]3/C=N/OC)cc2OC)c(OC)cc1C(=O)N1CC(=C)C[C@H]1C1OCCN1C(=O)OCC=C]

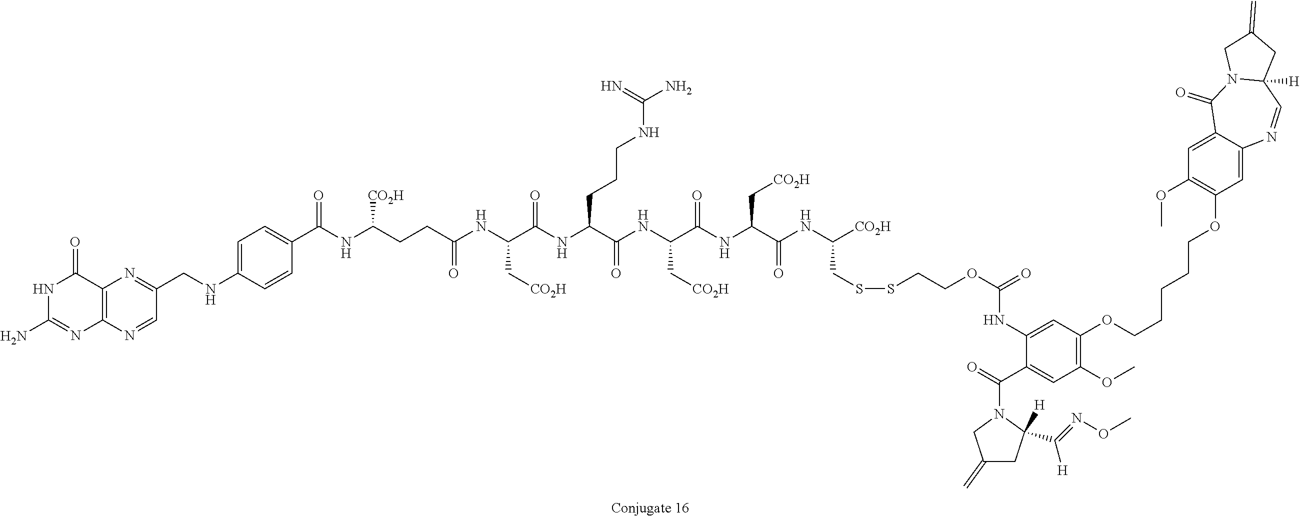 [SMILES: C=C1C[C@H]2C=Nc3cc(OCCCCCOc4cc(NC(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)C(=O)O)c(C(=O)N5CC(=C)C[C@H]5/C=N/OC)cc4OC)c(OC)cc3C(=O)N2C1]

### Step 1: Preparation of Compound 79

A mixture of Compound 73 (7.6 mg, 0.015 mmol), Compound 78 (9.3 mg, 0.015 mmol) and potassium carbonate (4.1 mg, 0.030 mmol) in DMF (1 mL) was stirred at 50° C. overnight under argon. The crude product was purified via preparative HPLC (10 to 100% MeCN/20 mM NH₄HCO₃ buffer, pH 7.4): LC/MS: (ESI-QMS): m/z=1075.13 (M+H).

### Step 2: Preparation of Conjugate 16

To a mixture of Compound 79 (24.6 mg, 0.023 mmol) and Et₃N (15.9 μl, 0.115 mmol) in DMSO (0.8 mL) was added Compound 16 (24.1 mg, 0.023 mmol) in MeOH (0.5 mL) at room temperature under argon. The mixture was stirred at room temperature for 3 h and then concentrated under high vacuum. CH₂Cl₂ (1 mL) was added to the crude residue followed by pyrrolidine (4.8 μl, 0.058 mmol) and Pd(PPh₃)₄ (1.33 mg, 0.0012 mmol) The reaction mixture was stirred at room temperature under argon for 4 h. The crude product was purified via preparative HPLC (10-100% MeCN/20 mM NH₄HCO₃ buffer, pH 7.4) to yield pure Conjugate 16: LC/MS: (ESI-QMS): m/z=890 (M+H).

### Example 48: Preparation of Compound 84

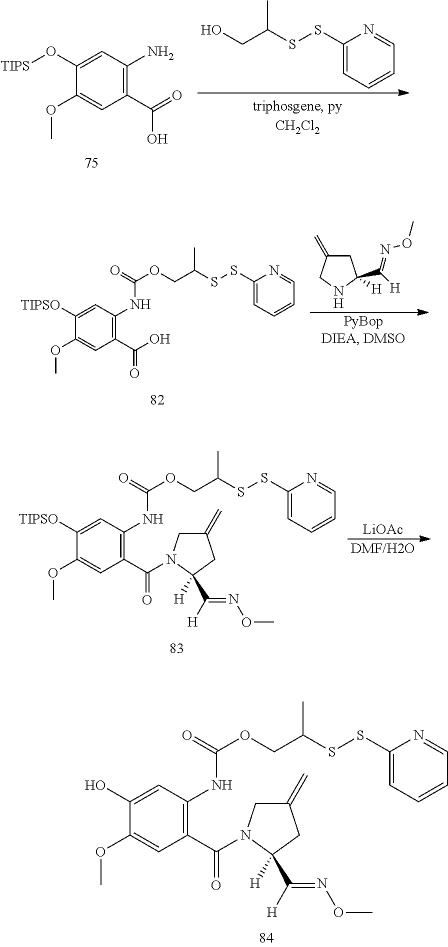

### Step 1: Preparation of Compound 82

Compound 82 was synthesized by following the procedure for Compound 76 from Compound 75: LC/MS: (ESI-QMS): m/z=553.62 (M+H), ¹H NMR (500 MHz, CDCl₃) δ 10.59 (s, 1H), 8.39 (s, 1H), 7.94 (s, 1H), 7.76 (d, J=7.4, 1H), 7.59 (t, J=7.8, 1H), 7.53 (s, 1H). 6.99-6.94 (m, 1H), 4.36 (d, J=5.8, 1H), 3.78 (s, 3H), 3.26-3.18 (m, 1H), 2.61 (s, 3H), 1.34 (d, J=1.34, 2H), 1.31-1.20 (m, 3H), 1.26 (d, J=6.8, 18H).

### Step 1: Preparation of Compound 83

Compound 83 was synthesized by following the procedure for Compound 77 from Compound 82 in lieu of Compound 76: LC/MS: [(ESI-QMS): m/z=675.77 (M+H), ¹H NMR (500 MHz, CDCl₃) δ 8.43 (s, 1H), 7.76 (d, J=7.4, 1H), 7.72-7.68 (m, 1H), 7.65 (t, J=7.9, 1H), 7.07-7.04 (m, 1H). 6.84 (s, 1H), 5.06 (s, 1H), 5.01 (s, br, 1H), 4.28-4.21 (m, 2H), 4.18-4.11 (m, 2H), 3.83 (s, 3H), 3.77 (s, 3H), 3.27-3.19 (m, 1H), 2.89-2.82 (m, 1H), 1.38 (m, 4H), 1.11 (d, J=7.4, 18H).

### Step 1: Preparation of Compound 84

Compound 84 was synthesized by following the procedure for Compound 78 from Compound 83 in lieu of Compound 77: LC/MS: (ESI-QMS): m/z=519.57 (M+H), ¹H NMR (500 MHz, CDCl₃) δ 8.43 (d, J=4.9 Hz, 1H), 7.76-7.72 (m, 1H), 7.72-7.67 (m, 1H), 7.63 (t, J=7.3, 1H), 7.45 (s, 1H), 7.05 (dd, J=7.3 Hz, J₂=7.3 Hz, 1H), 7.03 (s, 1H), 6.77 (d, J=6 Hz, 1H), 5.05 (s, 1H), 5.01 (s, br, 1H), 4.28-4.21 (m, 2H), 4.16-4.08 (m, 2H), 3.85, (s, 3H) 3.81 (s, 3H), 3.32 (dd, J₁=13.2, J₂=5.9, 1H), 2.87-2.79 (m, 2H), 1.34 (d, J=2.9, 3H).

### Example 49: Preparation of Conjugate 17

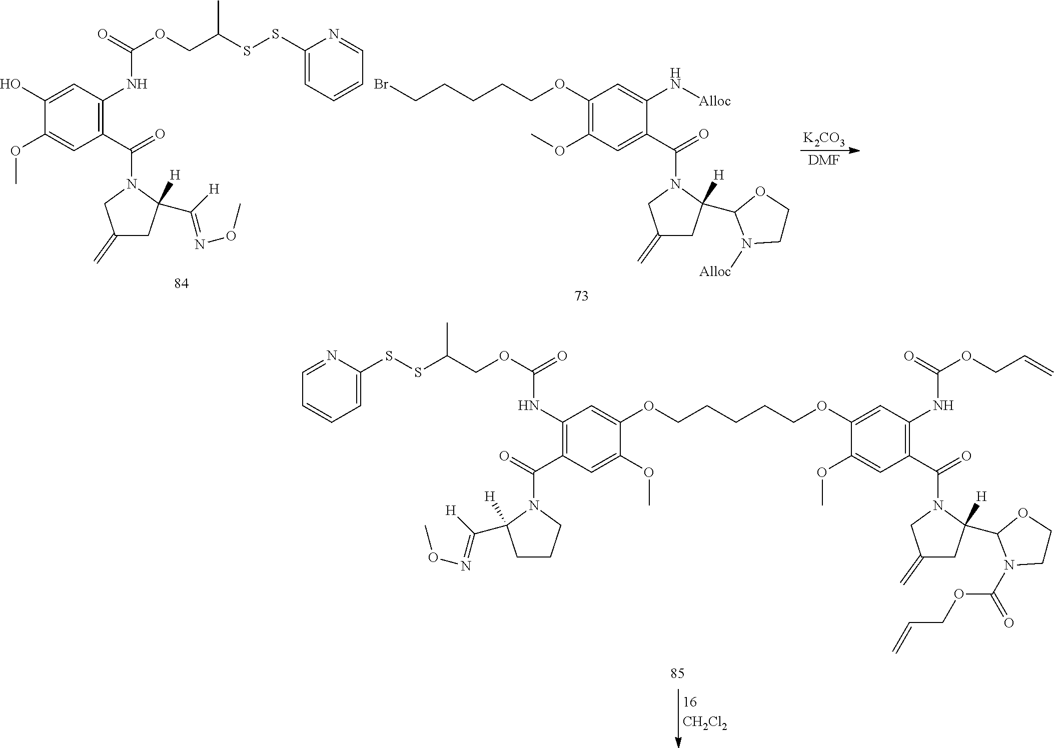

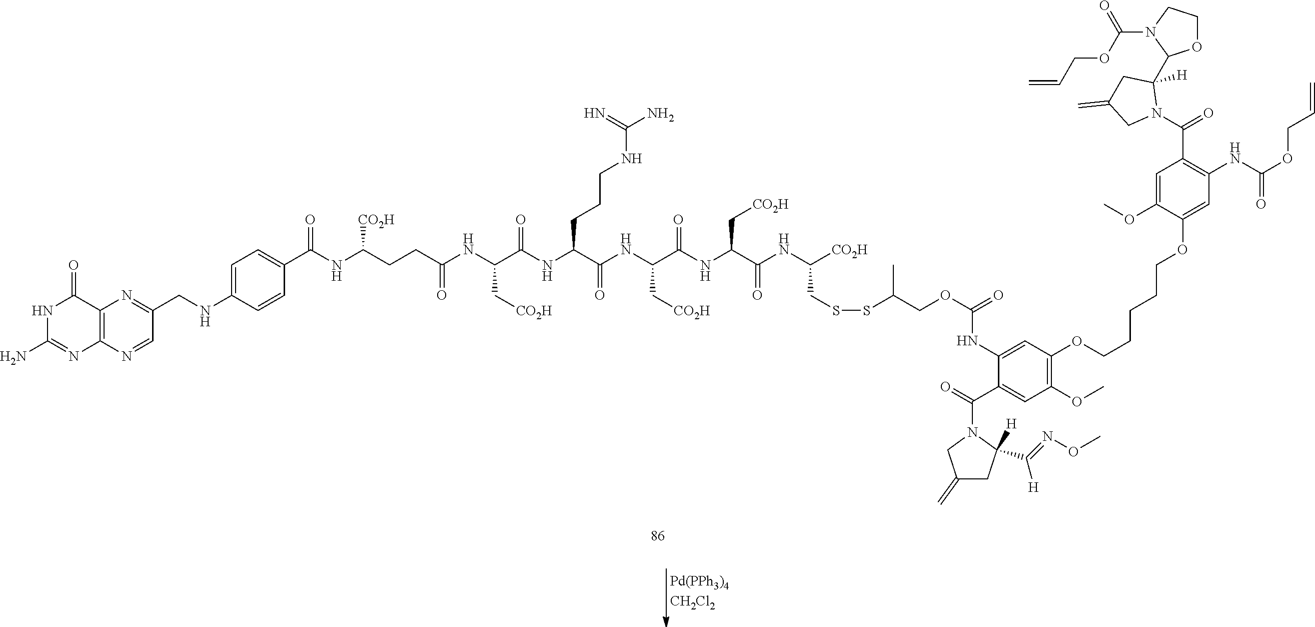 [SMILES: C=CCOC(=O)Nc1cc(OCCCCCOc2cc(NC(=O)OCC(C)SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c3ccc(NCc4cnc5nc(N)[nH]c(=O)c5n4)cc3)C(=O)O)C(=O)O)c(C(=O)N3CC(=C)C[C@H]3/C=N/OC)cc2OC)c(OC)cc1C(=O)N1CC(=C)C[C@H]1C1OCCN1C(=O)OCC=C]

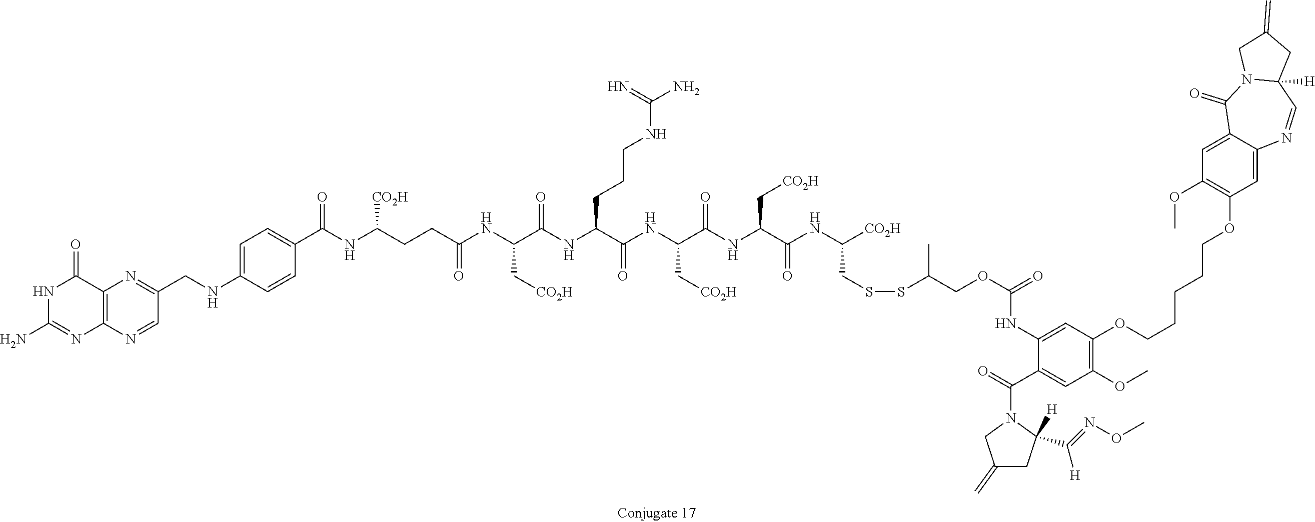 [SMILES: C=C1C[C@H]2C=Nc3cc(OCCCCCOc4cc(NC(=O)OCC(C)SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)C(=O)O)c(C(=O)N5CC(=C)C[C@H]5/C=N/OC)cc4OC)c(OC)cc3C(=O)N2C1]

### Step 1: Preparation of Compound 85

Compound 85 was synthesized by following the procedure for Compound 79 from Compound 84 in lieu of Compound 78: LC/MS: (ESI-QMS): m/z=1088.46 (M+H).

### Step 2: Preparation of Compound 86

Compound 86 was synthesized by following the procedure for Compound 80 from Compound 85 in lieu of Compound 79: LC/MS: (ESI-QMS): m/z=1011.84 (M+2H), 675.12 (M+3H).

### Step 3: Preparation of Conjugate 17

Conjugate 17 was synthesized by following the procedure for Conjugate 16 from Compound 86 in lieu of Compound 80: LC/MS: (ESI-QMS): m/z=897.82 (M+2H), 598.63 (M+3H).

### Example 50: Preparation of Compound 88

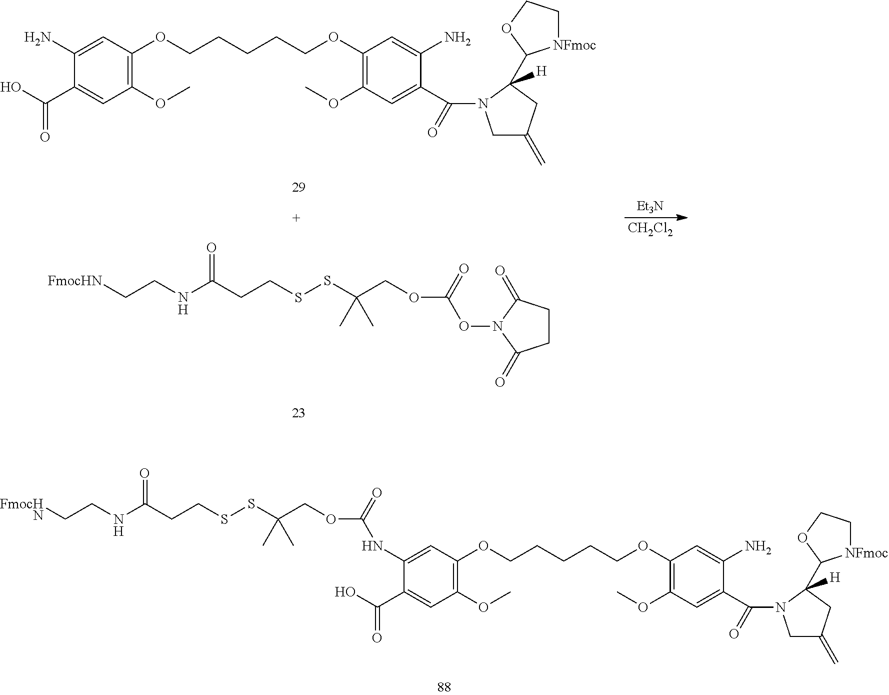

Et₃N (12.5 μL, 89.3 μmol) was added to a solution of Compound 29 (32.3 mg, 40.6 μmol) and Compound 23 (25.1 mg, 40.6 μmol) in anhydrous CH₂Cl₂ (1.5 mL). The reaction mixture was stirred at room temperature under argon for 3 h and then purified via silica chromatography (0-10%, MeOH/CH₂C₂) to yield Compound 88 as a white solid (42.6 mg, 81.1%): (ESI-QMS): m/z=1294.31 (M+H), Selective H NMR (500 MHz, 298 K, CD₂Cl₂) δ 7.765 (b, 4H), 7.584 (b, 4H), 7.487 (b, 2H), 7.386 (b, 4H), 7.305 (b, 6H).

### Example 51: Preparation of Compound 89

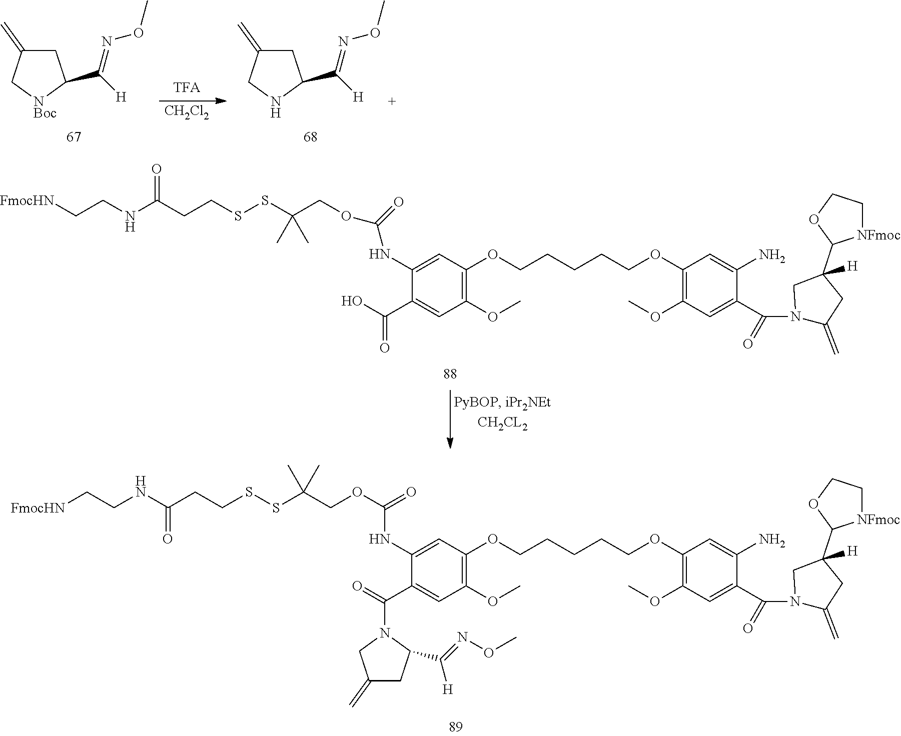

TFA (0.50 mL) was added to a solution of Compound 67 (10.1 mg, 40.2 μmol) in anhydrous CH₂Cl₂ (0.50 mL). The reaction mixture was stirred at room temperature under argon for 35 min and concentrated under reduced pressure. The residue was co-evaporated with CH₂Cl₂ (1 mL×3), and dried under high vacuum for 1 h. To the residue was added a solution of Compound 88 (40.0 mg, 30.9 μmol) and PyBOP (17.7 mg, 34.0 μmol) in anhydrous CH₂Cl₂ (1.0 mL) and ⁱPr₂NEt (30.0 μL, 5.5 170 μmol) in tandem. The reaction mixture was stirred at room temperature under argon for 1 h and purified via silica chromatography (0-10% MeOH/CH₂C₂) to yield Compound 89 as a beige solid (40.8 mg). The purity of the product was about 50-60% based on LC/MS analysis and was used without further purification. LC/MS: (ESI-QMS): m/z=1416.31 (M+H).

### Example 52: Preparation of Conjugate 18

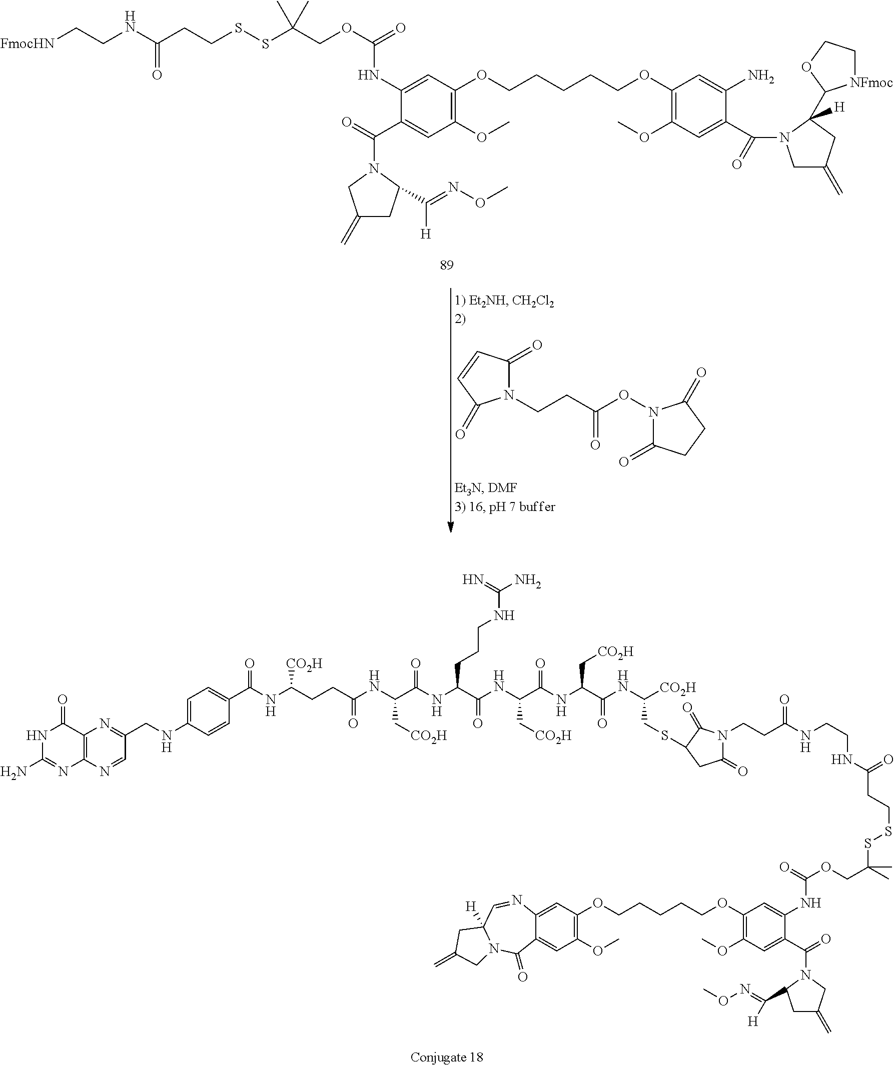

Diethylamine (0.75 mL) was added to a solution of Compound 89 (40.1 mg, 28.3 μmol) in anhydrous CH₂Cl₂ (1.0 mL). The reaction mixture was stirred at room temperature under argon for 3 h and concentrated under reduced pressure. The residue was co-evaporated with CH₂Cl₂ (1.5 mL×3), dried under high vacuum for 1 h, and dissolved in anhydrous DMF (2.0 mL). To the solution was added maleimidopropionic acid NHS ester (8.3 mg, 31.1 μmol) and Et₃N (8.0 μL, 57 μmol) in tandem. The reaction mixture was stirred at room temperature under argon for 1 h, diluted with DMF (2.5 mL), and to which was added a solution of Compound 16 (38.5 mg, 36.8 μmol) in 50 mM NH₄HCO₃ buffer, pH 7.0 (5.0 mL). The reaction mixture was stirred at room temperature under argon for 15 min and purified via preparative HPLC (10-100%, MeCN/50 mM NH₄HCO₃ buffer, pH 7.0 to yield Conjugate 18 as a fluffy yellow solid (18.3 mg, 30.7% over three steps): LC/MS: (ESI-QMS): m/z=1052.55 (M+2H), Selective H NMR (500 MHz, 298 K, DMSO-d6 with D₂O exchange) δ 8.607 (s, 1H), 7.569 (d, J=8.5 Hz, 2H), 7.003 (s, 1H), 6.865 (b, 1H), 6.625 (d, J=8.5 Hz, 2H).

### Example 53: Preparation of Conjugate 19

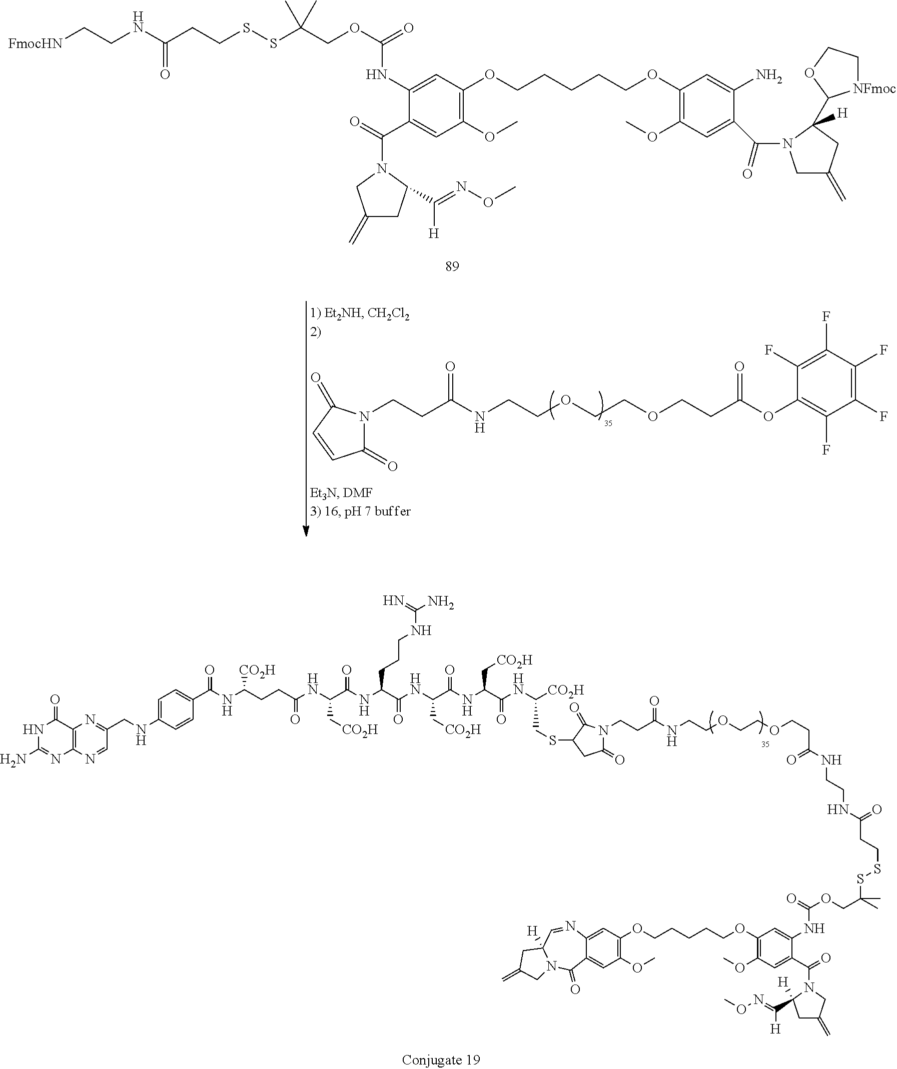

Mal-dPEG₄-TFP ester, Mal-dPEG₁₂-TFP ester, and Mal-dPEG₃₆-TFP ester were obtained from Quanta BioDesign Ltd.

Diethylamine (0.75 mL) was added to a solution of Compound 89 (50.2 mg, 35.5 μmol) in anhydrous CH₂Cl₂ (0.75 mL). The reaction mixture was stirred at room temperature under argon for 2.5 h and concentrated under reduced pressure. The residue was co-evaporated with CH₂Cl₂ (1 mL×3), dried under high vacuum for 1 h, and dissolved in anhydrous DMF (2.0 mL). To the solution was added MAL-dPEG₃₆-TFP ester (70.0 mg, 35.5 μmol) and Et₃N (10.0 μL, 71 μmol) in tandem. The reaction mixture was stirred at room temperature under argon for 45 min, diluted with DMF (2.5 mL), and to which was added a solution of Compound 16 (50.1 mg, 46.2 μmol) in 50 mM NH₄HCO₃ buffer, pH 7.0 (5.0 mL). The reaction mixture was then stirred at room temperature under argon for 15 min and purified via preparative HPLC (10-100%, MeCN/50 mM NH₄HCO₃ buffer, pH 7.0) to afford Conjugate 19 as a fluffy yellow solid (19.1 mg, 14.3% over three steps): LC/MS: (ESI-QMS): m/z=11880.76 (M+3H), Selective H NMR (500 MHz, 298 K, DMSO-d6 with D₂O exchange) δ 8.624 (s, 1H), 7.578 (d, J=8.5 Hz, 2H), 6.888 (b, 1H), 6.523 (d, J=8.5 Hz, 2H).

### Example 54: Preparation of Conjugate 20

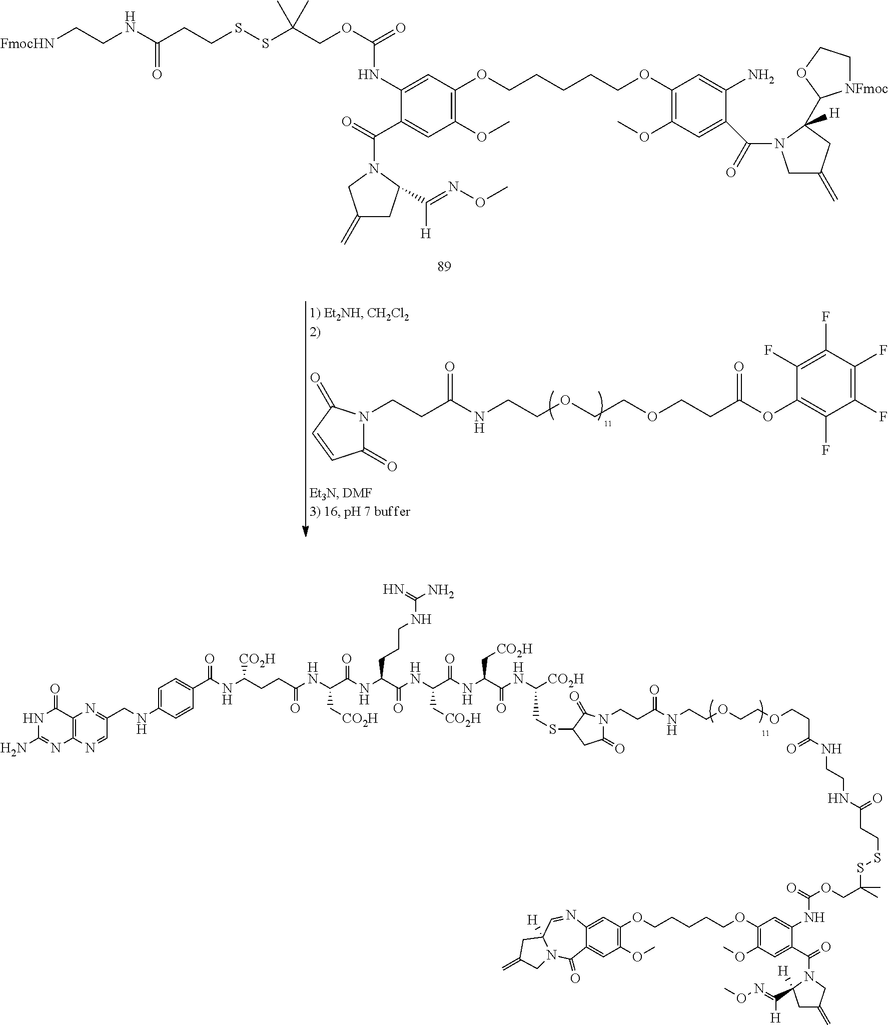

Diethylamine (0.75 mL) was added to a solution of Compound 89 (51.7 mg, 36.5 μmol) in anhydrous CH₂Cl₂ (0.75 mL). The reaction mixture was stirred at room temperature under argon for 2.5 h and concentrated under reduced pressure. The residue was co-evaporated with CH₂Cl₂ (1 mL×3), dried under high vacuum for 1 h, and dissolved in anhydrous DMF (2.0 mL). To the solution was added MAL-dPEG₁₂-TFP ester (34.8 mg, 40.2 μmol) and Et₃N (11.2 μL, 73 μmol) in tandem. The reaction mixture was stirred at room temperature under argon for 45 min, diluted with DMF (2.5 mL), and to which was added a solution of Compound 16 (53.5 mg, 51.1 μmol) in 50 mM NH₄HCO₃ buffer, pH 7.0 (5.0 mL). The reaction mixture was stirred at room temperature under argon for 15 min and purified via preparative HPLC (10-100%, MeCN/50 mM NH₄HCO₃ buffer, pH 7.0) to yield Conjugate 20 as a fluffy yellow solid (6.1 mg, 6.2% over three steps): LC/MS: (ESI-QMS): m/z=1354.57 (M+2H), Selective H NMR (500 MHz, 298 K, DMSO-d6 with D₂O exchange) δ 8.709 (b, 1H), 7.666 (b, 2H), 7.158 (s, 1H), 7.059 (s, 1H), 7.012 (s, 1H), 6.930 (s, 1H), 6.764 (b, 2H).

### Example 55: Preparation of Conjugate 21

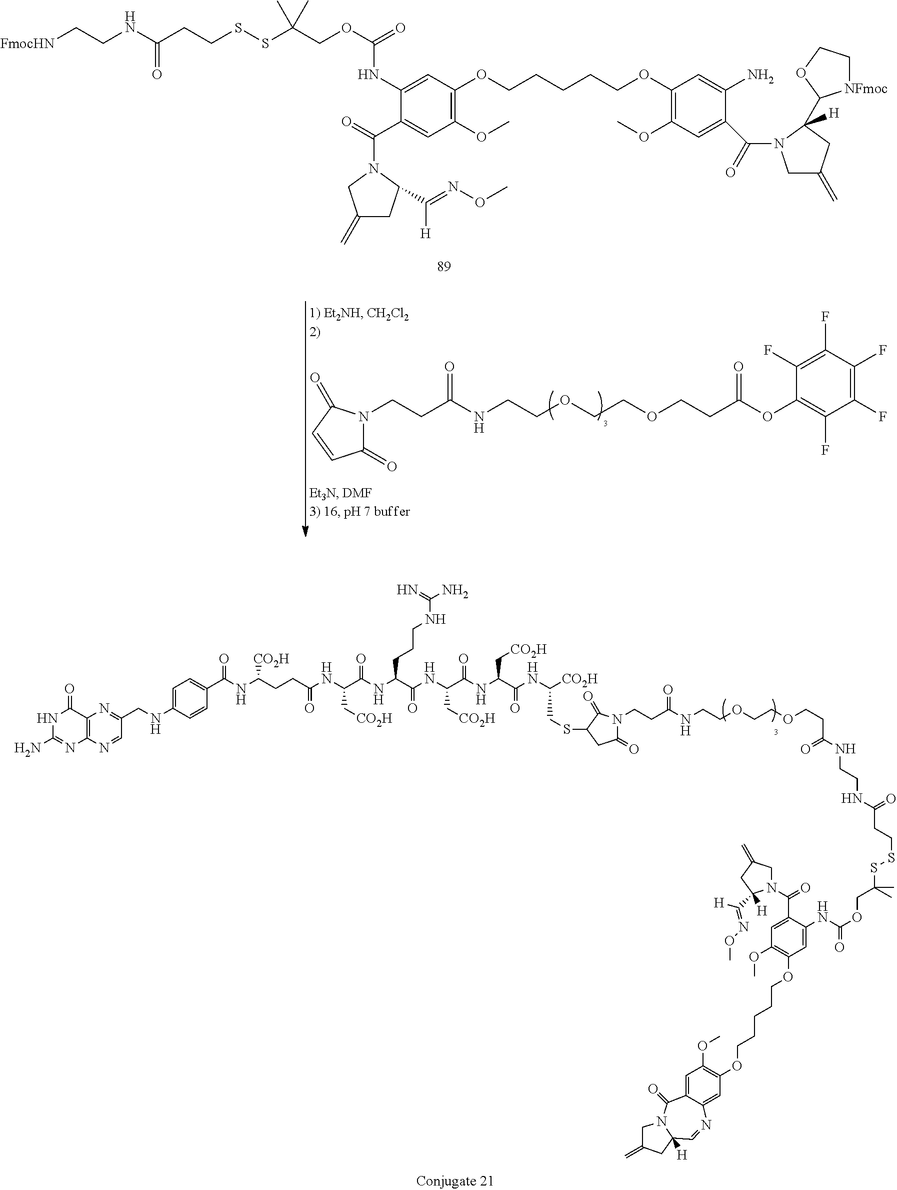

Diethylamine (0.70 mL) was added to a solution of Compound 89 (55.0 mg, 38.9 μmol) in anhydrous CH₂Cl₂ (0.70 mL). The reaction mixture was stirred at room temperature under argon for 2.5 h and concentrated under reduced pressure. The residue was co-evaporated with CH₂Cl₂ (1 mL×3), dried under high vacuum for 1 h, and dissolved in anhydrous DMF (2.0 mL). To the solution was added MAL-dPEG₄-TFP ester (23.9 mg, 46.7 μmol) and Et₃N (12.0 μL, 85.6 μmol) in tandem. The reaction mixture was then stirred at room temperature under argon for 45 min, diluted with DMF (2.5 mL), and to which was added a solution of Compound 16 (56.9 mg, 54.5 mmol) in 50 mM NH₄HCO₃ buffer, pH 7.0 (5.0 mL). The reaction mixture was stirred at room temperature under argon for 15 min and purified via preparative HPLC (10-100% MeCN/50 mM NH₄HCO₃ buffer, pH 7.0) to afford Conjugate 21 as a fluffy yellow solid (18.0 mg, 19.7% over three steps): LC/MS: (ESI-QMS): m/z=1176.17 (M+2H), Selective H NMR (500 MHz, 298 K, DMSO-d6 with D₂O exchange) δ 8.619 (s, 1H), 7.577 (d, J=8.5 Hz, 2H), 7.045 (s, 1H), 7.014 (s, 1H), 6.883 (b, 1H), 6.629 (d, J=8.5 Hz, 2H).

### Example 55: Preparation of Conjugate 21

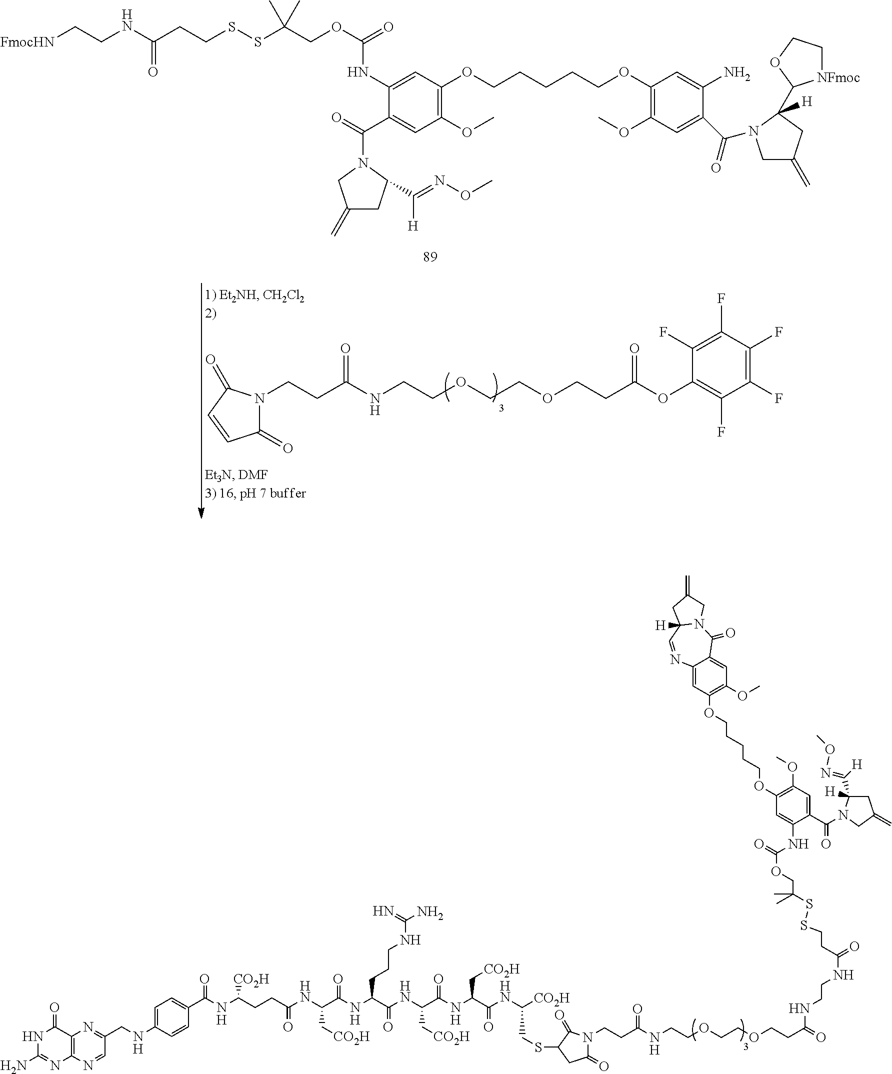

Diethylamine (0.70 mL) was added to a solution of Compound 89 (55.0 mg, 38.9 μmol) in anhydrous CH₂Cl₂ (0.70 mL). The reaction mixture was stirred at room temperature under argon for 2.5 h and concentrated under reduced pressure. The residue was co-evaporated with CH₂Cl₂ (1 mL×3), dried under high vacuum for 1 h, and dissolved in anhydrous DMF (2.0 mL). To the solution was added MAL-dPEG₄-TFP ester (23.9 mg, 46.7 μmol) and Et₃N (12.0 μL, 85.6 μmol) in tandem. The reaction mixture was then stirred at room temperature under argon for 45 min, diluted with DMF (2.5 mL), and to which was added a solution of Compound 16 (56.9 mg, 54.5 mmol) in 50 mM NH₄HCO₃ buffer, pH 7.0 (5.0 mL). The reaction mixture was stirred at room temperature under argon for 15 min and purified via preparative HPLC (10-100% MeCN/50 mM NH₄HCO₃ buffer, pH 7.0) to afford Conjugate 21 as a fluffy yellow solid (18.0 mg, 19.7% over three steps): LC/MS: (ESI-QMS): m/z=1176.17 (M+2H), Selective ¹H NMR (500 MHz, 298 K, DMSO-d6 with D₂O exchange) δ 8.619 (s, 1H), 7.577 (d, J=8.5 Hz, 2H), 7.045 (s, 1H), 7.014 (s, 1H), 6.883 (b, 1H), 6.629 (d, J=8.5 Hz, 2H).

### Example 56: Preparation of Conjugate 22

### Step 1: Preparation of Compound 90

Compound 90 was synthesized by following the same procedure as for the preparation of Compound 34 from EMCS in lieu of Mal-PEG4-NHS ester: LC/MS (ESI-QMS): m/z=1117 (M+H).

### Step 2: Preparation of Conjugate 22

Conjugate 22 was prepared according to the procedure described above for Conjugate 5 by reacting Compound 90 with Compound 16 instead of Compound 34. Yield: 9% for 3 steps. LC/MS (ESI-QMS), (M+2H)^{2+: 1082}.

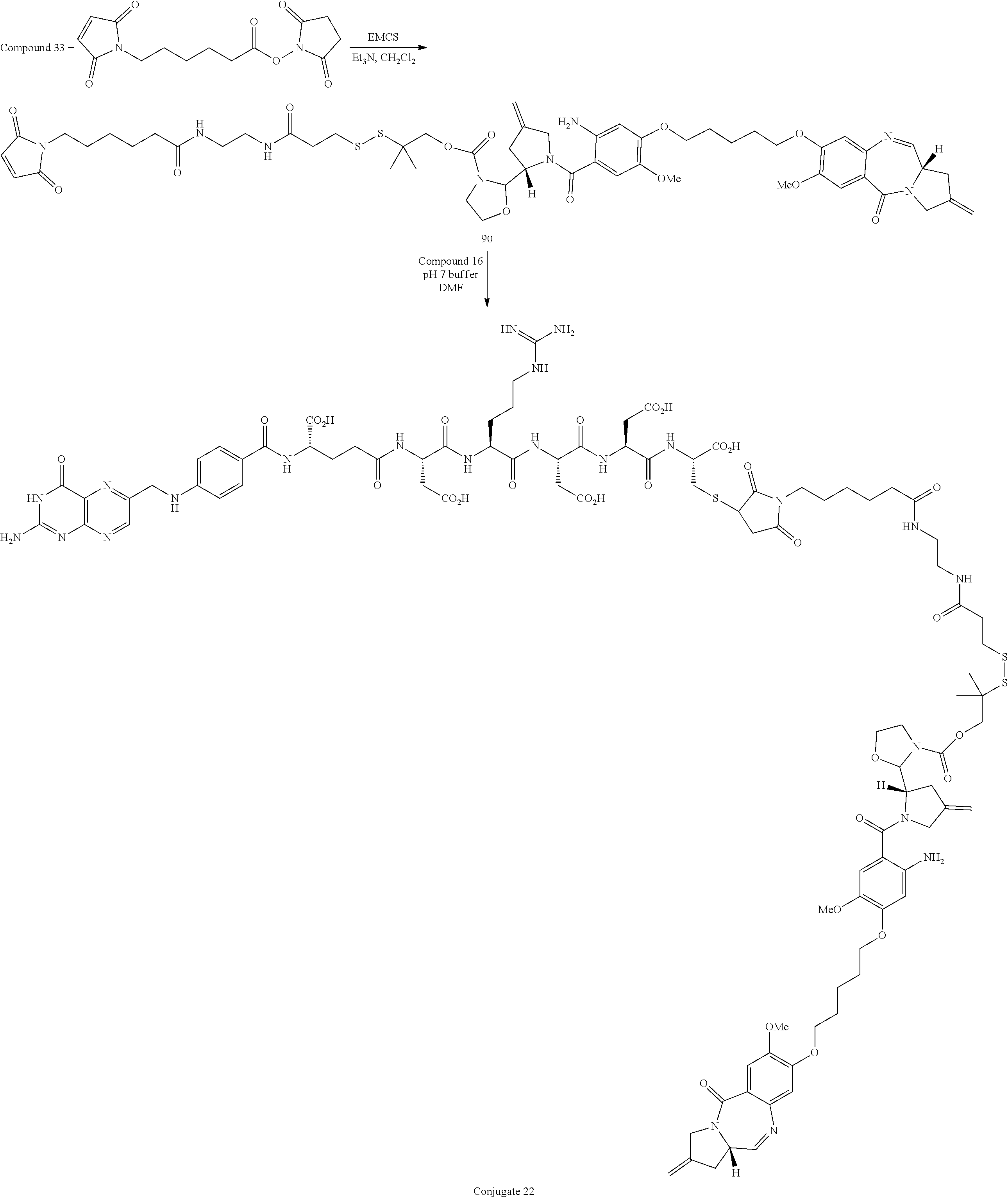 [SMILES: O=C(CCCCCN1C(=O)C=CC1=O)ON1C(=O)CCC1=O ; C=C1C[C@H]2C=Nc3cc(OCCCCCOc4cc(N)c(C(=O)N5CC(=C)C[C@H]5C5OCCN5C(=O)OCC(C)(C)SSCCC(=O)NCCNC(=O)CCCCCN5C(=O)C=CC5=O)cc4OC)c(OC)cc3C(=O)N2C1 ; C=C1C[C@H]2C=Nc3cc(OCCCCCOc4cc(N)c(C(=O)N5CC(=C)C[C@H]5C5OCCN5C(=O)OCC(C)(C)SSCCC(=O)NCCNC(=O)CCCCCN5C(=O)CC(SC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c6ccc(NCc7cnc8nc(N)[nH]c(=O)c8n7)cc6)C(=O)O)C(=O)O)C5=O)cc4OC)c(OC)cc3C(=O)N2C1]

### Example 57: Preparation of Conjugate 23

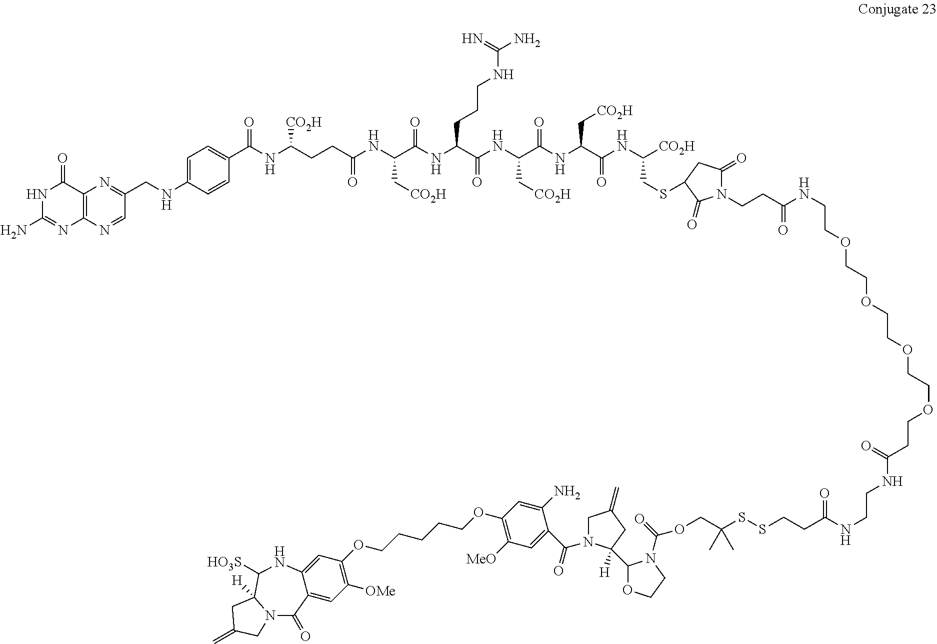 [SMILES: C=C1C[C@@H](C2OCCN2C(=O)OCC(C)(C)SSCCC(=O)NCCNC(=O)CCOCCOCCOCCOCCNC(=O)CCN2C(=O)CC(SC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c3ccc(NCc4cnc5nc(N)[nH]c(=O)c5n4)cc3)C(=O)O)C(=O)O)C2=O)N(C(=O)c2cc(OC)c(OCCCCCOc3cc4c(cc3OC)C(=O)N3CC(=C)C[C@H]3C(S(=O)(=O)O)N4)cc2N)C1]

To the solution of Conjugate 5 (5.7 mg, 0.0024 mmol) in DMSO (0.4 mL) and water (0.4 mL) was added NaHSO₃ (0.37 mg, 0.0036 mmol) and the reaction was stirred for 2 h. The reaction was then purified with prep-HPLC in 10-100% CH₃CN/NH₄HCO₃ buffer (pH 7.4, 50 mM) to provide Conjugate 23 (2.8 mg, 48% yield). LC/MS (ESI-QMS): (M+2H)²⁺: 1226.

### Example 58: Preparation of Conjugate 24

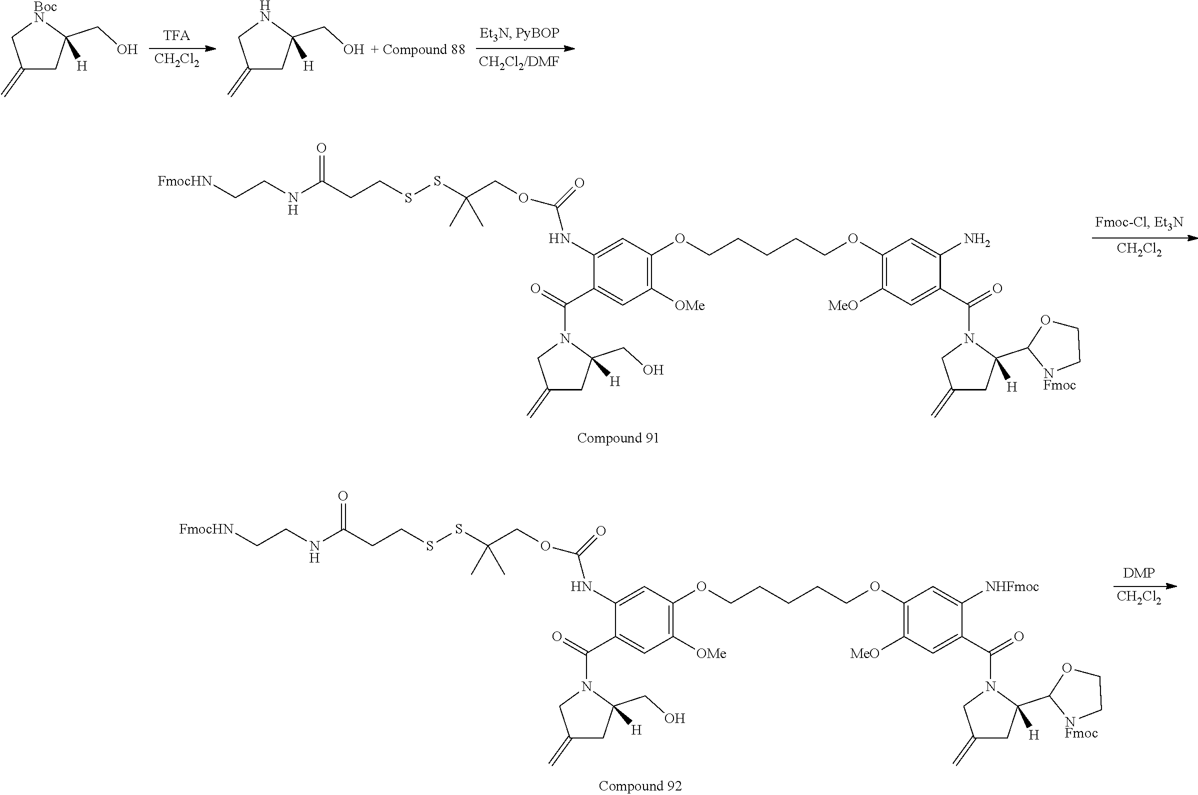

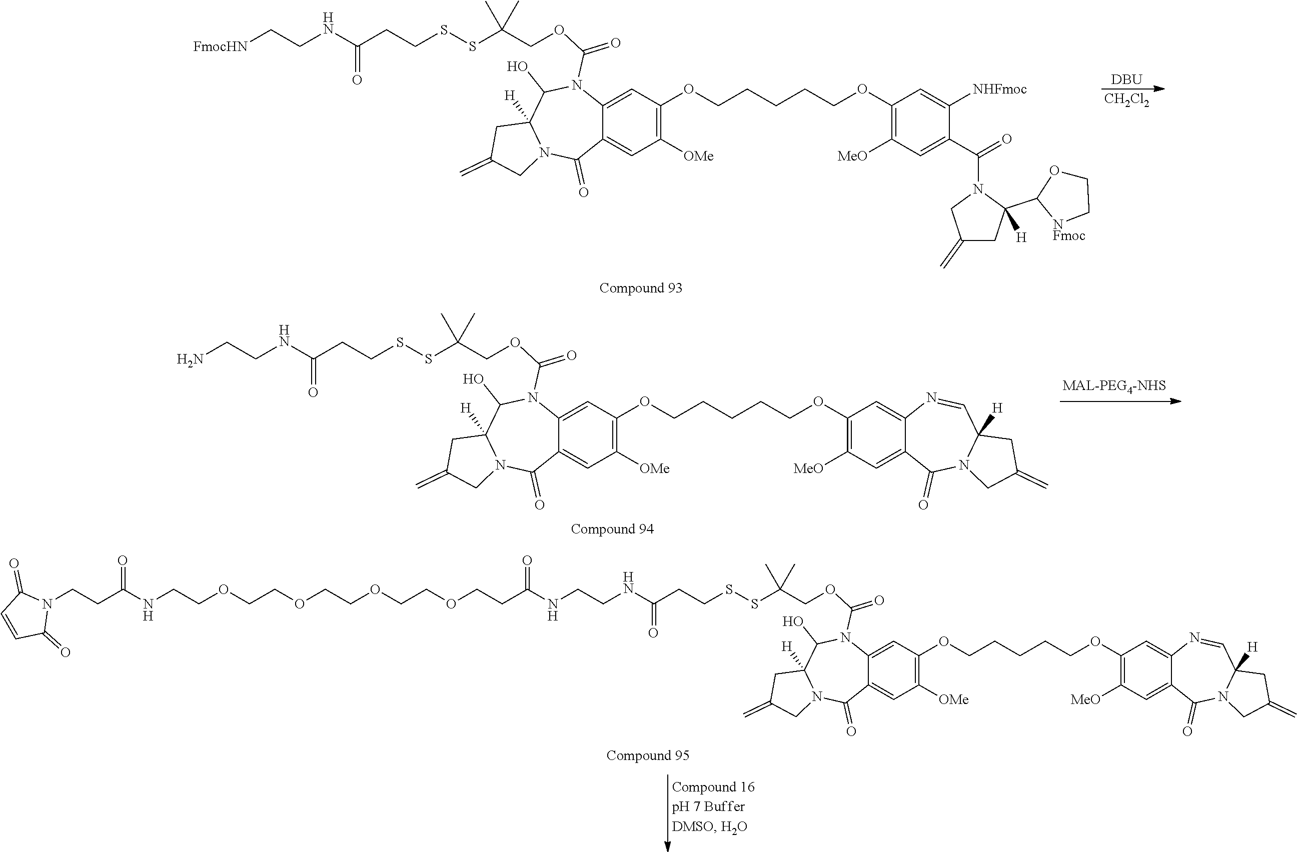

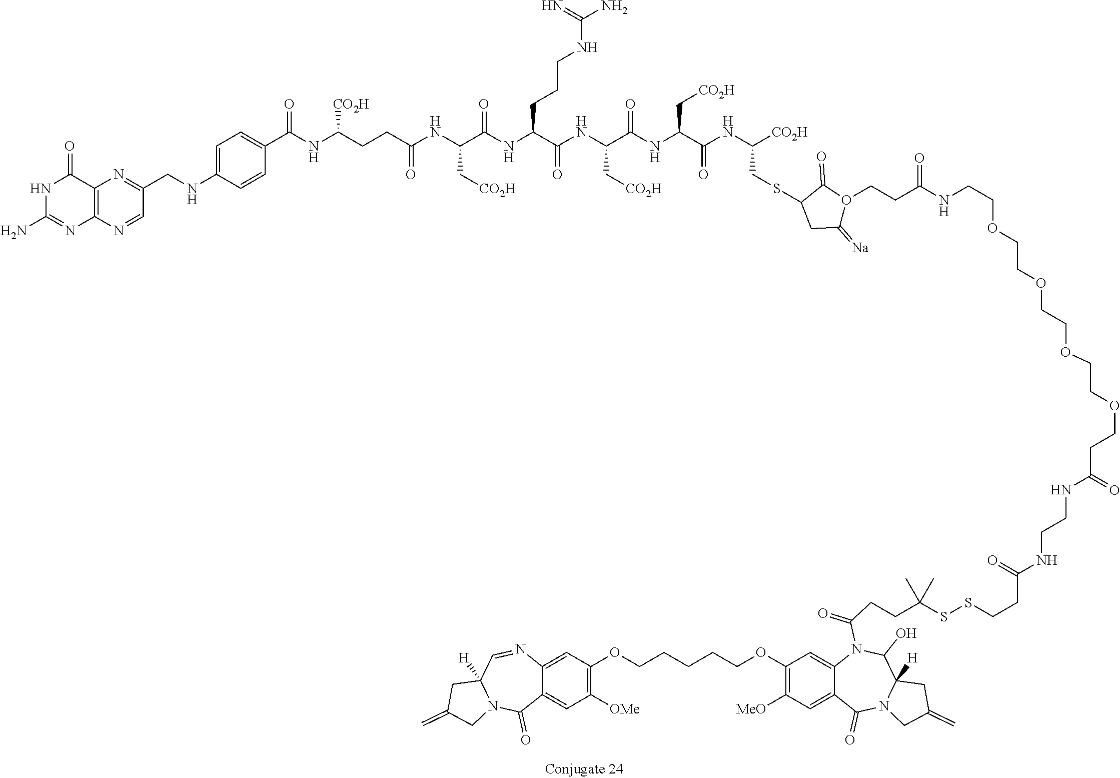

### Step 1: Preparation of Compound 91

Boc-protected prolinol derivative (6.72 mg, 0.0315 mmol) was added to TFA/CH₂Cl₂ (0.5 mL/0.5 mL) and stirred for 30 min at room temperature. The solvent was removed in vacuo. The residue was dissolved in CH₂Cl₂ (1.0 mL), and added to a solution of Compound 88 (40.74 mg, 0.0315 mmol) and Et₃N (8.8 μl, 0.063 mmol) in DMF (0.5 mL). The reaction mixture was treated with PyBOP (18.0 mg, 0.0347 mmol) at stirred for 1 h at room temperature. The desired product was isolated via silica chromatography in 0-10% CH₃OH/CH₂Cl₂, yielding 41.0 mg Compound 91 (94%). LC/MS (ESI-QMS): (M+H)⁺: 1389.

### Step 2: Preparation of Compound 92

FmocCl (11.46 mg, 0.0444 mmol) was added to a stirring solution of Compound 91 (20.5 mg, 0.0148 mmol) in CH₂Cl₂ (0.5 mL). The reaction mixture was then treated with Et₃N (2.1 uL, 0.0148 mmol) and stirred for 5 h. The desired product was purified via silica chromatography with 0-10% CH₃OH/CH₂Cl₂ to yield 14.2 mg of Compound 92 (60%): LC/MS (ESI-QMS): (M+H)⁺: 1611.

### Step 3: Preparation of Compound 93

Compound 92 (14.3 mg, 0.00888 mmol) was added to a solution of Dess-Martin-periodinane (5.65 mg, 0.0133 mmol) in CH₂Cl₂ (0.5 mL). The reaction mixture was stirred at room temperature for 2 h. The desired product was purified via silica chromatography with 0-10% CH₃OH/CH₂Cl₂ to yield 17.7 mg of Compound 93: LC/MS (ESI-QMS): (M+H)⁺: 1609.

### Step 4: Preparation of Conjugate 24

Compound 93 (17.7 mg, 0.0128 mmol) in CH₂Cl₂ (0.3 mL) was treated with DBU (1.9 L, 0.0128 mmol) for 30 min at room temperature. The reaction mixture was then neutralized with AcOH (0.7 μL, 0.0128 mmol). Compound 94 was observed via LC/MS: LC/MS (ESI-QMS): (M+H)+: 881. Mal-PEG₄-NHS ester (6.6 mg, 0.0128 mmol) was then added to the crude mixture of Compound 94 to give Compound 95, (M+H)+: 1280. The reaction mixture was then concentrated to dryness under high vacuum. The crude residue of Compound 95 was dissolved with a solution of Compound 16 (13.4 mg, 0.0128 mmol) in DMSO/PBS buffer (0.3 mL/0.3 mL). The desired product was purified with prep-HPLC in 10-100% CH₃CN/NH₄HCO₃ (pH7.4, 50.0 mM) to yield 1.5 mg of Conjugate 24 (5% for 4 steps): LC/MS (ESI-QMS): (M+2H)²⁺: 1163.

### Example 59: Preparation of Conjugate 25

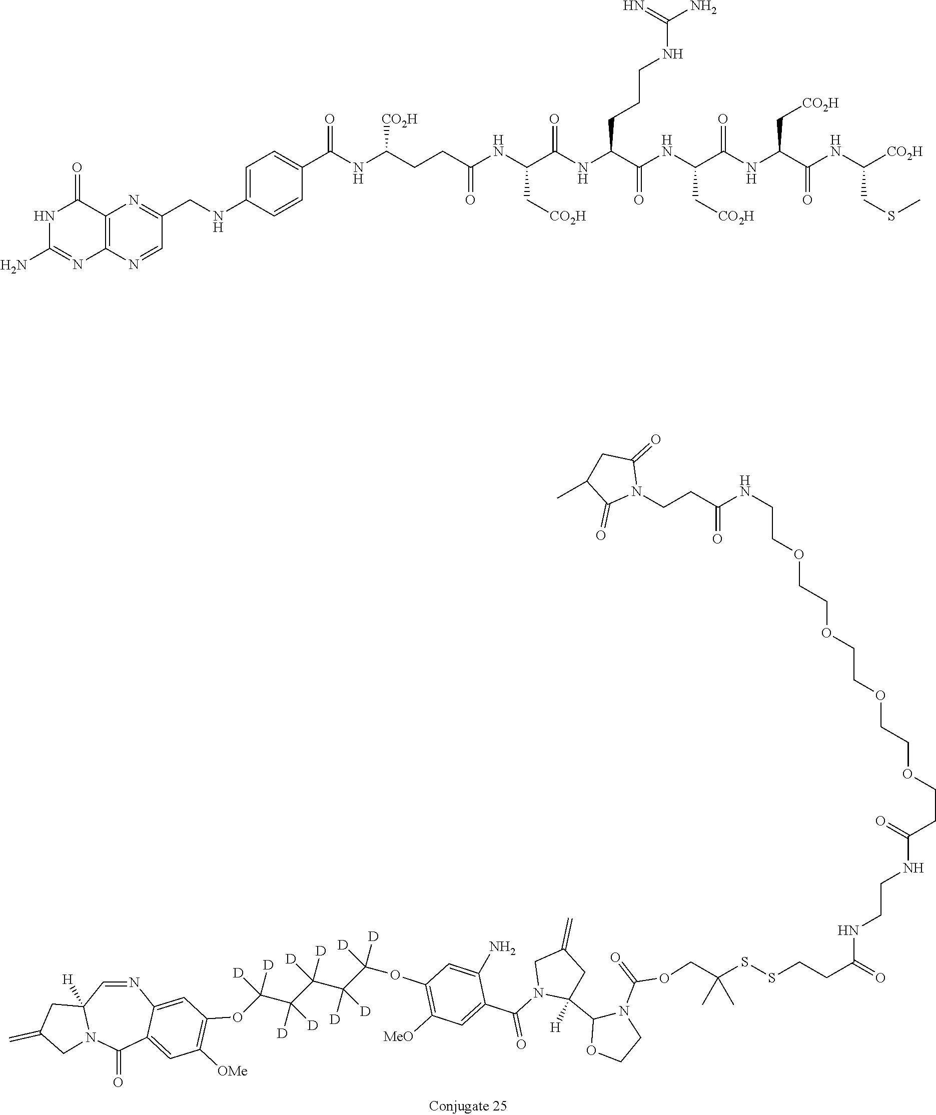

Conjugate 25 was synthesized by following the procedure for Conjugate 5 from d-10 Compound 6 in lieu of Compound 6: LC/MS (ESI-QMS): (M+3H)³⁺: 794. H NMR (500 MHz, 9:1 DMSO-d6:D₂₀₎ δ 8.62 (s, 1H), 7.58 (d, J=8.5 Hz, 2H), 7.13 (s, 1H), 7.01 (s, 1H), 6.63 (d, J=8.5 Hz, 3H), 6.51 (s, 1H), 6.32 (s, 1H), 5.09 (s, 1H), 5.06 (s, 1H), 4.99 (s, 1H), 4.94 (d, J=8.5 Hz, 2H), 4.89 (s, 1H), 4.53 (m, 2H), 4.47 (m, 3H), 4.38 (m, 1H) 4.23 (m, 1H), 4.0-4.2 (m, 4H), 3.99 (s, 2H), 3.83 (m, 6H), 3.66 (s, 3H), 3.61 (m, 4H), 3.55 (m, 6H), 3.35 (m, 3H), 3.13 (m 10H), 2.83 (m, 6H), 2.63 (m 3H), 2.41 (m, 8H), 2.28 (m, 5H), 2.14 (b, 3H), 1.80-1.95 (b, 6H), 1.59 (m, b, 2H), 1.30-1.50 (m, b, 4H), 1.22 (b, 6H).

### Example 60: Preparation of Conjugate 26

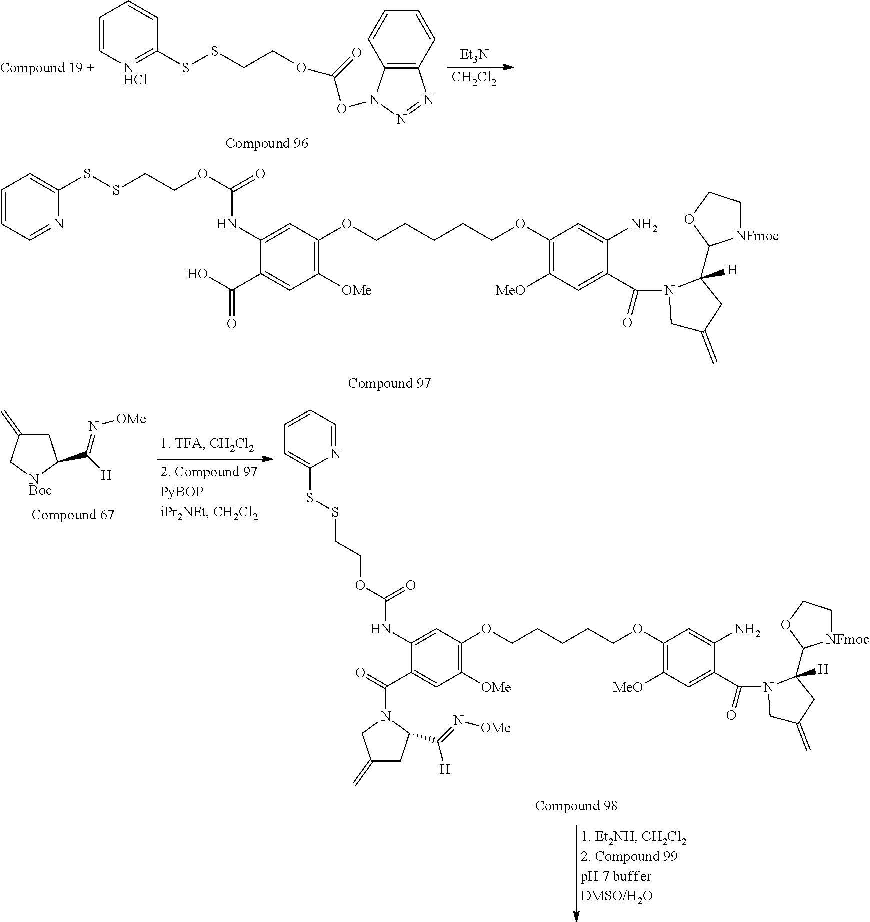

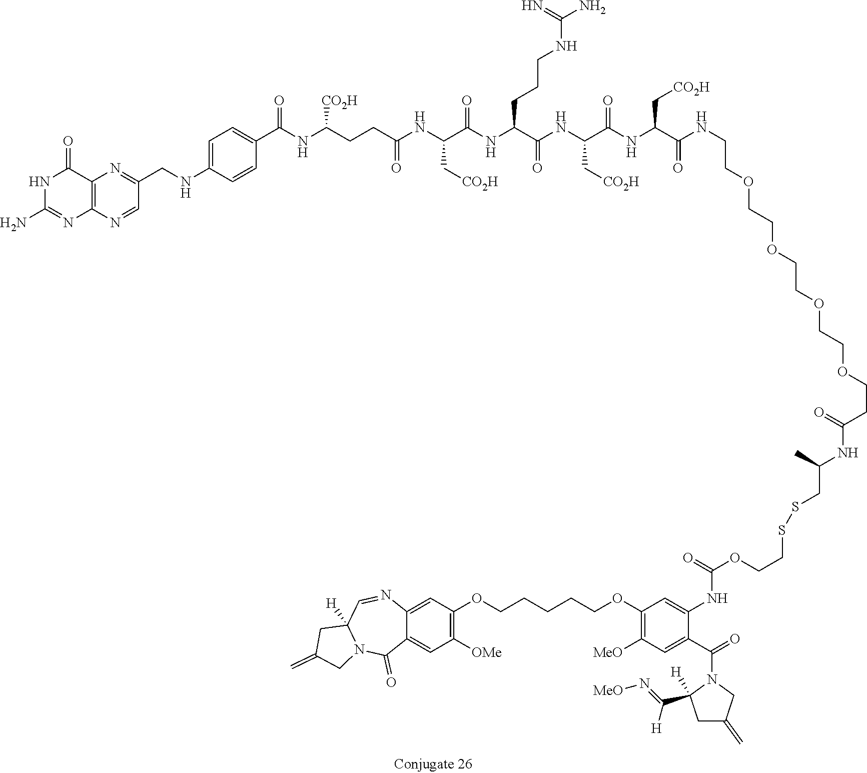 [SMILES: C=C1C[C@H]2C=Nc3cc(OCCCCCOc4cc(NC(=O)OCCSSC[C@@H](C)NC(=O)CCOCCOCCOCCOCCNC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)c(C(=O)N5CC(=C)C[C@H]5/C=N/OC)cc4OC)c(OC)cc3C(=O)N2C1]

### Step 1: Preparation of Compound 96

Compound 96 was prepared according to the procedure described for Compound 2, except 2-mercaptoethanol was used in lieu of 2-thiopropanol.

### Step 2: Preparation of Compound 97

Et₃N (36.8 μL, 2.1 equiv) is added to a solution of Compound 19 (99.8 mg, 126 μmol) and Compound 96 (50.9 mg, 1.05 equiv) in anhydrous CH₂Cl₂ (1.5 mL). After stirring at ambient temperature under argon for 25 min, the reaction mixture is loaded directly onto a CombiFlash system for purification (Silica gel column. Eluting with 0-10% CH₃OH in CH₂Cl₂. to yield 95.1 mg Compound 97 (mixture of two stereoisomers) as a light brown solid: (ESI-QMS): m/z=1006.80 (M+H).

### Step 3: Preparation of Compound 98

TFA (0.60 mL) is added to a solution of Compound 67 (48.5 mg, 1.2 equiv) in anhydrous CH₂Cl₂ (1.5 mL). The reaction mixture is stirred at ambient temperature under argon for 30 min and concentrated under reduced pressure. The residue is co-evaporated with CH₂Cl₂ (2 mL×3), concentrated under reduced pressure, dried under vacuum for 1 h, redissolved in anhydrous CH₂Cl₂ (1.5 mL), and transferred into a solution of Compound 97 (159 mg, 158 μmol) and PyBOP (86.3 mg, 1.05 equiv) in anhydrous CH₂Cl₂ (3.5 mL). To the solution is added iPr₂NEt (150 μL, 5.5 equiv). The reaction mixture is stirred at ambient temperature under argon for 1 h and loaded directly onto a CombiFlash system for purification (Silica gel column. Eluting with 0-10% CH₃OH in CH₂C₂) to give 125 mg Compound 98 (a mixture of stereoisomers) as a light brown solid: (ESI-QMS): m/z=1128.94 (M+H).

### Step 4: Preparation of Compound 99

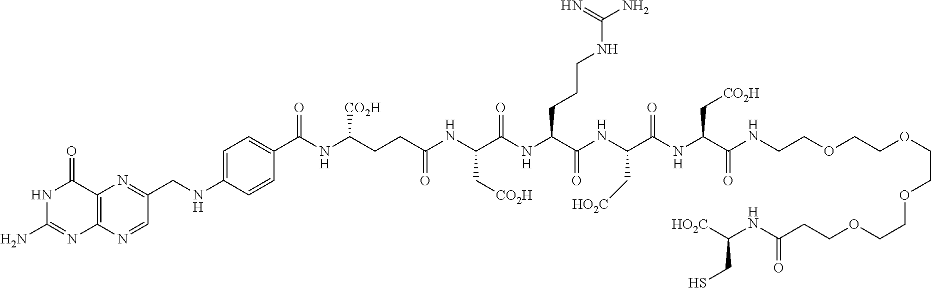 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)NCCOCCOCCOCCOCCC(=O)N[C@@H](CS)C(=O)O]

Compound 99 was synthesized by solid phase in five steps starting from H-Cys(4-methoxytrityl)-2-chlorotrityl-Resin.

| | mmol | equiv | MW | amount |
|---|---|---|---|---|
| H-Cys(4-methoxytrityl)- | 0.5 | | | 794 mg |
| 2-chlorotrityl-Resin | | | | |
| (loading 0.63 mmol/g) | | | | |
| Fmoc-NH-PEG4-COOH | 0.5 | 2 | 487.6 | 487.6 |
| (Dissolve in 15 ml DMF) | | | | |
| Fmoc-Asp(OtBu)—OH | 0.5 | 2 | 411.5 | 411.5 |
| (Dissolve in 15 ml DMF) | | | | |
| Fmoc-Asp(OtBu)—OH | 0.5 | 2 | 411.5 | 411.5 |
| (Dissolve in 15 ml DMF) | | | | |
| Fmoc-Arg(Pbf)-OH | 0.5 | 2 | 648 | 648 |
| (Dissolve in 15 ml DMF) | | | | |
| Fmoc-Asp(OtBu)—OH | 0.5 | 2 | 411.5 | 411.5 |
| (Dissolve in 15 ml DMF) | | | | |
| Fmoc-Glu-OtBu | 0.5 | 2 | 425.5 | 425.5 |
| (Dissolve in 15 ml DMF) | | | | |
| N¹⁰TFA Pteroic Acid | 0.5 | 1.25 | 408 | 255 |
| (dissolve in 15 ml DMF) | | | | |
| iPr₂NEt | 0.5 | 4 | 129 | 258 mg |
| PyBOP | 0.5 | 2 | 520 | 520 mg |

### Coupling Steps:

In a peptide synthesis vessel was added the resin, amino acid solution, iPr₂NEt, and PyBOP. Argon was bubbled through the solution for 1 h and then washed 3× with DMF and IPA. A solution of 20% piperdine in DMF for FMOC deprotection was added, 2× (10 min), before each amino acid coupling. This was continued to complete all seven coupling steps.

### Cleavage Step:

25 mL of cleavage reagent (92.5% TFA, 2.5% H₂O, 2.5% Triisopropylsaline, 2.5% (1.34 ml) ethandithiol) was added to the peptide synthesis vessel and Argon was bubbled for 1.5 h, drain, and wash 3× with cleavage reagent reagent. The reaction mixture was concentrated under reduced pressure until 10 ml remained. The product was triturated in ethyl ether and centrifuge. The resulting pellet was dried under high vacuum.

### Deprotection Step:

Crude protected Compound 99 was added to 10 ml water. The pH adjusted to 9.3 and maintained for 1 h using potassium carbonate. After 1 h the solution was adjusted to pH 5 with 1N HCl. The reaction mixture was load directly onto a C18 reverse phase column and purified via with 0-35% CH₃CN/50 mM NH₄HCO₃ buffer, pH 7.0 to yield 413 mg Compound 99 as a fluffy yellow solid.

### Step 5: Preparation of Conjugate 26

Conjugate 26 was synthesized by following the procedure for Conjugate 16 using Compound 98 and Compound 99. Conjugate 26 was isolated as a mixture of two stereoisomers: (ESI-QMS): m/z=1020.19 (M−2H)²⁻.

### Example 61: Preparation of Conjugate 27

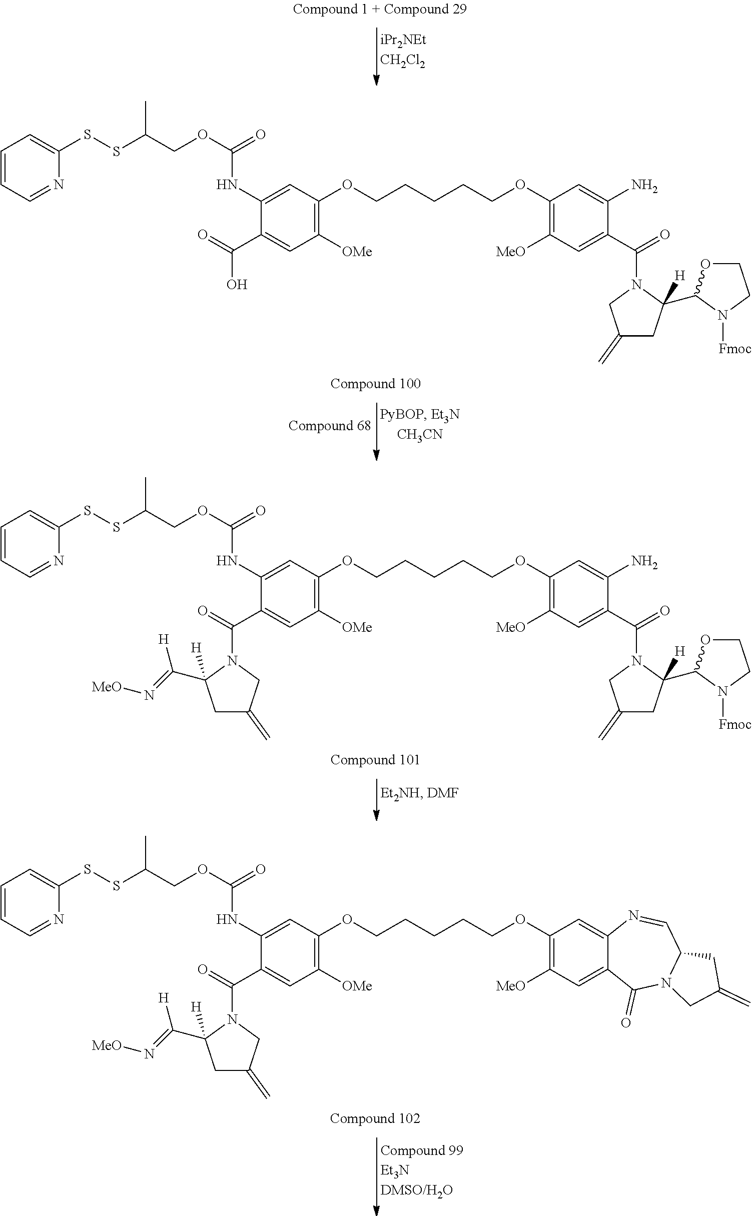

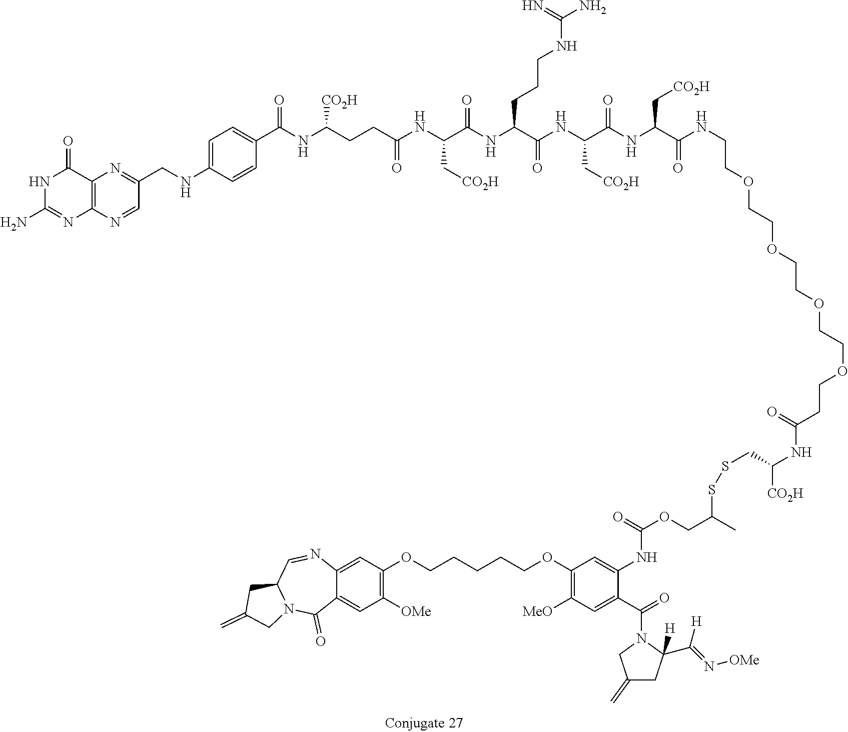 [SMILES: C=C1C[C@H]2C=Nc3cc(OCCCCCOc4cc(NC(=O)OCC(C)SSC[C@H](NC(=O)CCOCCOCCOCCOCCNC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)C(=O)O)c(C(=O)N5CC(=C)C[C@H]5/C=N/OC)cc4OC)c(OC)cc3C(=O)N2C1]

### Step 1: Preparation of Compound 100

iPr₂NEt (24 mL, 0.139 mmol) and Compound 1 (42 mg, 0.116 mmol) were added to a stirring solution of Compound 29 (92 mg, 0.116 mmol) in CH₂Cl₂ (1.16 mL). The reaction mixture was stirred for 2 h at room temperature. The progress of the reaction was monitored via LC/MS. The mixture was then concentrated and loaded directly to a silica gel column, and purified with 0-10% CH₃OH in CH₂C₂. 87 mg (85.0%) of desired product was collected as a white solid: LC/MS (ESI-QMS): m/z=1020.19 (M+H).

### Step 2: Preparation of Compound 101

A solution of Compound 67 (26 mg, 0.109 mmol) in anhydrous 50% TFA in CH₂Cl₂ (1.0 mL) was stirred at room temperature under argon for 30 min, after which the reaction mixture was concentrated under reduced pressure. The resulting residue was co-evaporated with CH₂Cl₂ (2 mL×3), and dried under high vacuum for 1 h to provide crude Compound 68. The residue was dissolved is CH₃CN (1 mL), and Et₃N (27 mL, 0.197 mmol) and Compound 100 (100 mg, 0.0986 mmol) were added. The reaction was allowed to stir for 5 min before PyBOP (56 mg, 0.109 mmol) was added. After stirring for 30 min the reaction mixture was concentrated under reduced pressure, and the crude residue was purified via silica chromatography (0-10% CH₃OH in CH₂Cl₂). 82.9 mg (72.2%) of desired product was collected as a white solid: LC/MS (ESI-QMS): m/z=1141.35 (M+H).

### Step 3: Preparation of Conjugate 27

Compound 101 (8.5 mg, 9.6 μmol was dissolved in 5% Et2NH in DMF (1 mL). The reaction mixture was stirred for 3 h. The reaction was monitored via LC/MS, and after the complete conversion of Compound 101 to Compound 102, a solution of Compound 99 (15.0 mg, 14.4 μmol) dissolved in DMSO (400 μl) and H₂O (100 μl) was added followed by Et₃N (2.6 μl, 19.2 μmol). The reaction mixture was stirred for an additional 1 h at room temperature. The reaction mixture was then filtered through a 0.45 micron PTFE membrane. Purification via preparative HPLC (10-100% MeCN/50 mM NH₄HCO₃ pH 7 buffer) yielded 5.6 mg (32.5% over two steps) of Conjugate 27 as a yellow powder: LC/MS (ESI-QMS): m/z=1020.98 (M+2H)²⁺.

### Example 62: Preparation of Conjugate 28

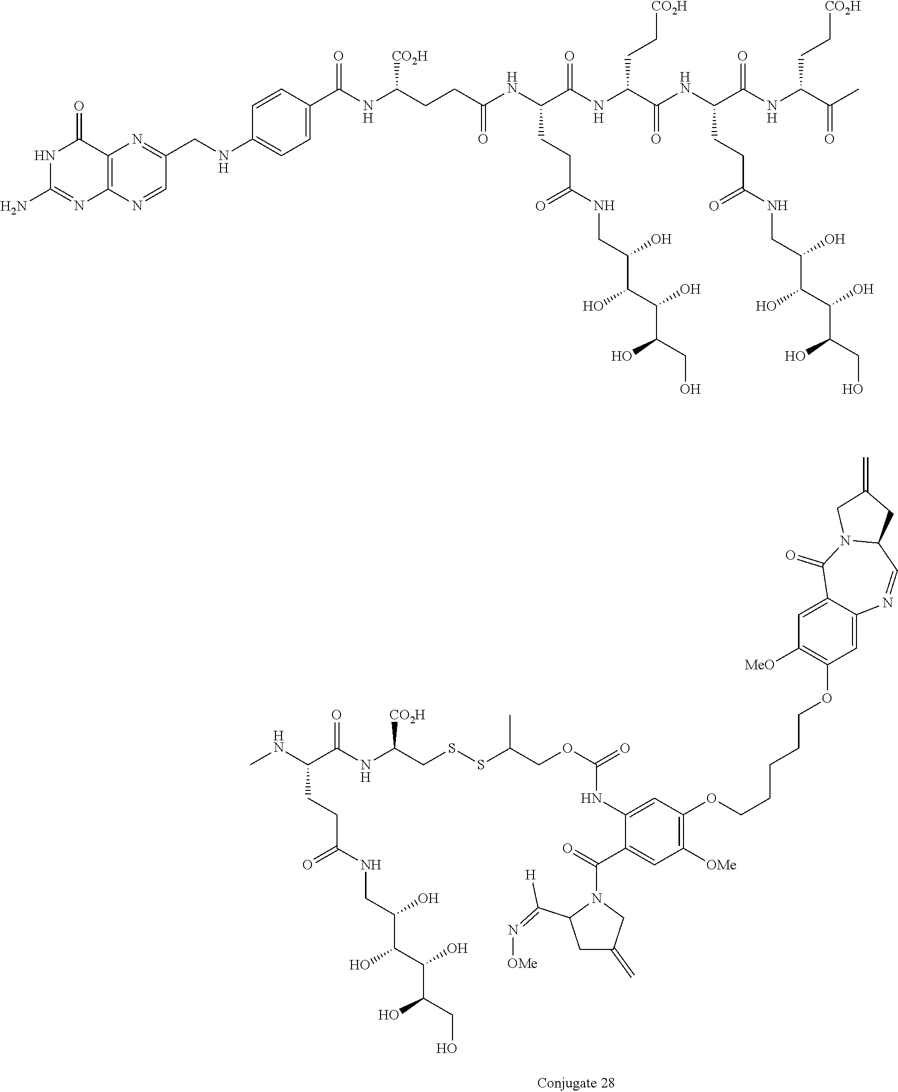

Compound 101 (8.5 mg, 9.6 μmol) was dissolved in 5% Et2NH in DMF (1 mL). The reaction mixture was stirred for 3 h. The reaction was monitored via LC/MS, and after the complete conversion of Compound 101 to Compound 102, a solution of Compound 38 (24.6 mg, 14.4 μmol) dissolved in DMSO (400 μL) and H₂O (100 μL) was added followed by Et₃N (2.6 ml, 19.2 μmol). The reaction mixture was stirred for an additional 1 h at room temperature. The reaction mixture was then filtered through a 0.45 micron PTFE membrane. Purification via preperative HPLC (10-100% MeCN/50 mM NH₄HCO₃ pH 7 buffer) provided 6.3 mg (27.0% over two steps) of desired product as a yellow powder LC/MS (ESI-QMS): m/z=1214.43 (M+H).

### Example 63: Preparation of Conjugate 29

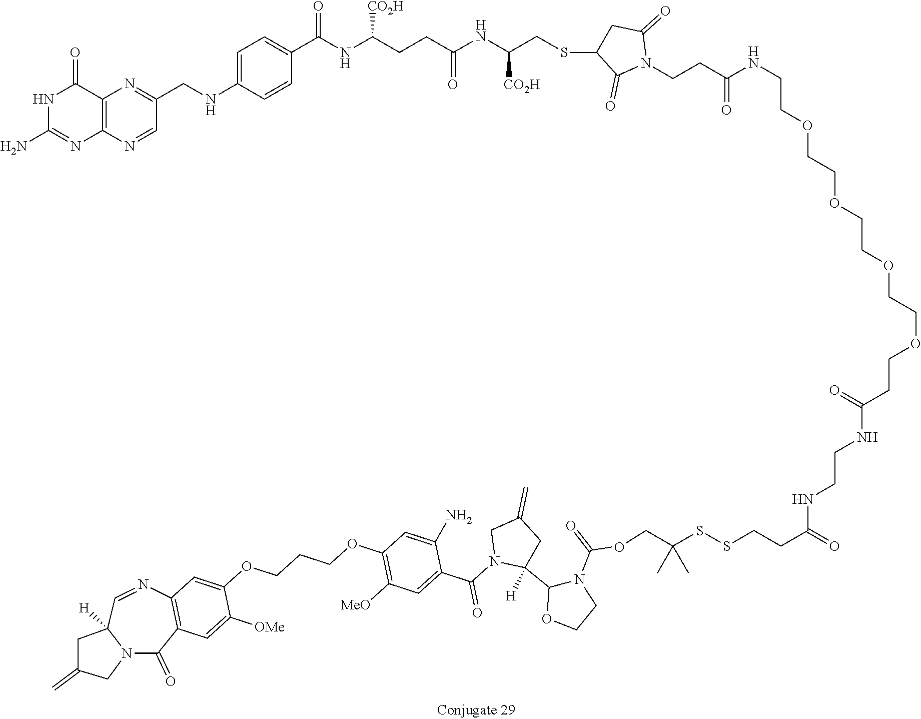 [SMILES: C=C1C[C@H]2C=Nc3cc(OCCCOc4cc(N)c(C(=O)N5CC(=C)C[C@H]5C5OCCN5C(=O)OCC(C)(C)SSCCC(=O)NCCNC(=O)CCOCCOCCOCCOCCNC(=O)CCN5C(=O)CC(SC[C@H](NC(=O)CC[C@H](NC(=O)c6ccc(NCc7cnc8nc(N)[nH]c(=O)c8n7)cc6)C(=O)O)C(=O)O)C5=O)cc4OC)c(OC)cc3C(=O)N2C1]

Conjugate 29 was synthesized by following the procedure for Conjugate 5 starting from N¹⁰-trifluoroacetyl protected folate-containing peptidyl fragment N¹⁰-TFA-Pte-Glu-Cys-OH as described in U.S. Pat. No. 7,601,332, incorporated herein by reference for the preparation of that compound, in lieu of EC119. LC/MS (ESI-QMS): (M+2H)²⁺: 1084, (M+3H)³⁺: 723.

### Example 64: Preparation of Conjugate 30

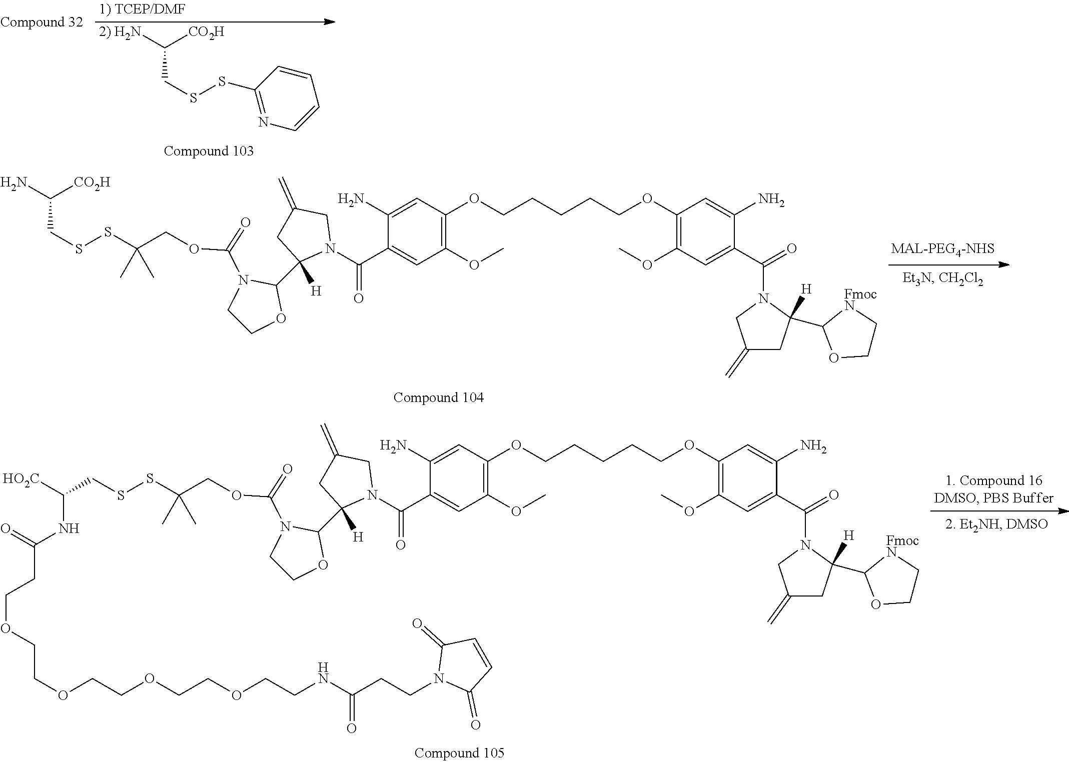

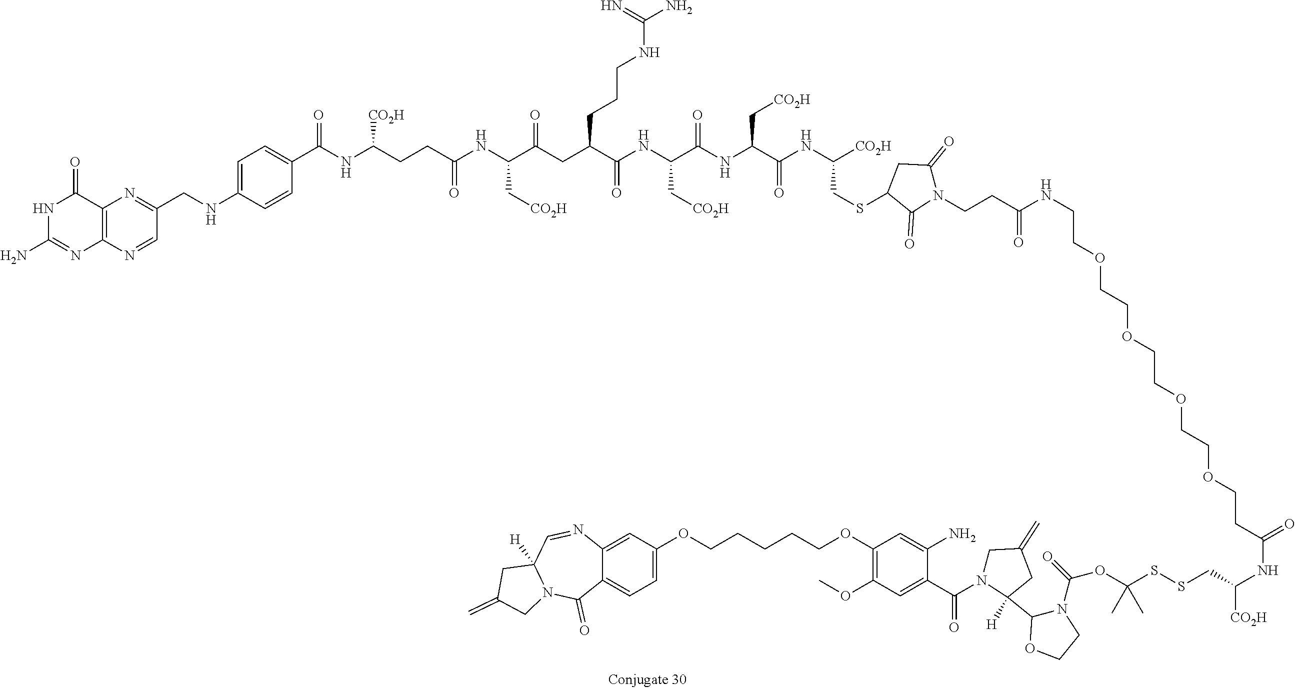 [SMILES: C=C1C[C@H]2C=Nc3cc(OCCCCCOc4cc(N)c(C(=O)N5CC(=C)C[C@H]5C5OCCN5C(=O)OC(C)(C)SSC[C@H](NC(=O)CCOCCOCCOCCOCCNC(=O)CCN5C(=O)CC(SC[C@H](NC(=O)[C@H](CC(=O)O)NC(=O)[C@H](CC(=O)O)NC(=O)[C@H](CCCNC(=N)N)CC(=O)[C@H](CC(=O)O)NC(=O)CC[C@H](NC(=O)c6ccc(NCc7cnc8nc(N)[nH]c(=O)c8n7)cc6)C(=O)O)C(=O)O)C5=O)C(=O)O)cc4OC)ccc3C(=O)N2C1]

### Step 1: Preparation of Compound 104

Compound 32 (49.1 mg, 0.034 mmol) was dissolved in DMF (1.2 mL) and treated with 0.5M TECP (74.8 μL, 0.0374 mmol). The reaction was stirred for 20 min at room temperature. Compound 103 (9.5 mg, 0.040 mmol), prepared according to the procedure described for Compound 1 except that cysteine was used in place of 2-mercaptopropanol, was added to the reaction mixture and stirred for an additional 1 h. The crude mixture was loaded directly on to a C18 reverse column and purified with 0-50% CH₃CN in H₂O) to yield 9 mg of the desired product Compound 104 (22% yield over two steps): LC/MS (ESI-QMS): m/z=1180 (M+H)¹⁺.

### Step 2: Preparation of Compound 105

Compound 104 (4.2 mg, 0.0036 mmol) was added to a solution of Maleimide-PEG-NHS Ester (2.01 mg, 0.0039 mmol, available from Sigma-Aldrich) and Et₃N (0.54 μL, 0.0039 mmol) in CH₂Cl₂ (0.5 mL). The reaction mixture was stirred for 30 min at room temperature and then concentrated to dryness.

### Step 3: Preparation of Conjugate 30

The crude residue of Compound 105 was carried forward without further purification. Compound 105 residue was dissolved in DMSO (0.3 mL) and to it was added a solution of EC119 (4.1 mg, 0.00396 mmol) in pH 7.4 PBS buffer (0.5 mL and DMSO (0.5 mL). Et₃N (3.0 μL, 0.0216 mmol) was added to the reaction mixture and stirred for 30 min at room temperature. The crude product was purified by prep-HPLC (10 to 100% acetonitrile in 50 mM NH₄HCO₃, pH 7.4) to yield the desired product: LC/MS (ESI-QMS): m/z=1313 (M+2H)²⁺.

The product of the preceding step (5.0 mg, 0.0019 mmol) was dissolved in DMSO (0.5 mL), and Et₂NH (0.25 mL) was added. The reaction mixture was stirred for 30 min at room temperature. The crude product was purified by prep-HPLC (10 to 100% acetonitrile in 50 mM NH₄HCO₃, pH 7.4) to yield 3.44 mg of the desired product Conjugate 30 (77% yield): LC/MS (ESI-QMS): m/z=1180 (M+2H+H₂O)²⁺.

### Example 65: Preparation of Conjugate 31

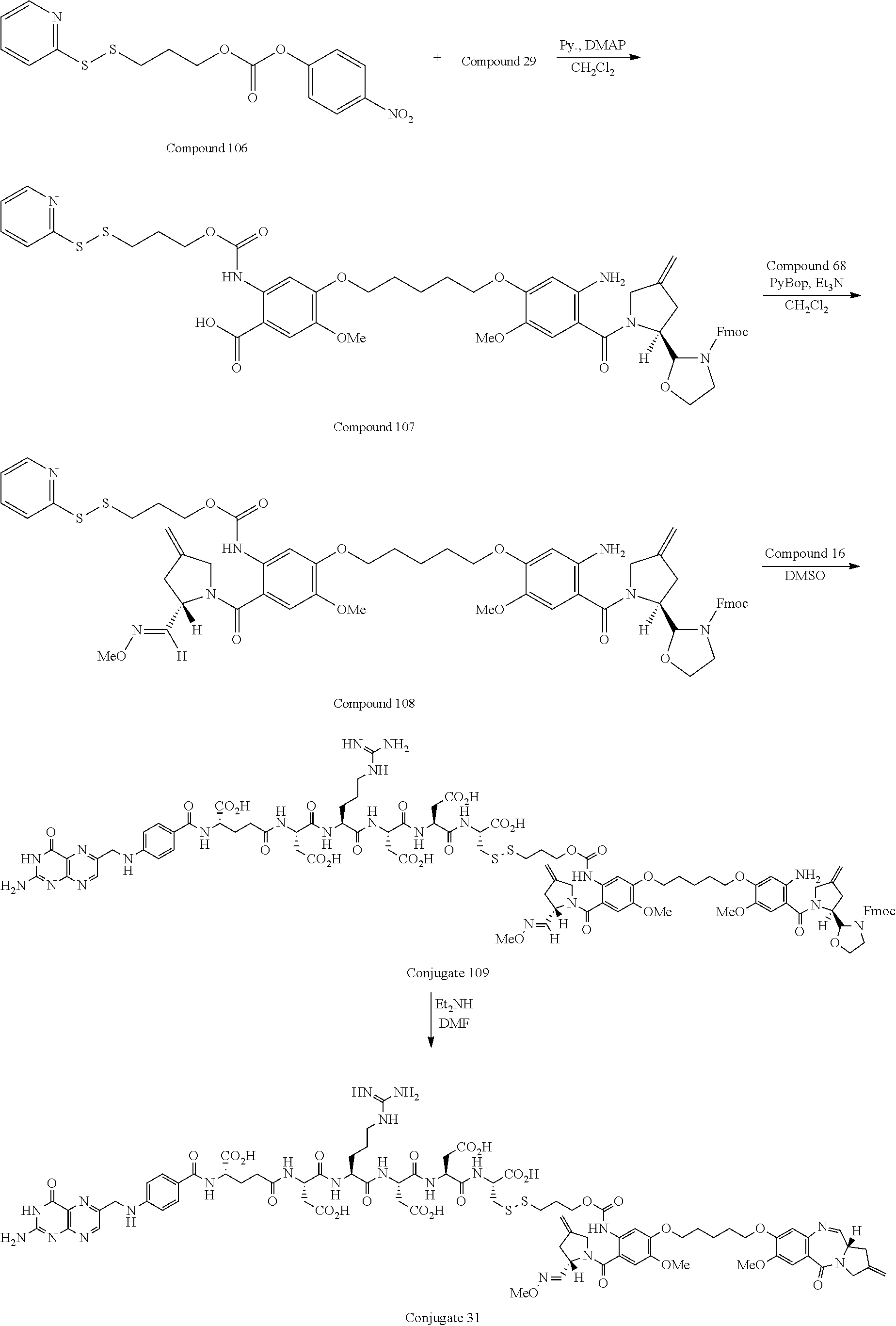

### Step 1: Preparation of Compound 106

Compound 106 was prepared according to the procedure described for Compound 59, except 3-mercaptopropanol was used in place of 2-mercapto-3-methylbutan-1-ol, and para-nitrophenol was used in place of hydroxybenzotriazole.

### Step 2: Preparation of Compound 107

A mixture of Compound 106 (11.0 mg, 0.03 mmol), Compound 29 (20.0 mg, 0.025 mmol), pyridine (6.1 μl, 0.075 mmol) and DMAP (0.3 mg, 0.003 mmol) in CH₂Cl₂ was stirred at room temperature overnight. The reaction mixture was concentrated in vacuo. The crude product was purified by Combiflash in 0-20% CH₃OH/CH₂Cl₂ to afford 8.1 mg of Compound 107: (ESI-QMS): m/z=1020.85 (M+H).

### Step 3: Preparation of Compound 108

To a mixture of Compound 107 (26.5 mg, 0.026 mmol), Compound 68 (3.64 mg, 0.026 mmol) and PyBOP (16.2 mg, 0.031 mmol) in CH₂Cl₂ (1 ml) was added Et₃N (18 μl, 0.13 mmol) at room temperature. The reaction mixture was stirred at room temperature for 4 h. The solvent was removed under reduced pressure. The crude product was purified by Combiflash in 0-20% CH₃OH/CH₂Cl₂ to afford 11.5 mg of Compound 108: (ESI-QMS): m/z=1142.98 (M+H).

### Step 4: Preparation of Compound 109

A mixture of Compound 108 (11.5 mg, 0.01 mmol) and Compound 16 (10.5 mg, 0.01 mmol) in DMSO (1 ml) was stirred at room temperature for 3 h. The reaction mixture was concentrated in vacuo. The crude product was purified by prep-HPLC HPLC (10 to 100% acetonitrile in 20 mM NH₄HCO₃, pH 7.4) to yield pure Compound 109: (ESI-QMS): m/z=1041.28 (M+2H)²⁺.

### Step 5: Preparation of Conjugate 31

To a mixture of Compound 109 (8 mg, 0.004 mmol) in DMF (1 ml) was added Et₂NH (6 μl, 0.058 mmol) at room temperature. The reaction mixture was stirred at room temperature for 2 h. The crude product was purified by prep-HPLC HPLC (10 to 100% acetonitrile in 20 mM NH₄HCO₃, pH 7.4) to yield 4.5 mg of pure Conjugate 31: (ESI-QMS): m/z=1794.99 (M+2H)²⁺.

### Example 66: Preparation of Conjugate 32

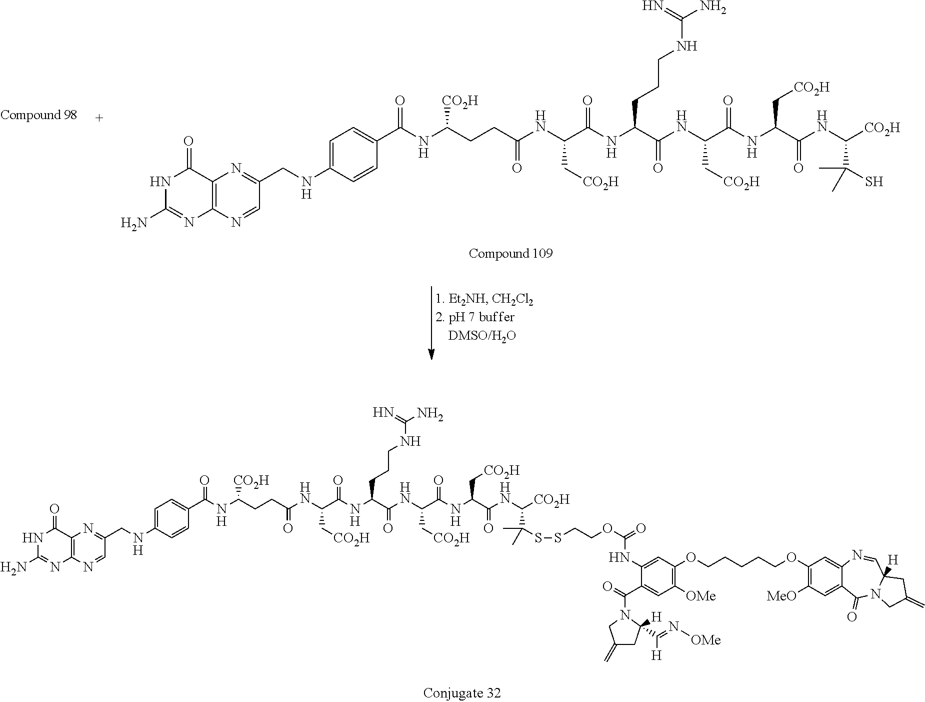 [SMILES: CC(C)(S)[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c1ccc(NCc2cnc3nc(N)[nH]c(=O)c3n2)cc1)C(=O)O)C(=O)O ; C=C1C[C@H]2C=Nc3cc(OCCCCCOc4cc(NC(=O)OCCSSC(C)(C)[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c5ccc(NCc6cnc7nc(N)[nH]c(=O)c7n6)cc5)C(=O)O)C(=O)O)c(C(=O)N5CC(=C)C[C@H]5/C=N/OC)cc4OC)c(OC)cc3C(=O)N2C1]

Compound 109 was prepared according the procedure described for Compound 99, except that the coupling step using Fmoc-NH-PEG4-COOH was omitted. Conjugate 32 was isolated as a mixture of stereoisomers: (ESI-QMS): m/z=1809.35 (M+H).

### BIOLOGICAL EXAMPLES

### General.

The following abbreviations are used herein: partial response (PR); complete response (CR), once weekly (SIW), biweekly (M/F) (BIW), three times per week (M/W/F) (TIW). A PR is observed where tumor volume, as defined herein, decreases from a previous high during the observation period, though regrowth may occur. A CR is observed where tumor volume, as defined herein, decreases to zero during the observation period, though regrowth may occur. A cure is observed where tumor volume, as defined herein, decreases to zero, and does not regrow during the observation period.

### Method 1. Inhibition of Cellular DNA Synthesis.

The conjugates described herein were evaluated using an in vitro cytotoxicity assay that predicted the ability of the drug to inhibit the growth of the corresponding targeted cells, such as, but not limited to the following

Cell Line

| | |
|---|---|
| KB | Human cervical carcinoma |
| NCl/ADR-RES-Cl₂ | Human ovarian carcinoma |
| IGROV1 | Human ovarian adenocarcinoma |
| MDA-MB-231 | Human breast adenocarcinoma (triple negative) |
| A549 | Human lung carcinoma |
| H23 | Human lung adenocarcinoma |
| HepG2 | Human hepatocellular carcinoma |
| AN3CA | Human endometrial adenocarcinoma |

It is to be understood that the choice of cell type can be made on the basis of the susceptibility of those selected cells to the drug that forms the conjugate, and the relative expression of the cell surface receptor or target antigen. The test conjugates were conjugates of a cell surface receptor or target antigen binding compound and PBD prodrugs, poly-PBD prodrugs, and mixed PBDs, as described herein. The test cells were exposed to varying concentrations of the conjugates, and optionally also in the absence or presence of at least a 100-fold excess of the unconjugated cell surface receptor or target antigen binding compound for competition studies to assess activity as being specific to the cell surface receptor or target antigen.

### Method 2: In Vitro Folate Receptor Specific Activity Assay of Folate Conjugates.

KB cells were seeded in individual 24-well Falcon plates and allowed to form nearly confluent monolayers overnight in folate free Roswell Park Memorial Institute (FFRPMI)/Heat-Inactivated Fetal Calf Serum (HIFCS). Thirty minutes prior to the addition of folate-conjugate, spent medium was aspirated from all wells and replaced with either fresh FFRPMI or FFRPMI supplemented with 100 μM folic acid. Each well then received 1 mL of medium containing increasing concentrations of folate-conjugate (3 wells per sample). Cells were pulsed for 2 h at 37° C., rinsed 4 times with 0.5 mL of medium and then chased in 1 mL of fresh medium up to 72 h. Spent medium was aspirated from all wells and replaced with fresh medium containing 5 μCi/mL of ³H-thymidine. Following a 2 h incubation at 37° C., cells were washed 3 times with 0.5 mL of PBS and then treated with 0.5 mL of ice-cold 5% trichloroacetic acid per well. After 15 min, the trichloroacetic acid was aspirated and the cells solubilized by the addition of 0.5 mL of 0.25 N sodium hydroxide for 15 min at room temperature. Four hundred and fifty L of each solubilized sample were transferred to scintillation vials containing 3 mL of Ecolume scintillation cocktail and counted in a liquid scintillation counter. Final results were expressed as the percentage of ³H-thymidine incorporation relative to untreated controls. For conjugates described herein, dose-dependent cytotoxicity was generally measurable, and in most cases, the IC₅₀ values (concentration of drug conjugate required to reduce ³H-thymidine incorporation into newly synthesized DNA by 50%) were in the picomolar to low nanomolar range.

### Example 1: Conjugate 9 In Vitro Activity

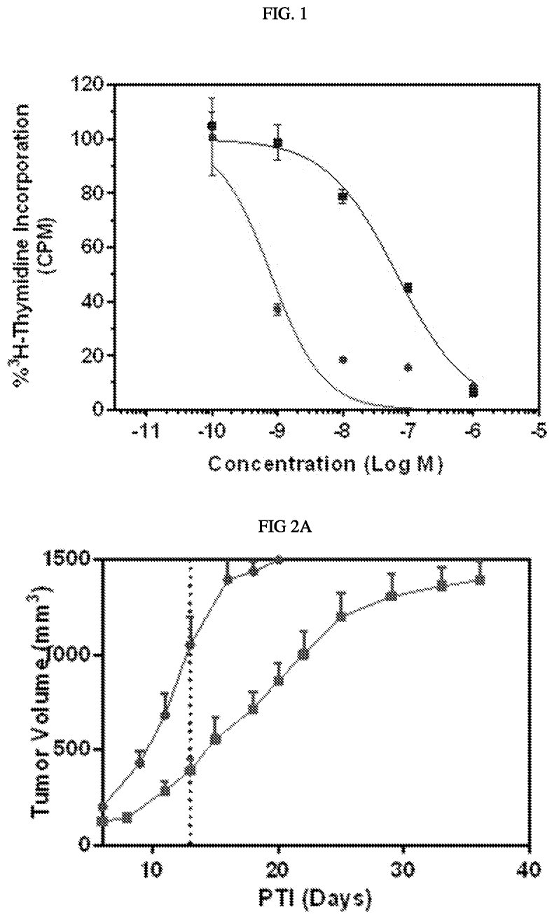

In FIG. 1, the percentage of ³H-thymidine incorporated into KB cells treated with Conjugate 9 (●) and with Conjugate 9 and excess folate (▪) is shown. The IC₅₀ value was 0.8 nM without excess folate and 67 nM with excess folate.

### Example 2: Conjugate 1 In Vitro Activity

In FIG. 3, the percentage of ³H-thymidine incorporated into KB cells treated with Conjugate 1 (●) and with Conjugate 1 and excess folate (▪) is shown. The IC₅₀ value was 0.02 nM without excess folate and 10 nM with excess folate.

### Example 3: Conjugate 2 In Vitro Activity

In FIG. 5, the percentage of ³H-thymidine incorporated into KB cells treated with Conjugate 2 (●) and with Conjugate 2 and excess folate (▪) is shown. The IC₅₀ value was 0.14 nM without excess folate and 16 nM with excess folate.

### Example 4: Conjugate 5 In Vitro Activity

In FIG. 7, the percentage of ³H-thymidine incorporated into KB cells treated with Conjugate 5 (●) and with Conjugate 5 and excess folate (▪) is shown.

### Example 5: Conjugate 3 In Vitro Activity

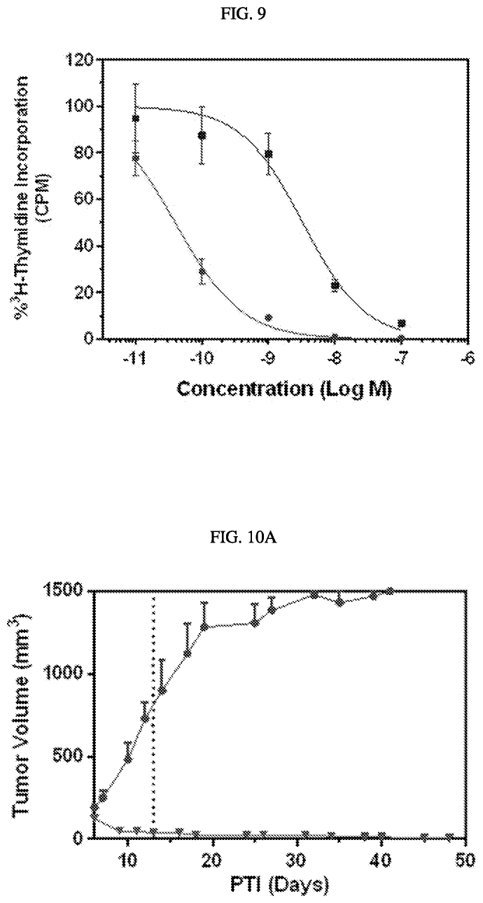

In FIG. 9, the percentage of ³H-thymidine incorporated into KB cells treated with Conjugate 3 (●) and with Conjugate 3 and excess folate (▪) is shown. The IC₅₀ value was 39 pM without excess folate and 3 nM with excess folate.

### Example 6: Conjugate 12 In Vitro Activity

In FIG. 11, the percentage of ³H-thymidine incorporated into KB cells treated with Conjugate 12 (▴) and with Conjugate 12 and excess folate (●) is shown. The IC₅₀ value was 0.05 nM without excess folate and 8 nM with excess folate.

### Example 7: Conjugate 4 In Vitro Activity

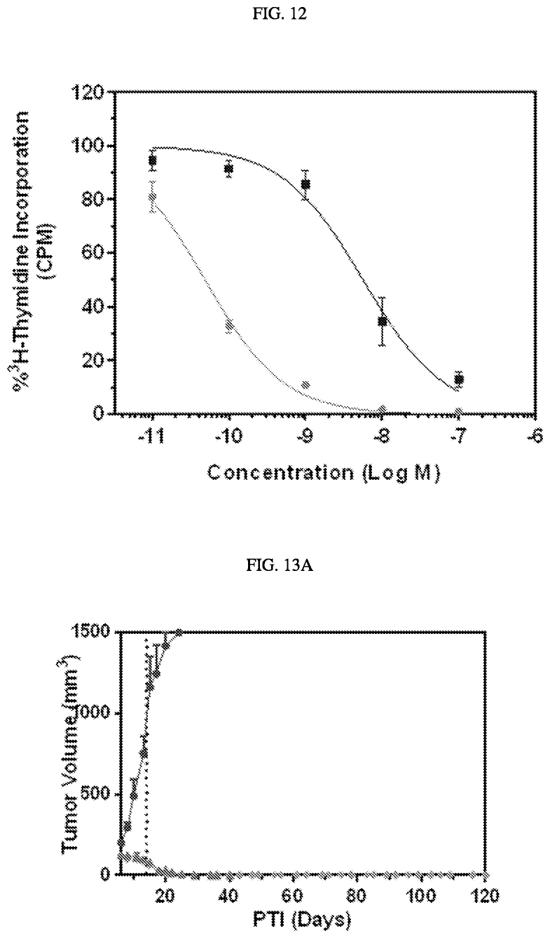

In FIG. 12, the percentage of ³H-thymidine incorporated into KB cells treated with Conjugate 4 (●) and with Conjugate 4 and excess folate (▪) is shown. The IC₅₀ value was 49 pM without excess folate and 6 nM with excess folate.

### Example 9: Conjugate 16 In Vitro Activity

In FIG. 14, the percentage of ³H-thymidine incorporated into KB cells treated with Conjugate 16 (●) and with Conjugate 16 and excess folate (▪) is shown. The IC₅₀ value was 70 pM without excess folate and 5 nM with excess folate.

### Example 10: Conjugate 6 In Vitro Activity

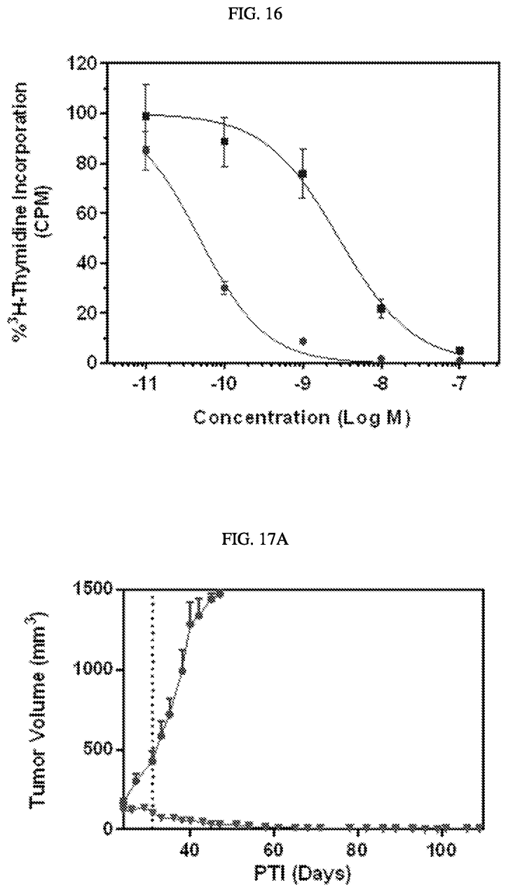

In FIG. 16, the percentage of ³H-thymidine incorporated into KB cells treated with Conjugate 6 (●) and with Conjugate 6 and excess folate (▪) is shown. The IC₅₀ value was 48 pM without excess folate and 3 nM with excess folate.

### Example 11: Conjugate 15 In Vitro Activity

In FIG. 18, the percentage of ³H-thymidine incorporated into KB cells treated with Conjugate 15 (●) and with Conjugate 15 and excess folate (▪) is shown. The IC₅₀ value was 81 pM without excess folate and 2 nM with excess folate.

### Example 12: Conjugate 7 In Vitro Activity

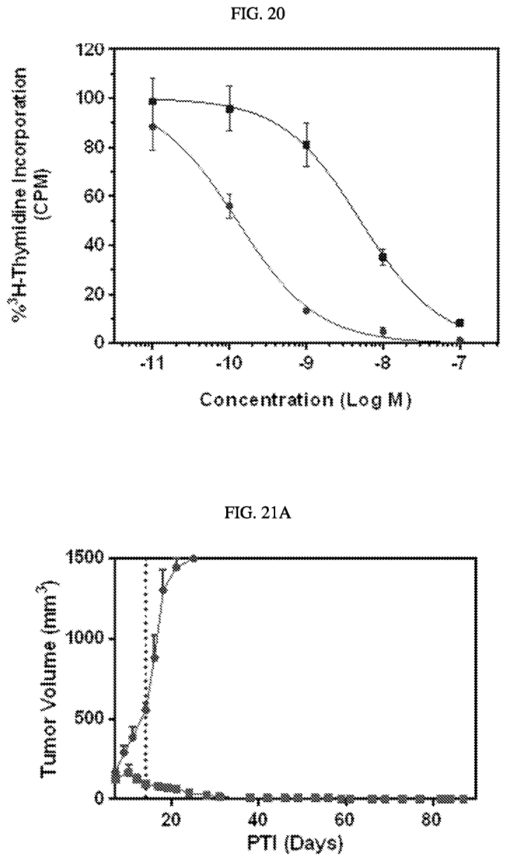

In FIG. 20, the percentage of ³H-thymidine incorporated into KB cells treated with Conjugate 7 (●) and with Conjugate 7 and excess folate (▪) is shown. The IC₅₀ value was 0.13 nM without excess folate and 5 nM with excess folate.

### Example 13: Conjugate 8 In Vitro Activity

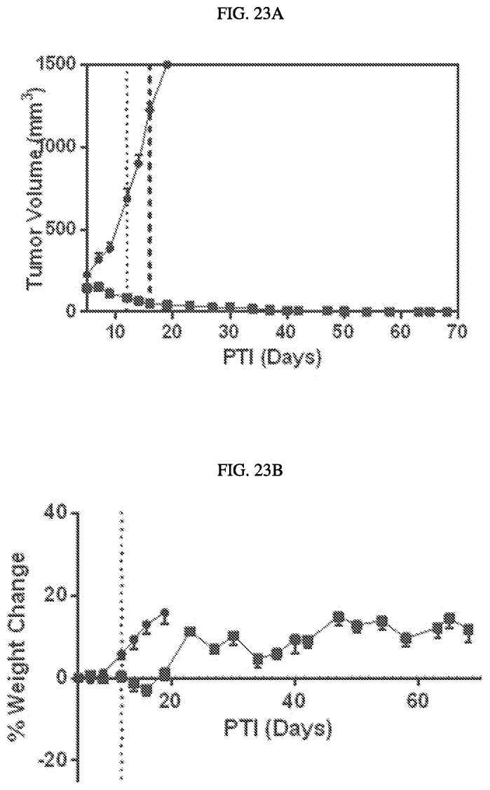

In FIG. 22, the percentage of ³H-thymidine incorporated into KB cells treated with Conjugate 8 (●) and with Conjugate 8 and excess folate (▪) is shown. The IC₅₀ value was 55 pM without excess folate and 0.3 nM with excess folate.

### Example 14: Conjugate 18 In Vitro Activity

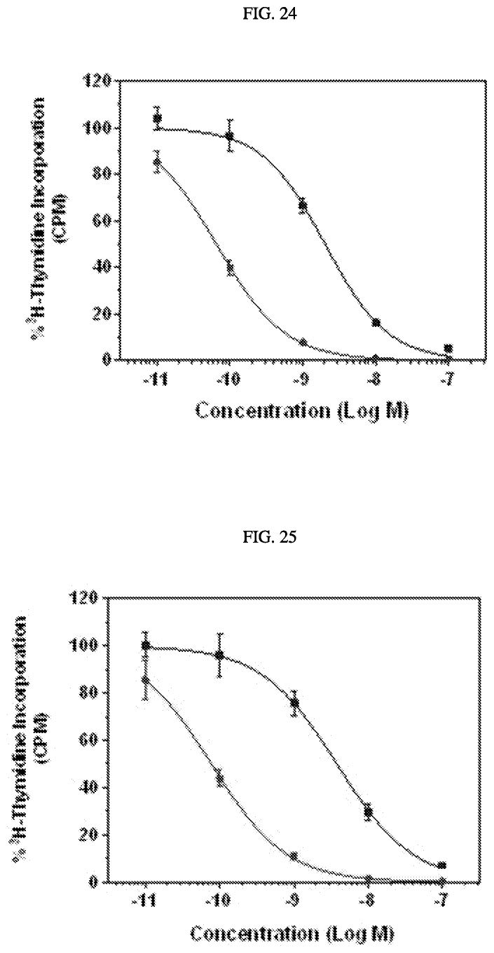

In FIG. 24, the percentage of ³H-thymidine incorporated into KB cells treated with Conjugate 18 (●) and with Conjugate 18 and excess folate (▪) is shown. The IC₅₀ value was 65 pM without excess folate and 2 nM with excess folate.

### Example 15: Conjugate 19 In Vitro Activity

In FIG. 25, the percentage of ³H-thymidine incorporated into KB cells treated with Conjugate 19 (●) and with Conjugate 19 and excess folate (▪) is shown. The IC₅₀ value was 77 pM without excess folate and 3.8 nM with excess folate.

### Example 16: Conjugate 20 In Vitro Activity

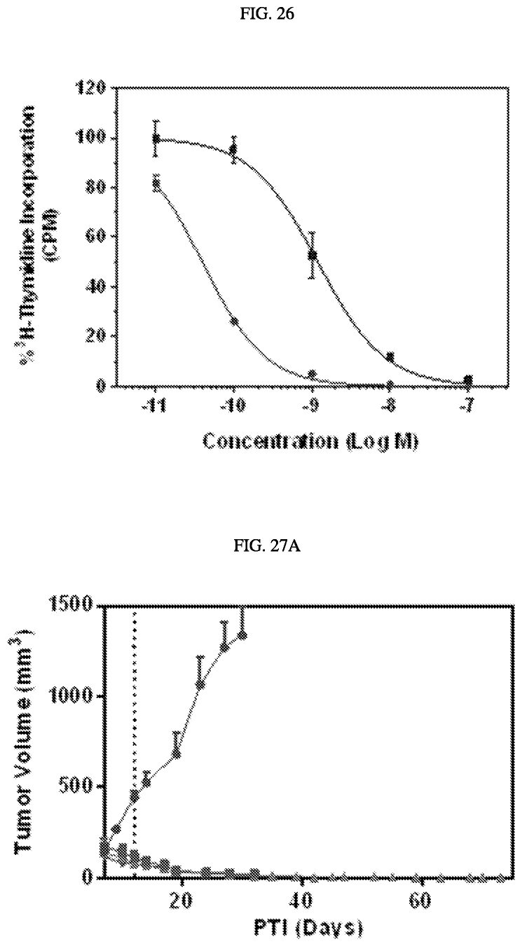

In FIG. 26, the percentage of ³H-thymidine incorporated into KB cells treated with Conjugate 20 (●) and with Conjugate 20 and excess folate (▪) is shown. The IC₅₀ value was 40 pM without excess folate and 0.7 nM with excess folate.

### Example 17: Conjugate 22 In Vitro Activity

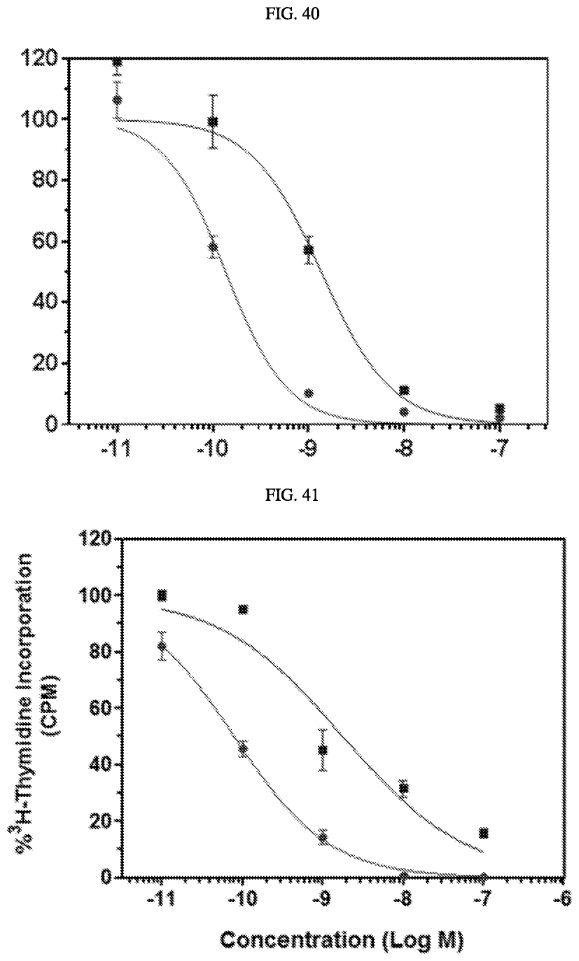

In FIG. 40, the percentage of ³H-thymidine incorporated into KB cells treated with Conjugate 20 (●) and with Conjugate 22 and excess folate (▪) is shown. The IC₅₀ value was 0.14 nM without excess folate and 1.4 nM with excess folate.

### Example 18: Conjugate 24 In Vitro Activity

In FIG. 41, the percentage of ³H-thymidine incorporated into KB cells treated with Conjugate 24 (●) and with Conjugate 24 and excess folate (▪) is shown. The IC₅₀ value was 79 pM without excess folate and 1.8 nM with excess folate.

### Example 19: Conjugate 25 In Vitro Activity

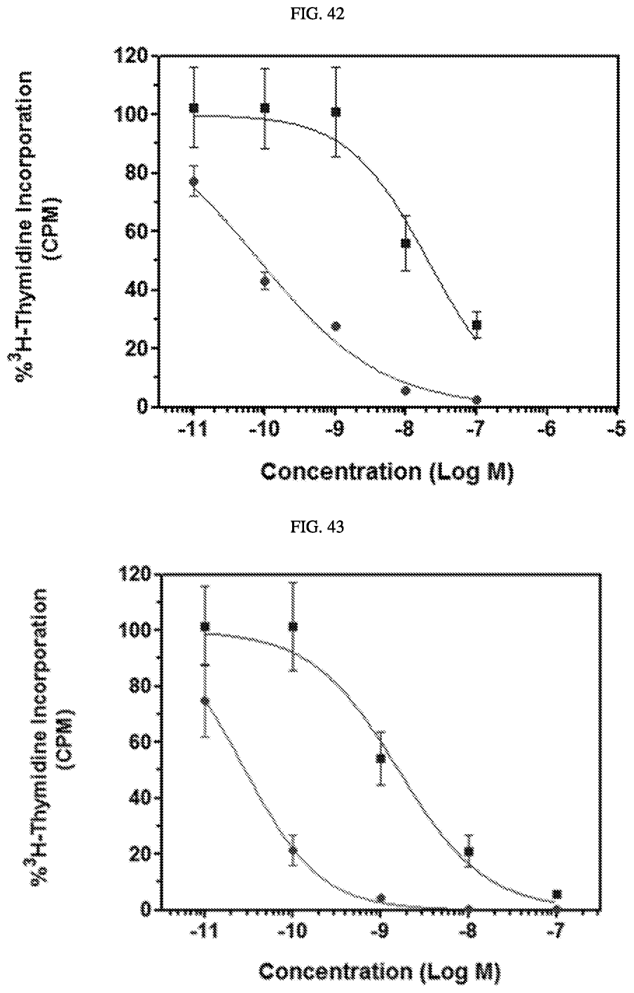

In FIG. 42, the percentage of ³H-thymidine incorporated into KB cells treated with Conjugate 25 (●) and with Conjugate 25 and excess folate (▪) is shown. The IC₅₀ value was 85 pM without excess folate and 20 nM with excess folate.

### Example 20: Conjugate 26 In Vitro Activity

In FIG. 43, the percentage of ³H-thymidine incorporated into KB cells treated with Conjugate 26 (●) and with Conjugate 26 and excess folate (▪) is shown. The IC₅₀ value was 28 pM without excess folate and 1.6 nM with excess folate.

### Example 21: Conjugate 27 In Vitro Activity

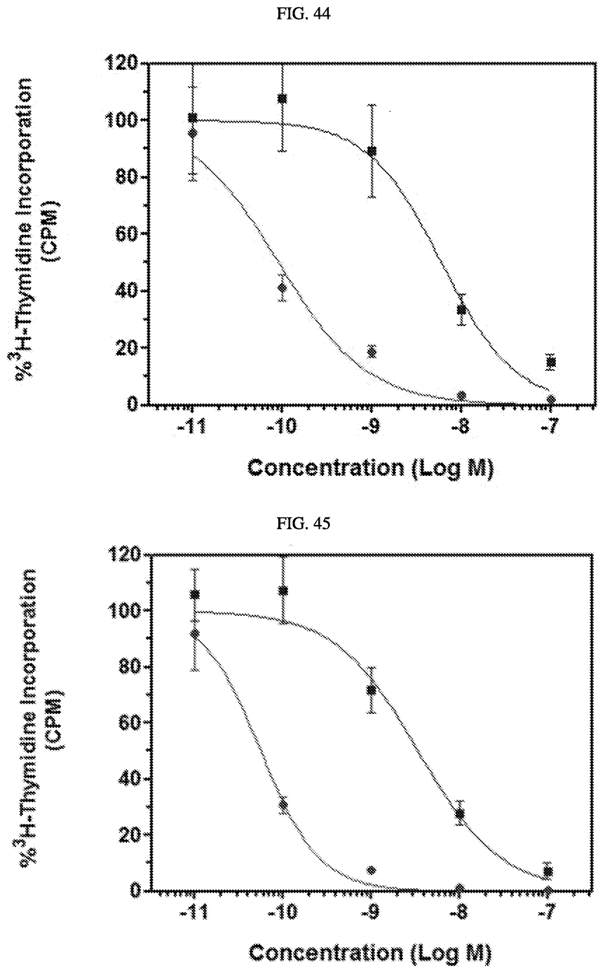

In FIG. 44, the percentage of ³H-thymidine incorporated into KB cells treated with Conjugate 27 (●) and with Conjugate 27 and excess folate (▪) is shown. The IC₅₀ value was 91 pM without excess folate and 6.1 nM with excess folate.

### Example 22: Conjugate 28 In Vitro Activity

In FIG. 45, the percentage of ³H-thymidine incorporated into KB cells treated with Conjugate 28 (●) and with Conjugate 28 and excess folate (▪) is shown. The IC₅₀ value was 56 pM without excess folate and 3.4 nM with excess folate.

### Example 23: Conjugate 31 In Vitro Activity

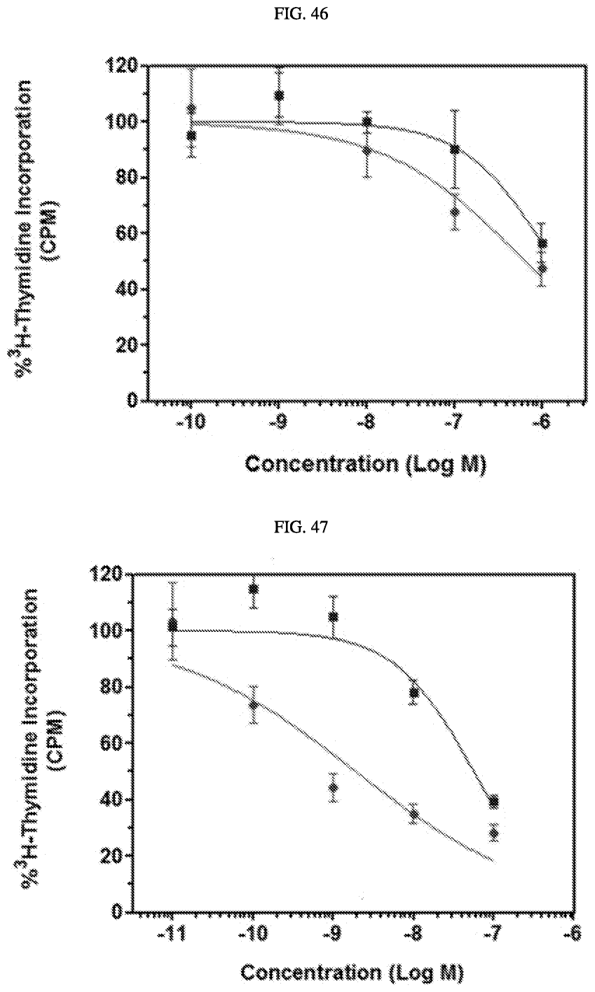

In FIG. 46, the percentage of ³H-thymidine incorporated into KB cells treated with Conjugate 31 (●) and with Conjugate 31 and excess folate (▪) is shown. The IC₅₀ value was 647 pM without excess folate.

### Example 24: Conjugate 32 In Vitro Activity

In FIG. 47, the percentage of ³H-thymidine incorporated into KB cells treated with Conjugate 32 (●) and with Conjugate 32 and excess folate (▪) is shown. The IC₅₀ value was 2 nM without excess folate and 57 nM with excess folate.

### Example 25: Relative Affinity Assay

FR-positive KB cells were seeded in 24-well Falcon plates and allowed to form adherent monolayers (>90% confluent) overnight in FFRPMI/HIFCS. Spent incubation medium was replaced with FFRPMI supplemented with 10% HIFCS and containing 100 nmol/L of [³H]FA in the absence and presence of increasing concentrations of unlabeled FA or the test conjugate. Cells were incubated for 1 h at 37° C. and then rinsed thrice with 0.5 mL PBS. Five hundred microliters of 1% SDS in PBS were added to each well; after 5 min, cell lysates were collected, transferred to individual vials containing 5 mL of scintillation cocktail, and then counted for radioactivity.

Cells exposed to only the [3H]FA in FFRPMI (no competitor) were designated as negative controls, whereas cells exposed to the [3H]FA plus 1 mmol/L unlabeled FA served as positive controls. Disintegrations per minute (DPM) measured in the latter samples (representing nonspecific binding of label) were subtracted from the DPM values from all samples. Notably, relative affinities were defined as the inverse molar ratio of compound required to displace 50% of [³H]FA bound to FR on KB cells, and the relative affinity of FA for the FR was set to 1.

Results for Conjugate 1 are shown in FIG. 28. The results show that linkage of a large drug molecule does not radically alter the vitamin's intrinsic binding affinity to its receptor.

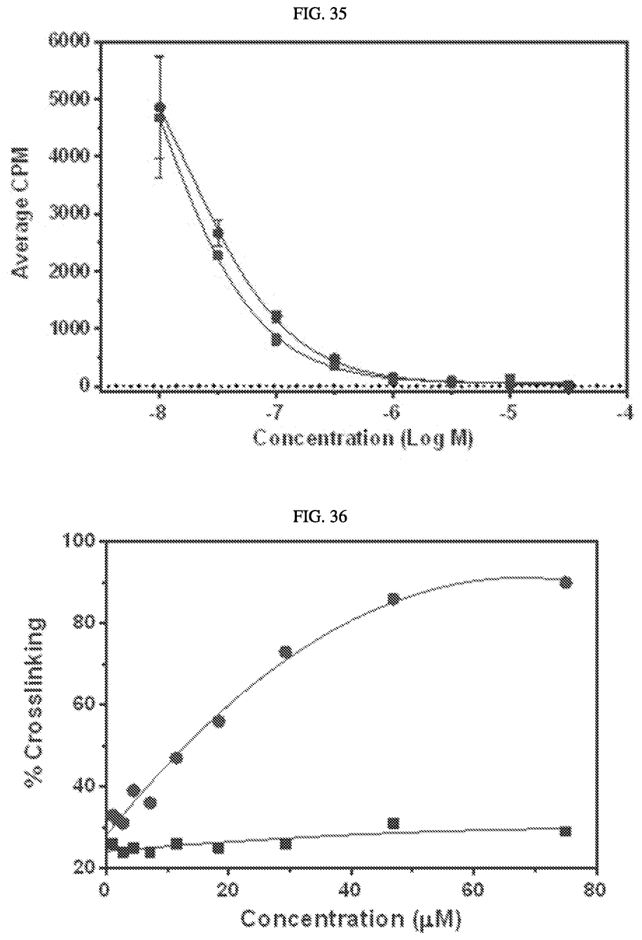

Results for Conjugate 5 are shown in FIG. 35. The results show that linkage of a large drug molecule does not radically alter the vitamin's intrinsic binding affinity to its receptor.

### Example 26: DNA Crosslinking Assay of Conjugate 1 or Conjugate 5

### Conjugate 1:

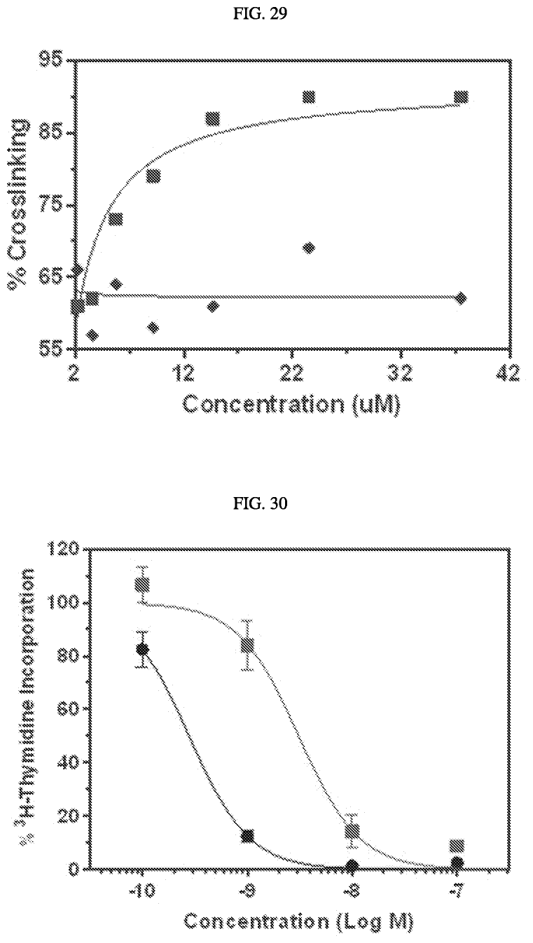

Calf thymus DNA (CT-DNA) was combined with increasing concentrations of Conjugate 1 (0.14 to 33.3 pM) or Conjugate 1+/− DTT. CT-DNA+Melphalan was used as a positive control and CT-DNA+DMSO was used as a negative control. These solutions were incubated at 37° C. for 2 hours. The solutions were then mixed with ethidium bromide and incubated for 2 hours at room temperature. Fluorescence (Ex: 535 nm, Em: 605 nm) from these samples was measured on the Fluoroskan II fluorimeter. Next, the samples were heated to 104° C. for 5 minutes, cooled on ice for 5 minutes, kept at RT for 15 minutes and fluorescence measured. % crosslinking of each sample was calculated using the fluorescence values from the positive and negative controls. Results are shown in FIG. 29.

### Conjugate 5:

Calf thymus DNA (CT-DNA) was combined with increasing concentrations of Conjugate 5 (1.1 to 75 pM) or Conjugate 5+/− DTT. These solutions were incubated at 37° C. for 2 hours. The solutions were then mixed with ethidium bromide and incubated for 2 hours at room temperature. Fluorescence (Ex: 535 nm, Em: 605 nm) from these samples was measured on the Fluoroskan II fluorimeter. Next, the samples were heated to 104° C. for 5 minutes, cooled on ice for 5 minutes, kept at RT for 15 minutes and fluorescence measured. % crosslinking of each sample was calculated using the fluorescence values from the positive and negative controls. Results are shown in FIG. 36.

### Example 27: In Vitro Analysis of Conjugate 1 in MDA-MB231 Cells

MDA-MB231 (human breast cancer) cells were seeded in 12-well Falcon plates and allowed to form nearly confluent monolayers overnight in FFRPMI/HIFCS. Designated wells received medium containing 100μM folic acid (nontoxic FR blocker) and were used to determine the targeting specificity. Each well then received increasing concentrations of Conjugate 1 (n=4). Cells were pulsed for 2 h at 37° C., rinsed with medium, and then chased in fresh medium up to 72 h. Spent medium was aspirated and replaced with medium containing [3H]thymidine. Following a 2 h incubation, cells were washed with PBS and then treated with 5% trichloroacetic acid. The trichloroacetic acid was aspirated and cells were solubilized in 0.25 N sodium hydroxide. Each solubilized sample were transferred to scintillation vials containing Ecolume scintillation cocktail and counted in a liquid scintillation counter. Final results were expressed as the percentage of [3H]thymidine incorporation relative to untreated controls and IC₅₀ were values calculated using GraphPad Prism software. The cell killing activity of Conjugate 1 was found to be concentration dependent with an IC₅₀ of 0.28 nM on MDA-MB-231 cells. The significant reduction in activity of Conjugate 1 in the presence of an excess of free folate indicates that the observed cytotoxic activity was folate receptor mediated. Results are shown in FIG. 30.

### Method 3: Antitumor Activity in Large KB Tumor Model.

Female Balb/c nu/nu mice were fed ad libitum with folate-deficient chow (Harlan diet #TD01013) for the duration of the experiment. KB tumor cells were inoculated subcutaneously at the right flank of each mouse. Mice were dosed after the tumors reached an average of 100 and 180 mm³ through the lateral tail vein under sterile conditions in a volume of 200 mL of phosphate-buffered saline (PBS).

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm.

### Method 4: Toxicity as Measured by Weight Loss.

The percentage weight change of the test animals was determined on selected days post-tumor inoculation (PTI), and during dosing. The results were graphed.

### Example 28: Conjugate 9 In Vivo Activity Against Tumors

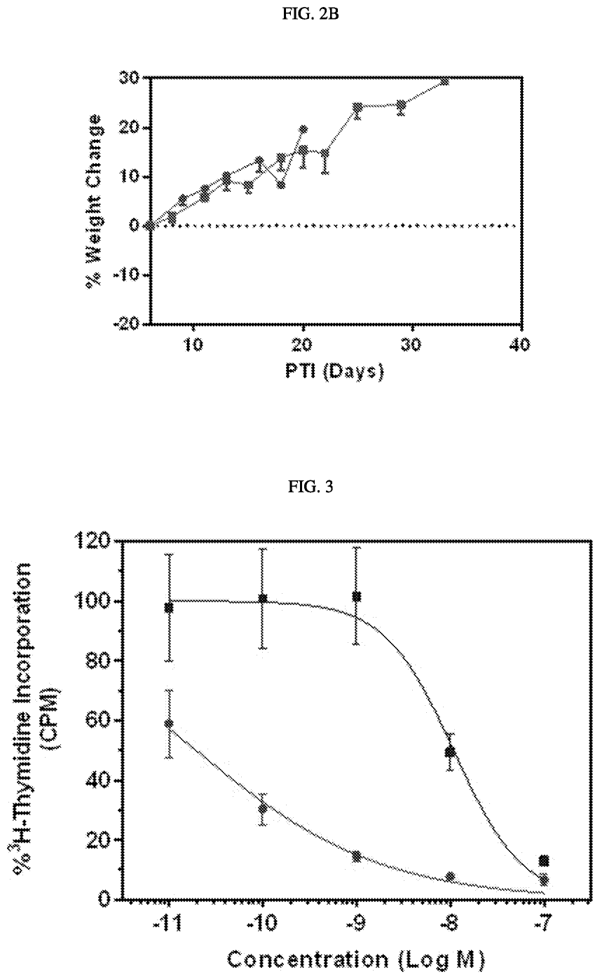

As shown in FIG. 2A, Conjugate 9 (▪) dosed at 1 μmol/kg SIW for two weeks decreased KB tumor size in test mice compared to untreated control (●). Treatment with 1 μmol/kg of Conjugate 9, once a week for two weeks produced minimal anti-tumor activity with 0% PRs. Change in weight is shown in FIG. 2B for mice dosed with Conjugate 9 SIW for two weeks (▪) compared to untreated control (●).

### Example 29: Conjugate 1 In Vivo Activity Against Tumors

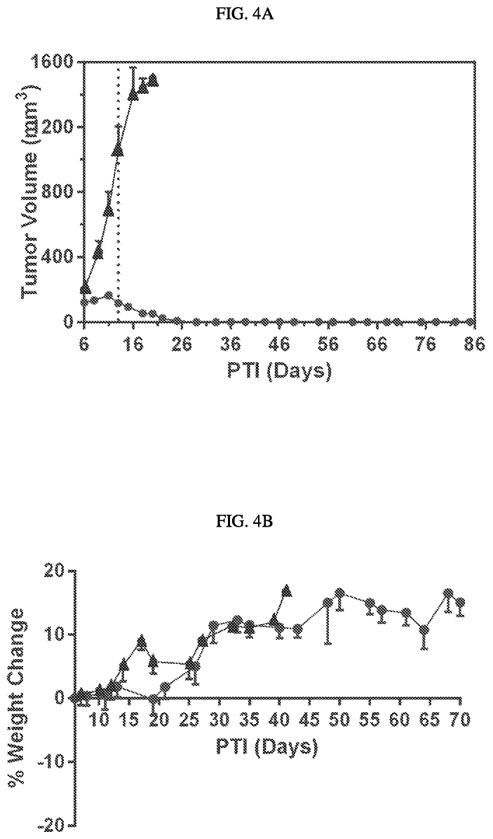

As shown in FIG. 4A, Conjugate 1 dosed at 0.5 μmol/kg SIW for two weeks (●) decreased KB tumor size in test mice compared to untreated control (▴). Treatment with 0.5 μmol/kg of Conjugate 1, once a week for two weeks produced maximal anti-tumor activity with 100% cures. Change in weight is shown in FIG. 4B for mice dosed with Conjugate 1 SIW for two weeks (●) compared to untreated control (▴).

### Example 30: Conjugate 2 In Vivo Activity Against Tumors

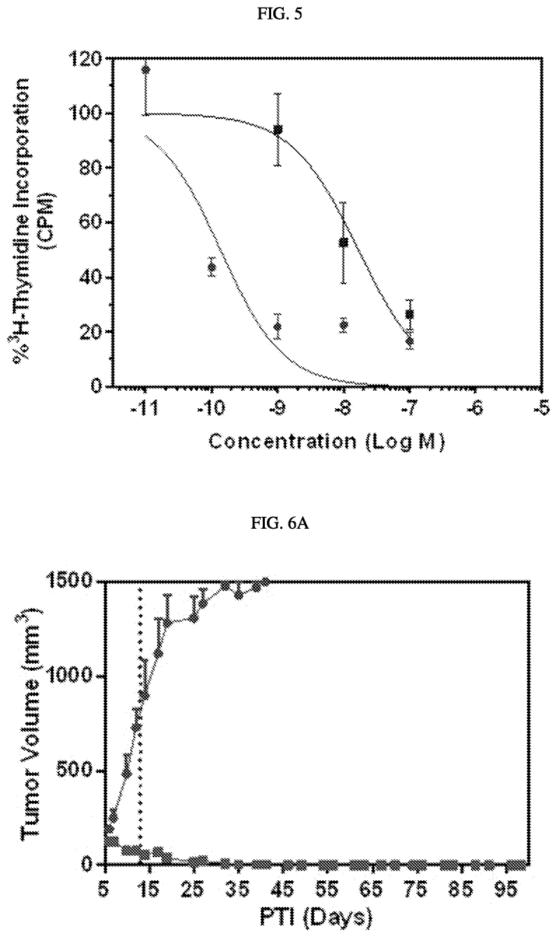

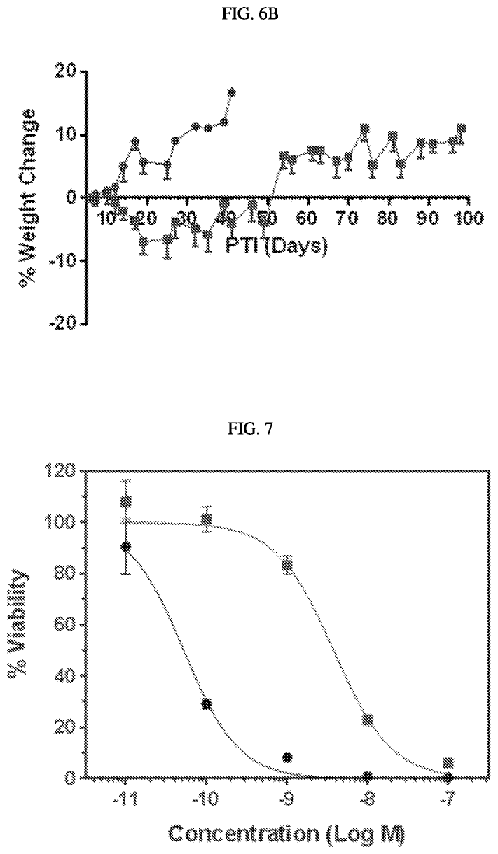

As shown in FIG. 6A, Conjugate 2 dosed at 0.5 μmol/kg SIW for two weeks (▪) decreased KB tumor size in test mice compared to untreated control (●). Conjugate 2 was highly active with 100% cures. Change in weight is shown in FIG. 6B for test mice dosed at 0.5 μmol/kg Conjugate 2 SIW for two weeks (▪) compared to untreated control (●).

### Example 31: Conjugate 5 In Vivo Activity Against Tumors

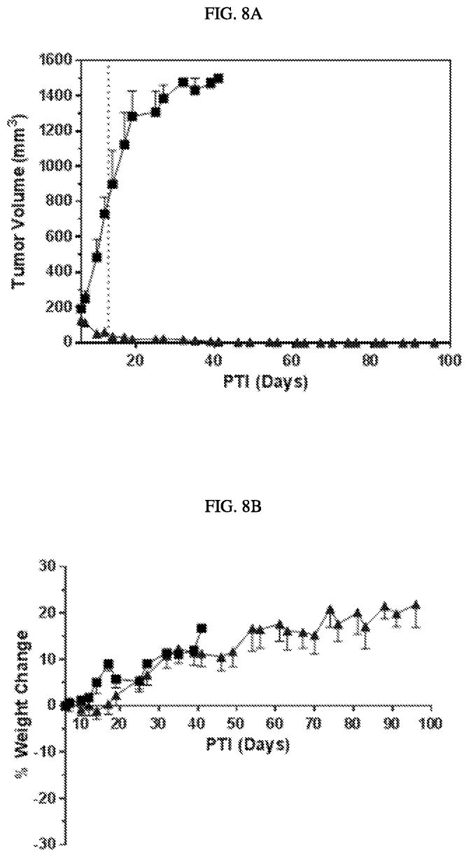

As shown in FIG. 8A, Conjugate 5 dosed at 0.5 μmol/kg SIW for two weeks (▴) decreased KB tumor size in test mice compared to untreated control (▪). Treatment with 0.5 μmol/kg of Conjugate 5, once a week for two weeks also produced maximal anti-tumor activity with 100% cures. Change in weight is shown in FIG. 8B for test mice dosed at 0.5 μmol/kg Conjugate 5 SIW for two weeks (▴) compared to untreated control (▪).

### Example 32: Conjugate 3 In Vivo Activity Against Tumors

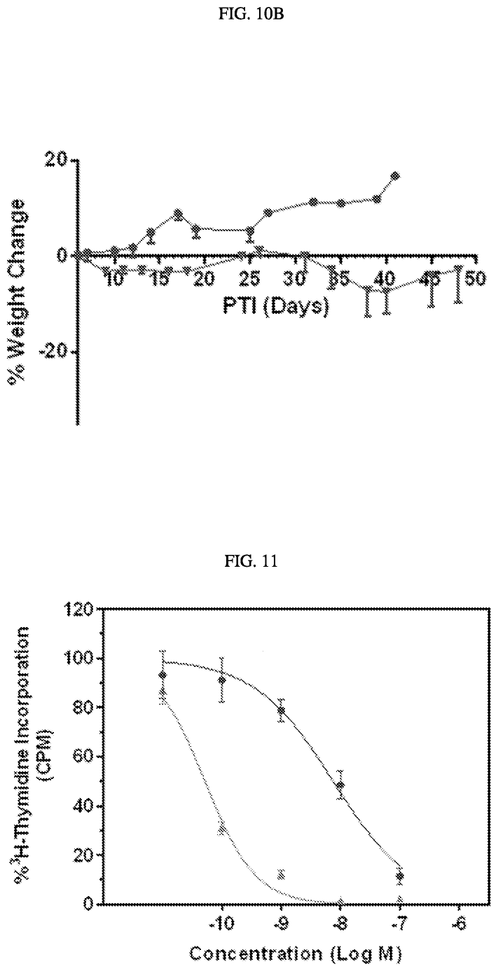

As shown in FIG. 10A, Conjugate 3 dosed at 0.5 μmol/kg SIW for two weeks (▾) decreased KB tumor size in test mice compared to untreated control (●). Treatment with 0.5 μmol/kg of Conjugate 3, once a week for two weeks produced 100% complete responses but mice had to be euthanized on day 48 due to toxicity. Change in weight is shown in FIG. 10B for test mice dosed at 0.5 μmol/kg Conjugate 3 SIW for two weeks (▾) compared to untreated control (●).

### Example 33: Conjugate 12 and Conjugate 4 In Vivo Activity Against Tumors

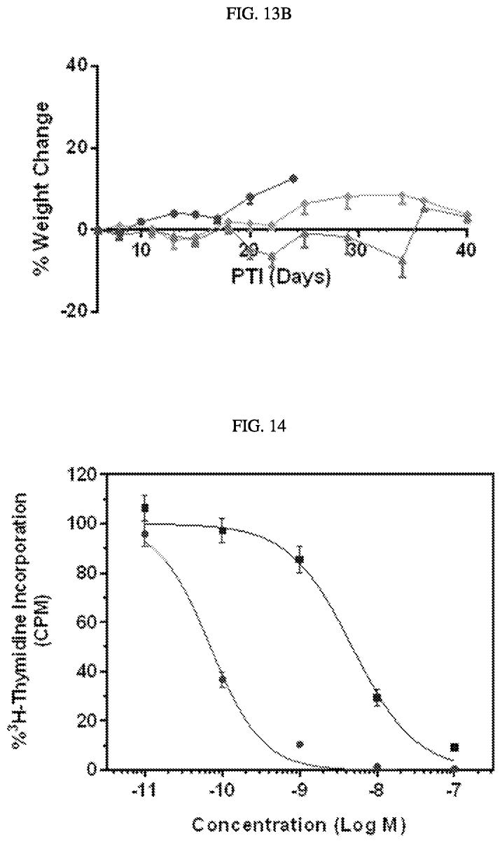

As shown in FIG. 13A, each Conjugate 12 dosed at 0.5 μmol/kg SIW for two weeks (▴) and Conjugate 4 dosed at 0.5 μmol/kg SIW for two weeks (♦) decreased KB tumor size in test mice compared to untreated control (●). Conjugate 4 was highly active with 100% cures at 0.5 μmol/kg, once a week for two weeks. At a similar dosing regimen, Conjugate 12 produced 100% PR's, but mice had to be euthanized on day 40 due to toxicity. Change in weight is shown in FIG. 13B for test mice dosed at 0.5 μmol/kg Conjugate 12 SIW for two weeks (▴) and test mice dosed at 0.5 μmol/kg Conjugate 4 SIW for two weeks (♦) compared to untreated control (●).

### Example 34: Conjugate 16 In Vivo Activity Against Tumors

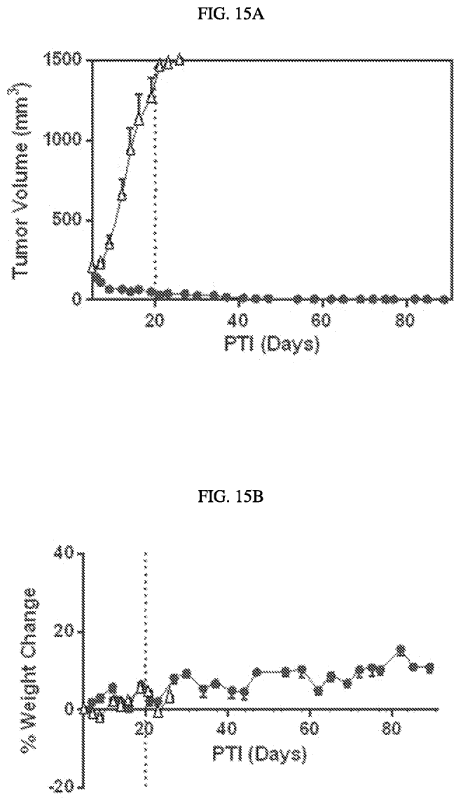

As shown in FIG. 15A, Conjugate 16 dosed at 0.5 μmol/kg SIW for two weeks (●) decreased KB tumor size in test mice compared to untreated control (▴). Treatment with 0.5 μmol/kg of Conjugate 16, once a week for two weeks produced 40% complete responses and 60% cures. Change in weight is shown in FIG. 15B for test mice dosed at 0.5 μmol/kg Conjugate 16 SIW for two weeks (●) compared to untreated control (▴).

### Example 35: Conjugate 6 In Vivo Activity Against Tumors

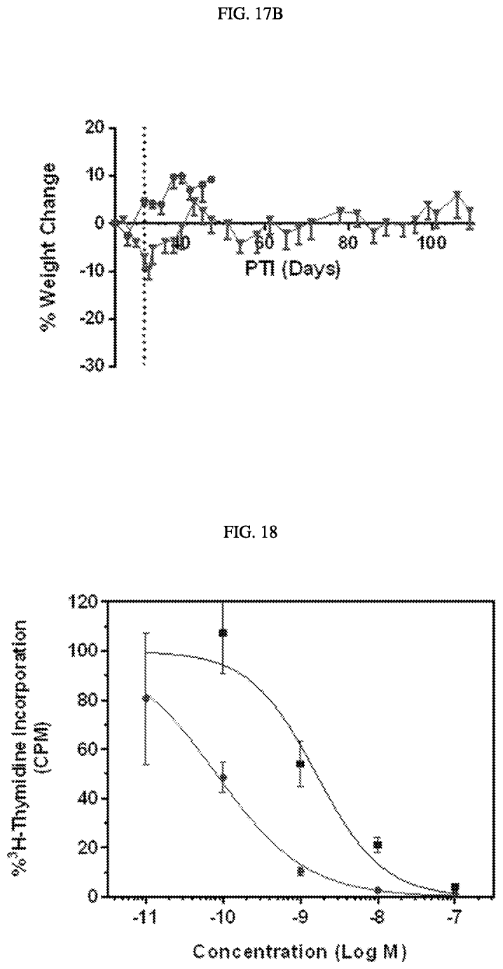

As shown in FIG. 17A, Conjugate 6 dosed at 0.5 μmol/kg SIW for two weeks (▾) decreased KB tumor size in test mice compared to untreated control (●). Treatment with 0.5 μmol/kg of Conjugate 6, once a week for two weeks produced 50% complete responses and 50% cures. Change in weight is shown in FIG. 17B for test mice dosed at 0.5 μmol/kg Conjugate 6 SIW for two weeks (▾) compared to untreated control (●).

### Example 36: Conjugate 15 In Vivo Activity Against Tumors

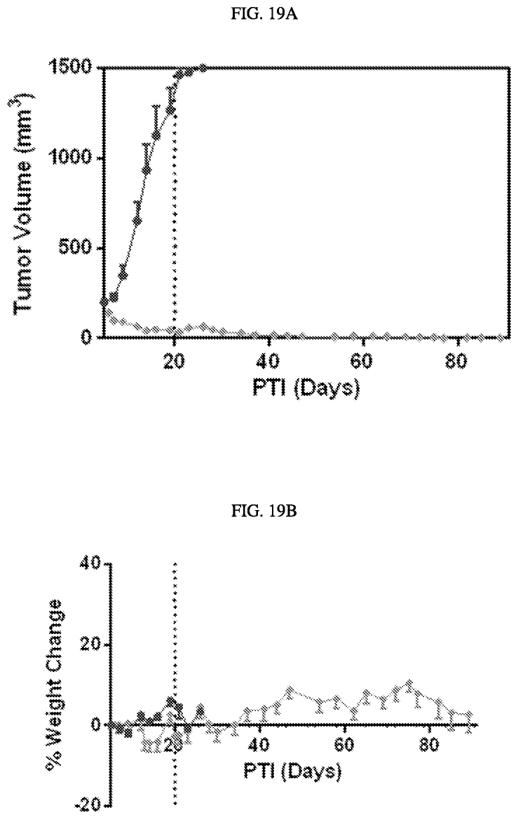

As shown in FIG. 19A, Conjugate 15 dosed at 0.5 μmol/kg SIW for two weeks (♦) decreased KB tumor size in test mice compared to untreated control (●). Conjugate 15 was highly active with 100% cures at just one 0.5 μmol/kg dose. Change in weight is shown in FIG. 19B for test mice dosed at 0.5 μmol/kg Conjugate 15 SIW for two weeks (♦) compared to untreated control (●).

### Example 37: Conjugate 7 In Vivo Activity Against Tumors

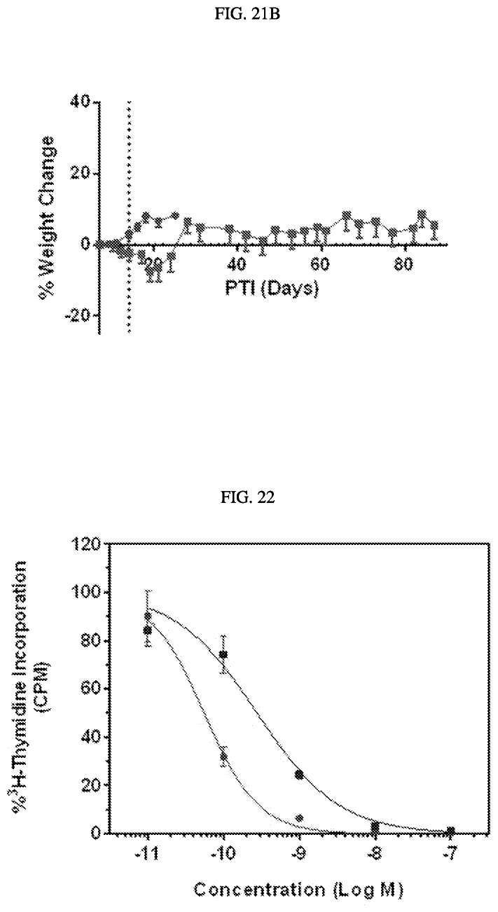

As shown in FIG. 21A, Conjugate 7 dosed at 0.5 μmol/kg SIW for two weeks (▪) decreased KB tumor size in test mice compared to untreated control (●). Conjugate 7 was highly active with 100% cures at 0.5 μmol/kg, once a week for two weeks. Change in weight is shown in FIG. 21B for test mice dosed at 0.5 μmol/kg Conjugate 7 SIW for two weeks (▪) compared to untreated control (●).

### Example 38: Conjugate 8 In Vivo Activity Against Tumors

As shown in FIG. 23A, Conjugate 8 dosed at 0.2 μmol/kg SIW for two weeks (▪) decreased KB tumor size in test mice compared to untreated control (●). Conjugate 8 was highly active with 100% cures at only 0.2 μmol/kg, once a week for two weeks. Change in weight is shown in FIG. 23B for test mice dosed at 0.2 μmol/kg Conjugate 8 SIW for two weeks (▪) compared to untreated control (●).

### Example 39: Conjugate 18, Conjugate 19, and Conjugate 20 In Vivo Activity Against Tumors

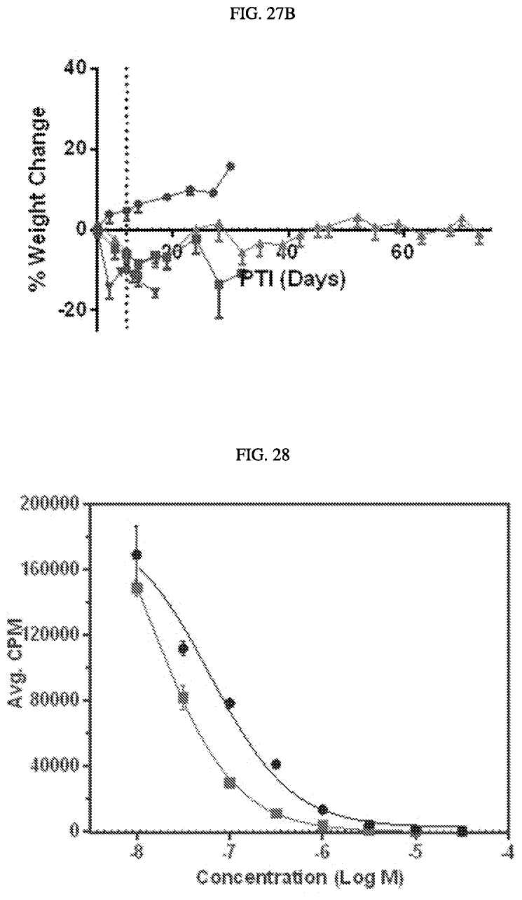

As shown in FIG. 27A, each of Conjugate 18 dosed at 0.5 μmol/kg SIW for two weeks (▪), Conjugate 19 dosed at 0.5 μmol/kg SIW for two weeks (▴), and Conjugate 20 dosed at 0.5 μmol/kg SIW for two weeks (▾) decreased KB tumor size in test mice compared to untreated control (●). Change in weight is shown in FIG. 27B for test mice dosed at 0.5 μmol/kg Conjugate 18 SIW for two weeks (▪), test mice dosed at 0.5 μmol/kg Conjugate 19 SIW for two weeks (▴), and test mice dosed at 0.5 μmol/kg Conjugate 20 SIW for two weeks (▾) compared to untreated control (●).

### Example 40: Conjugate 5 In Vivo Activity Against Paclitaxel Resistant Tumors

Mice were maintained and tumor volumes were measures according to Method 3.

KB-PR10 (paclitaxel resistant) tumor cells were inoculated subcutaneously at the right flank of each mouse. Mice were dosed through the lateral tail vein under sterile conditions in a volume of 200 μL of phosphate-buffered saline (PBS).

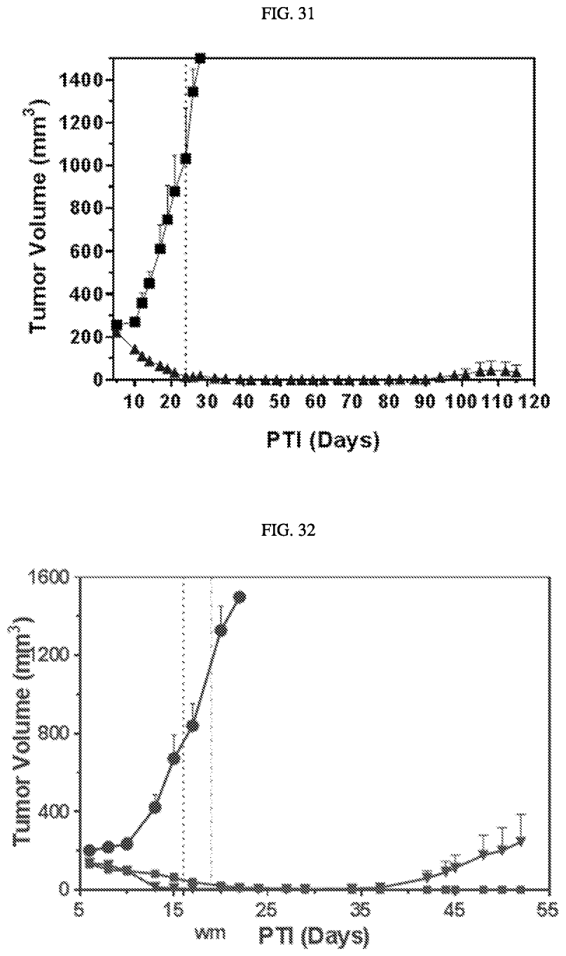

As shown in FIG. 31, Conjugate 5 dosed at 0.5 μmol/kg SIW for two weeks (▴) decreased paclitacel resistant KB tumor size in test mice compared to untreated control (▪).

### Example 41: Conjugate 5 In Vivo Activity Against Platinum Resistant Tumors

Mice were maintained and tumor volumes were measures according to Method 3.

KB-CR2000 (platin resistant) tumor cells were inoculated subcutaneously at the right flank of each mouse. Mice were dosed through the lateral tail vein under sterile conditions in a volume of 200 μL of phosphate-buffered saline (PBS).

As shown in FIG. 32, Conjugate 5 dosed at 0.5 μmol/kg SIW for two weeks (▪) and EC1456 dosed at 2.0 μmol/kg BIW for two weeks (▾) decreased paclitacel resistant KB tumor size in test mice compared to untreated control (●).

### Example 42: Conjugate 5 In Vivo Activity Against Triple Negative Breast Tumors

Mice were maintained and tumor volumes were measures according to Method 3.

Primary human TNBC model ST502 (2-4 mm in diameter) or primary human TNBC model ST738 (2-4 mm in diameter) were inoculated subcutaneously at the right flank of each mouse. Mice were randomized into experimental groups of 7 mice each and test articles were injected through the lateral tail vein under sterile conditions in a volume of 200 μL of phosphate-buffered saline (PBS).

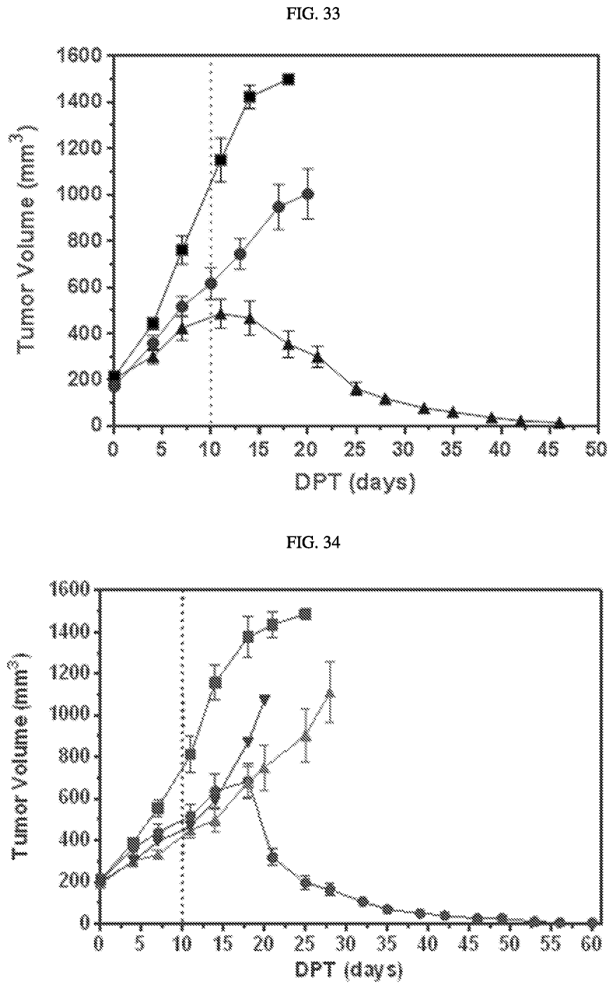

As shown in FIG. 33, Conjugate 5 dosed at 0.3 μmol/kg BIW for two weeks (▴) decreased TNBC PDX tumor size in test mice compared to untreated control (▪), whereas EC1456 dosed at 2.0 μmol/kg BIW for two weeks (●) did not decrease TNBC PDX tumor size.

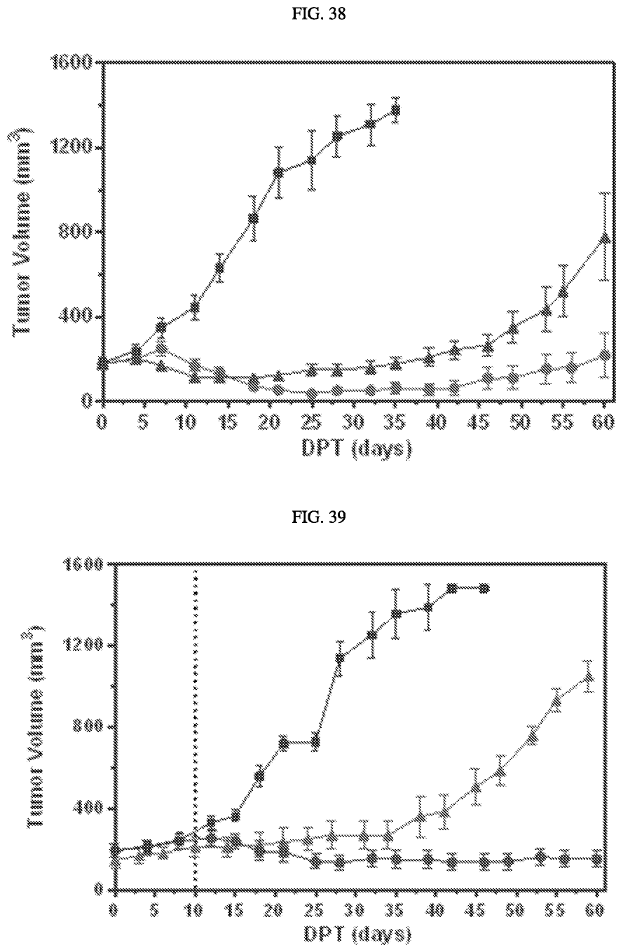

As shown in FIG. 38, Conjugate 5 dosed at 0.27 μmol/kg BIW for two weeks (▪) decreased TNBC PDX tumor size in test mice compared to untreated control (▪), whereas erubulin mesylate dosed at 1.0 μmol/kg SIW for two weeks (▴) did not decrease TNBC PDX tumor size.

### Example 43: Conjugate 5 In Vivo Activity Against Ovarian Tumors

Mice were maintained and tumor volumes were measures according to Method 3.

Primary human Ovarian model ST070 fragments (2-4 mm in diameter) were inoculated subcutaneously at the right flank of each mouse. Mice were randomized into experimental groups of 7 mice each and test articles were injected through the lateral tail vein under sterile conditions in a volume of 200 μL of phosphate-buffered saline (PBS).

As shown in FIG. 34, Conjugate 5 dosed at 0.5 μmol/kg SIW for two weeks (▪) decreased ovarian PDX tumor size in test mice compared to untreated control (▪), whereas EC1456 dosed at 4.0 μmol/kg SIW for two weeks (▴) and paclitaxel dosed at 15 mg/kg SIW for two weeks (▾) did not decrease ovarian PDX tumor size.

### Example 44: Conjugate 5 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

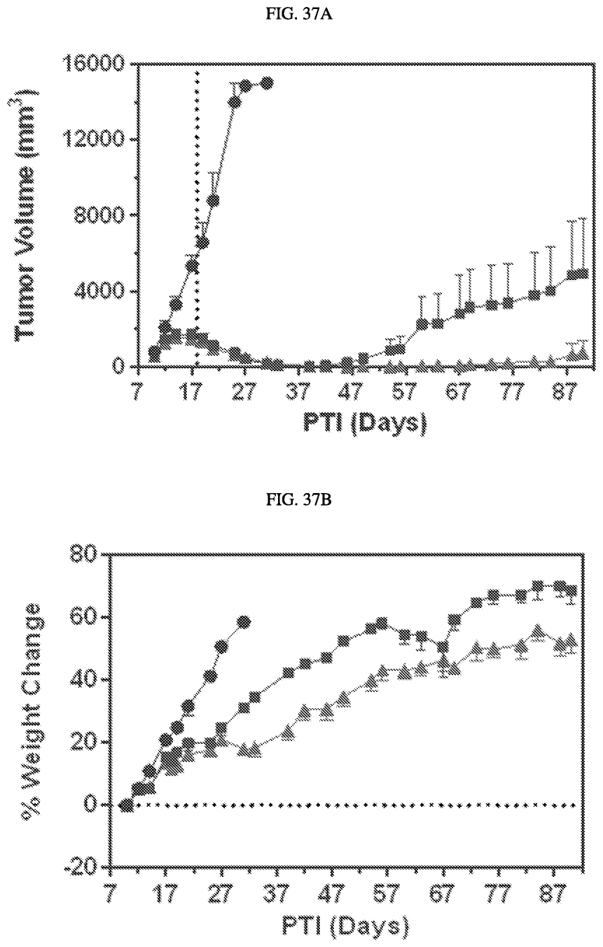

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 37A. Toxicity was measured as a function of animal weight gain or loss as shown in FIG. 37B.

### Example 45: Conjugate 5 In Vivo Activity Against Endometrial Tumors

Female Balb/c nu/nu mice were fed ad libitum with folate-deficient chow (Harlan diet #TD01013) for the duration of the experiment. Primary human Endometrial model ST040 fragments (2-4 mm in diameter) were inoculated subcutaneously at the right flank of each mouse. Mice were randomized into experimental groups of 7 mice each and test articles were injected through the lateral tail vein under sterile conditions in a volume of 200 μL of phosphate-buffered saline (PBS). These studies were performed at South Texas Accelerated Research Therapeutics, 4383 Medical Drive, San Antonio, Tex. 78229.

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm.

FIG. 39 shows that treatment with paclitaxel at 15 mg/kg SIW for two weeks produced 0% partial response subjects, while Compound 5 dosed at 0.27 μmol/kg BIW for two weeks produced 43% partial response subjects.

### Example 46: Conjugate 17 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

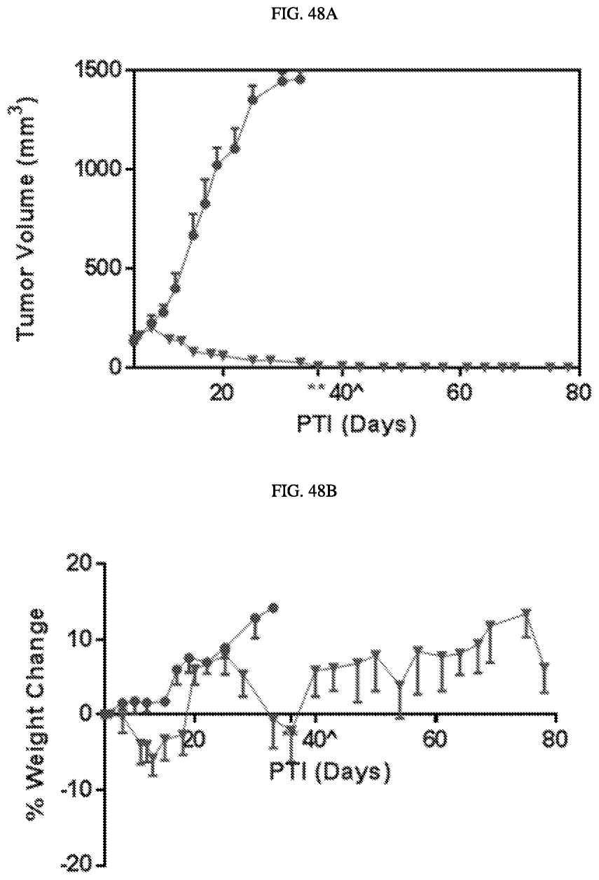

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 48A. Toxicity was measured as a function of animal weight gain or loss as shown in FIG. 48B.

### Example 47: Conjugate 22 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

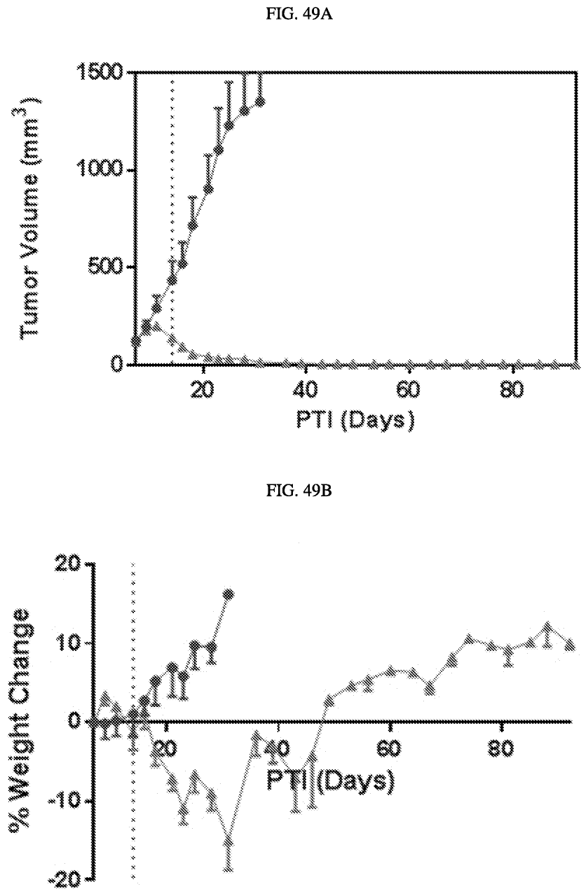

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 49A. Toxicity was measured as a function of animal weight gain or loss as shown in FIG. 49B.

### Example 48: Conjugate 24 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

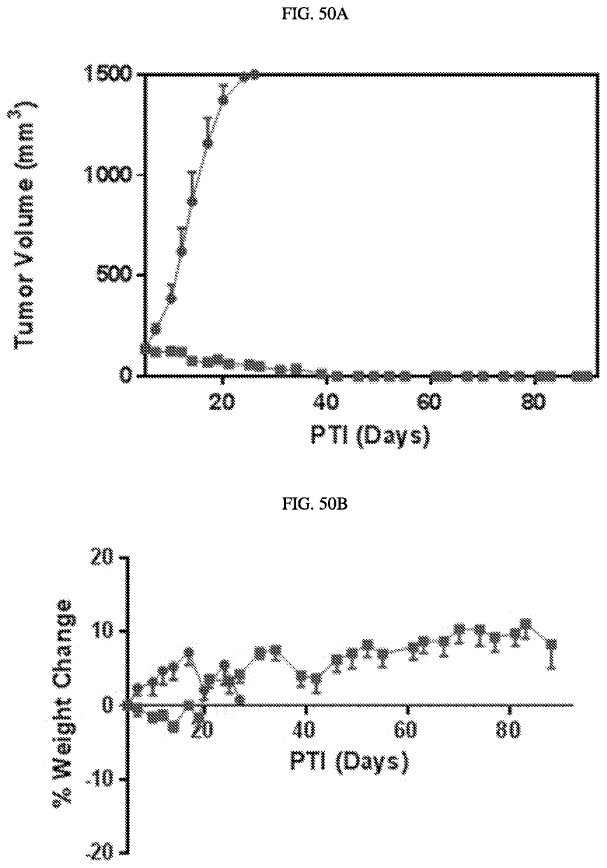

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 50A. Toxicity was measured as a function of animal weight gain or loss as shown in FIG. 50B.

### Example 49: Conjugate 26 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

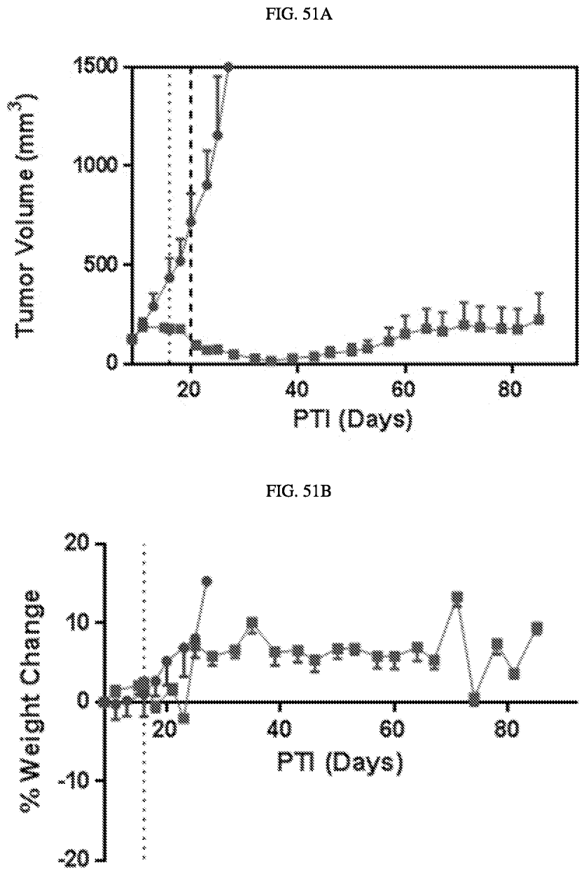

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 51A. Toxicity was measured as a function of animal weight gain or loss as shown in FIG. 51B.

### Example 50: Conjugate 27 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

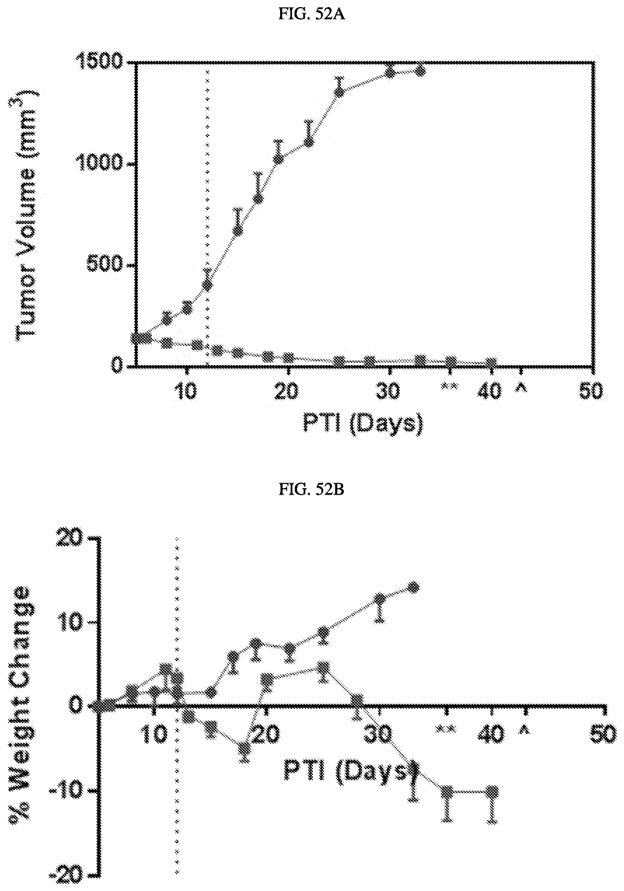

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 52A. Toxicity was measured as a function of animal weight gain or loss as shown in FIG. 52B.

### Example 51: Conjugate 28 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

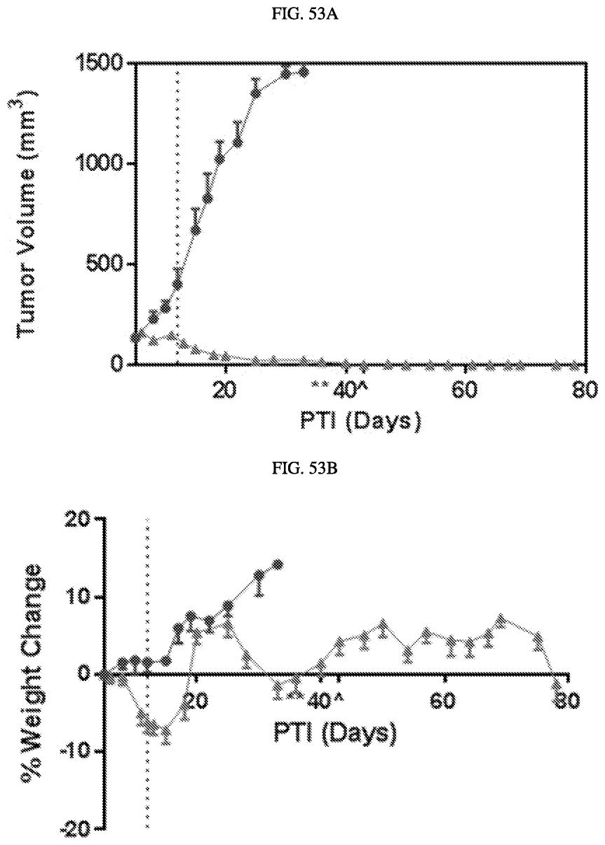

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 53A. Toxicity was measured as a function of animal weight gain or loss as shown in FIG. 53B.

### Example 52: Conjugate 30 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

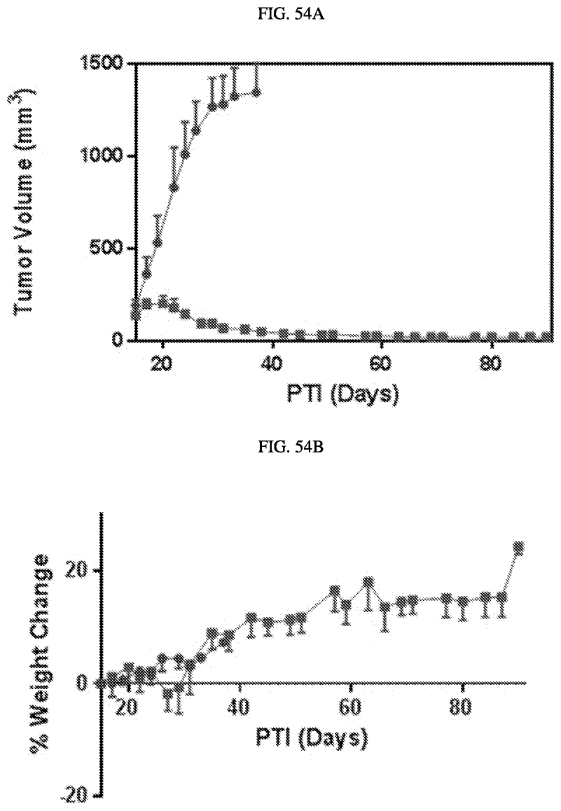

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 54A. Toxicity was measured as a function of animal weight gain or loss as shown in FIG. 54B.

### Example 53: Conjugate 32 In Vivo Activity in KB Rat Tumor Model

Female Balb/c nu/nu rats were fed ad libitum with folate-deficient chow (Harlan diet #TD01013) for the duration of the experiment. KB-tumor cells were inoculated subcutaneously at the right flank of each rat. Rats were dosed through the lateral tail vein under sterile conditions in a volume of 200 μL of phosphate-buffered saline (PBS).

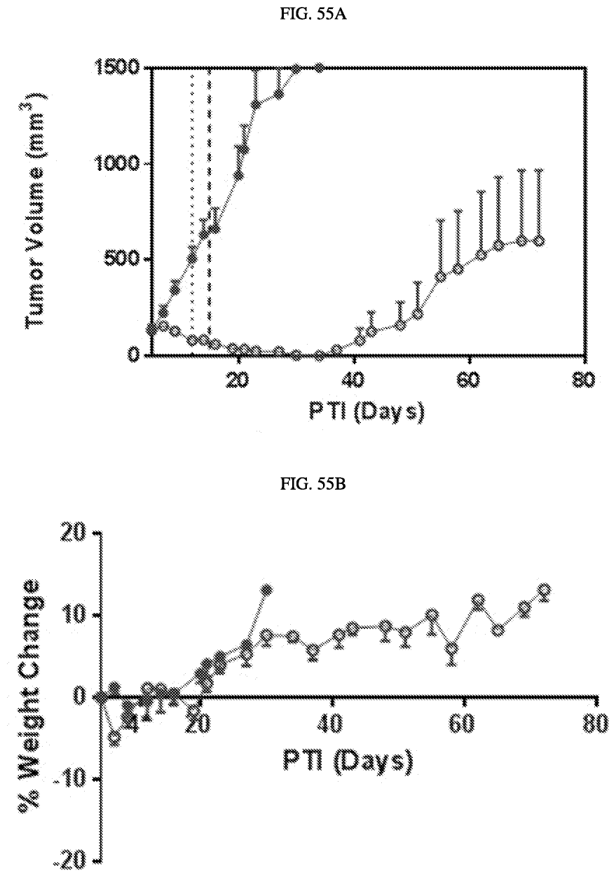

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 55A. Toxicity was measured as a function of animal weight gain or loss as shown in FIG. 55B.

### Example 54: In Vitro Studies of Conjugate 5 in Ovarian Cancer Cell Lines

### Reagents

The mouse and human folate binding protein 1 (FBP1, FOLR1) PicoKine™ ELSIA kits were purchased from Boster Biological Technology (Pleasanton, Calif.). Antibodies used for surface marker staining were purchased from eBioscience: PD-L¹ (clone MIH5; cat #25-5982), F4/80 (clone BM8; cat #12-4801), CD11b (clone M1/70; cat #48-0112), CD3ε (clone 145-2C11; cat #25-0031), CD4 (clone GK1.5; cat #46-0041), and CD8β (clone H3517.2; cat #11-0083).

### Cell Line

The FR-α expressing cell lines utilized to evaluate Conjugate 5 activity in in-vitro and ex-vivo studies were (1) ID8-Cl15, an ovarian carcinoma cell line transfected with the murine FR-α, and (2) IGROV1, a human ovarian carcinoma cell line that expresses the human FR-α. The FR-α negative ID8 parent (ID8p) cell line was used as controls in-vivo. ID8p and ID8-Cl15 cells were grown respectively in a folate-replete or folate-free RPMI1640 medium (Gibco BRL) (FFRPMI) containing 10% heat-inactivated fetal calf serum (HIFCS) and antibiotics, and maintained under a 5% CO₂ atmosphere using standard cell culture techniques. IGROV1 cells were grown in the same medium as ID8-C115 except that Corning® ultra-low attachment culture flasks (VWR, Cat. #89089-878) were used.

### ELISA Analysis

Following manufacturer's instructions, standards and test samples were added to 96-well ELISA plates that were pre-coated with a rat anti-FOLR1 monoclonal antibody. A biotinylated goat anti-FOLR1 polyclonal antibody was added and followed by a buffer wash. The avidin-biotin-peroxidase complex was then added and unbound conjugates were washed away. Subsequently, a horseradish peroxidase substrate, 3,3′,5,5′-Tetramethylbenzidine was added and catalyzed to produce a blue color product. The absorbance was read at 375 nm in a microplate reader at least two different time points.

### Clonogenic Assay

IGROV1 cells seeded in 6-well plates (1000 cells/well) were exposed for 2 h to Conjugate 5 at 1, 10, and 100 nM and followed by a 9-day chase in drug-free medium. Afterwards, the cells were washed with PBS and fixed for 5 min in a 3:1 methanol:acetic acid solution. The cells were then stained with 0.5% crystal violet/methanol solution for 15 min and washed with tap water. After a drying step, the colonies were photographed and counted using the ImageJ software.

### Flow Cytometry

The single-cell suspensions prepared from ascites were blocked in a FACS stain solution on ice for 20 minutes prior to staining for flow cytometry. The FACS stain solution consisted of 1% bovine serum albumin fraction V (Fisher scientific, cat # BP1600), 0.5 mg/mL human immunoglobulin (Equitech-Bio, cat # SLH66) and 0.05% sodium azide in PBS. For surface marker detections (PD-L¹, F4/80, CD11b, CD3, CD4, CD8), the tumor cells were stained in the FACS stain solution containing various fluorophore conjugated antibodies purchased from eBioscience at optimized concentrations (0.4-2.5 pg/mL). After 20 minutes on ice, the tumor cells were washed with PBS and re-suspended in PBS containing 3 pM propidium iodide for dead cell exclusion. Data was collected on the Gallios flow cytometer (Beckman Coulter) and analyzed using the Kaluza v 1.2 software (Beckman Coulter). Functional folate receptor was measured using a small molecule synthesized in house by coupling folic acid to Alexa Fluor 647.

### Results

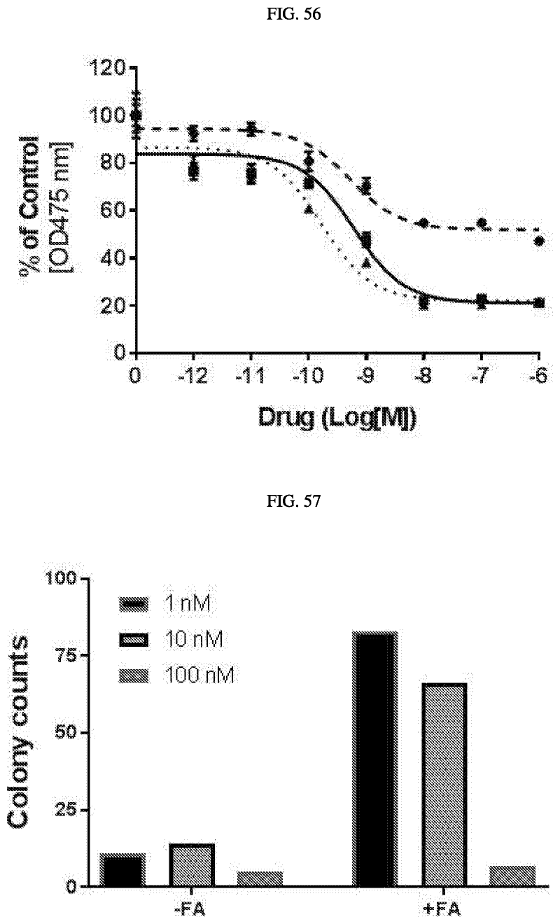

Conjugate 5 activity against ID8-Cl15 tumor cells was assessed using the XTT cell viability assay. The cells were exposed for 2 h to 10-fold serial dilutions of Conjugate 5 (up to 1 pM) and followed by a 72-120 h chase in drug-free medium. As determined by the XTT assay, Conjugate 5 showed a potent dose-dependent inhibition of cell proliferation with relative IC₅₀ values of ˜0.52 (72 h), 0.61 (96 h), and 0.17 (120 h) (FIG. 56). Importantly, the maximal cell kill was observed after 96-120 h chase, supporting the mechanism of action of this class of DNA-crosslinking compound.

Conjugate 5 activity against the slow-growing IGROV tumor cells was assessed using a clonogenic assay. After a 2 h exposure and 9-day chase (FIG. 57), Conjugate 5 demonstrated a potent activity at all concentrations (1-100 nM) tested. More importantly, Conjugate 5 anti-tumor activity was significantly reduced in the presence of excess amount of folic acid at both 1 and 10 nM concentrations.

### Example 55: In Vivo Studies of Conjugate 5 in Ovarian Tumor Model

### Mice

Female C57BL/6 (ID8p, ID8-Cl15) and nu/nu (IGROV1) mice were purchased from Envigo (Indianapolis, Ind.) and used when they reached 6-8 weeks of age. The mice were fed a folate-deficient diet (TestDiet, St. Louis, Mo.) on the day of arrival.

### Tumor Implantation

Mouse ascites tumors were generated by intra-peritoneal implantation of cultured cells at 5×10⁶ in C57BL/6 (ID8p, ID8-Cl15) and nu/nu (IGROV1) mice respectively.

Preparation of Single Cell Suspension from Tumor Bearing Mice

Ascites was collected via an I.P. injection of 5 mL of cold PBS containing 5 mM EDTA then removal of the intra-peritoneal fluid containing ascitic tumor cells. The cells were then collected by a 5 minute 400×g centrifugation, followed by an RBC lysis step, then a cold PBS wash and finally a 40 μm nylon filtration to remove tissue and large cellular aggregates.

Preparation of Acellular Ascitic Fluid from Ascites Bearing Mice

Upon euthanasia, total ascitic fluid was collected via an I.P. lavage of the intra-peritoneal fluid containing ascitic tumor cells. The acellular fraction of the ascitic fluid was obtained by a 5-minute 2200×g centrifugation and stored at −80° C. until future use.

### Conjugate 5 Plus Anti-CTLA-4 Combination Study

To test the effect of Conjugate 5 alone and in combination with anti-CTLA-4 antibody, ID8-C115 tumor cells (5×10⁶ cells per animal in 1% syngeneic mouse serum/folate-deficient RPMI1640 medium) were inoculated intraperitoneally 13 days post the date of arrival and start of the folate deficient diet. For comparison, EC1456 alone and in combination with the same regimen of anti-CTLA-4 antibody was also evaluated. Starting 7 days after tumor implant, mice were intravenously dosed BIW for a total of 6 doses with Conjugate 5 at 0.1 μmol/kg or EC1456 at 2 μmol/kg. The anti-CTLA-4 antibody dosing solution was prepared by diluting the stock solution (BioXcell, Clone UC10-4F10-11) to 1.25 mg/mL in PBS, pH 7.4. Anti-CTLA-4 (250 pg/dose) was i.p. administered BIW for a total of 5 doses starting 11 days after the tumor implant. In the Conjugate 5 plus anti-CTLA-4 and EC1456 plus anti-CTLA-4 combination groups, all compounds were dose- and schedule-matched with the single-agent dosing groups. Mice were weighed 3 times/week and assessed for any clinical sign of swollen bellies indicative of ascites formation and for the evidence of toxicity such as respiratory distress, mobility, weight loss, diarrhea, hunched posture, and failure to eat. Once the animals developed ascites, they were monitored daily and euthanized when ascites became severe (rounded and walking on tip toes). Healthy animals from the same cohort of mice were used as controls for normal weight gain.

### Results

### Quantification of FBP1 in Mouse Ascitic Fluids

The acellular ascitic fluid samples collected from ID8p, ID8-Cl15 and IGROV1 tumor-bearing mice at the time of euthanasia were assayed for soluble murine (ID8p, ID8-Cl15) and human (IGROV1) FBP1 levels. Murine FBP1 was detected in the ascitic fluid derived from mice intraperitoneally implanted with ID8-Cl15 tumor cells at 0.93-4.6 nM (Table 1). Similarly, human FBP1 was detected in the ascitic fluid derived from mice intraperitoneally implanted with IGROV1 tumor cells at 0.70-2.8 nM (Table 1). In contract, negligible amount of the murine FBP1 was found in the ascitic fluid derived from ID8p tumor-bearing mice (Table 1). This suggests that malignant ascites microenvironment renders FOLR1 shedding from cancer cells.

### Assessment of Functional FR in Mouse Models of Ovarian Cancer

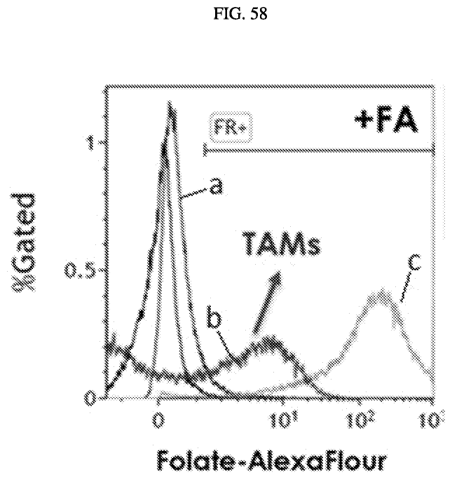

Functional FR levels were measured on the IGROV1 human ovarian cancer cells (FIG. 58; HLA+CD45−; label a) grown in the peritoneal cavity of nu/nu mice using a folate-fluorophore conjugate and compared to those on peritoneal macrophages (F480+CD11b+; label b) and freshly harvested IGROV1 cells from in vitro cultures (label c). There was only a small minority of mouse peritoneal ascites IGROV1 cells (˜6%) stained positive for FA-Alexa Fluor, suggesting a loss of FR-α either through shedding or down regulation or a combination of both. Shedding of FR-α by IGROV1 and ID8-Cl15 ascites cells likely occurred as soluble human and mouse FR-α (FBP1, FOLR1) were detected in acellular ascitic fluid by ELISA analysis (Table 1). The ID8p cell line derived ascitic fluid was used as a FR-negative control and indeed very little soluble murine FR-α was detected by ELISA (Table 1).

TABLE 1

| Tumor models | Mouse strain | Ascites fluid | Results |
|---|---|---|---|
| (Intraperitoneal) | (Female) | ELISA analysis | (nM) |
| IGROV1 | Nu/Nu | hFBP1 | 0.70-2.8 |
| ID8-Cl15 | C57BL/6 | mFBP1 | 0.93-4.6 |
| ID8p | C57BL/6 | mFBP1 | 0.066-0.092 |
| (FRα- control) | | | |

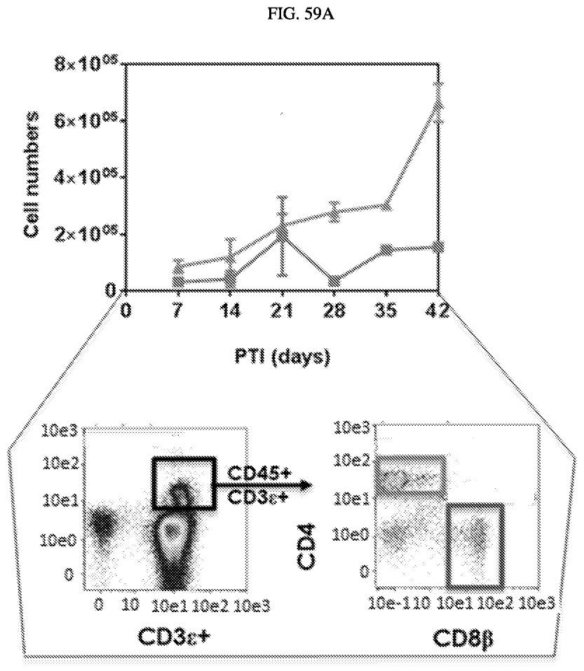

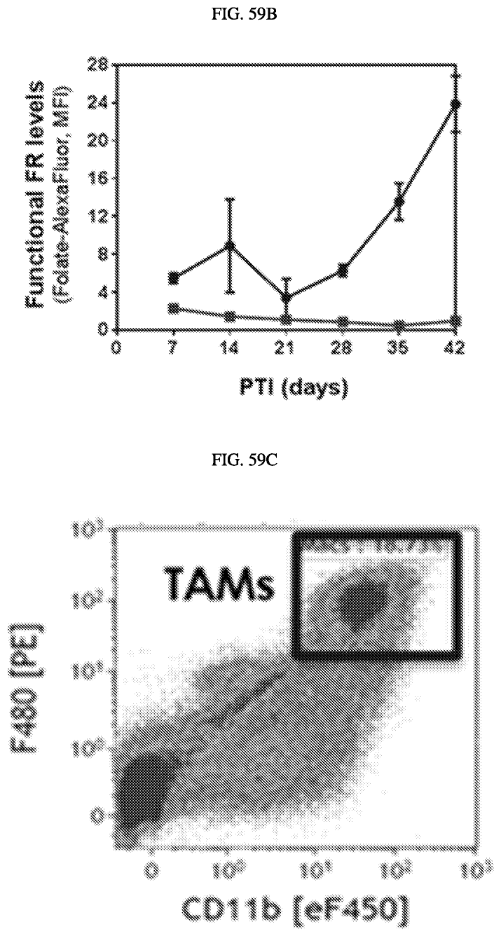

The presence of CD4+ and CD8+ T cells were also quantitated in total peritoneal cells of the immunocompetent C57BL6 mice at 7 day intervals post IP injection of the mouse ovarian cell line, ID8-CL15 (FIG. 59A). The CD45+CD3e+CD8+CD4− T cells (▪) slowly increased in number from day 7 to day 42 post implantation. The CD45+CD3e+CD4+CD8− T cells (▴) also increased in number from day 7 to day 35 with a more significant increase from day 35 to day 42 post implantation suggesting an immune response to the ovarian cancer cell had occurred. In addition, CD45− non bone-marrow derived ascites cells from ID8-CL15 implanted mice expressed very little functional FR (see FIG. 59B (▪)), whereas ascites macrophages (see FIGS. 59B (●) and 59C (insert box)) expressed a significant amount of a functional FR (likely, FRβ). These suggest that targeting of FR-β+ ovarian cancer stromal cells such as ascites macrophages could be alternative mechanism of action for compounds such as Conjugate 5.

Conjugate 5 In-Vivo Activity Alone and in Combination with Anti-CTLA-4

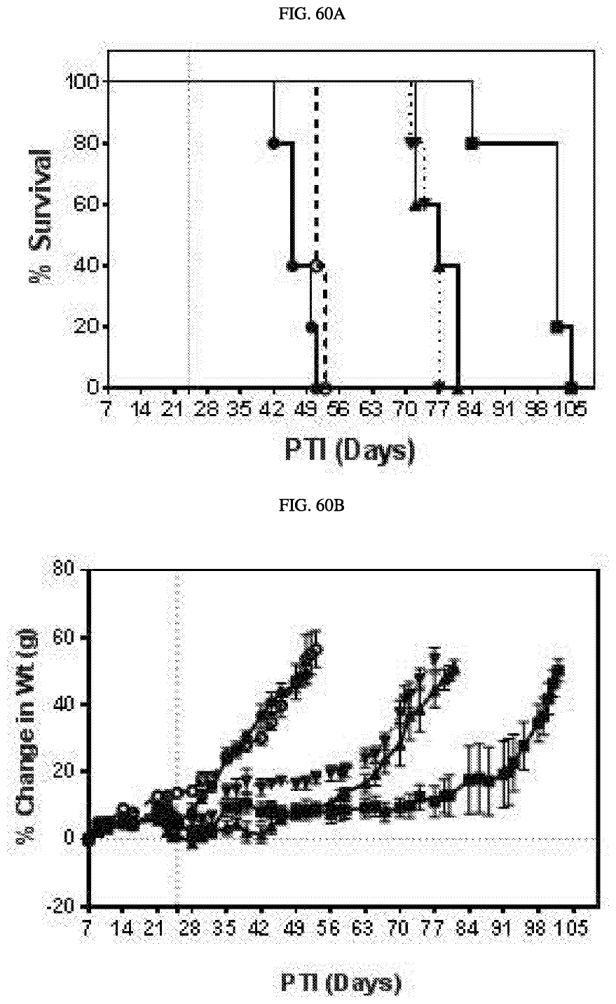

CTLA-4 (CD152) is a protein receptor that functions as an immune checkpoint to downregulate immune responses. CTLA-4 competes with CD28 for binding to B7 on antigen presentation cells in order to shut down T-cell activation. Recent studies showed that CTLA4 antagonists can enhance the activity of chemotherapy in certain tumor types. To examine the antitumor effect of Conjugate 5 alone and in combination anti-CTLA-4 antibody, we utilized syngeneic intraperitoneal ID8-C115 tumor bearing mice (FIG. 60A). For comparison, EC1456 was also tested as single agent or in combination with anti-CTLA-4 antibody. Here, untreated control mice had a median survival time of 46 days post tumor implant. Both EC1456 alone (i.v. 2 μmol/kg, BIW×6 doses) and Conjugate 5 alone (i.v. 0.1 μmol/kg, BIW×6 doses) produced significant anti-tumor effects in 5 animals each group, with ˜67% increase in the median survival time (˜77 days post tumor implant, P=0.0018, Log-Rank test). Anti-CTLA-4 antibody alone (i.p. 250 pg/dose, BIW×5 doses) displayed no significant anti-tumor effect in 5 animals, with ˜11% increase in the median survival time (˜51 days post tumor implant). EC1456 (i.v. 2 μmol/kg, BIW×6 doses) plus anti-CTLA-4 antibody (i.p. 250 pg/dose, BIW×5 doses) displayed no additional benefit in 5 animals with a median survival time of −81 days post tumor implant. On the other hand, Conjugate 5 (i.v. 0.1 μmol/kg, BIW×6 doses) plus anti-CTLA-4 antibody (i.p. 250 pg/dose, BIW×5 doses), displayed additional therapeutic benefit in 5 animals with a median survival time of −102 days post tumor implant.

1. A conjugate, or a pharmaceutically acceptable salt thereof, comprising a binding ligand (B), one or more linkers (L), at least one releasable group, a first drug (D¹) and a second drug (D²), wherein B is covalently attached to at least one L, at least one L is covalently attached to at least one of the first drug or the second drug, at least one of the first drug or the second drug is a PBD, and the one or more linkers comprises at least one releasable linker (L^{r}) of the formula 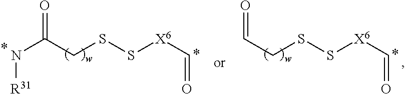
wherein
each R³ and R^{3′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each X⁶ is independently selected from the group consisting of —C₁-C₆ alkyl-, —C₆-C₁₀ aryl-(C₁-C₆ alkyl)-, —C₁-C₆ alkyl-O—, —C₆-C₁₀ aryl-(C₁-C₆ alkyl)-O—, —C₁-C₆ alkyl-NR^{31′}— and —C₆-C₁₀ aryl-(C₁-C₆ alkyl)-NR^{31′}—, wherein each hydrogen atom in —C₁-C₆ alkyl-, —C₆-C₁₀ aryl-(C₁-C₆alkyl)-, —C₁-C₆ alkyl-O—, —C₆-C₁₀ aryl-(C₁-C₆ alkyl)-O—, —C₁-C₆ alkyl-NR^{31′}— or —C₆-C₁₀ aryl-(C₁-C₆ alkyl)-NR^{31′} is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R³², R^{32′}, R³³, R^{33′}, R³⁴, R^{34′}, R³⁵ and R^{35′} are independently selected from the group consisting of H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl;
each w is independently an integer from 1 to 4; and
each * represents a covalent bond to the rest of the conjugate.

2. The conjugate of claim 1, wherein at least one of the first drug or the second drug is a PBD of the formula 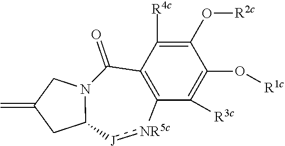
wherein
J is —C(O)—, —CR^{13c}═ or —(CR^{13c}R^{13c′})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R⁶, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c′}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R^{7c′}, —OS(O)NR^{7c}R^{7c′}, —OS(O)₂NR^{7c}R^{7c′}, —NR^{7c}R^{7c′}, —NR^{7c}C(O)R^{8c}, —NR^{7c}C(O)OR^{8c}, —NR^{7c}C(O)NR^{8c}R^{8c′}, —NR^{7c}S(O)R^{8c}, —NR^{7c}S(O)₂R^{8c}, —NR^{7c}S(O)NR^{8e}R^{8c′}, —NR^{7c}S(O)₂NR^{8c}R^{8c′}, —C(O)R^{7c}, —C(O)OR^{7c} or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c}, —OS(O)R^{9c}, —OS(O)₂R^{9c}, —SR^{9c}, —S(O)R^{9c}, —S(O)₂R^{9c}, —S(O)NR^{9c}R^{9c′}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{10c}, —NR^{9c}C(O)OR^{10c}, —NR^{9c}C(O)NR^{10c}R^{10c′}, —NR^{9c}S(O)R^{10c}, —NR^{9c}S(O)₂R^{10c}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c}.

3.-68. (canceled)

69. The conjugate of claim 1, or a pharmaceutically acceptable salt thereof, wherein B is of the formula 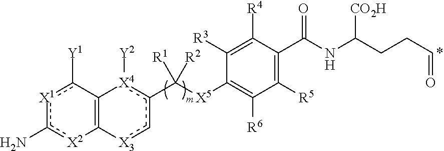
wherein
R¹ and R² in each instance are independently selected from the group consisting of H, halogen, C₁-C₆ alkyl, C₂-C₆ alkenyl, C₂_C₆ alkynyl, —OR⁷, —SR⁷ and —NR⁷R^{7′}, wherein each hydrogen atom in C₁-C₆ alkyl, C₂-C₆ alkenyl and C₂_C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, halogen, C₁-C₆ alkyl, C₂-C₆ alkenyl, C₂_C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂_C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, C₁-C₆ alkyl, C₂-C₆ alkenyl or C₂_C₆ alkynyl;
X¹ is —NR¹¹—, ═N—, —N═, —C(R¹¹)═ or ═C(R¹¹)—;
X² is —NR^{11′}— or ═N—;
X³ is —NR^{11′}—, —N═ or —C(R^{11′})═;
X⁴ is —N═ or —C═;
X⁵ is NR¹² or CR¹²R^{12′};
Y¹ is H, —OR¹³, —SR¹³ or —NR¹³R^{13′} when X¹ is —N═ or —C(R¹¹)═, or Y¹ is ═O when X¹ is —NR¹¹—, ═N— or ═C(R¹¹)—;
Y² is H, C₁-C₆ alkyl, C₂-C₆ alkenyl, —C(O)R¹⁴, —C(O)OR¹⁴, —C(O)NR¹⁴R^{14′} when X⁴ is —C═, or Y² is absent when X⁴ is —N═;
R¹¹, R^{11′}, R^{11″}, R¹², R^{12′}, R¹³, R^{13′}, R¹⁴ and R^{14′} are each independently selected from the group consisting of H, C₁-C₆ alkyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
wherein * represents a covalent bond to the rest of the conjugate.

70. The conjugate of claim 1, or a pharmaceutically acceptable salt thereof, wherein the one or more linkers (L) comprises at least one AA selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

71. The conjugate of claim 1, or a pharmaceutically acceptable salt thereof, wherein, when the one or more linkers (L) comprises a first spacer linker (L¹), the first spacer linker is of the formula 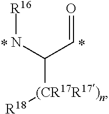
wherein
R¹⁶ is selected from the group consisting of H, C₁-C₆ alkyl, C₂-C₆ alkenyl, C₂_C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂_C₆ alkynyl is independently optionally substituted by halogen, C₁-C₆ alkyl, C₂-C₆ alkenyl, and C₂_C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}—S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂_C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26′})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃_C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂_C₉ alkynyl, C₃_C₆ cycloalkyl, —(CH₂)ₚ(sugar), —(CH₂)ₚ(OCH₂CH₂)-(sugar) and —(CH₂)ₚ(OCH₂CH₂CH₂)_{q}(sugar);
R²⁸ is a H, D, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5; and
each * represents a covalent bond to the rest of the conjugate.

72. The conjugate of claim 1, or a pharmaceutically acceptable salt thereof, wherein when the one or more linkers (L) comprises at least one second spacer linker (L²), each second spacer linker is independently selected from the group consisting of C₁-C₆ alkyl, —OC₁-C₆ alkyl, —SC₁-C₆ alkyl, 3- to 7-membered heterocycloalkyl, C₆-C₁₀ aryl, 5- to 7-membered heteroaryl, —NR³⁶(CR^{36′}R^{36″})ᵣ—S-(succinimid-1-yl)-, —(CR^{36′}R^{36″})ᵣC(O)NR³⁶—, —(CR³⁹R^{39′})ᵣC(O)—, —(CR³⁹R^{39′})ᵣOC(O)—, —S(CR³⁹R^{39′})ᵣOC(O)—, —C(O)(CR³⁹R^{39′})ᵣ, —C(O)O(CR³⁹R^{39′})ᵣ, —NR³⁹C(O)(CR^{39′}R^{39″})ᵣ, —NR³⁹C(O)(CR^{39′}R^{39″})ᵣS—, —(CH₂)ᵣNR³⁹—, —NR³⁹(CH₂)ᵣ—, —NR³⁹(CH₂)ᵣS—, —NR³⁹(CH₂)ᵣNR^{39′}—, —(OCR³⁹R^{39′}CR³⁹R^{39′})ᵣC(O)—, —(OCR³⁹R^{39′}CR³⁹R^{39′}CR³⁹R^{39′})ᵣC(O)—, —OC(O)(CR⁴⁴R^{44′})ₜ—, —C(O)(CR⁴⁴R^{44′})ₜ—, —NR⁴²CR⁴³R^{43′}CR⁴³R^{43′}(OCR⁴⁴R^{44′}CR⁴⁴R^{44′})ₜ—, —CR⁴³R^{43′}CR⁴³R^{43′}(OCR⁴⁴R^{44′}CR⁴⁴R^{44′})ₜNR⁴²—, —NR⁴²C₆-C₁₀ aryl(C₁-C₆ alkyl)OC(O)—, —C(O)CR⁴³R^{43′}CR⁴³R^{43′}(OCR⁴⁴R^{44′}CR⁴⁴R^{44′})ₜNR⁴²—, —NR⁴²CR⁴³R^{43′}CR⁴³R^{43′}(OCR⁴⁴R^{44′}CR⁴⁴R^{44′})ₜC(O)—, and —NR⁴²CR⁴³R^{43′}CR⁴³R^{43′}(CR⁴⁴═CR^{44′})ₜ—;
wherein
each R³⁶, R^{36′} and R^{36″} is independently selected from the group consisting of H, C₁-C₆ alkyl, C₂-C₆ alkenyl, C₂_C₆ alkynyl, C₃_C₆ cycloalkyl, —C(O)R³⁷, —C(O)OR³⁷ and —C(O)NR³⁷R^{37′} wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};
R³⁷, R^{37′}, R³⁸ and R^{38′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
each R³⁹ and R^{39′} is independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁰, —OC(O)R⁴⁰, —OC(O)NR⁴⁰R^{40′}, —OS(O)R⁴⁰, —OS(O)₂R⁴⁰, —SR⁴⁰, —S(O)R⁴⁰, —S(O)₂R⁴⁰, —S(O)NR⁴⁰R^{40″}, —S(O)₂NR⁴⁰R^{40′}, —OS(O)NR⁴⁰R^{40′}, —OS(O)₂NR⁴⁰R^{40′}, —NR⁴⁰R^{40′}, —NR⁴⁰C(O)R⁴¹, —NR⁴⁰C(O)OR⁴¹, —NR⁴⁰C(O)NR⁴¹R^{41′}, —NR⁴⁰S(O)R⁴¹, —NR⁴⁰S(O)₂R⁴¹, —NR⁴⁰S(O)NR⁴¹R^{41′}, —NR⁴⁰S(O)₂NR⁴¹R^{41′}, —C(O)R⁴⁰, —C(O)OR and —C(O)NR⁴⁰R^{40′};
R⁴⁰, R^{40′}′, R⁴¹ and R^{41′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, and 5- to 7-membered heteroaryl; and
R⁴² is selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁵, —OC(O)R⁴⁵, —OC(O)NR⁴⁵R^{45′}, —OS(O)R⁴⁵, —OS(O)₂R⁴⁵, —SR⁴⁵, —S(O)R⁴⁵, —S(O)₂R⁴⁵, —S(O)NR⁴⁵R^{45′}, —S(O)₂NR⁴⁵R^{45′}, —OS(O)NR⁴⁵R^{45′}, —OS(O)₂NR⁴⁵R^{45′}, —NR⁴⁵R^{45′}, —NR⁴⁵C(O)R⁴⁶, —NR⁴⁵C(O)OR⁴⁶, —NR⁴⁵C(O)NR⁴⁶R^{46′}, —NR⁴⁵S(O)R⁴⁶, —NR⁴⁵S(O)₂R⁴⁶, —NR⁴⁵S(O)NR⁴⁶R^{46′}, —NR⁴⁵S(O)₂NR⁴⁶R^{46′}, —C(O)R⁴⁵, —C(O)OR⁴⁵ or —C(O)NR⁴⁵R^{45′},
each R⁴³, R^{43′}, R⁴⁴ and R^{44′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁴⁷, —OC(O)R⁴⁷, —OC(O)NR⁴⁷R^{47′}, —OS(O)R⁴⁷, —OS(O)₂R⁴⁷, —SR⁴⁷, —S(O)R⁴⁷, —S(O)₂R⁴⁷, —S(O)NR⁴⁷R^{47′}, —S(O)₂NR⁴⁷R^{47′}, —OS(O)NR⁴⁷R^{47′}, —OS(O)₂NR⁴⁷R^{47′}, —NR⁴⁷R^{47′}, —NR⁴⁷C(O)R⁴⁸, —NR⁴⁷C(O)OR⁴⁸, —NR⁴⁷C(O)NR⁴⁸R^{48′}, —NR⁴⁷S(O)R⁴⁸, —NR⁴⁷S(O)₂R⁴⁸, —NR⁴⁷S(O)NR⁴⁸R^{48′}, —NR⁴⁷S(O)₂NR⁴⁸R^{48′}, —C(O)R⁴⁷, —C(O)OR⁴⁷ or —C(O)NR⁴⁷R^{47′};
R⁴⁵, R^{45′}, R⁴⁶, R^{46′}, R⁴⁷, R^{47′}, R⁴⁸ and R^{48′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
r in each instance is an integer from 1 to 40; and
t is in each instance is an integer from 1 to 40.

73. The conjugate of claim 1, or a pharmaceutically acceptable salt thereof, wherein when the one or more linkers (L) comprises at least one third spacer linker (L³), each third spacer linker is independently selected from the group consisting of C₁-C₁₀ alkyl, C₂-C₁₀ alkenyl, C₂_C₁₀ alkynyl, —(CR⁴⁹R^{49′})ᵤC(O)—, —CH₂CH₂(OCR⁴⁹R^{49′}CR⁴⁹R^{49′})ᵤ—, —CH₂CH₂CH₂(OCR⁴⁹R^{49′}CR⁴⁹R^{49′}CR⁴⁹R^{49′})ᵤ—, —CH₂CH₂(OCR⁴⁹R^{49′}CR⁴⁹R^{49′})ᵤC(O)— and —CH₂CH₂(OCR⁴⁹R^{49′}CR⁴⁹R^{49′}CR⁴⁹R^{49′})ᵤC(O)—,
wherein
each R⁴⁹ and R^{49′} is independently selected from the group consisting of H, C₁-C₆ alkyl, C₂-C₆ alkenyl, C₂_C₆ alkynyl and C₃_C₆ cycloalkyl, wherein each hydrogen atom in C₁-C₆ alkyl, C₂-C₆ alkenyl, C₂_C₆ alkynyl and C₃_C₆ cycloalkyl is independently optionally substituted by halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR⁵⁰, —OC(O)R⁵⁰, —OC(O)NR⁵⁰R^{50′}, —OS(O)R⁵⁰, —OS(O)₂R⁵⁰, —SR⁵⁰, —S(O)R⁵⁰, —S(O)₂R⁵⁰, —S(O)NR⁵⁰R^{50′}, —S(O)₂NR⁵⁰R^{50′}, —OS(O)NR⁵⁰R^{50′}, —OS(O)₂NR⁵⁰R^{50′}, —NR⁵⁰R^{50′}, —NR⁵⁰C(O)R⁵¹, —NR⁵⁰C(O)OR⁵¹, —NR⁵⁰C(O)NR⁵¹R^{51′}, —NR⁵⁰S(O)R⁵¹, —NR⁵⁰S(O)₂R⁵¹, —NR⁵⁰S(O)NR⁵¹R^{51′}, —NR⁵⁰S(O)₂NR⁵¹R^{51′}, —C(O)R⁵⁰, —C(O)OR⁵⁰ or —C(O)NR⁵⁰R^{50′};
R⁵⁰, R^{50′}, R⁵¹ and R^{51′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl; and
u is in each instance 0, 1, 2, 3, 4 or 5.

74. The conjugate of claim 1, or a pharmaceutically acceptable salt thereof, wherein the first drug is of the formula 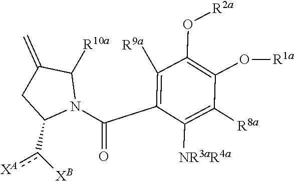
wherein
X^{A} is —OR^{6a}, ═N—OR^{5a} or —NR^{5a}R^{6a}—, provided that when the hash bond is a pi-bond, X^{A} is ═NR^{5a};
X^{B} is H or OR^{7a};
R^{1a}, R^{2a}, R^{3a} and R^{4a} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{1a}, —C(O)OR^{11a} and —C(O)NR^{11a}R^{11a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11a}, —OC(O)R^{11a}, —OC(O)NR^{11a}R^{11a′}, —OS(O)R^{11a}, —OS(O)₂R^{11a}, —SR^{11a}, —S(O)R^{11a}, —S(O)₂R^{11a}, —S(O)NR^{11a}R^{11a′}, —S(O)₂NR^{11a}R^{11a′}, —OS(O)NR^{11a}R^{11a′}, —OS(O)₂NR^{11a}R^{11a′}, —NR^{11a}R^{11a′}, —NR^{11a}C(O)R^{12a}, —NR^{11a}C(O)OR^{12a}, —NR^{11a}C(O)NR^{12a}R^{12a′}, —NR^{11a}S(O)R^{12a}, —NR^{11a}S(O)₂R^{12a}, —NR^{11a}S(O)NR^{12a}R^{12a}, —NR^{11a}S(O)₂NR^{12a}R^{12a}, —C(O)R^{11a}, —C(O)OR^{11a} or —C(O)NR^{11a}R^{11a}; or R^{1a} is a bond; or R^{4a} is a bond;
R^{5a}, R^{6a} and R^{7a} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{13a}, —C(O)OR^{13a} and —C(O)NR^{13a}R^{13a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{14a}, —OC(O)R^{14a}, —OC(O)NR^{14a}R^{14a′}, —OS(O)R^{14a}, —OS(O)₂R^{14a}, —SR^{14a}, —S(O)R^{14a}, —S(O)₂R^{14a}, —S(O)NR^{14a}R^{14a′}, —S(O)₂NR^{14a}R^{14a′}, —OS(O)NR^{14a}R^{14a′}, —OS(O)₂NR^{14a}R^{14a′}, —NR^{14a}R^{14a′}, —NR^{14a}C(O)R^{15a}, —NR^{14a}C(O)OR^{15a}, —NR^{14a}C(O)NR^{15a}R^{15a′}, —NR^{14a}S(O)R^{15a}, —NR^{14a}S(O)₂R^{15a}, —NR^{14a}S(O)NR^{15a}R^{15a′}, —NR^{14a}S(O)₂NR^{15a}R^{15a′}, —C(O)R^{14a}, —C(O)OR^{14a} or —C(O)NR^{14a}R^{14a′}; wherein R^{6a} and R^{7a} taken together with the atoms to which they are attached optionally combine to form a 3- to 7-membered heterocycloalkyl or a 3- to 7-membered heterocycloalkyl fused to a 6-membered aryl ring, or R^{5a} and R^{6a} taken together with the atoms to which they are attached optionally combine to form a 3- to 7-membered heterocycloalkyl or 5- to 7-membered heteroaryl, wherein each hydrogen atom in 3- to 7-membered heterocycloalkyl or 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{16a}, —OC(O)R^{16a}, —OC(O)NR^{16a}R^{16a′}, —OS(O)R^{16a}, —OS(O)₂R^{16a}, —SR^{16a}, —S(O)R^{16a}, —S(O)₂R^{16a}, —S(O)NR^{16a}R^{16a′}, —S(O)₂NR^{16a}R^{16a′}, —OS(O)NR^{16a}R^{16a′}, —OS(O)₂NR^{16a}R^{16a′}, —NR^{16a}R^{16a′}, —NR^{16a}C(O)R^{17a}, —NR^{16a}C(O)CH₂CH₂—, —NR^{16a}C(O)OR^{17a}, —NR^{16a}C(O)NR^{17a}R^{17a′}, —NR^{16a}S(O)R^{17a}, —NR^{16a}S(O)₂R^{17a}, —NR^{16a}S(O)NR^{17a}R^{17a′}, —NR^{16a}S(O)₂NR^{17a}R^{17a′}, —C(O)R^{16a}, —C(O)OR^{16a} or —C(O)NR^{16a}R^{16a′}, and wherein when R^{5a} and R^{6a} taken together with the atoms to which they are attached form a 5- to 7-membered heteroaryl, one hydrogen atom in 5- to 7-membered heteroaryl is optionally a bond, or when R^{6a} and R^{7a} taken together with the atoms to which they are attached optionally combine to form a 3- to 7-membered heterocycloalkyl fused to a 6-membered aryl, one hydrogen atom in the 6-membered aryl ring is optionally a bond; or R^{5a} is a bond;
R^{8a} and R^{9a} are each independently selected from the group consisting of H, halogen, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{18a}, —OC(O)R^{18a}, —OC(O)NR^{18a}R^{18a′}, —OS(O)R^{18a}, —OS(O)₂R^{18a}, —SR^{18a}, —S(O)R^{18a}, —S(O)₂R^{18a}, —S(O)NR^{18a}R^{18a′}, —S(O)₂NR^{18a}R^{18a′}, —OS(O)NR^{18a}R^{18a′}, —OS(O)₂NR^{18a}R^{18a′}, —NR^{18a}R^{18a′}, —NR^{18a}C(O)R^{19a}, —NR^{18a}C(O)OR^{19a}, —NR^{18a}C(O)NR^{19a}R^{19a′}, —NR^{18a}S(O)R^{19a}, —NR^{18a}S(O)₂R^{19a}, —NR^{18a}S(O)NR^{19a}R^{19a′}, —NR^{18a}S(O)₂NR^{19a}R^{19a′}, —C(O)R^{18a}, —C(O)OR^{18a} and —C(O)NR^{18a}R^{18a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{20a}, —OC(O)R^{20a}, —OC(O)NR^{20a}R^{20a′}, —OS(O)R^{20a}, —OS(O)₂R^{20a}, —SR^{20a}, —S(O)R^{20a}, —S(O)₂R^{20a}, —S(O)NR^{20a}R^{20a′}—S(O)₂NR^{20a}R^{20a′}, —OS(O)NR^{20a}R^{20a′}—OS(O)₂NR^{20a}R^{20a′}, —NR^{20a}R^{20a′}, —NR^{20a}C(O)R^{21a′}, —NR^{20a}C(O)OR^{21a}, —NR^{20a}C(O)NR^{21a}R^{21a′}, —NR^{20a}S(O)R^{21a}, —NR^{20a}S(O)₂R^{21a}, —NR^{20a}S(O)NR^{21a}R^{21a′}, —NR^{20a}S(O)₂NR^{21a}R^{21a′}—C(O)R^{2a}, —C(O)OR^{20a} or —C(O)NR^{20a}R^{20a′};
R^{10a} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{22a}, —OC(O)R^{22a}, —OC(O)NR^{22a}R^{22a′}, —OS(O)R^{22a}, —OS(O)₂R^{22a}, —SR^{22a}, —S(O)R^{22a}, —S(O)₂R^{22a}, —S(O)NR^{22a}R^{22a′}, —S(O)₂NR^{22a}R^{22a′}, —OS(O)NR^{22a}R^{22a′}, —OS(O)₂NR^{22a}R^{22a}, —NR^{22a}R^{22a′}, —NR^{22a}C(O)R^{23a}, —NR^{22a}C(O)OR^{23a}, —NR^{22a}C(O)NR^{23a}R^{23a′}, —NR^{22a}S(O)R^{23a}, —NR^{22a}R^{22a′}, —NR^{22a}S(O)NR^{23a}R^{23a′}, —NR^{22a}S(O)₂NR^{23a}R^{23a}, —C(O)R^{22a}, —C(O)OR^{23a} and —C(O)NR^{22a}R^{22a′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{24a}, —OC(O)R^{24a}, —OC(O)NR^{24a}R^{24a′}, —OS(O)R^{24a}, —OS(O)₂R^{24a}, —SR^{24a}, —S(O)R^{24a}, —S(O)₂R^{24a}, —S(O)NR^{24a}R^{24a′}—S(O)₂NR^{24a}R^{24a′}, —OS(O)NR^{24a}R^{24a′}, —OS(O)₂NR^{24a}R^{24a′}, —NR^{24a}R^{24a′}—NR^{24a}C(O)R^{25a}, —NR^{24a}C(O)OR^{25a}, —NR^{24a}C(O)NR^{25a}R^{25a′}, —NR^{24a}S(O)R^{25a}, —NR^{24a}S(O)₂R^{25a}, —NR^{24a}S(O)NR^{25a}R^{25a′}, —NR^{24a}S(O)₂NR^{25a}R^{25a′}, —C(O)R^{24a}, —C(O)OR^{24a} or —C(O)NR^{24a}R^{24a′}; and
each R^{11a}, R^{11a′}, R^{12a}, R^{12a′}, R^{13a}, R^{13a′}, R^{14a}, R^{14a′}, R^{15a}, R^{15a′}, R^{16a}, R^{16a′}, R^{17a}, R^{17a′}, R^{18a}, R^{18a′}, R^{19a}, R^{19a′}, R^{20a}, R^{20a′}, R^{21a}, R^{21a′}, R^{22a}, R^{22a′}, R^{23a}, R^{23a′}, R^{24a}, R^{24a′}, R^{25a} and R^{25a′} is independently selected from the group consisting of H, C₁-C₇ alkyl, C₂-C₇ alkenyl, C₂_C₇ alkynyl, C₃-C₁₃ cycloalkyl, 3- to 7-membered heterocycloalkyl, C₆-C₁₀ aryl, and 5- to 7-membered heteroaryl; and
provided that at least two of R, R^{4a}, R^{5a} are a bond; or when R^{5a} and R^{6a} taken together with the atoms to which they are attached optionally combine to form a 5- to 7-membered heteroaryl, one hydrogen atom in 5- to 7-membered heteroaryl is a bond and one of R^{1a} or R^{4a} is a bond.

75. The conjugate of claim 74, or a pharmaceutically acceptable salt thereof, wherein the first drug is covalently attached to the second drug by a third spacer linker (L³).

76. The conjugate of claim 75, or a pharmaceutically acceptable salt thereof, wherein the second drug is selected from the group consisting of 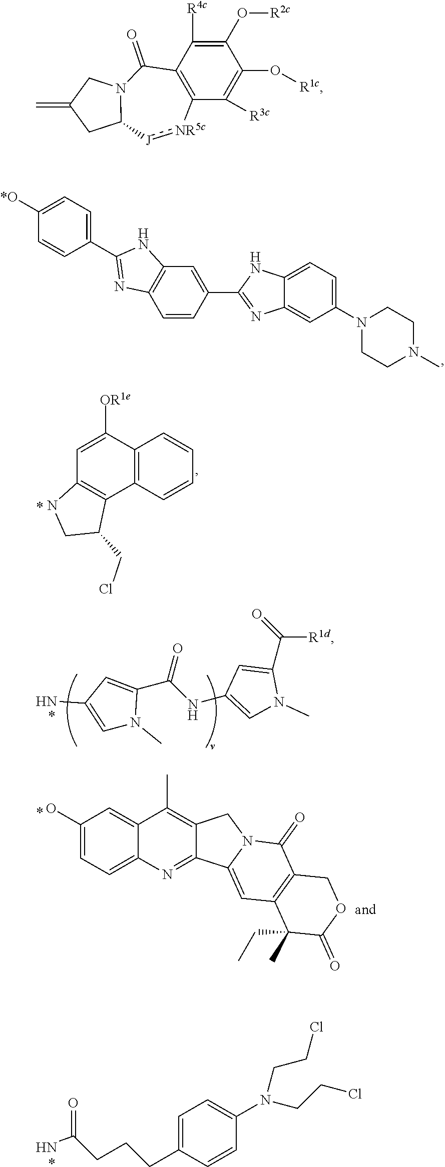
wherein
J is —C(O)—, —CR^{13c}═ or —(CR^{13c}R^{13c′})—;
R^{1c}, R^{2c} and R^{5c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —C(O)R^{6c}, —C(O)OR^{6c} and —C(O)NR^{6c}R⁶, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{7c}, —OC(O)R^{7c}, —OC(O)NR^{7c}R^{7c′}, —OS(O)R^{7c}, —OS(O)₂R^{7c}, —SR^{7c}, —S(O)R^{7c}, —S(O)₂R^{7c}, —S(O)₂OR^{7c}, —S(O)NR^{7c}R^{7c′}, —S(O)₂NR^{7c}R^{7c′}, —OS(O)NR^{7c}R^{7c′}, —OS(O)₂NR^{7c}R^{7c′}, —NR^{7c}R^{7c′}, —NR^{7c}C(O)R^{8c}, —NR^{7c}C(O)OR^{8c}, —NR^{7c}C(O)NR^{8c}R^{8c′}, —NR^{7c}S(O)R^{8c}, —NR^{7c}S(O)₂R^{8c}, —NR^{7c}S(O)NR^{8c}R^{8c′}, —NR^{7c}S(O)₂NR^{8c}R^{8c′}, —C(O)R^{7c}, —C(O)OR^{7c} or —C(O)NR^{7c}R^{7c′}; or when J is —CR^{13c}═, R^{5c} is absent; provided that at least one of R^{1c}, R^{2c} or R^{5c} is a covalent bond to the rest of the conjugate;
R^{3c} and R^{4c} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —CN, —NO₂, —NCO, —OR^{9c}, —OC(O)R^{9c}, —OC(O)NR^{9c}R^{9c}, —OS(O)R^{9c}, —OS(O)₂R^{9c}, —SR^{9c}, —S(O)R^{9c}, —S(O)₂R^{9c}, —S(O)NR^{9c}R^{9c}, —S(O)₂NR^{9c}R^{9c′}, —OS(O)NR^{9c}R^{9c′}, —OS(O)₂NR^{9c}R^{9c′}, —NR^{9c}R^{9c′}, —NR^{9c}C(O)R^{9c′}, —NR^{9c}C(O)OR^{10c}, —NR^{9c}C(O)NR^{10c}R^{10c′}, —NR^{9c}S(O)R^{10c}, —NR^{9c}S(O)₂R^{10c}, —NR^{9c}S(O)NR^{10c}R^{10c′}, —NR^{9c}S(O)₂NR^{10c}R^{10c′}, —C(O)R^{9c}, —C(O)OR^{9c} and —C(O)NR^{9c}R^{9c′}, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{11c}, —OC(O)R^{11c}, —OC(O)NR^{11c}R^{11c′}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} or —C(O)NR^{11c}R^{11c};
each R^{6c}, R^{6c′}, R^{7c}, R^{7c′}, R^{8c}, R^{8c′}, R^{9c}, R^{9c′}, R^{10c}, R^{10c′}, R^{11c}, R^{11c′}, R^{12c} and R^{12c′} is independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl and 5- to 7-membered heteroaryl;
R^{13c} and R^{13c′} are each independently selected from the group consisting of H, C₁-C₇ alkyl, C₂-C₇ alkenyl, C₂_C₇ alkynyl, C₃_C₆ cycloalkyl, 3- to 7-membered heterocycloalkyl, C₆-C₁₀ aryl, 5- to 7-membered heteroaryl, —OR^{11c}—OC(O)R^{1c}, —OC(O)NR^{1c}R^{1c}, —OS(O)R^{11c}, —OS(O)₂R^{11c}, —SR^{11c}, —S(O)R^{11c}, —S(O)₂R^{11c}, —S(O)NR^{11c}R^{11c′}, —S(O)₂NR^{11c}R^{11c′}, —OS(O)NR^{11c}R^{11c′}, —OS(O)₂NR^{11c}R^{11c′}, —NR^{11c}R^{11c′}, —NR^{11c}C(O)R^{12c}, —NR^{11c}C(O)OR^{12c}, —NR^{11c}C(O)NR^{12c}R^{12c′}, —NR^{11c}S(O)R^{12c}, —NR^{11c}S(O)₂R^{12c}, —NR^{11c}S(O)NR^{12c}R^{12c′}, —NR^{11c}S(O)₂NR^{12c}R^{12c′}, —C(O)R^{11c}, —C(O)OR^{11c} and —C(O)NR^{11c}R^{11c};
R^{1d} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{2d}, —SR^{2d} and —NR^{2d}R^{2d′},
R^{2d} and R^{2d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{3d}, —SR^{3d}, and —NR^{3d}R^{3d′};
R^{3d} and R^{3d′} are each independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
R^{1e} is selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂_C₆ alkynyl, C₃_C₆ cycloalkyl, 3- to 7-membered heterocycloalkyl, C₆-C₁₀ aryl, 5- to 7-membered heteroaryl, —OR^{2e}, —OC(O)R^{2e}, —OC(O)NR^{2e}R^{2e}, —OS(O)R^{2e}, —OS(O)₂R^{2e}, —SR^{2e}, —S(O)R^{2e}, —S(O)₂R^{2e}, —S(O)NR^{2e}R^{2e′}, —S(O)₂NR^{2e}R^{2e′}, —OS(O)NR^{2e}R^{2e′}, —OS(O)₂NR^{2e}R^{2e′}—NR^{2e}R^{2e′}, —NR^{2e}C(O)R^{3e}, —NR^{2e}C(O)OR^{3e}, —NR^{2e}C(O)NR^{3e}R^{3e′}, —NR^{2e}S(O)R^{3e}, —NR^{2e}S(O)₂R^{3e}, —NR^{2e}S(O)NR^{2e}R^{2e}, —NR^{2e}S(O)₂NR^{3e}R^{3e′}, —C(O)R^{2e}, —C(O)OR^{2e} or —C(O)NR^{2e}R^{2e};
each R^{2e}, R^{2e′}, R^{3e}, and R^{3e′} is independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl is optionally substituted by —OR^{4e}, —SR^{4e} or —NR^{4e}R^{4e′};
R^{4e} and R^{4e′} are independently selected from the group consisting of H, C₁-C₆ alkyl, C₂-C₆ alkenyl, C₂_C₆ alkynyl, C₃_C₆ cycloalkyl, 3- to 7-membered heterocycloalkyl, C₆-C₁₀ aryl and 5- to 7-membered heteroaryl;
v is 1, 2 or 3; and
each * represents a covalent bond to the rest of the conjugate.

77. The conjugate of claim 76, wherein the second drug is of the formula 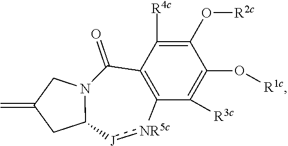
or a pharmaceutically acceptable salt thereof.

78. The conjugate of claim 1, having the formula B-(L¹)_{z1}-(AA)_{z2}-(L¹)_{z3}-(AA)_{z4}-(L¹)_{z5}-(AA)_{z6}-(L²)_{z7}-(L^{r})_{z8}-(L²)_{z9}-D-L³-D-(L²)_{y9}-L^{r})_{y8}-(L²)_{y7}-(AA)_{y6}-(L¹)_{y5}-(AA)_{y4}-(L¹)_{y3}-(AA)_{y2}-(L¹)_{y1}-X,
wherein
z1 is an integer from 0 to 2, z2 is an integer from 0 to 3, z3 is an integer from 0 to 2, z4 is an integer from 0 to 3, z5 is an integer from 0 to 2, z6 is an integer from 0 to 3, z7 is an integer from 0 to 8, z8 is 0 or 1, z9 is an integer from 0 to 8, y1 is an integer from 0 to 2, y2 is an integer from 0 to 3, y3 is an integer from 0 to 2, y4 is an integer from 0 to 3, y5 is an integer from 0 to 2, y6 is 0 or 1, y7 is an integer from 0 to 8, y8 is 0 or 1; y9 is an integer from 0 to 8;
each D is independently D¹ or D²;
X is H or B;
each B is independently a binding ligand;
each AA is independently an amino acid;
each L is independently a first spacer linker;
each L² is independently a second spacer linker;
each L³ is independently a third spacer linker; and
each L^{r} is independently a releasable linker;
or a pharmaceutically acceptable salt thereof.

79. The conjugate of claim 78, or a pharmaceutically acceptable salt thereof, wherein y is 0, y2 is 0, y3 is 0, y4 is 0, y5 is 0, y6 is 0, y7 is 0, y8 is 0, y9 is 0 and X is H.

80. The conjugate of claim 78, or a pharmaceutically acceptable salt thereof, wherein z1 is 0, z2 is 2, z3 is 0, z4 is 1, z5 is 0 and z6 is 1.

81. The conjugate of claim 78, or a pharmaceutically acceptable salt thereof, wherein z1 is 0, z2 is 2, z3 is 0, z4 is 2, z5 is 0 and z6 is 1.

82. The conjugate of claim 78, or a pharmaceutically acceptable salt thereof, wherein z1 is 1, z2 is 1, z3 is 1, z4 is 1, z5 is 1 and z6 is 1.

83. The conjugate of claim 78, or a pharmaceutically acceptable salt thereof, wherein z1 is 1, z2 is 1, z3 is 1, z4 is 1, z5 is 1 and z6 is 0.

84. The conjugate of claim 78, or a pharmaceutically acceptable salt thereof, wherein z1 is 0, z2 is 2, z3 is 0, z4 is 1, z5 is 0, z6 is 1, y1 is 0, y2 is 2, y3 is 0, y4 is 1, y5 is 0 and y6 is 1.

85. The conjugate of claim 78, or a pharmaceutically acceptable salt thereof, wherein z is 0, z2 is 2, z3 is 0, z4 is 2, z5 is 0, z6 is 1, y1 is 0, y2 is 2, y3 is 0, y4 is 2, y5 is 0 and y6 is 1.

86. The conjugate of claim 1, or a pharmaceutically acceptable salt thereof, comprising the formula 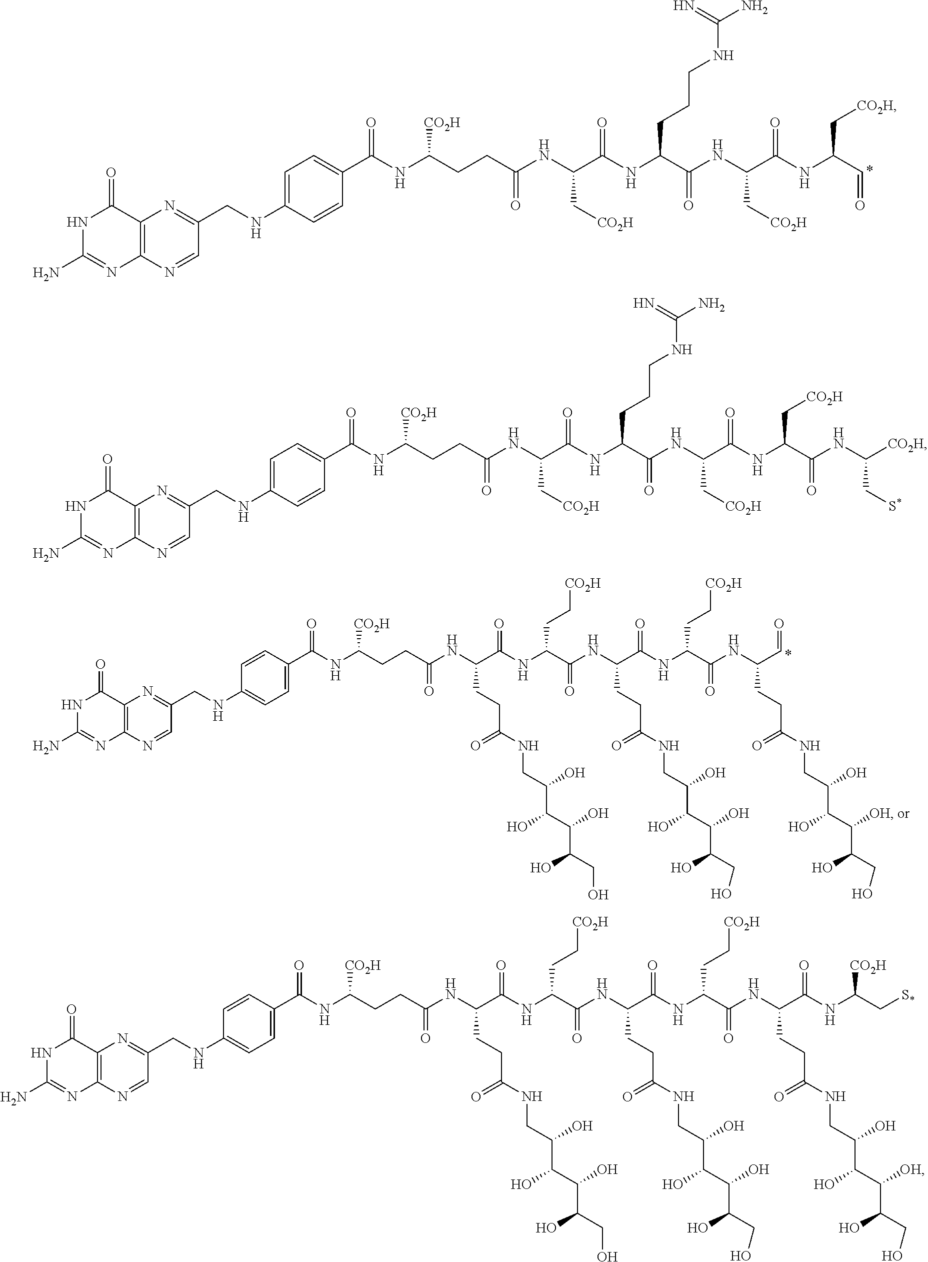
wherein * represents a covalent bond to the rest of the conjugate.

87. The conjugate of claim 77, or a pharmaceutically acceptable salt thereof, comprising the formula 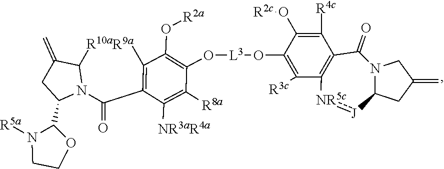
wherein R^{5a} is a covalent bond to the rest of the conjugate; 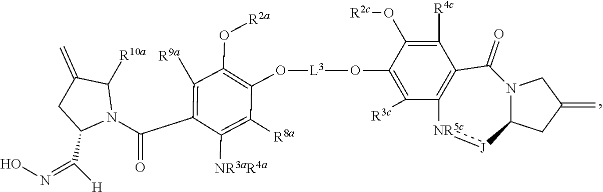
wherein R^{4a} is a covalent bond to the rest of the conjugate; 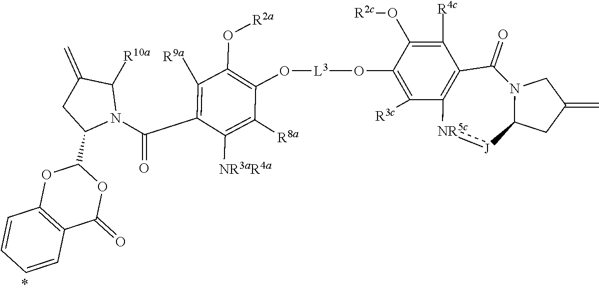
wherein * represents a covalent bond to the rest of the conjugate; or 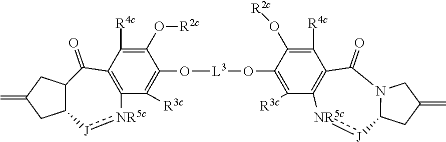
wherein at least one R^{5c} is a covalent bond to the rest of the conjugate.

88. A conjugate selected from the group consisting of [symbol]
or a pharmaceutically acceptable salt thereof.

89. A pharmaceutical composition comprising a therapeutically effective amount of a conjugate according to claim 1, or a pharmaceutically acceptable salt thereof, and optionally at least one pharmaceutically acceptable excipient.

90. A method of treating abnormal cell growth in a patient, comprising
a. administering to the patient a therapeutically effective amount of a conjugate, or a pharmaceutically acceptable salt thereof, or pharmaceutical composition, of claim 1.
